(19) United States
(12) Patent Application Publication
(10) Pub. No.: US 2025/0263662 A1
(43) Pub. Date: Aug. 21, 2025

(54) ENGINEERED BOVINE CELL LINES FOR SUSPENSION CULTURE

(71) Applicant: Upside Foods, Inc., Berkeley, CA (US)

(72) Inventors: Katherine GORA, San Leandro, CA (US); Alexi GORANOV, San Leandro, CA (US); Dakota KATZ, San Leandro, CA (US); Ann-Kathrin LAMB, San Leandro, CA (US); Swati DALVI, San Leandro, CA (US); Mauro SALINAS, San Leandro, CA (US); Andre LAZAR, San Leandro, CA (US)

(21) Appl. No.: 18/857,791

(22) PCT Filed: Apr. 19, 2023
(86) PCT No.: PCT/US2023/065934

§ 371 Date: Oct. 17, 2024

Related U.S. Application Data
(60) Provisional application No. 63/332,554, filed on Apr. 19, 2022.
(60) Provisional application No. 63/356,716, filed on Jun. 29, 2022.

(51) Int. Cl. C12N 5/077; A23J 3/14; A23L 13/00

(52) U.S. Cl. CPC C12N 5/0656; A23J 3/14; A23L 13/00; C12N 5/0658; C12N 2510/00

(57) ABSTRACT

The present disclosure provides genetically modified cell lines comprising i) reduced protein activity of one or more of PTEN, CASP3, CASP8, CDH1, ITGB1, RB1, IGFBP4, p21, p27, p16, and p18; ii) increased protein activity of SRC; iii) increased protein activity of TERT; and/or iv) reduced protein activity of p53. Further provided are cell-based meat products comprising an ingredient derived from such genetically modified cell lines. The disclosure relates to culturing myogenic cells in suspension state. Methods of generating such cell lines, cell culture and preparation of corresponding cell-based meat products are also provided herein.

### CROSS-REFERENCE TO RELATED APPLICATIONS

This application claims the benefit of U.S. Provisional Application No. 63/332,554, filed Apr. 19, 2022 and U.S. Provisional Application No. 63/356,716, filed Jun. 29, 2022, the content of each of which is herein incorporated by reference in its entirety.

### REFERENCE TO AN ELECTRONIC SEQUENCE LISTING

The content of the electronic sequence listing (ARFO_023_02WO_SeqList_ST26.xml; Size: 115,862 bytes; and Date of Creation: Apr. 17, 2023) is herein incorporated by reference in its entirety.

### BACKGROUND

Animal meat is one of the most versatile protein-rich food products available, and a common staple in Western diets. However, the practice of intensive animal agriculture poses an increasingly significant environmental problem due to its heavy use of water and land resources, as well as its high greenhouse gas emissions.

To meet the rising demand for animal meat, cell-based meat products such as those containing muscle cells cultured in vitro have become an attractive choice. Production of these cell-based meat products has been projected to require fewer resources, convert biomass with a higher caloric efficiency and result in reduced environmental impacts relative to conventional in vivo methods. Cellular biomass produced by such a cell culture method can also be used in medical applications such as organ or tissue transplant and grafts. Beyond the positive environmental impact, cultivated meat has the potential to reduce cruelty to animals and prevent the emergence of zoonotic diseases associated with animal agriculture. Cell-based meat can also improve food security by allowing nations to produce meat through cell culture without the need for arable land, especially for countries that have high local demand for meat but very limited land resources for agriculture.

Myogenic (i.e., “muscle forming”) cell lines have been used as fundamental models for understanding skeletal muscle biology since their derivation was first described nearly 50 years ago. Beyond basic research, myoblast cell lines have prospective industrial applications in biological robotics, bioartificial muscle constructs, pharmacological compounds screening, therapeutic correction of hereditary muscle disease, and the ex vivo production of edible biomass for dietary consumption. However, primary muscle cell isolation from donor tissues as a cell source for applications for commercial-scale production of animal biomass requires technical and material resources that currently preclude large scale manufacturing that is commercially viable. Furthermore, myogenic cell lines have been propagated as an adherent culture or a stationary culture which limits the scalability of the cultivation process. To date, the difficulty of growing the myogenic cells in suspension culture has limited both the scale of production and the ability to lower the unit economics of the cell culture process.

There is a need in the art for metazoan cell lines that can be cultured in suspension for the manufacture of cell-based meat products. The present disclosure provides such cell lines, compositions, processes for producing muscle cells using such cells lines, and more.

### SUMMARY

In one aspect, the disclosure provides clonal, metazoan cell lines comprising a genetic modification resulting in:
(A) reduced protein activity of at least one of i) Phosphatase and Tensin Homolog (PTEN), ii) Caspase 3 (CASP3), iii) Caspase 8 (CASP8), iv) Cadherin 1 (CDH1), v) Integrin beta-1 (ITGB1), vi) Retinoblastoma-associated protein (RB1), vii) Insulin-like growth factor-binding protein 4 (IGFBP4), and viii) a Cyclin-dependent kinase inhibitor; and/or
(B) increased protein activity of SRC tyrosine-protein kinase (SRC), compared to a control cell line without said genetic modification.

In some embodiments, the cell line comprises a genetic modification resulting in increased protein activity of Telomerase Reverse Transcriptase (TERT). In some embodiments, the cell line comprises a genetic modification resulting in reduced protein activity of Tumor Suppressor p53 (p53).

In some embodiments, the cell line comprises the genetic modifications resulting in: (i) reduced protein activity of CASP3; and (ii) reduced protein activity of ITGB1; compared to the control cell line without said genetic modifications.

In some embodiments, the cell line comprises the genetic modifications resulting in: (i) reduced protein activity of CASP3; and (ii) reduced protein activity of Cyclin-dependent kinase inhibitor 1B (p27); compared to the control cell line without said genetic modifications.

In some embodiments, the cell line comprises the genetic modifications resulting in: (i) reduced protein activity of CDH1; and (ii) reduced protein activity of Cyclin-dependent kinase inhibitor 1A (p21); compared to the control cell line without said genetic modifications.

In some embodiments, the cell line comprises the genetic modifications resulting in: (i) reduced protein activity of CDH1; and (ii) reduced protein activity of Cyclin-dependent kinase inhibitor 2A (p16); compared to the control cell line without said genetic modifications.

In some embodiments, the cell line comprises the genetic modification(s) resulting in: (iii) increased protein activity of TERT; and/or (iv) reduced protein activity of p53, compared to the control cell line without said genetic modification(s).

In some embodiments, the Cyclin-dependent kinase inhibitor comprises one or more of Cyclin-dependent kinase inhibitor 1A (p21), Cyclin-dependent kinase inhibitor 1B (p27), Cyclin-dependent kinase inhibitor 2A (p16), and Cyclin-dependent kinase 4 inhibitor C (p18).

In one aspect, the disclosure provides a clonal, metazoan cell line comprising: i) a genetic modification resulting in increased protein activity of TERT; and/or ii) a genetic modification resulting in reduced protein activity of p53, compared to a control cell line without said genetic modification. In some embodiments, the cell line comprises both i) and ii).

In some embodiments, the cell line comprises a genetic modification resulting in reduced protein activity of PTEN. In some embodiments, the cell line comprises a genetic modification resulting in reduced protein activity of CASP3. In some embodiments, the cell line comprises a genetic modification resulting in reduced protein activity of CASP8. In some embodiments, the cell line comprises a genetic modification resulting in reduced protein activity of CDH1. In some embodiments, the cell line comprises a genetic modification resulting in reduced protein activity of ITGB1. In some embodiments, the cell line comprises a genetic modification resulting in reduced protein activity of RB1. In some embodiments, the cell line comprises a genetic modification resulting in reduced protein activity of IGFBP4. In some embodiments, the cell line comprises a genetic modification resulting in reduced protein activity of the Cyclin-dependent kinase inhibitor. In some embodiments, the cell line comprises a genetic modification resulting in reduced protein activity of Cyclin-dependent kinase inhibitor 1A (p21). In some embodiments, the cell line comprises a genetic modification resulting in reduced protein activity of, Cyclin-dependent kinase inhibitor 1B (p27). In some embodiments, the cell line comprises a genetic modification resulting in reduced protein activity of Cyclin-dependent kinase inhibitor 2A (p16). In some embodiments, the cell line comprises a genetic modification resulting in reduced protein activity of Cyclin-dependent kinase 4 inhibitor C (p18). In some embodiments, the cell line comprises a genetic modification resulting in increased protein activity of SRC.

In some embodiments, the cell line is capable of sustained suspension culture. In some embodiments, the cell line is capable of reaching a viable cell density (VCD) of at least 0.4 million, at least 0.5 million, at least 0.6 million, at least 0.7 million, at least 0.8 million, at least 0.9 million, at least 1 million, at least 2 million, at least 5 million, at least 10 million, 15 million or 20 million cells per mL in the suspension culture.

In some embodiments, the cell line is in suspension culture. In some embodiments, the cell line is stable in the suspension culture for at least 20, 30, 40, 50, or 60 generations.

In some embodiments, the control cell line without at least one of said genetic modifications is unstable or less stable in a suspension culture. In some embodiments, the control cell line without at least one of said genetic modifications is not viable in a suspension culture.

In some embodiments, the cell line is capable of reaching at least 10%, 20%, 30%, 40%, 50%, 70%, 100%, 150%, 200%, 300%, or 400% higher maximum viable cell density (VCD) compared to the control cell line without at least one of said genetic modifications in the suspension culture. In some embodiments, the cell line exhibits at least 5%, 10%, 20%, 30%, or 40% higher cell viability than the control cell line without at least one of said genetic modifications at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture.

In some embodiments, the cell line exhibits a cell viability of at least 70%, at least 75%, at least 80%, at least 85%, or at least 90% at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture. In some embodiments, the cell line is capable of reaching maximum VCD at least 10%, 20%, 30%, 40%, 50%, 70%, 100%, 150%, 200%, 300%, or 400% faster than the control cell line without at least one of said genetic modifications in the suspension culture. In some embodiments, the cell line is capable of reaching a viable cell density of 0.4 million, 0.5 million, 0.6 million, 0.7 million, 0.8 million, 0.9 million, 1 million, 2 million, 5 million, 10 million, 15 million, 20 million, 25 million, 30 million, 35 million, 40 million, 45 million, or 50 million, cells per mL in the suspension culture at least 10%, 20%, 30%, 40%, 50%, 70%, 100%, 150%, 200%, 300%, or 400% faster than the control cell line without at least one of said genetic modifications. In some embodiments, the cell line exhibits a doubling time of less than 12 hours, 18 hours, 24 hours, 36 hours, or 48 hours when the viable cell density of the cell line is at 50% of its maximum VCD in the suspension culture.

In some embodiments, the cell line exhibits increased proliferation rate and/or decreased differentiation rate compared to the control cell line without at least one of said genetic modifications. In some embodiments, the proliferation rate of the cell line is at least 20% higher than the control cell line without at least one of said genetic modifications when both cell lines are at a viable cell density of about 50%, 60%, 70%, 80%, 90%, or 95% of their maximum VCD. In some embodiments, the differentiation rate of the cell line is at least 20% lower than the control cell line without at least one of said genetic modifications when both cell lines are at a viable cell density of about 50%, 60%, 70%, 80%, 90%, or 95% of their maximum VCD.

In some embodiments, cells of the suspension culture of the cell line comprises less than 50%, 40%, 30%, 20%, 10%, 5%, 3%, 2%, or 1% of cells in the form of cell aggregates having a diameter of greater than 100, 200, 300 or 400 microns. In some embodiments, the cell aggregates have a diameter of greater than 200 microns.

In some embodiments, cells of the suspension culture of the cell line comprises at least 50%, 60%, 70%, 80%, or 90% of cells in the form of single cells. In some embodiments, the single cells have an average diameter of less than 30, 25, 20, 15, 10, or 5 microns. In some embodiments, the single cells have an average circularity of greater than 0.5, 0.55, 0.6, 0.65, 0.7, 0.75, 0.8, 0.85, 0.9 or 0.95.

In some embodiments, the cell line exhibits increased anoikis resistance compared to the control cell line without at least one of said genetic modifications. In some embodiments, the percentage of apoptotic cells of the cell line is at least 10%, 20%, 30%, 40%, 50%, 60%, 70%, 80%, 90%, 95%, 97%, or 99% lower than the control cell line without at least one of said genetic modifications when both cell lines are at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture. In some embodiments, cells in the suspension culture of the cell line comprise less than 50%, 40%, 30%, 20%, 10%, 5%, 3%, 2%, or 1% of apoptotic cells. In some embodiments, the apoptotic cells are characterized using a biomarker selected from the group consisting of cytochrome C, caspase-3/7, DNA fragmentation and phosphatidylserine.

In some embodiments, less than 30%, 20%, 10%, 5%, 3%, 2% or 1% of cells in the suspension culture of the cell line are adherent cells. In some embodiments, expression level of E-cadherin in the cell line is decreased by at least 30%, at least 50%, at least 70%, or at least 90%, compared to the control cell line without at least one of said genetic modifications. In some embodiments, expression level of N-cadherin in the cell line is increased by at least 20%, at least 50%, at least 100%, or at least 2-fold, compared to the control cell line without at least one of said genetic modifications.

In some embodiments, expression level of vimentin in the cell line is increased by at least 20%, at least 50%, at least 100%, or at least 2-fold, compared to the control cell line without at least one of said genetic modifications.

In some embodiments, the cell line is a myoblast cell line. In some embodiments, the cell line is a fibroblast cell line. In some embodiments, the cell line is a preadipocyte cell line. In some embodiments, the cell line lineage is skeletal muscle, subcutaneous adipose tissue, or connective tissue. In some embodiments, the cell line lineage is skeletal muscle.

In some embodiments, the cell line is an immortalized cell line.

In some embodiments, the cell line species identity is selected from the group consisting of Bos Taurus, Sus scrofa, Ovis aries, Capra hircus, Oryctolagus cuniculus, Gallus gallus, Anas platyrhynchos, and Meleagris gallopavo. In some embodiments, the cell line species identity is Bos taurus, Bos indicus, or a hybrid thereof. In some embodiments, the cell line species identity is Gallus gallus.

In some embodiments, the suspension cell culture comprises a minimum regular cell growth medium. In some embodiments, the suspension cell culture comprises a cell growth medium lacking (or having reduced levels of) serum and/or one or more growth factors. In some embodiments, the one or more growth factors are selected from the group consisting of fibroblast growth factor (FGF), epidermal growth factor (EGF), insulin-like growth factor (IGF), insulin, hepatocyte growth factor (HGF), transforming growth factor beta (TGF-β), tumor necrosis factor alpha (TNF-α), leukemia inhibitory factor (LIF) and interleukin-6 (IL6). In some embodiments, the cell growth medium comprises no exogenous FGF, EGF, IGF, HGF, TGF-β, TNF-α, LIF and/or IL6. In some embodiments, the cell growth medium comprises no serum.

In some embodiments, the genetic modification resulting in reduced protein activity comprises knocking-out of a corresponding endogenous gene. In some embodiments, the genetic modification is a homozygous knock-out of the corresponding endogenous gene. In some embodiments, the genetic modification is a heterozygous knock-out of the corresponding endogenous gene. In some embodiments, the knock-out comprises a deletion of all or a part of coding region of the corresponding endogenous gene. In some embodiments, the knock-out comprises a substitution of all or a part of coding region of the corresponding endogenous gene with one or more nucleotides and/or an insertion of one or more nucleotides to the coding region of the corresponding endogenous gene. In some embodiments, the knock-out comprises a premature stop codon in the coding region of the corresponding endogenous gene. In some embodiments, the knock-out comprises a frameshift mutation in the coding region of the corresponding endogenous gene.

In some embodiments, the genetic modification comprises knock-down of the corresponding endogenous gene. In some embodiments, expression level of the endogenous PTEN, CASP3, CASP8, CDH1, p53, ITGB1, RB1, IGFBP4, and/or the Cyclin-dependent kinase inhibitor protein in the cell line is no more than 50%, 40%, 30%, 20%, 10%, 5%, 3%, or 1% compared to the control cell line without at least one of said genetic modifications. In some embodiments, the knock-down comprises an antisense molecule in the cell line, wherein the antisense molecule downregulates the expression of the endogenous PTEN, CASP3, CASP8, CDH1, p53, ITGB1, RB1, IGFBP4, and/or the Cyclin-dependent kinase inhibitor gene via RNA interference (RNAi). In some embodiments, the antisense molecule is selected from the group consisting of a siRNA, a shRNA, and a miRNA. In some embodiments, the knock-down comprises deleting all or a part of the promoter sequence of the corresponding endogenous gene. In some embodiments, the knock-down comprises disrupting the promoter sequence of the corresponding endogenous gene by inserting, deleting and/or mutating one or more nucleotides.

In some embodiments, the genetic modification results in the expression of an endogenous PTEN, CASP3, CASP8, CDH1, p53, ITGB1, RB1, IGFBP4, and/or the Cyclin-dependent kinase inhibitor protein comprising an inactivating mutation.

In some embodiments, the cell line comprises a knock-in PTEN, CASP3, CASP8, CDH1, p53, ITGB1, RB1, IGFBP4, and/or the Cyclin-dependent kinase inhibitor allele encoding a knock-in PTEN, CASP3, CASP8, CDH1, p53, ITGB1, RB1, IGFBP4, and/or the Cyclin-dependent kinase inhibitor protein comprising an inactivating mutation. In some embodiments, the PTEN, CASP3, CASP8, CDH1, p53, ITGB1, RB1, IGFBP4, and/or the Cyclin-dependent kinase inhibitor protein comprising the inactivating mutation is a dominant negative mutant protein.

In some embodiments, the cell line expresses a knock-in TERT and/or SRC protein.

In some embodiments, the cell line expresses an endogenous TERT and/or SRC protein. In some embodiments, expression level of the endogenous TERT and/or SRC protein in the cell line is increased by at least 50%, at least 100%, at least 2-fold, or at least 5-fold, compared to the control cell line without said genetic modification.

In some embodiments, expression level of total TERT and/or SRC protein in the cell line is increased by at least 50%, at least 100%, at least 2-fold, or at least 5-fold, compared to the control cell line without said genetic modification.

In some embodiments, the genetic modification comprises disrupting a targeting sequence of a microRNA within endogenous TERT and/or SRC gene locus.

In some embodiments, the cell line expresses a TERT and/or SRC protein with an activating mutation.

In some embodiments, the PTEN protein comprises a polypeptide sequence having at least 80%, at least 85%, at least 90%, at least 95%, or 100% identity to SEQ ID NO: 1, or wherein the corresponding gene encoding PTEN encodes a polynucleotide sequence having at least 80%, at least 85%, at least 90%, at least 95%, or 100% identity to SEQ ID NO: 9. In some embodiments, the CASP3 protein comprises a polypeptide sequence having at least 80%, at least 85%, at least 90%, at least 95%, or 100% identity to SEQ ID NO: 42, or wherein the corresponding gene encoding CASP3 encodes a polynucleotide sequence having at least 80%, at least 85%, at least 90%, at least 95%, or 100% identity to SEQ ID NO: 43. In some embodiments, the CASP8 protein comprises a polypeptide sequence having at least 80%, at least 85%, at least 90%, at least 95%, or 100% identity to SEQ ID NO: 44, or wherein the corresponding gene encoding CASP8 encodes a polynucleotide sequence having at least 80%, at least 85%, at least 90%, at least 95%, or 100% identity to SEQ ID NO: 45. In some embodiments, the CDH1 protein comprises a polypeptide sequence having at least 80%, at least 85%, at least 90%, at least 95%, or 100% identity to SEQ ID NO: 46, or wherein the corresponding gene encoding CDH1 encodes a polynucleotide sequence having at least 80%, at least 85%, at least 90%, at least 95%, or 100% identity to SEQ ID NO: 47. In some embodiments, the TERT protein comprises a polypeptide sequence having at least 80%, at least 85%, at least 90%, at least 95%, or 100% identity to SEQ ID NO: 48, or wherein the corresponding gene encoding TERT encodes a polynucleotide sequence having at least 80%, at least 85%, at least 90%, at least 95%, or 100% identity to SEQ ID NO: 49. In some embodiments, the p53 protein comprises a polypeptide sequence having at least 80%, at least 85%, at least 90%, at least 95%, or 100% identity to SEQ ID NO: 50, or wherein the corresponding gene encoding p53 encodes a polynucleotide sequence having at least 80%, at least 85%, at least 90%, at least 95%, or 100% identity to SEQ ID NO: 51. In some embodiments, the ITGB1 protein comprises a polypeptide sequence having at least 80%, at least 85%, at least 90%, at least 95%, or 100% identity to SEQ ID NO: 52, or wherein the corresponding gene encoding ITGB1 encodes a polynucleotide sequence having at least 80%, at least 85%, at least 90%, at least 95%, or 100% identity to SEQ ID NO: 53. In some embodiments, the IGFBP4 protein comprises a polypeptide sequence having at least 80%, at least 85%, at least 90%, at least 95%, or 100% identity to SEQ ID NO: 54, or wherein the corresponding gene encoding IGFBP4 encodes a polynucleotide sequence having at least 80%, at least 85%, at least 90%, at least 95%, or 100% identity to SEQ ID NO: 55. In some embodiments, the RB1 protein comprises a polypeptide sequence having at least 80%, at least 85%, at least 90%, at least 95%, or 100% identity to SEQ ID NO: 66, or wherein the corresponding gene encoding RB1 encodes a polynucleotide sequence having at least 80%, at least 85%, at least 90%, at least 95%, or 100% identity to SEQ ID NO: 67. In some embodiments, the SRC protein comprises a polypeptide sequence having at least 80%, at least 85%, at least 90%, at least 95%, or 100% identity to SEQ ID NO: 64, or wherein the corresponding gene encoding SRC encodes a polynucleotide sequence having at least 80%, at least 85%, at least 90%, at least 95%, or 100% identity to SEQ ID NO: 65. In some embodiments, the p21 protein comprises a polypeptide sequence having at least 80%, at least 85%, at least 90%, at least 95%, or 100% identity to SEQ ID NO: 56, or wherein the corresponding gene encoding p21 encodes a polynucleotide sequence having at least 80%, at least 85%, at least 90%, at least 95%, or 100% identity to SEQ ID NO: 57. In some embodiments, the p27 protein comprises a polypeptide sequence having at least 80%, at least 85%, at least 90%, at least 95%, or 100% identity to SEQ ID NO: 58, or wherein the corresponding gene encoding p27 encodes a polynucleotide sequence having at least 80%, at least 85%, at least 90%, at least 95%, or 100% identity to SEQ ID NO: 59. In some embodiments, the p16 protein comprises a polypeptide sequence having at least 80%, at least 85%, at least 90%, at least 95%, or 100% identity to SEQ ID NO: 60, or wherein the corresponding gene encoding p16 encodes a polynucleotide sequence having at least 80%, at least 85%, at least 90%, at least 95%, or 100% identity to SEQ ID NO: 61. In some embodiments, the p18 protein comprises a polypeptide sequence having at least 80%, at least 85%, at least 90%, at least 95%, or 100% identity to SEQ ID NO: 62, or wherein the corresponding gene encoding p18 encodes a polynucleotide sequence having at least 80%, at least 85%, at least 90%, at least 95%, or 100% identity to SEQ ID NO: 63.

In one aspect, the disclosure provides methods of cell culturing, comprising culturing cells derived from the cell line of the disclosure. In some embodiments, the cell culturing is suspension culturing.

In one aspect, the disclosure provides cell population derived from the cell line of the disclosure.

In some embodiments, the cell population is substantially undifferentiated. In some embodiments, more than 70% of the cell population is undifferentiated.

In some embodiments, the cell population is substantially differentiated. In some embodiments, more than 70% of the cell population is differentiated.

In one aspect, the disclosure provides clonal, suspension cell culture comprising the cell population of the disclosure. In some embodiments, the cell population is in contact with a cell growth medium lacking or having reduced level of serum and/or one or more exogenous growth factors. In some embodiments, the cell population has a viable cell density (VCD) that is at least 20% higher than the maximum VCD of a control cell population without at least one of said genetic modifications. In some embodiments, the cell population exhibits a maximum VCD that is at least 10%, 20%, 30%, 40%, 50%, 70%, 100%, 150%, 200%, 300%, or 400% higher than the maximum VCD of a control cell population without at least one of said genetic modifications. In some embodiments, the cell population is capable of reaching maximum VCD at least 10%, 20%, 30%, 40%, 50%, 70%, 100%, 150%, 200%, 300%, or 400% faster than a control cell population without at least one of said genetic modifications. In some embodiments, the cell population is at or capable of reaching a viable cell density of at least 0.4 million, at least 0.5 million, at least 0.6 million, at least 0.7 million, at least 0.8 million, at least 0.9 million, at least 1 million, 2 million, 5 million, 10 million, 15 million or 20 million cells per mL. In some embodiments, the cell population exhibits at least 5%, 10%, 15%, 20%, 30%, or 40% higher cell viability than a control cell population without at least one of said genetic modifications at a viable cell density of at least 0.4 million, at least 0.5 million, at least 0.6 million, at least 0.7 million, at least 0.8 million, at least 0.9 million, at least 1 million, 2 million, 5 million, 10 million, 15 million or 20 million cells per mL. In some embodiments, the cell population is capable of reaching a viable cell density of at least 0.4 million, at least 0.5 million, at least 0.6 million, at least 0.7 million, at least 0.8 million, at least 0.9 million, 1 million, 2 million, 5 million, 10 million, 15 million or 20 million cells per mL at least 10%, 20%, 30%, 40%, 50%, 70%, 100%, 150%, 200%, 300%, or 400% faster than a control cell population without at least one of said genetic modifications.

In one aspect, the disclosure provides methods of generating the cell line of the disclosure, comprising introducing a recombinant nucleic acid into a corresponding parental cell to cause the genetic modification.

In one aspect, the disclosure provides methods of increasing maximum viable cell density of a metazoan cell line in a suspension culture, comprising introducing a genetic modification to the cell line, wherein said genetic modification results in: (i) reduced protein activity of at least one of PTEN, CASP3, CASP8, CDH1, ITGB1, RB1, IGFBP4, p21, p27, p16, and p18; (ii) increased protein activity of SRC; (iii) increased protein activity of TERT; and/or (iv) reduced protein activity of p53, compared to a control cell line without at least one of said genetic modifications in the suspension culture.

In one aspect, the disclosure provides methods of adapting a metazoan cell line to suspension culture, comprising the steps of:
(a) introducing a genetic modification to the cell line, wherein said genetic modification results in (i) reduced protein activity of at least one of PTEN, CASP3, CASP8, CDH1, ITGB1, RB1, IGFBP4, p21, p27, p16, and p18; (ii) increased protein activity of SRC; (iii) increased protein activity of TERT; and/or (iv) reduced protein activity of p53, compared to a control cell line without at least one of said genetic modifications; and
(b) introducing the cell line to suspension culture.

In some embodiments, the method further comprises weaning the cell line from a regular cell growth medium to a cell growth medium lacking (or having reduced levels of) serum and/or one or more growth factors. In some embodiments, the method further comprises the step of: (c) monitoring and removing cell aggregates. In some embodiments, the step (b) comprises an acute treatment of the cell line. In some embodiments, the the acute treatment comprises treating the cell line with a small molecule selected from the group consisting of a Rho-associated kinase (ROCK) inhibitor, an inhibitor of microtubule polymerization (e.g., nocodazole) and a myosin inhibitor (e.g., blebbistatin).

In one aspect, the disclosure provides cell-based meat products comprising: the cell population of the disclosure; and a plant-based protein or product. In some embodiments, the cell population has undergone one or more food processing steps selected from heating, refrigerating, freezing, and smoking. In some embodiments, the the plant product is selected from alfalfa, bamboo, barley, beets, black beans, broccoli, cabbage, canola, carrot, cauliflower, celery, celery root, chickpeas, corn, cotton, cow peas, fava beans, flax, garbanzo beans, green beans, kale, kidney beans, lupin, mung beans, navy beans, northern beans, nuts, oats, parsley, pearl millet, peas, pine nuts, pinto beans, potato, quinoa, red beans, rice, sesame, soy, spelt, sugarbeet, sunflowers, sweet potato, tobacco, wheat, white beans, whole grains, wild rice, zucchini, and a mixture thereof. In some embodiments, the cell-based meat product further comprises a binding agent, a carbohydrate-based gel, a non-animal fat, and/or a flavoring agent.

In one aspect, the disclosure provides ingredients of a food product, comprising the cell population of the disclosure, wherein the cell population has undergone one or more food processing steps selected from heating, refrigerating, freezing, and smoking. In some embodiments, said cell population has been heated at least 70 Celsius temperature for at least 10 minutes. In some embodiments, the cell population is grilled. In some embodiments, the cell population is boiled. In some embodiments, the cell population is fried. In some embodiments, the cell population is baked.

In one aspect, the disclosure provides kits for generating the cell line of the disclosure, comprising: (a) a recombinant nucleic acid that can generate the genetic modification upon introduction into a corresponding parental cell; (b) an agent for introducing the recombinant nucleic acid into the corresponding parental cell.

In some embodiments, the VCD, apoptosis, adherent cells, and other measures of suspension culture are assessed in cultures of at least 5 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL, 500 mL, 525 mL, 550 mL, 575 mL, 600 mL, 625 mL, 650 mL, 675 mL, 700 mL, 725 mL, 750 mL, 775 mL, 800 mL, 825 mL, 850 mL, 875 mL, 900 mL, 925 mL, 950 mL, 975 mL, 1000 mL, 1.5 L, 2 L, 2.5 L, 3 L, 3.5 L, 4 L, 4.5 L, 5 L, 5.5 L, 6 L, 6.5 L, 7L, 7.5 L, 8 L, 8.5 L, 9 L, 9.5 L, 10 L, 50 L, 100 L, 200 L, 300 L, 400 L, 500 L, 600 L, 700 L, 800 L, 900 L, 1000 L, 1500 L, 2000 L, 2500 L, 3000 L, 3500 L, 4000 L, 4500 L, 5000 L, 5500 L, 6000 L, 6500 L, 7000 L, 7500 L, 8000 L, 8500 L, 9000 L, 9500 L, or 1000 L.

### BRIEF DESCRIPTION OF DRAWINGS

FIG. 1 shows the overall workflow of suspension adaptation.

FIG. 2 shows the genetic modifications of fibroblast cell lines of the present disclosure.

FIG. 3 shows the viability of genetically modified fibroblast cell lines of the present disclosure.

FIG. 4 shows the proliferation rate of genetically modified fibroblast cell lines of the present disclosure.

FIG. 5 shows the genetic modifications of myoblast cell lines of the present disclosure.

FIG. 6 shows the viability of genetically modified myoblasts of the present disclosure.

FIG. 7 shows the proliferation rate of genetically modified myoblasts of the present disclosure.

FIG. 8A shows the apoptosis rate of genetically modified fibroblasts. FIG. 8B shows the apoptosis rate of genetically modified myoblasts of the present disclosure.

FIGS. 9A and 9B show results of direct CYQUANT analysis of the indicated cell lines.

FIG. 9C shows results of direct CYQUANT analysis of the cell lines containing (i) the genetic modifications of p53-knockout (KO), TERT-overexpression, and CASP3-KO and (ii) the indicated modification. FIG. 9D shows results of direct CYQUANT analysis of the cell lines containing (i) the genetic modifications of p53-knockout (KO), TERT-overexpression, and CDH1-KO and (ii) the indicated modification. FIG. 9E shows results of direct CYQUANT analysis of the cell lines containing (i) the genetic modifications of p53-knockout (KO), TERT-overexpression, and CASP8-KO and (ii) the indicated modification.

FIG. 10 shows the maximum viable cell density of selected cell lines during the suspension growth in the bioreactor setting.

### DETAILED DESCRIPTION

The details of the disclosure are set forth in the accompanying description below. Although methods and materials similar or equivalent to those described herein can be used in the practice or testing of the present disclosure, illustrative methods, and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete and will fully convey the scope of the invention to those skilled in the art. Unless explicitly indicated otherwise, all specified some embodiments, features, and terms intend to include both the recited embodiment, feature, or term and biological equivalents thereof. The terminology used in the description herein is for the purpose of describing particular embodiments only and is not intended to be limiting.

### Definitions

As used in this specification and the appended claims, the singular forms “a,” “an” and “the” include plural references unless the content clearly dictates otherwise.

As used in this specification and the appended claims, the term “and/or” is used in this disclosure to either “and” or “or” unless indicated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “animal meat” as used herein refers to flesh derived (i.e., harvested) from skeletal muscle or from other organs (e.g., kidney, heart, liver, gallbladder, intestine, stomach, bone marrow, brain, thymus, lung, tongue), or parts thereof, derived from an animal. The animal meat can be dark or white meat. Suitable animals from which the animal meat can be derived include but are not limited to cattle, lamb, mutton, horse, poultry (e.g., chicken, duck, goose, turkey), fowl (any bird species, pigeon, dove, grouse, partridge, ostrich, emu, pheasant, quail), fresh or saltwater fish (e.g., catfish, tuna, spearfish, shark, halibut, sturgeon, salmon, bass, muskie, pike, bowfin, gar, eel, paddlefish, bream, carp, trout, walleye, snakehead, crap-pie, sister, mussel, scallop, abalone, squid, octopus, sea urchin, cuttlefish, tunicate), crustacean (e.g., crab, lobster, shrimp, barnacle), game animals (e.g., deer, fox, wild pig, elk, moose, reindeer, caribou, antelope, zebra, squirrel, marmot, rabbit, bear, beaver, muskrat, opossum, raccoon, armadillo, porcupine, bison, buffalo, boar, lynx, bobcat, bat), reptiles (e.g., snakes, turtles, lizards, alligators, crocodiles), any insect or other arthropod, rodent (nutria, guinea pig, rat, mice, vole, groundhog, capybara), kangaroo, whale, and seal. The term also refers to ground, chopped, shredded, or otherwise processed animal meat. The term encompasses both uncooked, cooking, and cooked animal meat unless otherwise indicated herein or clearly contradicted by context. The meat may be intact, in chunks, in steak form, ground, finely textured, trim or residues derived from processing frozen animals, low temperature rendered, mechanically separated or deboned, MDM, which is a meat paste that is recovered from animal bones, and a comminuted product that is devoid of the natural fibrous texture found in intact muscles (i.e., meat removed from bone by various mechanical means), cooked, or combinations thereof. The meat may include muscle, skin, fat (including rendered fat such as lard and tallow, flavor enhanced animal fats, fractionated or further processed animal fat tissue), or other animal components. For clarity, cultivated animal cells produced outside of an animal (e.g., in an in vitro culture), are not “animal meat” and are not considered to be “derived” from an animal.

The term “binding agent” as used herein refers to an agent that promotes, supports, or enables holding together ingredients in one cohesive mass.

The term “dough” as used herein refers to a blend of dry ingredients (“dry mix”; e.g., proteins, carbohydrates, and lipids including liquid oils) and liquid ingredients (“liquid mix”; e.g., water or juice [i.e., liquid based extract from a non-animal source such as a plant or any part of a plant]) from which a meat structured protein product is produced through the application of mechanical energy (e.g., spinning, agitating, shaking, shearing, pressure, turbulence, impingement, confluence, beating, friction, wave), radiation energy (e.g., microwave, electromagnetic), thermal energy (e.g., heating, steam texturizing), enzymatic activity (e.g., crosslinking activity), chemical reagents (e.g., pH and/or ionic strength adjusting agents, kosmotropic salts, chaotropic salts, gypsum, surfactants, emulsifiers, fatty acids, amino acids), other methods that lead to protein denaturation and protein fiber alignment, or combinations of these methods, followed by fixation of the fibrous structure (e.g., by rapid temperature and/or pressure change, rapid dehydration, chemical fixation, redox).

The term “meat-like” as used herein refers to resemblance to animal meat.

The term “meat-like food product” as used herein refers to a food product that is not animal meat but has structure, texture, and/or other properties comparable to those of animal meat. Meat-like food product includes cell-based meat. The term refers to uncooked, cooking, and cooked meat-like food product unless otherwise indicated herein or clearly contradicted by context.

The terms “cultured meat”, “manufactured meat”, “cell-based meat”, and “cultivated meat” generally refer to meat that contains animal cells grown outside the animal in bioreactor systems or other similar production or cell culture systems. Cell-based meat products of the disclosure may comprise plant-based protein/cell/product or may be composed purely of animal cells, may contain other food ingredients, and may be ground meats such as ground beef, or tissue engineered/synthesized tissue such as bacon or steak.

The terms “genetically modified cell line” or “cell line comprising a genetic modification” refer to a cell line that has been genetically altered, modified, or engineered, such that it exhibits an altered, modified, or different genotype and/or phenotype (e.g., when the genetic modification affects coding nucleic acid sequences of the cell), as compared to the cell from which it was derived. It is understood that in some embodiments, the terms refer not only to the particular cell line in question, but also to the progeny or potential progeny of such a cell line.

The terms “genetically modified”, “genetically engineered or “genetic modification” may refer to any manipulation of a host cell's genome (e.g. by insertion, deletion, mutation, or replacement of nucleic acids). Genetically modified cells include cells harboring artificially added extra recombinant DNA, such as plasmids.

The term “immortalization” generally refers to increasing the Hayflick limit of a cell. “Hayflick limit” generally refers to the finite number of divisions a cell can undergo before the cell becomes senescent. Each time a cell undergoes mitosis, the telomeres on the ends of each chromosome may shorten. Generally cell division ceases once telomeres shorten to a critical length. In some cases, an immortalized cell may undergo a finite number of mitoses. In some cases, an immortalized cell may undergo mitosis indefinitely. For the purposes of this disclosure, cells capable of dividing at least 40 times, are considered immortal.

The term “differentiation” generally refers to a change from a relatively generalized type of cell to a more specialized kind of cell. In some cases, this may comprise an event where either a mononuclear myogenic cell (skeletal muscle cell) fuses with more myogenic cells into a multinucleated muscle fiber capable of generating increased contractile force, or the transition of a fibroblast, mesenchymal stem cell, or an adipose progenitor cell to a mature adipocyte that contains intracellular fat droplets. Myogenic cells can be induced to differentiate when they reach a sufficiently high density. The differentiation of myogenic cells is called “myogenesis”, and the differentiation of fat progenitor cells is called “adipogenesis”.

The term “operably linked” refers to a first polynucleotide molecule, such as a promoter, connected with a second transcribable polynucleotide molecule, such as coding sequence of a gene of interest, where the polynucleotide molecules are so arranged that the first polynucleotide molecule affects the function of the second polynucleotide molecule. The two polynucleotide molecules may be part of a single contiguous polynucleotide molecule and may be adjacent. However, polynucleotide molecules need not be contiguous to be operably linked.

As used herein the term “sequence identity” refers to the extent to which two optimally aligned polynucleotides or polypeptide sequences are invariant throughout a window of alignment of residues, e.g. nucleotides or amino acids. An “identity fraction” for aligned segments of a test sequence and a reference sequence is the number of identical residues which are shared by the two aligned sequences divided by the total number of residues in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as, for example, those in the BLAST suite of sequence analysis programs. Unless noted otherwise, the term “sequence identity” in the claims refers to sequence identity as calculated by Clustal Omega® using default parameters.

As used herein in relation to the position of an amino acid mutation, the terms “corresponding to” or “correspond to” refer to an amino acid in a first polypeptide sequence that aligns with a given amino acid in a reference polypeptide sequence when the first polypeptide and reference polypeptide sequences are aligned. Alignment is performed by one of skill in the art using software designed for this purpose, for example, Clustal Omega version 1.2.4 with the default parameters for that version. As an example, the amino acid position D92 in SEQ ID NO: 2 corresponds to D77 of SEQ ID NO: 1.

When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). Similarity is a more sensitive measure of relatedness between sequences than identity; it takes into account not only identical (i.e. 100% conserved) residues but also non-identical yet similar (in size, charge, etc.) residues. % similarity is a little tricky since its exact numerical value depends on parameters such as the substitution matrix one uses (e.g. permissive BLOSUM45 vs. stringent BLOSUM90) to estimate it.

The terms “conservative amino acid substitution” and “conservative substitution” when used in reference to amino acid substitution denote the replacement of an amino acid residue by another, biologically or chemically similar residue. Examples of conservative substitutions include the substitution of one hydrophobic amino acid residue such as isoleucine, valine, leucine, alanine, cysteine, glycine, phenylalanine, proline, tryptophan, tyrosine, norleucine, or methionine for another, or the substitution of one polar residue for another, such as the substitution of arginine for lysine and vice versa, of glutamic acid for aspartic acid, and vice versa, glutamine for asparagine, and vice versa, and the like. Neutral hydrophilic amino acids which can be substituted for one another include asparagine, glutamine, serine and threonine. The terms “conservative amino acid substitution” and “conservative substitution” also includes the use of a substituted amino acid in place of an unsubstituted parent amino acid provided that the biologic activity of the peptide is maintained. Biological similarity between amino acid residues refers to similarities between properties such as, but not limited to, hydrophobicity, mutation frequency, charge, side chain length, size chain volume, pKa, polarity, aromaticity, solubility, surface area, peptide bond geometry, secondary structure propensity, average solvent accessibility, etc.

The term “dominant negative” refers to a mutant gene or expressed protein that adversely affects the normal functioning of the wildtype gene/protein. In some embodiments, a dominant negative protein or mutant gene substantially or even completely inhibits the activity of the corresponding wildtype protein (for example, by competing with wildtype proteins for substrates, ligands, binding partners, etc.). In some embodiments, a dominant negative mutant gene or protein mimics the phenotype of a knockout or knockdown of the corresponding wildtype gene/protein.

The term “RNAi” refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNA, shRNA, endogenous microRNA and artificial microRNA. In some embodiments, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors).

The term “siRNA” refers to a nucleic acid that forms a double stranded RNA, which has the ability to reduce or inhibit expression of a gene or target gene when the siRNA is present or expressed in the same cell as the target gene. The double stranded siRNA can be formed by the complementary strands. The sequence of the siRNA can correspond to the full length target gene, or a subsequence thereof. Typically, siRNA is at least about 15-50 nucleotides in length (e.g., each complementary sequence of the double stranded siRNA is about 15-50 nucleotides in length, and the double stranded siRNA is about 15-50 base pairs in length, or about 20-30 nucleotides in length, e.g., 20, 21, 22, 23, 24, 25, 26, 27, 28, 29, or 30 nucleotides in length). A siRNA can be chemically synthesized, it can be produced by in vitro transcription, or it can be produced within a cell specifically utilized for such production.

The terms “shRNA” and “small hairpin RNA” are used interchangeably and refer to a type of siRNA. In some embodiment, shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi and/or siRNA species but differs in that shRNA species are double stranded hairpin-like structure for increased stability. These shRNAs can be contained in and expressed from various expression constructs, plasmids, retroviruses, and lentiviruses.

The terms “microRNA” and “miRNA” are used interchangeably herein. Endogenous microRNA are small RNAs naturally present in the genome which are capable of modulating the productive utilization of mRNA. IN some embodiments, the miRNA is an artificial miRNA which is capable of modulating the productive utilization of mRNA. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and/or RNAi pathways.

The term “suspension culture” or “suspension cell culture” refers to cell culture in which the majority or all of cells in a culture vessel are present in suspension e.g., are not attached to any substratum or surface, the vessel surface, or to another surface within the vessel. The suspension culture may be shaken, rocked, agitated, rolled or stirred to maintain the cells in suspension. In some embodiments, suspension cultures may contain a small amount of aggregated cells (e.g. small clusters of less than 5 cells).

The number of “generations” when used in reference to the cell line refers to the number of population doublings that a cell culture has undergone. The calculation of population doublings is well known in the art (see, e.g., Patterson, Methods in Enzymology, eds. Jakoby and Pastan, Academic, New York, 58:150-151 (1979)). In one embodiment, the in vitro cell age or generation number of a culture is determined by calculating the number of cell divisions during the culture period, following the formula, 1n(fold of increase in cell mass)/1n2. In one embodiment, the increase in cell mass is measured by the method disclosed in Tsao et al. (Biotechnol. Prog. 16:809-814 (2000)).

The term “cell viability” refers to the ability of cells in culture to survive under a given set of culture conditions or experimental variations. The term can be expressed by the percentage of cells which are viable at a particular time in relation to the total number of cells, living and dead, in the culture at that time. A cell is considered viable if it has the ability to grow and develop. Viability assays may be based on any known test for viability, including those based on physical properties of viable cells, such as membrane integrity, or on their metabolic activity.

The term “myogenesis” refers generally to muscle formation, a complex and highly orchestrated process that involves the determination of multipotential mesodermal cells to give rise to myoblasts, exit of myoblasts from the cell cycle, and their eventual differentiation into skeletal muscle fibers.

### Cultures of Myogenic and Fibroblasts Cells

The present disclosure relates to cell lines and methods for enhancing cell-based meat production via in vitro culture of fibroblast and myogenic cells. In one aspect, the present disclosure enables lower cost production of meat culture, which is based in part on the discovery that genetic modification of fibroblast or myoblasts renders resistance to cell death (e.g., anoikis) and allows the cells to maintain proliferation status in suspension culture.

A primary hurdle in the industrial production of mammalian biomass is the adaptation of an adherent cell line to suspension growth in a bioreactor. The ability to grow cells in three dimensions rather than two is tremendously advantageous for cost reduction in an industrial bioprocess. Traditionally, cell lines could be adapted for suspension growth by placing selective pressure on formerly adherent cells (e.g., by forcing them to remain in suspension). Most of the cells subjected to this selective pressure either senesce or die by apoptosis. Any cells that survive this process, and eventually begin to proliferate are considered adapted.

Very few non-stem cell mammalian lines have been successfully adapted to suspension growth via this method. One of the downsides of this approach is that there is a high failure rate. Another major downside is that the resulting lines are often unstable, prone to losing their ability to grow in suspension if exposed to changes in their culture conditions, or other modifications in their morphology (e.g., a triggered differentiation or de-differentiation). Thus, there is a need for modified cell lines that are capable of stable suspension growth, ideally based on genetic improvements, as described in this disclosure.

One major obstacle in establishing suspension culture is that adherent cells (e.g., fibroblasts or myoblasts) that detach from fixed growth surfaces and enter suspension will typically trigger apoptosis. Apoptosis is a mechanism for programmed cell death and a necessary component of development and homeostasis in multicellular organisms including mammals. There are several different apoptotic triggers, including DNA damage, growth factor deprivation, ER stress, and integrin detachment from the extracellular matrix. These inputs share apoptotic signaling pathways which lead to the activation of caspases, cysteine-aspartic proteases that digest intracellular proteins but do not affect nearby cells. Specifically, the detachment-induced apoptosis triggered during suspension adaptation is called anoikis.

Another obstacle to achieving reliable suspension culture lines is that, suspension culture medium often lacks—or has a reduced level of—serum and/or one or more exogenous growth factors, which sometimes also triggers apoptosis or an alternative programmed cell death mechanism induced by growth factor deprivation. Thus, desirable phenotypes of adapted cells include a rounded morphology, and the ability to proliferate in reduced serum conditions and tolerance to shear stress.

The present disclosure describes the enhanced resistance to programmed cell death (e.g., anoikis) by introducing one or more of genetic modifications to the cells for suspension culture. These modifications are made to facilitate the adaptation of the cell lines to suspension growth in a bioreactor by reducing or eliminating detachment-induced apoptosis. In some embodiments, such genetically modified cells also exhibit improved proliferation rates in suspensions and/or adherent growth conditions. In some embodiments, such genetically modified cells also exhibit reduced needs for serum and/or growth factors in the culture medium. In some embodiments, the cells are mammalian cells. In some embodiments, the cells are bovine cells.

The present disclosure provides genetically modified cell lines comprising one or more genetic modifications and the use of such cell lines for producing cell culture-based meat. Without being bound by any particular theory, it is contemplated that the one or more genetic modifications disclosed in this specification result in cell lines that exhibit one or more of the following characteristics: 1) capability of being adapted to suspension culture; 2) stability in suspension culture for an extended period of time; 3) capability of reaching a higher maximum viable cell density in suspension culture; 4) enhanced proliferation rate/mitotic potential in suspension culture; 5) reduced tendency to form cell aggregates in suspension culture; 6) resistance to apoptosis (e.g., anoikis); 7) tolerance of cell medium with reduced or no serum/growth factors (which otherwise would typically induce apoptosis); 8) tolerance of industrial culture process (i.e., cultures of greater than 10 gallons in total volume). These characteristics are beneficial for cell culture in adherent and/or suspended states. In some embodiments, such characteristics are especially beneficial for industrial-scale production of cells (e.g., fibroblasts or myoblasts) in suspension culture.

### Genetic Modification

In one aspect, the disclosure provides a metazoan cell line comprising one or more genetic modifications resulting in 1) reduced protein activity of at least one of PTEN, CASP3, CASP8, CDH1, ITGB1, RB1, IGFBP4, p21, p27, p16, and p18; 2) increased protein activity of SRC; 3) increased protein activity of TERT; and/or 4) reduced protein activity of p53, compared to a control cell line without said genetic modifications. In some embodiments, the genetically modified cell line is adapted for suspension culture. Thus, in some embodiments, the present disclosure teaches metazoan (e.g., bovine) cell lines adapted to suspension culture, said cell lines comprising one or more genetic modifications resulting selected from the group consisting of PTEN (RA), CASP3 (RA), CASP8 (RA), CDH1 (RA), ITGB1 (RA), RB1 (RA), IGFBP4 (RA), p21 (RA), p27 (RA), p16 (RA), p18 (RA), and SRC (IA); wherein RA means reduced activity and IA means increased activity. In some embodiments, these cell lines further comprise reduced protein activity of p53 or increased protein activity of TERT. Further descriptions of each of these recited modifications, including specific genetic modifications for each, follow in the sections below.

A person skilled in the art will readily recognize proper control cell lines for the genetically modified cell lines of the disclosure. In some embodiments, the control cell line is genetically identical to the genetically modified cell line except for the one or more distinguishing genetic modifications (e.g., those recited in the claims). In some embodiments, the control cell line is a wild type cell line without any genetic modifications. In all cases, references to a control cell line is meant to indicate that the recited property (e.g., physiological improvement) is the result of the genetic modification of the cell lines of the disclosure. The same applies to the corresponding cell populations and cell culture. A person skilled in the art will also recognize that comparison of cell properties such as proliferation rate and maximum viable cell density should be conducted at similar starting points (e.g., equal seeding cells amount).

In some embodiments, such a genetically modified cell line is a clonal somatic cell line.

In some embodiments, a genetically modification that results in reduced protein activity of a gene comprises a modification selected from the group consisting of: a) knock-out of the endogenous gene; b) knock-down of the endogenous gene (e.g., via CRISPRi or RNAi); c) knock-in of a gene encoding a dominant negative of the protein; d) inactivation of the endogenous gene (e.g., through mutation in one or more residues); and e) any combination thereof.

In some embodiments, a genetically modification that results in increased protein activity of a gene comprises a modification selected from the group consisting of: a) knock-in of a gene encoding the corresponding protein (e.g., multiple gene copies); b) introduction of one or more activating mutations to the endogenous protein; c) overexpression of endogenous protein; and d) any combination thereof.

In some embodiments, the one or more genetic modifications impart and/or enhance anoikis resistance to the genetically modified cell line and therefore facilitates its adaptation to suspension culture. In some embodiments, the one or more genetic modifications impart and/or enhance resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

The term “knock-out” is well-understood by those having skill in the art. In some embodiments, “knock-out” refers to the elimination of a gene or the expression of the gene. Elimination in some embodiments refers to the abrogation of expression of a functional or full-length protein or mRNA encoded by the gene, despite the fact that a non-functional or truncated product may be produced. Knock-out may involve introducing one or more insertions, deletions or mutations within or near the target gene or other DNA sequences that encode regulatory elements of the target gene. Typically, in a metazoan cell, the knock-out can be a heterozygous knock-out or a homozygous knock-out. In heterozygous knock-out, only one of two gene copies (alleles) is knocked out, whereas in the homozygous knock-out both copies (alleles) are knocked out. Strategies for gene knock-out are well known in the art.

In some embodiments, knock-out comprises deleting all or a part of the target gene (e.g., an exon) from the cell genome. In some embodiments, knock-out comprises replacing all or a part of the target gene in the cell genome with one or more nucleotides. In some embodiments, knock-out comprises introducing a premature stop codon or a frameshift mutation into the coding region of the target gene. In some embodiments, knock-out comprises introducing into the cell a DNA binding protein or DNA-binding nucleic acid that specifically binds to or hybridizes to the gene. In some embodiments, the knock-out is a homozygous knock-out. In some embodiments, the knock-out is a heterozygous knock-out.

The term “knock-down” as used herein refers to reduction in the expression of a gene or its gene product(s). As a result of a gene knock-down, the protein activity or function may be attenuated, or the protein levels may be reduced or eliminated. Strategies for gene knock-down are well known in the art. In some embodiments, the knock-down comprises modification of the endogenous gene locus. In some embodiments, the knock-down is a heterozygous knock-down (only expression of one allele is knocked down). In some embodiments, the knock-down is a homozygous knock-down (expression of both alleles is knocked down).

In some embodiments, knock-down comprises introducing one or more insertions, deletions and/or mutations within or near the target gene or other DNA sequences that encode regulatory elements of the target gene. In some embodiment, knock-down comprises introducing one or more microRNA binding sequences into the coding region of the target gene and/or its non-coding region (e.g., 5′-UTR and/or 3′-UTR), which facilitates the degradation of the corresponding mRNA transcribed from the target gene. In some embodiments, knock-down comprises modifying one or more regulatory elements operably linked to the target gene, for example disrupting or replacing the promoter sequence, the enhancer sequence, and/or the poly-A sequence. In some embodiments, knock-down comprises deletion of the promoter sequence of the corresponding gene, or a portion thereof. In some embodiments, knock-down comprises disrupting the promoter sequence by inserting, deleting and/or mutating one or more nucleotides.

In some embodiments, knock-down comprises introducing or upregulating the expression of one or more antisense molecules to the cells to downregulate the expression level of the target gene. In some embodiments, the antisense molecule downregulates the expression of the target gene via RNA interference (RNAi). In some embodiments, the one or more antisense molecules comprises a short interfering RNA (siRNA), a short hairpin RNA (shRNA), a microRNA (miRNA), or a ribozyme. In some embodiments, the microRNA is miR-21. In some embodiments, the antisense molecule degrades the mRNA transcript of the target gene, or otherwise suppress its expression or function. In some embodiments, introduction of the antisense molecule is achieved by introducing a recombinant DNA encoding the antisense molecule to the cell. Methods and applications of antisense molecules and RNAi are well known in the art, for example, see U.S. Pat. Nos. 8,309,704; 10,633,654; 10,760,079; Fakhr et al., Cancer Gene Therapy (2016) 23, 73-82; and Jessica Chery, Postdoc J. 2016 July; 4 (7): 35-50; the contents of which are incorporated by reference herein in their entireties.

In some embodiments, knock-down comprises introducing one or more CRISPRi module to the cells to downregulate the expression level of the target gene. CRISPRi (CRISPR interference) employs an enzymatically deficient Cas9 (dCas9) fused to or interacting with transcriptional effector(s). Typically, dCas9 contains mutations in two active endonuclease domains and loses the capability to cut DNA but can still target a specific DNA location when coupled with a guide RNA (gRNA). Accordingly, in CRISPRi, dCas9 can be recruited to the target site using a gRNA complementary to the promoter region or transcriptional start site, and the presence of transcriptional repressor(s) fused to or interacting with dCas9 results in repression of gene expression. Illustrative approaches of CRISPRi are provided in Larson et al., Nature Protocols (2013) volume 8, pages 2180-2196; Qi et al., Cell. 2013 152 (5): 1173-1183; Macleod et al., Sci Rep. 2019; 9:17312, the contents of which are incorporated by reference in their entireties.

The term “knock-in” with regard to DNA refers to an addition of a DNA sequence into a cell (e.g., into the cell genome). Such DNA sequence to be knocked-in may comprise an entire gene or a functional fragment thereof, and may further comprise one or more regulatory sequences (e.g., promoter) operably linked to the gene or the functional fragment thereof. Strategies for gene knock-in are well known in the art. And the term “knock-in” with regard to a protein refers to a protein that is expressed from a recombinant nucleic acid introduced into the cell line via genetic engineering (i.e., “knock-in” DNA). In other words, the knock-in protein is not expressed from the original gene locus endogenous to the cell genome. On the other hand, the term “endogenous” with regard to a protein refers to a protein that is expressed from the original gene locus endogenous to the cell genome even though it may contain one or more inactivating or activating mutations due to genetically modification of the endogenous gene locus. Unless clearly specified as “knock-in” or “endogenous”, the term “protein” does not differentiate between “knock-in” and “endogenous” proteins, and terms such as “expression of the protein” and “activity of the protein” within a cell may refer to the overall expression/activity of the protein within the cell.

The knock-in strategy may increase or reduce the overall activity of the corresponding protein in the cell. In some embodiments, the knock-in strategy increases the overall activity of the corresponding protein (e.g., by increases the total expression level of the protein). In some embodiments, the knock-in protein comprises one or more activating mutations. On the other hand, in some embodiments, the knock-in strategy decreases the overall activity of the corresponding protein. In some embodiments, the corresponding knock-in protein comprises one or more inactivating mutations. In some embodiments the corresponding knock-in protein is a dominant negative form. Knock-in of the dominant negative protein can result in the overall decrease of protein activity. Additionally, expressing a dominant negative protein can result in a phenotype that is similar to that of a knockout and/or knockdown.

This knock-in protein can be expressed in a nucleic acid controlled by any promoter (e.g., a promoter of the present disclosure). For example, a promoter can be a ubiquitous promoter. A promoter can also be an inducible promoter, tissue specific promoter, cell specific promoter, and/or developmental specific promoter.

In some embodiments, the corresponding knock-in protein is derived from the same animal species as the genetically modified cell line. In some embodiments, the knock-in protein is derived from a different animal species from the genetically modified cell line. In some embodiments, the genetically modified cell line comprises knock-in protein ectopically expressed from constructs that are introduced into the cells, for example expressed from a plasmid, or other expression vector. In some embodiments, the recombinant nucleic acid is integrated into the cell's genome, and the expression is driven in that manner (e.g., homologous recombination, introduction mediated by CRISPR-based technology).

In some embodiments, the genetic modification comprises inactivation of the endogenous gene. In some embodiments, such inactivation comprises introducing one or more inactivating mutations to the endogenous gene. In some embodiments, such inactivation comprises modifying the endogenous gene so that it expresses a dominant negative protein. In some embodiments, only one copy of the endogenous gene/allele is inactivated (i.e., heterozygous inactivation). In some embodiments, both copies of the endogenous gene/alleles are inactivated (i.e., homozygous inactivation).

While mutations of the proteins/genes described herein are most described in the context of human sequences in the literature, a skilled person would be able to introduce the corresponding mutations into the protein/gene sequences of other species (e.g., a bovine protein sequence) and verify the mutational effect (e.g., activating, inactivating) using assays known in the art.

### PTEN

Phosphatase and Tensin Homolog (PTEN) is a tumor suppressor protein that is frequently lost or mutated in cancers and in a number of tumor syndromes. PTEN functions as a dual-specificity protein phosphatase with predominant enzymatic activity on phosphoinositides. As a phospholipid phosphatase, PTEN catalyzes the hydrolysis of the second messenger PtdIns (3,4,5) P3 (PIP3) and counteracts the activation of the PI3K/Akt pathway, thus regulating cellular growth, proliferation, and metabolism. PTEN protein sequence is highly conserved evolutionarily, with only small amino acid variations across various species.

Structurally, PTEN belongs to the Class I Cys-based protein tyrosine phosphatase (PTP) and, more specifically, to the VH1-like family. PTEN contains, from N-terminus to C-terminus, a PIP2-binding domain (PBD), a phosphatase domain, a C2 domain, a C-tail domain and a PDZ-binding motif. The phosphatase domain contains the active site, which carries out the enzymatic function of the protein. The C2 domain binds the phospholipid membrane, bringing the active site to the membrane-bound PIP3 to dephosphorylate it. The PIP2-binding domain (PBD) increases PTEN's affinity for the plasma membrane by binding to Phosphatidylinositol 4,5-bisphosphate, or possibly anionic lipid in general. The C-tail domain is intrinsically disordered but contains multiple phosphorylation sites which participate in the regulation of protein activity. Phosphorylations of PTEN in general increase its stability but reduce its activity.

The structure-function relationship of PTEN protein mutation and its function has been established in the literature, for example, see Lee et al., Cell. 1999 Oct. 29;99 (3): 323-34.; Smith and Briggs, Proteins. 2016 November; 84 (11): 1625-1643., the contents of which are incorporated by reference in their entirety. Using SEQ ID NO: 2 as the reference sequence, the active site pocket of the phosphatase domain is delimited in part by the signature motif P loop (residues 123-130), WPD loop (residues 88-98), and TI loop (residues 160-171), which contain residues that are responsible for catalysis (D92, C124 and R130), for supplying positive charge within the active site (H93, K125, K128), for mediation of loop motion (H123 and G127), and for governing the depth and width of pocket (K163, K164, G165 and V166). The tumor suppressor function of PTEN is dependent on its phospholipid phosphatase activity and the loss-of-function of the phosphatase catalytic domain is commonly associated with oncogenic PTEN mutations.

Regulation of PTEN function occurs through various posttranslational modifications implicated in PTEN membrane recruitment, subcellular localization, or protein-protein interactions. For example, PTEN interacts with a number of PDZ-domain bearing proteins via its PDZ-binding motif to achieve higher levels of complex formation.

PTEN has been shown to self-interact and form dimers. Wildtype PTEN dimer is active toward its phosphoinositide substrate PIP3 and thereby inhibits the activation of the PI3K/Akt signaling pathway. Importantly, by dimerizing with wildtype PTEN protein, PTEN mutants with disrupted activity can act in a dominant-negative manner to suppress the function of wildtype PTEN protein and thereby facilitate tumorigenesis. See Papa et al., Cell. 2014 Apr. 24;157 (3): 595-610, the content of which is incorporated by reference in its entirety.

Additionally, PTEN can be expressed as longer isoforms. The best characterized alternative isoform is known as PTEN-Long or PTEN-α, which in many species has an additional 173 amino acids to the N-terminus of PTEN utilizing a leucine initiator alternative start site variant. While the N-terminal extension is predicted to be largely disordered, there are evidences that it allows PTEN-Long protein to be secreted from the cells and/or interact with the mitochondria. See Hopkins et al., Trends Biochem Sci. 2014 April;39 (4): 183-90, the content of which is incorporated by reference in its entirety.

Without being bound by any particular theory, it is contemplated that the activity of PTEN protein facilitates detachment-induced apoptosis at least partly through regulation of cell motility signaling. Cell motility can be affected by several inputs including cytokines, growth factors, tensile strength of the extracellular matrix, and epigenetic regulation of the actin cytoskeleton. A structural unit of cell motility is the focal adhesion, which is formed by the coordinated self-assembly of transmembrane integrins, integrin binding proteins, and the actin cytoskeleton. Contractile force generated by the actin-myosin network through focal adhesions enables cells to sense the tensile strength of their environment and can generate critical signals for proliferation or differentiation. Large, mature focal adhesions correlate with a less motile cell that exerts increased force on its environment, while smaller, immature focal adhesions correlate with highly motile cells that can travel large distances.

PTEN protein inhibits PI3K/Akt pathway activity through reducing the intracellular levels of PIP3. Accumulation of PIP3 serves as a major signal for growth factor stimulation. PIP3 binds to the pleckstrin homology (PH) domain of downstream proteins (e.g., Akt) and provides a lipid moiety and recruits these proteins to the plasma membrane. Binding of PIP3 to the PH domain also changes the confirmation of these proteins so they can later be activated by phosphorylation. By reducing the intracellular levels of PIP3, PTEN inhibits the activation of downstream proteins of the PI3K pathway, including the serine/threonine kinase Akt and the protein kinase C (PKC).

Akt plays a critical role in regulating a number of cellular activities including cell growth, survival, cell migration and differentiation, cell and organ size control, metabolism, etc. Akt, also known as Protein Kinase B (PKB) is a serine/threonine kinase. Following PI3K activation, accumulation of PIP3 allows recruitment of Akt to the plasma membrane via direct interaction with its PH domain. This binding of Akt to PIP3 not only allows Akt to be translocated to the membrane but also exposes sites on Akt where it can be further modified. It has been shown that Akt is phosphorylated by another PH domain-containing kinase 3-phosphoinositide-dependent protein kinase 1 (PDK1) at Thr308. This phosphorylation on Thr308 is important for initial activation of Akt whereas phosphorylation of Ser473 by mTORC2 is required for maximal Akt activation.

Additional discussions of the PTEN signaling can be found, for example, in Chen et al., Front Endocrinol (Lausanne), 2018; 9:338.; Leslie et al., Oncogene, 2008 Sep. 18;27 (41): 5464-76; Wang and Jiang, Oncogene, 2008 Sep. 18;27 (41): 5454-63; Paoli et al., Biochim Biophys Acta. 2013 December;1833 (12): 3481-3498; Wang et al., Neoplasia. 2018 June; 20 (6): 574-593; Rodríguez-Escudero et al., Hum Mol Genet. 2011 Nov. 1;20 (21): 4132-42; Davies et al., Cancer Res. 1999 Jun. 1;59 (11): 2551-6; Vitolo et al., Cancer Res. 2009 Nov. 1;69 (21): 8275-83; Yang et al., Zhonghua Zhong Liu Za Zhi. 2005 May;27 (5): 273-5, the contents of which are incorporated by reference in their entireties.

In some embodiments, such a genetically modified cell line comprises a genetic modification selected from the group consisting of: a) knock-out of the endogenous PTEN gene; b) knock-down of the endogenous PTEN gene (e.g., via CRISPRi or RNAi); c) knock-in of a gene encoding a dominant negative PTEN protein; d) inactivation of the endogenous PTEN gene; and e) any combination thereof.

In some embodiments, the reduction of PTEN activity renders anoikis resistance to the genetically modified cell line and therefore facilitates its adaptation to suspension culture. In some embodiments, the reduction of PTEN activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the genetically modified cell line with PTEN knock-down expresses/accumulates the endogenous PTEN protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous PTEN protein in a control cell line. In some embodiments, the genetically modified cell line with PTEN knock-down expresses the endogenous PTEN protein at a level that is no more than 10% of the expression level of the endogenous PTEN protein in a control cell line.

In some embodiments, the disclosure provides a genetically modified cell line comprising a recombinant nucleic acid encoding a PTEN protein (i.e., knock-in PTEN protein). In some embodiments, the knock-in PTEN protein comprises one or more inactivating mutations. In some embodiments the knock-in PTEN protein is a dominant negative form. Knock-in of the dominant negative protein can result in overall decreased function of a wild-type gene. Additionally, expressing a dominant negative protein can result in a phenotype that is similar to that of a knockout and/or knockdown.

This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter.

In some embodiments, the knock-in PTEN protein is derived from the same animal species as the genetically modified cell line. In some embodiments, the knock-in PTEN protein is derived from a different animal species from the genetically modified cell line. In some embodiments, the genetically modified cell line comprises knock-in PTEN protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous PTEN gene. In some embodiments, such inactivation comprises introducing one or more inactivating mutations to the endogenous PTEN protein coding gene. In some embodiments, such inactivation comprises modifying the endogenous PTEN gene so that it expresses a dominant negative PTEN protein. In some embodiments, only one copy of the endogenous PTEN gene/allele is inactivated (i.e., heterozygous inactivation). In some embodiments, both copies of the endogenous PTEN gene/alleles are inactivated (i.e., homozygous inactivation).

Exemplary PTEN protein sequences are provided herein as SEQ ID NO: 1-8.

In some embodiments, the PTEN protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with any one of SEQ ID NO: 1-8, including all ranges and subranges therebetween. In some embodiments, the PTEN protein of the disclosure comprises or consists of an amino acid sequence having up to 1, 2, 3, 4, 5, 6, 7, 8, 9, or 10 amino acids different from the amino acid sequence according to any one of SEQ ID NO: 1-8.

In some embodiments, the PTEN protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with any one of SEQ ID NO: 1-8, including all ranges or subranges therebetween.

In some embodiments, the PTEN protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween, wherein the sequence difference with any one of SEQ ID NO: 1-8 consists of conservative amino acid substitutions (other than the one or more inactivating mutations of the disclosure).

In some embodiments, the PTEN protein of the disclosure comprises or consists of an amino acid sequence at least 60%, at least 65%, at least 70%, at least 75%, at least 80%, at least 85%, at least 90%, at least 95%, at least 96%, at least 97%, at least 98%, at least 99%, at least 99.5%, or 100% identical to SEQ ID NO: 1, including all ranges and subranges therebetween. In some embodiments, the PTEN protein of the disclosure comprises or consists of an amino acid sequence having up to 1, 2, 3, 4, 5, 6, 7, 8, 9, or 10 amino acids different from the amino acid sequence according to SEQ ID NO: 1.

In some embodiments, the PTEN protein (knock-in and/or endogenous) of the disclosure comprises one or more inactivating mutations. In some embodiments, the PTEN protein comprising the one or more inactivating mutations is a dominant negative protein. In some embodiments, the genetic modification comprises a knock in gene encoding for a PTEN protein with a dominant negative inactivating mutation.

In some embodiments, the inactivating mutation of PTEN protein comprises a single amino acid substitution. In some embodiments, the inactivating mutation of PTEN protein comprises a single amino acid insertion or deletion. In some embodiments, the inactivating mutation of PTEN protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues).

In some embodiments, the PTEN protein of the disclosure comprises or consists of an inactivating mutation at one or more amino acid positions corresponding to those in SEQ ID NO: 1 or 2. In some embodiments, the inactivating mutation of PTEN protein reduces the phosphatase activity of PTEN by at least 5%, at least 10%, at least 15, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to the corresponding wildtype PTEN protein. In some embodiments, the inactivating mutation of PTEN protein reduces the phosphatase activity of PTEN by at least 50% compared to the corresponding wildtype PTEN protein. In some embodiments, the phosphatase activity is phospholipid phosphatase activity (e.g., for PIP3). In some embodiments, the phosphatase activity is protein phosphatase activity.

In some embodiments, the inactivating mutation of PTEN protein comprises or consists of a mutation at one or more amino acid positions within the active site pocket of the PTEN phosphatase domain formed by the P loop, the WPD loop and the TI loop. In some embodiments, the inactivating mutation of PTEN protein comprises or consists of a mutation within the P loop, which corresponds to residues 108-115 of SEQ ID NO: 1 or 123-130 of SEQ ID NO: 2. In some embodiments, the inactivating mutation of PTEN protein comprises or consists of a mutation within the WPD loop, which corresponds to residues 73-83 of SEQ ID NO: 1 or 88-98 of SEQ ID NO: 2. In some embodiments, the inactivating mutation of PTEN protein comprises or consists of a mutation within the TI loop, which corresponds to residues 145-156 of SEQ ID NO: 1 or 160-171 of SEQ ID NO: 2.

In some embodiments, the inactivating mutation of PTEN protein comprises or consists of a mutation at one or more amino acid positions selected from the group consisting of those corresponding to K13, R15, D24, Y27, 133, M35, G36, P38, L42, G44, V45, C56, Y61, A64, F66, Y73, D77, H78, P80, P81, 186, C90, D92, L97, A105, H108, C109, A111, G112, G114, R115, T116, G117, M119, 1120, C121, R127, A136, F139, Y140, R144, D147, G150, T152, P154, S155, Q156, R158, Y159, Y162, Y163, V176, M184, P189, R219, F226, P231, G236, D237, 1238, H257, F263, L305, L310, P324, F326, and T333 of SEQ ID NO: 1. In some embodiments, the inactivating mutation of PTEN protein comprises or consists of one or more amino acid substitutions at one or more positions selected from the group consisting of those corresponding to K13, R15, D24, Y27, 133, M35, G36, P38, L42, G44, V45, C56, Y61, A64, F66, Y73, D77, H78, P80, P81, 186, C90, D92, L97, A105, H108, C109, Alll, G112, G114, R115, T116, G117, M119, 1120, C121, R127, A136, F139, Y140, R144, D147, G150, T152, P154, S155, Q156, R158, Y159, Y162, Y163, V176, M184, P189, R219, F226, P231, G236, D237, 1238, H257, F263, L305, L310, P324, F326, and T333 of SEQ ID NO: 1, wherein the amino acid substitutions correspond to those selected from the middle column of Table 1 below. In some embodiments, the inactivating mutation of PTEN protein comprises or consists of one or more amino acid substitutions at one or more positions selected from the group consisting of those corresponding to K13, R15, D24, Y27, 133, M35, G36, P38, L42, G44, V45, C56, Y61, A64, F66, Y73, D77, H78, P80, P81, 186, C90, D92, L97, A105, H108, C109, A111, G112, G114, R115, T116, G117, M119, 1120, C121, R127, A136, F139, Y140, R144, D147, G150, T152, P154, S155, Q156, R158, Y159, Y162, Y163, V176, M184, P189, R219, F226, P231, G236, D237, 1238, H257, F263, L305, L310, P324, F326, and T333 of SEQ ID NO: 1, wherein the amino acid substitution is different from those provided in the right column of Table 1 below.

In some embodiments, the inactivating mutation of PTEN protein comprises or consists of a mutation at one or more amino acid positions selected from the group consisting of those corresponding to K13, R15, D24, Y27, 133, M35, G36, P38, L42, G44, V45, H61, K66, 167, Y68, C71, Y76, A79, F81, Y88, D92, H93, P95, P96, 1101, C105, D107, L112, A120, H123, C124, A126, G127, G129, R130, T131, G132, M134, 1135, C136, R142, A151, F154, Y155, R159, D162, G165, T167, P169, S170, Q171, R173, Y174, Y177, Y178, V191, M199, P204, R234, F241, P246, G251, D252, 1253, H272, F278, L320, L325, P339, F341, and T348 of SEQ ID NO: 2. In some embodiments, the inactivating mutation of PTEN protein comprises or consists of one or more amino acid substitutions at one or more positions selected from the group consisting of those corresponding to K13, R15, D24, Y27, 133, M35, G36, P38, L42, G44, V45, H61, K66, 167, Y68, C71, Y76, A79, F81, Y88, D92, H93, P95, P96, 1101, C105, D107, L112, A120, H123, C124, A126, G127, G129, R130, T131, G132, M134, 1135, C136, R142, A151, F154, Y155, R159, D162, G165, T167, P169, S170, Q171, R173, Y174, Y177, Y178, V191, M199, P204, R234, F241, P246, G251, D252, 1253, H272, F278, L320, L325, P339, F341, and T348 of SEQ ID NO: 2, wherein the amino acid substitutions correspond to those selected from the middle column of Table 1 below. In some embodiments, the inactivating mutation of PTEN protein comprises or consists of one or more amino acid substitutions at one or more positions selected from the group consisting of those corresponding to K13, R15, D24, Y27, 133, M35, G36, P38, L42, G44, V45, H61, K66, 167, Y68, C71, Y76, A79, F81, Y88, D92, H93, P95, P96, 1101, C105, D107, L112, A120, H123, C124, A126, G127, G129, R130, T131, G132, M134, 1135, C136, R142, A151, F154, Y155, R159, D162, G165, T167, P169, S170, Q171, R173, Y174, Y177, Y178, V191, M199, P204, R234, F241, P246, G251, D252, 1253, H272, F278, L320, L325, P339, F341, and T348 of SEQ ID NO: 2, wherein the amino acid substitution is different from those provided in the right column of Table 1 below.

TABLE 1
Non-Limiting Examples of Amino Acid Mutations of PTEN Protein

| Amino Acid | Non-limiting Examples of Amino Acid | Amino Acid |
|---|---|---|
| Position According | Mutation(s) At Each Position | Position According |
| to SEQ ID NO: 2 | Corresponding to SEQ ID NO: 2 | to SEQ ID NO: 1 |
| K13 | K13E | K13 |
| R15 | R15I, R15K | R15 |
| D24 | D24G, D24H, D24N | D24 |
| Y27 | Y27C, Y27N | Y27 |
| I33 | I33S, I33 deletion | I33 |
| M35 | M35V | M35 |
| G36 | G36R, G36E | G36 |
| P38 | P38S, P38L | P38 |
| L42 | L42R | L42 |
| G44 | G44D | G44 |
| V45 | V45A | V45 |
| H61 | H61R | |
| K66 | K66N | |
| I67 | I67K | |
| Y68 | Y68H, Y68C, Y68N | |
| C71 | C71Y | C56 |
| Y76 | Y76 deletion | Y61 |
| A79 | A79T | A64 |
| F81 | F81C | F66 |
| Y88 | Y88C | Y73 |
| D92 | D92N, D92H, D92G, D92E, D92Y, D92V, | D77 |
| | D92A | |
| H93 | H93R, H93D, H93Q, H93Y | H78 |
| P95 | P95S, P95L | P80 |
| P96 | P96L | P81 |
| I101 | I101T | I86 |
| C105 | C105Y, C105F | C90 |
| D107 | D107Y | D92 |
| L112 | L112V, L112P | L97 |
| A120 | A120T | A105 |
| H123 | H123Y | H108 |
| C124 | C124S, C124Y, C124A | C109 |
| A126 | A126T, A126S, A126V, A126D | A111 |
| G127 | G127E, G127V, G127R | G112 |
| G129 | G129R, G129E, G129V | G114 |
| R130 | R130Q, R130G, R130L, R130P | R115 |
| T131 | T131N | T116 |
| G132 | G132D, G132V, G132S | G117 |
| M134 | M134I | M119 |
| I135 | I135K, I135V | I120 |
| C136 | C136R, C136Y, C136F | C121 |
| R142 | R142W, R142Q | R127 |
| A151 | A151T | A136 |
| F154 | F154L | F139 |
| Y155 | Y155C, Y155H | Y140 |
| R159 | R159S, R159K, | R144 |
| D162 | D162V | D147 |
| G165 | G165R, G165E | G150 |
| T167 | T167A, | T152 |
| P169 | P169S | P154 |
| S170 | S170N, S170I | S155 |
| Q171 | Q171R | Q156 |
| R173 | R173C, R173H | R158 |
| Y174 | Y174D | Y159 |
| Y177 | Y177C | Y162 |
| Y178 | Y178 deletion | Y163 |
| V191 | V191A | V176 |
| M199 | M199 deletion | M184 |
| P204 | P204L | P189 |
| R234 | R234W | R219 |
| F241 | F241S | F226 |
| P246 | P246L | P231 |
| G251 | G251C, G251V | G236 |
| D252 | D252Y | D237 |
| I253 | I253N | I238 |
| H272 | H272P | H257 |
| F278 | F278L | F263 |
| L320 | L320S | L305 |
| L325 | L325R | L310 |
| P339 | P339S | P324 |
| F341 | F341C, F341V | F326 |
| T348 | T348I | T333 |

In some embodiments, the inactivating mutation of PTEN protein comprises or consists of a mutation at the amino acid position corresponding to D77 of SEQ ID NO: 1 (which also corresponds to D92 of SEQ ID NO: 2). In some embodiments, the inactivating mutation is an amino acid substitution of D77 of SEQ ID NO: 1. In some embodiments, the inactivating mutation corresponds to D77N, D77H, D77G, D77E, D77Y, D77V, or D77A of SEQ ID NO: 1. In some embodiments, the inactivating mutation corresponds to D77A of SEQ ID NO: 1. In some embodiments, the inactivating mutation corresponds to D77C of SEQ ID NO: 1. In some embodiments, the inactivating mutation corresponds to D77E of SEQ ID NO: 1. In some embodiments, the inactivating mutation corresponds to D77F of SEQ ID NO: 1. In some embodiments, the inactivating mutation corresponds to D77G of SEQ ID NO: 1. In some embodiments, the inactivating mutation corresponds to D77H of SEQ ID NO: 1. In some embodiments, the inactivating mutation corresponds to D771 of SEQ ID NO: 1. In some embodiments, the inactivating mutation corresponds to D77K of SEQ ID NO: 1. In some embodiments, the inactivating mutation corresponds to D77L of SEQ ID NO: 1. In some embodiments, the inactivating mutation corresponds to D77M of SEQ ID NO: 1. In some embodiments, the inactivating mutation corresponds to D77N of SEQ ID NO: 1. In some embodiments, the inactivating mutation corresponds to D77P of SEQ ID NO: 1. In some embodiments, the inactivating mutation corresponds to D77Q of SEQ ID NO: 1. In some embodiments, the inactivating mutation corresponds to D77R of SEQ ID NO: 1. In some embodiments, the inactivating mutation corresponds to D77S of SEQ ID NO: 1. In some embodiments, the inactivating mutation corresponds to D77T of SEQ ID NO: 1. In some embodiments, the inactivating mutation corresponds to D77V of SEQ ID NO: 1. In some embodiments, the inactivating mutation corresponds to D77W of SEQ ID NO: 1. In some embodiments, the inactivating mutation corresponds to D77Y of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to D77 of SEQ ID NO: 1 (which also corresponds to D92 of SEQ ID NO: 2).

In some embodiments, the inactivating mutation of PTEN protein comprises or consists of a mutation at the amino acid position corresponding to C109 of SEQ ID NO: 1 (which also corresponds to C124 of SEQ ID NO: 2). In some embodiments, the inactivating mutation is an amino acid substitution of C109 of SEQ ID NO: 1. In some embodiments, the inactivating mutation corresponds to C109A, C109S or C109Y of SEQ ID NO: 1. In some embodiments, the inactivating mutation corresponds to C109A of SEQ ID NO: 1. In some embodiments, the inactivating mutation corresponds to C109D of SEQ ID NO: 1. In some embodiments, the inactivating mutation corresponds to C109E of SEQ ID NO: 1. In some embodiments, the inactivating mutation corresponds to C109F of SEQ ID NO: 1. In some embodiments, the inactivating mutation corresponds to C109G of SEQ ID NO: 1. In some embodiments, the inactivating mutation corresponds to C109H of SEQ ID NO: 1. In some embodiments, the inactivating mutation corresponds to C1091 of SEQ ID NO: 1. In some embodiments, the inactivating mutation corresponds to C109K of SEQ ID NO: 1. In some embodiments, the inactivating mutation corresponds to C109L of SEQ ID NO: 1. In some embodiments, the inactivating mutation corresponds to C109M of SEQ ID NO: 1. In some embodiments, the inactivating mutation corresponds to C109N of SEQ ID NO: 1. In some embodiments, the inactivating mutation corresponds to C109P of SEQ ID NO: 1. In some embodiments, the inactivating mutation corresponds to C109Q of SEQ ID NO: 1. In some embodiments, the inactivating mutation corresponds to C109R of SEQ ID NO: 1. In some embodiments, the inactivating mutation corresponds to C109S of SEQ ID NO: 1. In some embodiments, the inactivating mutation corresponds to C109T of SEQ ID NO: 1. In some embodiments, the inactivating mutation corresponds to C109V of SEQ ID NO: 1. In some embodiments, the inactivating mutation corresponds to C109W of SEQ ID NO: 1. In some embodiments, the inactivating mutation corresponds to C109Y of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to C109 of SEQ ID NO: 1 (which also corresponds to C124 of SEQ ID NO: 2).

In some embodiments, the inactivating mutation of PTEN protein comprises or consists of a mutation at the amino acid position corresponding to G36 of SEQ ID NO: 1 (which also corresponds to G36 of SEQ ID NO: 2). In some embodiments, the inactivating mutation is an amino acid substitution of G36 of SEQ ID NO: 1. In some embodiments, the inactivating mutation corresponds to G36R or G36E of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to G36 of SEQ ID NO: 1.

In some embodiments, the inactivating mutation of PTEN protein comprises or consists of a mutation at the amino acid position corresponding to H108 of SEQ ID NO: 1 (which also corresponds to H123 of SEQ ID NO: 2). In some embodiments, the inactivating mutation is an amino acid substitution of H108 of SEQ ID NO: 1. In some embodiments, the inactivating mutation corresponds to H108Y of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to H108 of SEQ ID NO: 1.

In some embodiments, the inactivating mutation of PTEN protein comprises or consists of a mutation at the amino acid position corresponding to G114 of SEQ ID NO: 1 (which also corresponds to G129 of SEQ ID NO: 2). In some embodiments, the inactivating mutation is an amino acid substitution of G114 of SEQ ID NO: 1. In some embodiments, the inactivating mutation corresponds to G114R or G114E of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to G114 of SEQ ID NO: 1.

In some embodiments, the inactivating mutation of PTEN protein comprises or consists of a mutation at the amino acid position corresponding to R115 of SEQ ID NO: 1 (which also corresponds to R130 of SEQ ID NO: 2). In some embodiments, the inactivating mutation is an amino acid substitution of R115 of SEQ ID NO: 1. In some embodiments, the inactivating mutation corresponds to R115G, R115Q or R115L of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to R115 of SEQ ID NO: 1.

In some embodiments, the inactivating mutation of PTEN protein comprises or consists of a mutation at the amino acid position corresponding to R115 of SEQ ID NO: 1 (which also corresponds to R130 of SEQ ID NO: 2). In some embodiments, the inactivating mutation is an amino acid substitution of R115 of SEQ ID NO: 1. In some embodiments, the inactivating mutation corresponds to R115G, R115Q or R115L of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to R115 of SEQ ID NO: 1.

In some embodiments, the inactivating mutation of PTEN protein comprises or consists of a mutation at the amino acid position corresponding to R158 of SEQ ID NO: 1 (which also corresponds to R173 of SEQ ID NO: 2). In some embodiments, the inactivating mutation is an amino acid substitution of R158 of SEQ ID NO: 1. In some embodiments, the inactivating mutation corresponds to R158C or R158H of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to R158 of SEQ ID NO: 1.

In some embodiments, the inactivating mutation of PTEN protein comprises or consists of a mutation at the amino acid position corresponding to V176 of SEQ ID NO: 1 (which also corresponds to V191 of SEQ ID NO: 2). In some embodiments, the inactivating mutation is an amino acid substitution of V176 of SEQ ID NO: 1. In some embodiments, the inactivating mutation corresponds to V176A of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to V176 of SEQ ID NO: 1.

In some embodiments, the inactivating mutation of PTEN protein comprises or consists of a mutation at the amino acid position corresponding to T333 of SEQ ID NO: 1 (which also corresponds to T348 of SEQ ID NO: 2). In some embodiments, the inactivating mutation is an amino acid substitution of T333 of SEQ ID NO: 1. In some embodiments, the inactivating mutation corresponds to T3331 of SEQ ID NO: 1. In some embodiments, such an inactivation mutation results in a dominant negative PTEN protein. In some embodiments, the PTEN protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with any one of SEQ ID NO: 1-8, including all ranges and subranges therebetween. That is, in some embodiments, the present disclosure teaches a mutant PTEN protein with an amino acid substitution corresponding to T333 of SEQ ID NO: 1.

Non-limiting examples of nucleotide sequences encoding PTEN protein include SEQ ID NO: 9 (Bos Taurus), SEQ ID NO: 10 (Sus scrofa), SEQ ID NO: 11 (Ovis aries), SEQ ID NO: 12 ((′apra hircus), SEQ ID NO: 13 (Oryctolagus cuniculus), SEQ ID NO: 14 (Gallus gallus), SEQ ID NO: 15 (Anas platyrhynchos), and SEQ ID NO: 16 (Meleagris gallopavo). In some embodiments, the PTEN protein of the disclosure is encoded by a nucleotide sequence having at least 60%, at least 65%, at least 70%, at least 75%, at least 80%, at least 85%, at least 90%, at least 95%, at least 96%, at least 97%, at least 98%, at least 99%, at least 99.5%, or 100% identity to any one of SEQ ID NO: 9-16, including all ranges and subranges therebetween. In some embodiments, the PTEN protein of the disclosure is encoded by a nucleotide sequence having at least 60%, at least 65%, at least 70%, at least 75%, at least 80%, at least 85%, at least 90%, at least 95%, at least 96%, at least 97%, at least 98%, at least 99%, at least 99.5%, or 100% identity to SEQ ID NO: 9, including all ranges and subranges therebetween.

In some embodiments, the genetically modified cell line encodes a PTEN protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the PTEN activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the PTEN activity in the genetically modified cell line is reduced by at least 50% compared to that of a control cell line. In some embodiments, the PTEN activity in the genetically modified cell line is reduced by 100% (i.e., the PTEN activity is eliminated).

The PTEN activity refers generally to the signaling activity of PTEN. In some embodiments, the PTEN activity is determined by the expression level of PTEN in the cell (for example, when only wildtype PTEN protein is expressed in the cell). In some embodiments, the PTEN activity is determined by the PTEN phosphatase activity in the cell. In some embodiments, the PTEN activity is determined by the level/status of a related cell signaling molecule other than PTEN, by comparing the level/status of the related signaling molecule in the genetically modified cell line and in the control cell line without said genetic modification. In some embodiments, the PTEN activity is determined by the level of phosphatidylinositol-3, 4, 5-phosphate (PIP3) in the cell. In some embodiments, the PTEN activity is determined by the level of phosphorylated Akt in the cell. In some embodiments, the PTEN activity is determined by the level of phosphorylated FAK in the cell.

In some embodiments, PTEN activity is determined by the expression level of PTEN protein in the cell. In some embodiments, the expression level of PTEN protein is determined by Western blotting or ELISA using an anti-PTEN antibody. In some embodiments, the PTEN protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the PTEN protein expression/accumulation level in the genetically modified cell line is reduced by at least 50% compared to that of a control cell line.

In some embodiments, PTEN activity is determined by the PTEN phosphatase activity in the cell. In some embodiments, PTEN phosphatase activity is determined by a lipid phosphatase activity assay using immunoprecipitated PTEN from the cell line. In some embodiments, the lipid phosphatase activity assay utilizes PI (3,4,5) P3 as substrate and monitor the conversion of PI (3,4,5) P3 to PI (4,5) P2 or the release of phosphate as a result of PTEN phosphatase activity. Methods for determining PTEN lipid phosphatase activity in the cell immunoprecipitant are known in the art—for example, see Spinelli and Leslie, Methods Mol Biol. 2016; 1447:95-105., the content of which is incorporated by reference in its entirety. Alternatively, a commercially available PTEN phosphatase activity assay kit may be used following the manufacturer's recommended protocols. Examples of such kits include the PTEN Activity ELISA Kits offered by Echelon Biosciences (Product Number: K-4700) or Creative Diagnostics (Catalog No. DEIA-XYZ23). In some embodiments, the PTEN phosphatase activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the PTEN phosphatase activity in the genetically modified cell line is reduced by at least 50% compared to that of a control cell line.

In some embodiments, the PTEN activity is determined by the level of phosphatidylinositol-3, 4, 5-phosphate (PIP3) in the cell. In some embodiments, the cellular level of PIP3 can be determined using a PIP3 specific antibody (e.g., anti-PIP3 mouse IgM monoclonal RC6F8 by Invitrogen, Product #A-21328). In some embodiments, the cellular level of PIP3 can be determined using the mass assay described by van der Kaay et al., J Biol Chem. 1997 Feb. 28;272 (9): 5477-81, the content of which is incorporated by reference in its entirety. In some embodiments, the cellular level of PIP3 can be determined by a PIP3 specific biosensor—for example, an Akt-PH domain based biosensor described by Pierre-Eugene et al., PLOS One. 2012; 7 (7): e41992. the content of which is incorporated by reference in its entirety. In some embodiments, the cellular PIP3 level is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, for the genetically modified cell line compared to that of a control cell line. In some embodiments, the cellular level of PIP3 in the genetically modified cell line is increased by at least 50% compared to that of a control cell line.

In some embodiments, the PTEN activity is determined by the level of phosphorylated Akt proteins. In some embodiments, the phosphorylated Akt proteins comprise phosphorylation at a serine residue corresponding to Ser473 of human Akt protein. In some embodiments, the phosphorylated Akt protein comprises phosphorylation at a threonine residue corresponding to Thr308 of human Akt protein. In some embodiments, the level of phosphorylated Akt proteins can be determined by western blotting using an anti-phospho-Akt specific antibody (e.g., anti phosphor-Ser473 Akt antibody). In some embodiments, the level of phosphorylated Akt proteins is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, for the genetically modified cell line compared to that of a control cell line. In some embodiments, the level of phosphorylated Akt proteins in the genetically modified cell line is increased by at least 50% compared to that of a control cell line.

In some embodiments, the PTEN activity is determined by the tyrosine phosphorylation level of FAK. In some embodiments, the tyrosine phosphorylation level of FAK in the cells can be determined by immunoprecipitation of FAK protein followed by Western blotting analysis using pan-phosphotyrosine antibody. In some embodiments, the tyrosine phosphorylation level of FAK is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, for the genetically modified cell line compared to that of a control cell line. In some embodiments, the tyrosine phosphorylation level of FAK in the genetically modified cell line is increased by at least 50% compared to that of a control cell line.

In some embodiments, the PTEN activity is determined when the cell line is cultured in a minimum regular growth media. In some embodiments, the PTEN activity is determined when the cell line is cultured in a cell growth medium lacking or having reduced level of serum and/or one or more exogenous growth factors (e.g., as described infra). In some embodiments, the PTEN activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the PTEN activity is determined at basal level (i.e., without growth factor stimulation). In some embodiments, the PTEN activity is determined following growth factor stimulation. In some embodiments, the PTEN activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### CASP3 and CASP8

Caspases (cysteine-aspartic proteases) are proteolytic enzymes largely known for their role in controlling cell death and inflammation. Based on their function, mammalian caspase-2, -3, -7, -8, -9 and -10 are apoptotic caspases, where as caspase-1, -4, -5, -11 and -12 are involved in inflammation. The apoptotic caspases are subdivided into the initiators and the effectors based on the presence or absence of specific-protein interaction domains toward the N-terminus. Initiator caspases such as caspase-8 (CASP8) and -10 comprise death effector domains (DED) or caspase-recruitment domains (CARD; caspase-2, -9, -1 and -11), which mediate their dimerization and/or recruitment into larger complexes to facilitate their activation. On the other hand, effectors such as caspase-3 (CASP3) do not comprise DED or CARD domain. Initiator caspase activation during apoptosis is mediated by two main pathways; the mitochondrial or Bcl-2-regulated (intrinsic) pathway and the death receptor (extrinsic) pathway. The intrinsic pathway is activated in response to cellular stress (e.g., cytotoxic drugs, DNA damage) and is regulated by the Bcl-2 family of proteins. This pathway involves activation of the pro-apoptotic effectors BAX and BAK, which induce mitochondrial outer membrane permeabilization (MOMP) and cytochrome c release. Apaf-1 (apoptotic protease-activating factor 1) associates with cytochrome c into a large multimeric complex called the apoptosome to activate caspase-9. Death receptor-mediated apoptosis is initiated following ligand -binding and activation of the death-domain-containing tumor necrosis receptor superfamily (e.g., TNFR, Fas, TRAIL). This mediates recruitment and activation of caspase-8 or -10, through the death-inducing signaling complex (DISC) comprising FAS-associated death domain protein (FADD) and/or TNFR-associated death domain protein (TRADD) and other components. Caspase-8 also cleaves BID to a truncated form (tBID), which engages the mitochondrial pathway to amplify the apoptotic response. Once initiator caspases are activated through the extrinsic or intrinsic apoptosis pathways, they mediate activation of effector caspases-3, -6 and -7. See Shalini et. al., Cell Death Differ. 2015 April; 22 (4): 526-539; Stennicke and Salvesen, J Biol Chem. 1997 Oct. 10;272 (41): 25719-23; Kim et al., Gastroenterology. 2003 September; 125 (3): 708-15; and Ponder and Boise, Cell Death Discovery, volume 5:56 (2019), the content of each of which is incorporated by reference in its entirety.

In some embodiments, the cell line of the disclosure comprises a genetic modification selected from the group consisting of: a) knock-out of the endogenous CASP3 gene; b) knock-down of the endogenous CASP3 gene (e.g., via CRISPRi or RNAi); c) knock-in of a gene encoding a dominant negative CASP3 protein; d) inactivation of the endogenous CASP3 gene; and e) any combination thereof.

In some embodiments, the genetically modified cell line with CASP3 knock-down expresses the endogenous CASP3 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous CASP3 protein in a control cell line. In some embodiments, the genetically modified cell line with CASP3 knock-down expresses the endogenous CASP3 protein at a level that is no more than 10% of the expression level of the endogenous CASP3 protein in a control cell line.

In some embodiments, the disclosure provides a genetically modified cell line comprising a recombinant nucleic acid encoding a CASP3 protein (i.e., knock-in CASP3 protein). In some embodiments, the knock-in CASP3 protein comprises one or more inactivating mutations. In some embodiments the knock-in CASP3 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in CASP3 protein is derived from the same animal species as the genetically modified cell line. In some embodiments, the knock-in CASP3 protein is derived from a different animal species from the genetically modified cell line. In some embodiments, the genetically modified cell line comprises knock-in CASP3 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous CASP3 gene. In some embodiments, such inactivation comprises introducing one or more inactivating mutations to the endogenous CASP3 protein coding gene. In some embodiments, such inactivation comprises modifying the endogenous CASP3 gene so that it expresses a dominant negative CASP3 protein. In some embodiments, only one copy of the endogenous CASP3 gene/allele is inactivated (i.e., heterozygous inactivation). In some embodiments, both copies of the endogenous CASP3 gene/alleles are inactivated (i.e., homozygous inactivation).

An exemplary CASP3 protein sequence is provided herein as SEQ ID NO: 42 (Bos Taurus). In some embodiments, the CASP3 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 42, including all ranges and subranges therebetween. In some embodiments, the CASP3 protein of the disclosure comprises or consists of an amino acid sequence having up to 1, 2, 3, 4, 5, 6, 7, 8, 9, or 10 amino acids different from the amino acid sequence according to SEQ ID NO: 42. In some embodiments, the CASP3 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 42, including all ranges or subranges therebetween. In some embodiments, the CASP3 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 42, including all ranges and subranges therebetween, wherein the sequence difference with SEQ ID NO: 42 consists of conservative amino acid substitutions (other than the one or more inactivating mutations of the disclosure).

In some embodiments, the CASP3 protein (knock-in and/or endogenous) of the disclosure comprises one or more inactivating mutations. In some embodiments, the CASP3 protein comprising the one or more inactivating mutations is a dominant negative protein. In some embodiments, the genetic modification comprises a knock in gene encoding for a CASP3 protein with a dominant negative inactivating mutation.

In some embodiments, the inactivating mutation of CASP3 protein comprises a single amino acid substitution. In some embodiments, the inactivating mutation of CASP3 protein comprises a single amino acid insertion or deletion. In some embodiments, the inactivating mutation of CASP3 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues).

In some embodiments, the CASP3 protein of the disclosure comprises or consists of an inactivating mutation at one or more amino acid positions corresponding to those in SEQ ID NO: 42. In some embodiments, the inactivating mutation of CASP3 protein reduces the protease activity of CASP3 by at least 5%, at least 10%, at least 15, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to the corresponding wildtype CASP3 protein. In some embodiments, the inactivating mutation of CASP3 protein reduces the protease activity of CASP3 by at least 50% compared to the corresponding wildtype CASP3 protein.

In some embodiments, the inactivating mutation of CASP3 protein comprises or consists of a mutation at one or more amino acid positions within the active center of the protease domain. In some embodiments, the inactivating mutation of CASP3 protein comprises or consists of a mutation at the amino acid position corresponding to C163 of SEQ ID NO: 42. In some embodiments, the inactivating mutation corresponds to an amino acid substitution of C163 of SEQ ID NO: 42. In some embodiments, the inactivating mutation corresponds to C163A of SEQ ID NO: 42. In some embodiments, the inactivating mutation corresponds to C163S of SEQ ID NO: 42. In some embodiments, the CASP3 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 42, including all ranges and subranges therebetween.

In some embodiments, the genetically modified cell line encodes a CASP3 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the CASP3 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the CASP3 activity in the genetically modified cell line is reduced by at least 50% compared to that of a control cell line. In some embodiments, the CASP3 activity in the genetically modified cell line is reduced by 100% (i.e., the CASP3 activity is eliminated).

In some embodiments, the cell line of the disclosure comprises a genetic modification selected from the group consisting of: a) knock-out of the endogenous CASP8 gene; b) knock-down of the endogenous CASP8 gene (e.g., via CRISPRi or RNAi); c) knock-in of a gene encoding a dominant negative CASP8 protein; d) inactivation of the endogenous CASP8 gene; and e) any combination thereof.

In some embodiments, the genetically modified cell line with CASP8 knock-down expresses the endogenous CASP8 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous CASP8 protein in a control cell line. In some embodiments, the genetically modified cell line with CASP8 knock-down expresses the endogenous CASP8 protein at a level that is no more than 10% of the expression level of the endogenous CASP8 protein in a control cell line.

In some embodiments, the disclosure provides a genetically modified cell line comprising a recombinant nucleic acid encoding a CASP8 protein (i.e., knock-in CASP8 protein). In some embodiments, the knock-in CASP8 protein comprises one or more inactivating mutations. In some embodiments the knock-in CASP8 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in CASP8 protein is derived from the same animal species as the genetically modified cell line. In some embodiments, the knock-in CASP8 protein is derived from a different animal species from the genetically modified cell line. In some embodiments, the genetically modified cell line comprises knock-in CASP8 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous CASP8 gene. In some embodiments, such inactivation comprises introducing one or more inactivating mutations to the endogenous CASP8 protein coding gene. In some embodiments, such inactivation comprises modifying the endogenous CASP8 gene so that it expresses a dominant negative CASP8 protein. In some embodiments, only one copy of the endogenous CASP8 gene/allele is inactivated (i.e., heterozygous inactivation). In some embodiments, both copies of the endogenous CASP8 gene/alleles are inactivated (i.e., homozygous inactivation).

An exemplary CASP8 protein sequence is provided herein as SEQ ID NO: 44 (Bos Taurus). In some embodiments, the CASP8 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 44, including all ranges and subranges therebetween. In some embodiments, the CASP8 protein of the disclosure comprises or consists of an amino acid sequence having up to 1, 2, 3, 4, 5, 6, 7, 8, 9, or 10 amino acids different from the amino acid sequence according to SEQ ID NO: 44. In some embodiments, the CASP8 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 44, including all ranges or subranges therebetween. In some embodiments, the CASP8 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 44, including all ranges and subranges therebetween, wherein the sequence difference with SEQ ID NO: 44 consists of conservative amino acid substitutions (other than the one or more inactivating mutations of the disclosure).

In some embodiments, the CASP8 protein (knock-in and/or endogenous) of the disclosure comprises one or more inactivating mutations. In some embodiments, the CASP8 protein comprising the one or more inactivating mutations is a dominant negative protein. In some embodiments, the genetic modification comprises a knock in gene encoding for a CASP8 protein with a dominant negative inactivating mutation.

In some embodiments, the inactivating mutation of CASP8 protein comprises a single amino acid substitution. In some embodiments, the inactivating mutation of CASP8 protein comprises a single amino acid insertion or deletion. In some embodiments, the inactivating mutation of CASP8 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues).

In some embodiments, the CASP8 protein of the disclosure comprises or consists of an inactivating mutation at one or more amino acid positions corresponding to those in SEQ ID NO: 44. In some embodiments, the inactivating mutation of CASP8 protein reduces the protease activity of CASP8 by at least 5%, at least 10%, at least 15, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to the corresponding wildtype CASP8 protein. In some embodiments, the inactivating mutation of CASP8 protein reduces the protease activity of CASP8 by at least 50% compared to the corresponding wildtype CASP8 protein.

In some embodiments, the inactivating mutation of CASP8 protein comprises or consists of a mutation at one or more amino acid positions within the active center of the protease domain. In some embodiments, the inactivating mutation of CASP8 protein comprises or consists of a mutation at the amino acid position corresponding to C366 of SEQ ID NO: 44. In some embodiments, the inactivating mutation corresponds to an amino acid substitution C366 of SEQ ID NO: 44. In some embodiments, the inactivating mutation corresponds to C366A of SEQ ID NO: 44. In some embodiments, the inactivating mutation corresponds to C366S of SEQ ID NO: 44. In some embodiments, the CASP8 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 44, including all ranges and subranges therebetween.

In some embodiments, the inactivating mutation of CASP8 protein comprises or consists of a mutation at the amino acid position corresponding to R419 of SEQ ID NO: 44. In some embodiments, the inactivating mutation corresponds to an amino acid substitution R419 of SEQ ID NO: 44. In some embodiments, the inactivating mutation corresponds to R419G of SEQ ID NO: 44. In some embodiments, the CASP8 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 44, including all ranges and subranges therebetween.

In some embodiments, the inactivating mutation of CASP8 protein comprises or consists of a mutation at the amino acid position corresponding to Q372 of SEQ ID NO: 44. In some embodiments, the inactivating mutation corresponds to an amino acid substitution Q372 of SEQ ID NO: 44. In some embodiments, the inactivating mutation corresponds to Q372H of SEQ ID NO: 44. In some embodiments, the CASP8 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 44, including all ranges and subranges therebetween.

In some embodiments, the inactivating mutation of CASP8 protein comprises or consists of a mutation at the amino acid position corresponding to R162 of SEQ ID NO: 44. In some embodiments, the inactivating mutation corresponds to an amino acid substitution R162 of SEQ ID NO: 44. In some embodiments, the inactivating mutation corresponds to R1621 of SEQ ID NO: 44. In some embodiments, the CASP8 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 44, including all ranges and subranges therebetween.

In some embodiments, the genetically modified cell line encodes a CASP8 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the CASP8 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the CASP8 activity in the genetically modified cell line is reduced by at least 50% compared to that of a control cell line. In some embodiments, the CASP8 activity in the genetically modified cell line is reduced by 100% (i.e., the CASP8 activity is eliminated).

In some embodiments, the reduction of CASP3 and/or CASP8 activity renders anoikis resistance to the genetically modified cell line and therefore facilitates its adaptation to suspension culture. In some embodiments, the reduction of CASP3 and/or CASP8 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

The CASP3/CASP8 activity refers generally to the signaling activity of CASP3/CASP8. In some embodiments, the CASP3/CASP8 activity is determined by the expression level of CASP3/CASP8 in the cell (for example, when only wildtype CASP3/CASP8 protein is expressed in the cell). In some embodiments, the CASP3/CASP8 activity is determined by the protease activity in the cell. In some embodiments, the CASP3/CASP8 activity is determined by the level/status of a related cell signaling molecule other than CASP3/CASP8, by comparing the level/status of the related signaling molecule in the genetically modified cell line and in the control cell line without said genetic modification.

In some embodiments, the CASP3/CASP8 activity is determined by the expression level of CASP3/CASP8 protein in the cell. In some embodiments, the expression level of CASP3/CASP8 protein is determined by Western blotting or ELISA using a specific antibody. In some embodiments, the CASP3 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the CASP3 expression level in the genetically modified cell line is reduced by at least 50% compared to that of a control cell line. In some embodiments, the CASP8 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the CASP8 protein expression/accumulation level in the genetically modified cell line is reduced by at least 50% compared to that of a control cell line.

In some embodiments, the CASP3/CASP8 activity is determined by the corresponding protease activity. In some embodiments, the caspase protease activity is determined using the biochemical method described in Stennicke and Salvesen, J Biol Chem. 1997 Oct. 10;272 (41): 25719-23. In some embodiments, the protease activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the protease activity in the genetically modified cell line is reduced by at least 50% compared to that of a control cell line.

In some embodiments, the caspase activity is determined when the cell line is cultured in a minimum regular growth media. In some embodiments, the caspase activity is determined when the cell line is cultured in a cell growth medium lacking or having reduced level of serum and/or one or more exogenous growth factors (e.g., as described infra). In some embodiments, the caspase activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the caspase activity is determined at basal level (i.e., without growth factor stimulation). In some embodiments, the caspase activity is determined following growth factor stimulation. In some embodiments, the caspase activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### CDH1

Cadherin 1 (CDH1), also known as E-cadherin, belongs to class I classical cadherin and plays an important role in the intercellular adhesion connection. Cadherin 1 protein is a transmembrane glycoprotein consisting of three domains: extracellular domains (ECD), transmembrane domains and intracellular domains (ICD). ECD is composed of multiple cadherin repeats and calcium ion binding sites, which mediate the adhesion function; ICD interacts with α-, β-catenin and other catenin family members. It binds and connects to the cytoskeleton of actin to maintain the stability of cell structure, inhibiting the movement of individual cells, and participating in cell signal transduction. The abnormal expression of cadherin 1 (CDH1) protein has a significant impact on the interaction between cells, leading to the destruction of the dynamic balance of epithelial tissues, making it easier for cells to gain mobility and invasiveness. See Zhao et. al., Front Mol Biosci. 2021; 8:689139, the content of which is incorporated by reference in its entirety.

In some embodiments, the cell line of the disclosure comprises a genetic modification selected from the group consisting of: a) knock-out of the endogenous CDH1 gene; b) knock-down of the endogenous CDH1 gene (e.g., via CRISPRi or RNAi); c) knock-in of a gene encoding a dominant negative CDH1 protein; d) inactivation of the endogenous CDH1 gene; and e) any combination thereof.

In some embodiments, the genetically modified cell line with CDH1 knock-down expresses the endogenous CDH1 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous CDH1 protein in a control cell line. In some embodiments, the genetically modified cell line with CDH1 knock-down expresses the endogenous CDH1 protein at a level that is no more than 10% of the expression level of the endogenous CDH1 protein in a control cell line.

In some embodiments, the disclosure provides a genetically modified cell line comprising a recombinant nucleic acid encoding a CDH1 protein (i.e., knock-in CDH1 protein). In some embodiments, the knock-in CDH1 protein comprises one or more inactivating mutations. In some embodiments the knock-in CDH1 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in CDH1 protein is derived from the same animal species as the genetically modified cell line. In some embodiments, the knock-in CDH1 protein is derived from a different animal species from the genetically modified cell line. In some embodiments, the genetically modified cell line comprises knock-in CDH1 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous CDH1 gene. In some embodiments, such inactivation comprises introducing one or more inactivating mutations to the endogenous CDH1 protein coding gene. In some embodiments, such inactivation comprises modifying the endogenous CDH1 gene so that it expresses a dominant negative CDH1 protein. In some embodiments, only one copy of the endogenous CDH1 gene/allele is inactivated (i.e., heterozygous inactivation). In some embodiments, both copies of the endogenous CDH1 gene/alleles are inactivated (i.e., homozygous inactivation).

An exemplary CDH1 protein sequence is provided herein as SEQ ID NO: 46 (Bos Taurus). In some embodiments, the CDH1 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 46, including all ranges and subranges therebetween. In some embodiments, the CDH1 protein of the disclosure comprises or consists of an amino acid sequence having up to 1, 2, 3, 4, 5, 6, 7, 8, 9, or 10 amino acids different from the amino acid sequence according to SEQ ID NO: 46. In some embodiments, the CDH1 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 46, including all ranges or subranges therebetween. In some embodiments, the CDH1 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 46, including all ranges and subranges therebetween, wherein the sequence difference with SEQ ID NO: 46 consists of conservative amino acid substitutions (other than the one or more inactivating mutations of the disclosure).

In some embodiments, the CDH1 protein (knock-in and/or endogenous) of the disclosure comprises one or more inactivating mutations. In some embodiments, the CDH1 protein comprising the one or more inactivating mutations is a dominant negative protein. In some embodiments, the genetic modification comprises a knock in gene encoding for a CDH1 protein with a dominant negative inactivating mutation.

In some embodiments, the inactivating mutation of CDH1 protein comprises a single amino acid substitution. In some embodiments, the inactivating mutation of CDH1 protein comprises a single amino acid insertion or deletion. In some embodiments, the inactivating mutation of CDH1 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). Non-limiting examples of CDH1 protein mutations are described in Figueiredo et. al., J Med Genet. 2019 April;56 (4): 199-208, the content of which is incorporated in its entirety.

In some embodiments, the genetically modified cell line encodes a CDH1 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the CDH1 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the CDH1 activity in the genetically modified cell line is reduced by at least 50% compared to that of a control cell line. In some embodiments, the CDH1 activity in the genetically modified cell line is reduced by 100% (i.e., the CDH1 activity is eliminated).

In some embodiments, the reduction of CDH1 activity renders anoikis resistance to the genetically modified cell line and therefore facilitates its adaptation to suspension culture. In some embodiments, the reduction of CDH1 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the CDH1 activity is determined by the expression level of CDH1 protein in the cell. In some embodiments, the expression level of CDH1 protein is determined by Western blotting or ELISA using a specific antibody. In some embodiments, the CDH1 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the CDH1 protein expression/accumulation level in the genetically modified cell line is reduced by at least 50% compared to that of a control cell line.

In some embodiments, the CDH1 activity is determined when the cell line is cultured in a minimum regular growth media. In some embodiments, the CDH1 activity is determined when the cell line is cultured in a cell growth medium lacking or having reduced level of serum and/or one or more exogenous growth factors (e.g., as described infra). In some embodiments, the CDH1 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the CDH1 activity is determined at basal level (i.e., without growth factor stimulation). In some embodiments, the CDH1 activity is determined following growth factor stimulation. In some embodiments, the CDH1 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### ITGB1

Integrin beta-1 (ITGB1), also known as CD29, is a cell surface receptor that in humans is encoded by the ITGB1 gene. It may associate with integrin alpha 1 and integrin alpha 2 to form integrin complexes which function as collagen receptors. It may also form dimers with integrin alpha 3 to form integrin receptors for netrin 1 and reelin. Integrin beta-1 may exist as different isoforms via alternative splicing and often plays a role in cell adhesion. See Chen et al., PLOS One. 2012; 7 (12): e52886, the content of which is incorporated by reference in its entirety.

In some embodiments, the cell line of the disclosure comprises a genetic modification selected from the group consisting of: a) knock-out of the endogenous ITGB1 gene; b) knock-down of the endogenous ITGB1 gene (e.g., via CRISPRi or RNAi); c) knock-in of a gene encoding a dominant negative ITGB1 protein; d) inactivation of the endogenous ITGB1 gene; and e) any combination thereof.

In some embodiments, the genetically modified cell line with ITGB1 knock-down expresses the endogenous ITGB1 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous ITGB1 protein in a control cell line. In some embodiments, the genetically modified cell line with ITGB1 knock-down expresses the endogenous ITGB1 protein at a level that is no more than 10% of the expression level of the endogenous ITGB1 protein in a control cell line.

In some embodiments, the disclosure provides a genetically modified cell line comprising a recombinant nucleic acid encoding a ITGB1 protein (i.e., knock-in ITGB1 protein). In some embodiments, the knock-in ITGB1 protein comprises one or more inactivating mutations. In some embodiments the knock-in ITGB1 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in ITGB1 protein is derived from the same animal species as the genetically modified cell line. In some embodiments, the knock-in ITGB1 protein is derived from a different animal species from the genetically modified cell line. In some embodiments, the genetically modified cell line comprises knock-in ITGB1 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous ITGB1 gene. In some embodiments, such inactivation comprises introducing one or more inactivating mutations to the endogenous ITGB1 protein coding gene. In some embodiments, such inactivation comprises modifying the endogenous ITGB1 gene so that it expresses a dominant negative ITGB1 protein. In some embodiments, only one copy of the endogenous ITGB1 gene/allele is inactivated (i.e., heterozygous inactivation). In some embodiments, both copies of the endogenous ITGB1 gene/alleles are inactivated (i.e., homozygous inactivation).

An exemplary ITGB1 protein sequence is provided herein as SEQ ID NO: 52 (Bos Taurus). In some embodiments, the ITGB1 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 52, including all ranges and subranges therebetween. In some embodiments, the ITGB1 protein of the disclosure comprises or consists of an amino acid sequence having up to 1, 2, 3, 4, 5, 6, 7, 8, 9, or 10 amino acids different from the amino acid sequence according to SEQ ID NO: 52. In some embodiments, the ITGB1 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 52, including all ranges or subranges therebetween. In some embodiments, the ITGB1 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 52, including all ranges and subranges therebetween, wherein the sequence difference with SEQ ID NO: 52 consists of conservative amino acid substitutions (other than the one or more inactivating mutations of the disclosure).

In some embodiments, the ITGB1 protein (knock-in and/or endogenous) of the disclosure comprises one or more inactivating mutations. In some embodiments, the ITGB1 protein comprising the one or more inactivating mutations is a dominant negative protein. In some embodiments, the genetic modification comprises a knock in gene encoding for a ITGB1 protein with a dominant negative inactivating mutation.

In some embodiments, the inactivating mutation of ITGB1 protein comprises a single amino acid substitution. In some embodiments, the inactivating mutation of ITGB1 protein comprises a single amino acid insertion or deletion. In some embodiments, the inactivating mutation of ITGB1 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). Non-limiting examples of ITGB1 protein mutations are described in Liu et al., Mol Cell. 2013 Feb. 21;49 (4): 719-29, the content of which is incorporated in its entirety. In some embodiments, the inactivating mutation of ITGB1 protein comprises one or more mutations in the C-terminal region corresponding to amino acids 781-802 of SEQ ID NO: 52, for example at N793 and/or Y796 of SEQ ID NO: 52, wherein the inactivating mutation disrupts its binding to Integrin Cytoplasmic Associated Protein-1 (ICAP1).

In some embodiments, the genetically modified cell line encodes a ITGB1 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure.

In some embodiments, the ITGB1 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the ITGB1 activity in the genetically modified cell line is reduced by at least 50% compared to that of a control cell line. In some embodiments, the ITGB1 activity in the genetically modified cell line is reduced by 100% (i.e., the ITGB1 activity is eliminated).

In some embodiments, the reduction of ITGB1 activity renders anoikis resistance to the genetically modified cell line and therefore facilitates its adaptation to suspension culture. In some embodiments, the reduction of ITGB1 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the ITGB1 activity is determined by the expression level of ITGB1 protein in the cell. In some embodiments, the expression level of ITGB1 protein is determined by Western blotting or ELISA using a specific antibody. In some embodiments, the ITGB1 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the ITGB1 protein expression/accumulation level in the genetically modified cell line is reduced by at least 50% compared to that of a control cell line.

In some embodiments, the ITGB1 activity is determined when the cell line is cultured in a minimum regular growth media. In some embodiments, the ITGB1 activity is determined when the cell line is cultured in a cell growth medium lacking or having reduced level of serum and/or one or more exogenous growth factors (e.g., as described infra). In some embodiments, the ITGB1 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the ITGB1 activity is determined at basal level (i.e., without growth factor stimulation). In some embodiments, the ITGB1 activity is determined following growth factor stimulation. In some embodiments, the ITGB1 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### CDKN1A/p21

Cyclin-dependent kinase inhibitor 1A (CDKN1A), also known as p21, is encoded by the gene CDKN1A (interchangeably referred to as “p21 gene” herein). It is a pivotal cell cycle regulator ensuring genomic stability and often deregulated in cancer. Due to the lack of a defined tertiary structure, p21 is able to interact with a number of proteins involved in many key biological processes, acting through a “folding-on-binding mechanism” with a prominent binding promiscuity. The short-lived p21 protein is degraded via different ubiquitin-dependent and -independent pathways. Reversible protein phosphorylation by diverse kinases serves as an additional posttranslational mechanism controlling p21's function, localization, binding partners, stability and degradation. In particular, phosphorylation at distinct sites by various kinases can cause the cytoplasmic translocation of p21 greatly affecting its functionality and the therapeutic response of many chemotherapeutic interventions. Importantly, p21 is not solely acting as a tumor suppressor but also as an oncogene depending on the cellular context leading to a negative or rather positive impact on tumor development and progression. See Kreis et al., Cancers (Basel). 2019 Aug. 21; 11 (9): 1220; Roninson et al., Cancer Lett. 2002 May 8;179 (1): 1-14, the content of each of which is incorporated by reference in its entirety.

In some embodiments, the cell line of the disclosure comprises a genetic modification selected from the group consisting of: a) knock-out of the endogenous p21 gene; b) knock-down of the endogenous p21 gene (e.g., via CRISPRi or RNAi); c) knock-in of a gene encoding a dominant negative p21 protein; d) inactivation of the endogenous p21 gene; and e) any combination thereof.

In some embodiments, the genetically modified cell line with p21 knock-down expresses the endogenous p21 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous p21 protein in a control cell line. In some embodiments, the genetically modified cell line with p21 knock-down expresses the endogenous p21 protein at a level that is no more than 10% of the expression level of the endogenous p21 protein in a control cell line.

In some embodiments, the disclosure provides a genetically modified cell line comprising a recombinant nucleic acid encoding a p21 protein (i.e., knock-in p21 protein). In some embodiments, the knock-in p21 protein comprises one or more inactivating mutations. In some embodiments the knock-in p21 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in p21 protein is derived from the same animal species as the genetically modified cell line. In some embodiments, the knock-in p21 protein is derived from a different animal species from the genetically modified cell line. In some embodiments, the genetically modified cell line comprises knock-in p21 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous p21 gene. In some embodiments, such inactivation comprises introducing one or more inactivating mutations to the endogenous p21 protein coding gene. In some embodiments, such inactivation comprises modifying the endogenous p21 gene so that it expresses a dominant negative p21 protein. In some embodiments, only one copy of the endogenous p21 gene/allele is inactivated (i.e., heterozygous inactivation). In some embodiments, both copies of the endogenous p21 gene/alleles are inactivated (i.e., homozygous inactivation).

An exemplary p21 protein sequence is provided herein as SEQ ID NO: 56 (Bos Taurus). In some embodiments, the p21 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 56, including all ranges and subranges therebetween. In some embodiments, the p21 protein of the disclosure comprises or consists of an amino acid sequence having up to 1, 2, 3, 4, 5, 6, 7, 8, 9, or 10 amino acids different from the amino acid sequence according to SEQ ID NO: 56. In some embodiments, the p21 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 56, including all ranges or subranges therebetween. In some embodiments, the p21 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 56, including all ranges and subranges therebetween, wherein the sequence difference with SEQ ID NO: 56 consists of conservative amino acid substitutions (other than the one or more inactivating mutations of the disclosure).

In some embodiments, the p21 protein (knock-in and/or endogenous) of the disclosure comprises one or more inactivating mutations. In some embodiments, the p21 protein comprising the one or more inactivating mutations is a dominant negative protein. In some embodiments, the genetic modification comprises a knock in gene encoding for a p21 protein with a dominant negative inactivating mutation.

In some embodiments, the inactivating mutation of p21 protein comprises a single amino acid substitution. In some embodiments, the inactivating mutation of p21 protein comprises a single amino acid insertion or deletion. In some embodiments, the inactivating mutation of p21 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). Non-limiting examples of p21 protein mutations are described in Welcker et al., Cancer Res. 1998 Nov. 15;58 (22): 5053-6, the content of which is incorporated in its entirety.

In some embodiments, the genetically modified cell line encodes a p21 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the p21 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p21 activity in the genetically modified cell line is reduced by at least 50% compared to that of a control cell line. In some embodiments, the p21 activity in the genetically modified cell line is reduced by 100% (i.e., the p21 activity is eliminated).

In some embodiments, the reduction of p21 activity renders anoikis resistance to the genetically modified cell line and therefore facilitates its adaptation to suspension culture. In some embodiments, the reduction of p21 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the p21 activity is determined by the expression level of p21 protein in the cell. In some embodiments, the expression level of p21 protein is determined by Western blotting or ELISA using a specific antibody. In some embodiments, the p21 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p21 protein expression/accumulation level in the genetically modified cell line is reduced by at least 50% compared to that of a control cell line.

In some embodiments, the p21 activity is determined when the cell line is cultured in a minimum regular growth media. In some embodiments, the p21 activity is determined when the cell line is cultured in a cell growth medium lacking or having reduced level of serum and/or one or more exogenous growth factors (e.g., as described infra). In some embodiments, the p21 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the p21 activity is determined at basal level (i.e., without growth factor stimulation). In some embodiments, the p21 activity is determined following growth factor stimulation. In some embodiments, the p21 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### CDKN1B/p27

Cyclin-dependent kinase inhibitor 1B (CDKN1B), also known as p27, is an enzyme inhibitor that in humans is encoded by the CDKN1B gene (used interchangeably with “p27 gene” herein). p27 protein belongs to the Cip/Kip family of cyclin dependent kinase (CDK) inhibitor proteins. It binds to and prevents the activation of cyclin E-CDK2 or cyclin D-CDK4 complexes, and thus controls the cell cycle progression at G1. It is often referred to as a cell cycle inhibitor protein because its major function is to stop or slow down the cell division cycle. See Chiarlie et al., Breast Cancer Research. 3 (2): 91-4. the content of which is incorporated by reference in its entirety.

In some embodiments, the cell line of the disclosure comprises a genetic modification selected from the group consisting of: a) knock-out of the endogenous p27 gene; b) knock-down of the endogenous p27 gene (e.g., via CRISPRi or RNAi); c) knock-in of a gene encoding a dominant negative p27 protein; d) inactivation of the endogenous p27 gene; and e) any combination thereof.

In some embodiments, the genetically modified cell line with p27 knock-down expresses the endogenous p27 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous p27 protein in a control cell line. In some embodiments, the genetically modified cell line with p27 knock-down expresses the endogenous p27 protein at a level that is no more than 10% of the expression level of the endogenous p27 protein in a control cell line.

In some embodiments, the disclosure provides a genetically modified cell line comprising a recombinant nucleic acid encoding a p27 protein (i.e., knock-in p27 protein). In some embodiments, the knock-in p27 protein comprises one or more inactivating mutations. In some embodiments the knock-in p27 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in p27 protein is derived from the same animal species as the genetically modified cell line. In some embodiments, the knock-in p27 protein is derived from a different animal species from the genetically modified cell line. In some embodiments, the genetically modified cell line comprises knock-in p27 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous p27 gene. In some embodiments, such inactivation comprises introducing one or more inactivating mutations to the endogenous p27 protein coding gene. In some embodiments, such inactivation comprises modifying the endogenous p27 gene so that it expresses a dominant negative p27 protein. In some embodiments, only one copy of the endogenous p27 gene/allele is inactivated (i.e., heterozygous inactivation). In some embodiments, both copies of the endogenous p27 gene/alleles are inactivated (i.e., homozygous inactivation).

An exemplary p27 protein sequence is provided herein as SEQ ID NO: 58 (Bos Taurus). In some embodiments, the p27 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 58, including all ranges and subranges therebetween. In some embodiments, the p27 protein of the disclosure comprises or consists of an amino acid sequence having up to 1, 2, 3, 4, 5, 6, 7, 8, 9, or 10 amino acids different from the amino acid sequence according to SEQ ID NO: 58. In some embodiments, the p27 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 58, including all ranges or subranges therebetween. In some embodiments, the p27 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 58, including all ranges and subranges therebetween, wherein the sequence difference with SEQ ID NO: 58 consists of conservative amino acid substitutions (other than the one or more inactivating mutations of the disclosure).

In some embodiments, the p27 protein (knock-in and/or endogenous) of the disclosure comprises one or more inactivating mutations. In some embodiments, the p27 protein comprising the one or more inactivating mutations is a dominant negative protein. In some embodiments, the genetic modification comprises a knock in gene encoding for a p27 protein with a dominant negative inactivating mutation.

In some embodiments, the inactivating mutation of p27 protein comprises a single amino acid substitution. In some embodiments, the inactivating mutation of p27 protein comprises a single amino acid insertion or deletion. In some embodiments, the inactivating mutation of p27 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). Non-limiting examples of p27 protein mutations are described in Cusan et. al., Front Endocrinol (Lausanne). 2018 Jul. 17; 9:393; Alrezk et al., Endocr Relat Cancer. 2017 October; 24 (10): T195-T208, the content of each which is incorporated in its entirety.

In some embodiments, the genetically modified cell line encodes a p27 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the p27 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p27 activity in the genetically modified cell line is reduced by at least 50% compared to that of a control cell line. In some embodiments, the p27 activity in the genetically modified cell line is reduced by 100% (i.e., the p27 activity is eliminated).

In some embodiments, the reduction of p27 activity renders anoikis resistance to the genetically modified cell line and therefore facilitates its adaptation to suspension culture. In some embodiments, the reduction of p27 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the p27 activity is determined by the expression level of p27 protein in the cell. In some embodiments, the expression level of p27 protein is determined by Western blotting or ELISA using a specific antibody. In some embodiments, the p27 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p27 protein expression/accumulation level in the genetically modified cell line is reduced by at least 50% compared to that of a control cell line.

In some embodiments, the p27 activity is determined when the cell line is cultured in a minimum regular growth media. In some embodiments, the p27 activity is determined when the cell line is cultured in a cell growth medium lacking or having reduced level of serum and/or one or more exogenous growth factors (e.g., as described infra). In some embodiments, the p27 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the p27 activity is determined at basal level (i.e., without growth factor stimulation). In some embodiments, the p27 activity is determined following growth factor stimulation. In some embodiments, the p27 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### CDKN2A/p16

Cyclin-dependent kinase inhibitor 2A (CDKN2A), also known as p16, is encoded by the CDKN2A gene (interchangeably referred to as “p16 gene” herein). p16 is a tumor suppressor, functions as an inhibitor of CDK4 and CDK6, the D-type cyclin-dependent kinases that initiate the phosphorylation of the retinoblastoma tumor suppressor protein RB, and induces cell-cycle arrest. 18 Both alleles must be inactivated before its function is eliminated. Three mechanisms have been implicated in its inactivation: deletion such as homozygous deletion (HD), hypermethylation in the promoter CpG island (methylation), and point mutation. See Zhao et. al., EBioMedicine. 2016 June; 8:30-39, the content of which is incorporated by reference in its entirety.

In some embodiments, the cell line of the disclosure comprises a genetic modification selected from the group consisting of: a) knock-out of the endogenous p16 gene; b) knock-down of the endogenous p16 gene (e.g., via CRISPRi or RNAi); c) knock-in of a gene encoding a dominant negative p16 protein; d) inactivation of the endogenous p16 gene; and e) any combination thereof.

In some embodiments, the genetically modified cell line with p16 knock-down expresses the endogenous p16 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous p16 protein in a control cell line. In some embodiments, the genetically modified cell line with p16 knock-down expresses the endogenous p16 protein at a level that is no more than 10% of the expression level of the endogenous p16 protein in a control cell line.

In some embodiments, the disclosure provides a genetically modified cell line comprising a recombinant nucleic acid encoding a p16 protein (i.e., knock-in p16 protein). In some embodiments, the knock-in p16 protein comprises one or more inactivating mutations. In some embodiments the knock-in p16 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in p16 protein is derived from the same animal species as the genetically modified cell line. In some embodiments, the knock-in p16 protein is derived from a different animal species from the genetically modified cell line. In some embodiments, the genetically modified cell line comprises knock-in p16 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous p16 gene. In some embodiments, such inactivation comprises introducing one or more inactivating mutations to the endogenous p16 protein coding gene. In some embodiments, such inactivation comprises modifying the endogenous p16 gene so that it expresses a dominant negative p16 protein. In some embodiments, only one copy of the endogenous p16 gene/allele is inactivated (i.e., heterozygous inactivation). In some embodiments, both copies of the endogenous p16 gene/alleles are inactivated (i.e., homozygous inactivation).

An exemplary p16 protein sequence is provided herein as SEQ ID NO: 60 (Bos Taurus). In some embodiments, the p16 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 60, including all ranges and subranges therebetween. In some embodiments, the p16 protein of the disclosure comprises or consists of an amino acid sequence having up to 1, 2, 3, 4, 5, 6, 7, 8, 9, or 10 amino acids different from the amino acid sequence according to SEQ ID NO: 60. In some embodiments, the p16 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 60, including all ranges or subranges therebetween. In some embodiments, the p16 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 60, including all ranges and subranges therebetween, wherein the sequence difference with SEQ ID NO: 60 consists of conservative amino acid substitutions (other than the one or more inactivating mutations of the disclosure).

In some embodiments, the p16 protein (knock-in and/or endogenous) of the disclosure comprises one or more inactivating mutations. In some embodiments, the p16 protein comprising the one or more inactivating mutations is a dominant negative protein. In some embodiments, the genetic modification comprises a knock in gene encoding for a p16 protein with a dominant negative inactivating mutation.

In some embodiments, the inactivating mutation of p16 protein comprises a single amino acid substitution. In some embodiments, the inactivating mutation of p16 protein comprises a single amino acid insertion or deletion. In some embodiments, the inactivating mutation of p16 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). p16 protein is one of the most commonly inactivated tumor suppressor in human cancers; and thus its mutations and mechanism of inactivation have been extensively characterized. Non-limiting examples of p16 protein mutations and inactivation mechanism are described in Tam et al., J Thorac Oncol. 2013 November;8 (11): 1378-88; Chan et al., Hered Cancer Clin Pract. 2021 Mar. 25;19 (1): 21, the content of each of which is incorporated in its entirety.

In some embodiments, the genetically modified cell line encodes a p16 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the p16 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p16 activity in the genetically modified cell line is reduced by at least 50% compared to that of a control cell line. In some embodiments, the p16 activity in the genetically modified cell line is reduced by 100% (i.e., the p16 activity is eliminated).

In some embodiments, the reduction of p16 activity renders anoikis resistance to the genetically modified cell line and therefore facilitates its adaptation to suspension culture. In some embodiments, the reduction of p16 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the p16 activity is determined by the expression level of p16 protein in the cell. In some embodiments, the expression level of p16 protein is determined by Western blotting or ELISA using a specific antibody. In some embodiments, the p16 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p16 protein expression/accumulation level in the genetically modified cell line is reduced by at least 50% compared to that of a control cell line.

In some embodiments, the p16 activity is determined when the cell line is cultured in a minimum regular growth media. In some embodiments, the p16 activity is determined when the cell line is cultured in a cell growth medium lacking or having reduced level of serum and/or one or more exogenous growth factors (e.g., as described infra). In some embodiments, the p16 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the p16 activity is determined at basal level (i.e., without growth factor stimulation). In some embodiments, the p16 activity is determined following growth factor stimulation. In some embodiments, the p16 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### CDKN2C/p18

Cyclin-dependent kinase 4 inhibitor C (CDKN2C), also known as p18, is encoded by the CDKN2C gene (interchangeably referred to as “p18 gene” herein). It's a member of the INK4 family of cyclin-dependent kinase inhibitors. In human, p18 has been shown to interact with CDK4 or CDK6, and prevent the activation of the CDK kinases, thus function as a cell growth regulator that controls cell cycle G1 progression. See Guan et al., Genes Dev. 1995; 8 (24): 2939-52; Blais et a., Biochem. Biophys. Res. Commun. 1998; 247 (1): 146-53, the content of each of which is incorporated by reference in its entirety.

In some embodiments, the cell line of the disclosure comprises a genetic modification selected from the group consisting of: a) knock-out of the endogenous p18 gene; b) knock-down of the endogenous p18 gene (e.g., via CRISPRi or RNAi); c) knock-in of a gene encoding a dominant negative p18 protein; d) inactivation of the endogenous p18 gene; and e) any combination thereof.

In some embodiments, the genetically modified cell line with p18 knock-down expresses the endogenous p18 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous p18 protein in a control cell line. In some embodiments, the genetically modified cell line with p18 knock-down expresses the endogenous p18 protein at a level that is no more than 10% of the expression level of the endogenous p18 protein in a control cell line.

In some embodiments, the disclosure provides a genetically modified cell line comprising a recombinant nucleic acid encoding a p18 protein (i.e., knock-in p18 protein). In some embodiments, the knock-in p18 protein comprises one or more inactivating mutations. In some embodiments the knock-in p18 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in p18 protein is derived from the same animal species as the genetically modified cell line. In some embodiments, the knock-in p18 protein is derived from a different animal species from the genetically modified cell line. In some embodiments, the genetically modified cell line comprises knock-in p18 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous p18 gene. In some embodiments, such inactivation comprises introducing one or more inactivating mutations to the endogenous p 18 protein coding gene. In some embodiments, such inactivation comprises modifying the endogenous p18 gene so that it expresses a dominant negative p18 protein. In some embodiments, only one copy of the endogenous p18 gene/allele is inactivated (i.e., heterozygous inactivation). In some embodiments, both copies of the endogenous p18 gene/alleles are inactivated (i.e., homozygous inactivation).

An exemplary p18 protein sequence is provided herein as SEQ ID NO: 62 (Bos Taurus). In some embodiments, the p18 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 62, including all ranges and subranges therebetween. In some embodiments, the p18 protein of the disclosure comprises or consists of an amino acid sequence having up to 1, 2, 3, 4, 5, 6, 7, 8, 9, or 10 amino acids different from the amino acid sequence according to SEQ ID NO: 62. In some embodiments, the p18 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 62, including all ranges or subranges therebetween. In some embodiments, the p18 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 62, including all ranges and subranges therebetween, wherein the sequence difference with SEQ ID NO: 62 consists of conservative amino acid substitutions (other than the one or more inactivating mutations of the disclosure).

In some embodiments, the p18 protein (knock-in and/or endogenous) of the disclosure comprises one or more inactivating mutations. In some embodiments, the p18 protein comprising the one or more inactivating mutations is a dominant negative protein. In some embodiments, the genetic modification comprises a knock in gene encoding for a p18 protein with a dominant negative inactivating mutation.

In some embodiments, the inactivating mutation of p18 protein comprises a single amino acid substitution. In some embodiments, the inactivating mutation of p18 protein comprises a single amino acid insertion or deletion. In some embodiments, the inactivating mutation of p18 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). Non-limiting examples of p18 protein mutations are described in Gluick et. al., Endocr Relat Cancer. 2013 Nov. 6;20 (6): L27-9, the content of which is incorporated in its entirety.

In some embodiments, the genetically modified cell line encodes a p18 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the p18 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p18 activity in the genetically modified cell line is reduced by at least 50% compared to that of a control cell line. In some embodiments, the p18 activity in the genetically modified cell line is reduced by 100% (i.e., the p18 activity is eliminated).

In some embodiments, the reduction of p18 activity renders anoikis resistance to the genetically modified cell line and therefore facilitates its adaptation to suspension culture. In some embodiments, the reduction of p18 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the p18 activity is determined by the expression level of p18 protein in the cell. In some embodiments, the expression level of p18 protein is determined by Western blotting or ELISA using a specific antibody. In some embodiments, the p18 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p18 protein expression/accumulation level in the genetically modified cell line is reduced by at least 50% compared to that of a control cell line.

In some embodiments, the p18 activity is determined when the cell line is cultured in a minimum regular growth media. In some embodiments, the p18 activity is determined when the cell line is cultured in a cell growth medium lacking or having reduced level of serum and/or one or more exogenous growth factors (e.g., as described infra). In some embodiments, the p18 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the p18 activity is determined at basal level (i.e., without growth factor stimulation). In some embodiments, the p18 activity is determined following growth factor stimulation. In some embodiments, the p18 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### IGFBP4

Insulin-like growth factor-binding protein 4 (IGFBP4) has three domains of which the N-terminal sequence is important for the binding of insulin-like growth factors (IGFs). It acts as a transport protein for IGF-I and IGF-II and modulates their biological effects. See Durai et al., Int J Oncol. 2006 June;28 (6): 1317-25; Mazerbourg et al., Growth Horm IGF Res. 2004 April; 14 (2): 71-84, the content of each of which is incorporated by reference in its entirety.

In some embodiments, the cell line of the disclosure comprises a genetic modification selected from the group consisting of: a) knock-out of the endogenous IGFBP4 gene; b) knock-down of the endogenous IGFBP4 gene (e.g., via CRISPRi or RNAi); c) knock-in of a gene encoding a dominant negative IGFBP4 protein; d) inactivation of the endogenous IGFBP4 gene; and e) any combination thereof.

In some embodiments, the genetically modified cell line with IGFBP4 knock-down expresses the endogenous IGFBP4 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous IGFBP4 protein in a control cell line. In some embodiments, the genetically modified cell line with IGFBP4 knock-down expresses the endogenous IGFBP4 protein at a level that is no more than 10% of the expression level of the endogenous IGFBP4 protein in a control cell line.

In some embodiments, the disclosure provides a genetically modified cell line comprising a recombinant nucleic acid encoding a IGFBP4 protein (i.e., knock-in IGFBP4 protein). In some embodiments, the knock-in IGFBP4 protein comprises one or more inactivating mutations. In some embodiments the knock-in IGFBP4 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in IGFBP4 protein is derived from the same animal species as the genetically modified cell line. In some embodiments, the knock-in IGFBP4 protein is derived from a different animal species from the genetically modified cell line. In some embodiments, the genetically modified cell line comprises knock-in IGFBP4 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous IGFBP4 gene. In some embodiments, such inactivation comprises introducing one or more inactivating mutations to the endogenous IGFBP4 protein coding gene. In some embodiments, such inactivation comprises modifying the endogenous IGFBP4 gene so that it expresses a dominant negative IGFBP4 protein. In some embodiments, only one copy of the endogenous IGFBP4 gene/allele is inactivated (i.e., heterozygous inactivation). In some embodiments, both copies of the endogenous IGFBP4 gene/alleles are inactivated (i.e., homozygous inactivation).

An exemplary IGFBP4 protein sequence is provided herein as SEQ ID NO: 54 (Bos Taurus). In some embodiments, the IGFBP4 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 54, including all ranges and subranges therebetween. In some embodiments, the IGFBP4 protein of the disclosure comprises or consists of an amino acid sequence having up to 1, 2, 3, 4, 5, 6, 7, 8, 9, or 10 amino acids different from the amino acid sequence according to SEQ ID NO: 54. In some embodiments, the IGFBP4 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 54, including all ranges or subranges therebetween. In some embodiments, the IGFBP4 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 54, including all ranges and subranges therebetween, wherein the sequence difference with SEQ ID NO: 54 consists of conservative amino acid substitutions (other than the one or more inactivating mutations of the disclosure).

In some embodiments, the IGFBP4 protein (knock-in and/or endogenous) of the disclosure comprises one or more inactivating mutations. In some embodiments, the IGFBP4 protein comprising the one or more inactivating mutations is a dominant negative protein. In some embodiments, the genetic modification comprises a knock in gene encoding for a IGFBP4 protein with a dominant negative inactivating mutation.

In some embodiments, the inactivating mutation of IGFBP4 protein comprises a single amino acid substitution. In some embodiments, the inactivating mutation of IGFBP4 protein comprises a single amino acid insertion or deletion. In some embodiments, the inactivating mutation of IGFBP4 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). In some embodiments, the inactivating mutation of IGFBP4 protein reduces or abolishes its binding to IGF.

In some embodiments, the genetically modified cell line encodes a IGFBP4 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the IGFBP4 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the IGFBP4 activity in the genetically modified cell line is reduced by at least 50% compared to that of a control cell line. In some embodiments, the IGFBP4 activity in the genetically modified cell line is reduced by 100% (i.e., the IGFBP4 activity is eliminated).

In some embodiments, the reduction of IGFBP4 activity renders anoikis resistance to the genetically modified cell line and therefore facilitates its adaptation to suspension culture. In some embodiments, the reduction of IGFBP4 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the IGFBP4 activity is determined by the expression level of IGFBP4 protein in the cell. In some embodiments, the expression level of IGFBP4 protein is determined by Western blotting or ELISA using a specific antibody. In some embodiments, the IGFBP4 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the IGFBP4 protein expression/accumulation level in the genetically modified cell line is reduced by at least 50% compared to that of a control cell line.

In some embodiments, the IGFBP4 activity is determined when the cell line is cultured in a minimum regular growth media. In some embodiments, the IGFBP4 activity is determined when the cell line is cultured in a cell growth medium lacking or having reduced level of serum and/or one or more exogenous growth factors (e.g., as described infra). In some embodiments, the IGFBP4 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the IGFBP4 activity is determined at basal level (i.e., without growth factor stimulation). In some embodiments, the IGFBP4 activity is determined following growth factor stimulation. In some embodiments, the IGFBP4 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### RB1

Retinoblastoma 1 (Rb1) gene is the first cloned tumor suppressor gene. The RB1 protein encoded by this gene is a negative regulator of the cell cycle. The encoded protein also stabilizes constitutive heterochromatin to maintain the overall chromatin structure. The active, hypophosphorylated form of the protein binds transcription factor E2F1. See Yun et. al., Int J Ophthalmol. 2011; 4 (1): 103-109, the content of which is incorporated by reference in its entirety.

In some embodiments, the cell line of the disclosure comprises a genetic modification selected from the group consisting of: a) knock-out of the endogenous RB1 gene; b) knock-down of the endogenous RB1 gene (e.g., via CRISPRi or RNAi); c) knock-in of a gene encoding a dominant negative RB1 protein; d) inactivation of the endogenous RB1 gene; and e) any combination thereof.

In some embodiments, the genetically modified cell line with RB1 knock-down expresses the endogenous RB1 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous RB1 protein in a control cell line. In some embodiments, the genetically modified cell line with RB1 knock-down expresses the endogenous RB 1 protein at a level that is no more than 10% of the expression level of the endogenous RB1 protein in a control cell line.

In some embodiments, the disclosure provides a genetically modified cell line comprising a recombinant nucleic acid encoding a RB1 protein (i.e., knock-in RB1 protein). In some embodiments, the knock-in RB1 protein comprises one or more inactivating mutations. In some embodiments the knock-in RB1 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in RB1 protein is derived from the same animal species as the genetically modified cell line. In some embodiments, the knock-in RB1 protein is derived from a different animal species from the genetically modified cell line. In some embodiments, the genetically modified cell line comprises knock-in RB1 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous RB1 gene. In some embodiments, such inactivation comprises introducing one or more inactivating mutations to the endogenous RB1 protein coding gene. In some embodiments, such inactivation comprises modifying the endogenous RB1 gene so that it expresses a dominant negative RB1 protein. In some embodiments, only one copy of the endogenous RB1 gene/allele is inactivated (i.e., heterozygous inactivation). In some embodiments, both copies of the endogenous RB1 gene/alleles are inactivated (i.e., homozygous inactivation).

An exemplary RB1 protein sequence is provided herein as SEQ ID NO: 66 (Bos Taurus). In some embodiments, the RB1 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 66, including all ranges and subranges therebetween. In some embodiments, the RB1 protein of the disclosure comprises or consists of an amino acid sequence having up to 1, 2, 3, 4, 5, 6, 7, 8, 9, or 10 amino acids different from the amino acid sequence according to SEQ ID NO: 66. In some embodiments, the RB1 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 66, including all ranges or subranges therebetween. In some embodiments, the RB1 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 66, including all ranges and subranges therebetween, wherein the sequence difference with SEQ ID NO: 66 consists of conservative amino acid substitutions (other than the one or more inactivating mutations of the disclosure).

In some embodiments, the RB1 protein (knock-in and/or endogenous) of the disclosure comprises one or more inactivating mutations. In some embodiments, the RB1 protein comprising the one or more inactivating mutations is a dominant negative protein. In some embodiments, the genetic modification comprises a knock in gene encoding for a RB1 protein with a dominant negative inactivating mutation.

In some embodiments, the inactivating mutation of RB1 protein comprises a single amino acid substitution. In some embodiments, the inactivating mutation of RB1 protein comprises a single amino acid insertion or deletion. In some embodiments, the inactivating mutation of RB1 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). Non-limiting examples of RB1 protein mutations are described in Tomar et al., PLOS One. 2017 Jun. 2; 12 (6): e0178776, the content of which is incorporated in its entirety.

In some embodiments, the genetically modified cell line encodes a RB1 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the RB1 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the RB1 activity in the genetically modified cell line is reduced by at least 50% compared to that of a control cell line. In some embodiments, the RB1 activity in the genetically modified cell line is reduced by 100% (i.e., the RB1 activity is eliminated).

In some embodiments, the reduction of RB1 activity renders anoikis resistance to the genetically modified cell line and therefore facilitates its adaptation to suspension culture. In some embodiments, the reduction of RB1 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the RB1 activity is determined by the expression level of RB1 protein in the cell. In some embodiments, the expression level of RB1 protein is determined by Western blotting or ELISA using a specific antibody. In some embodiments, the RB1 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the RB1 protein expression/accumulation level in the genetically modified cell line is reduced by at least 50% compared to that of a control cell line.

In some embodiments, the RB1 activity is determined when the cell line is cultured in a minimum regular growth media. In some embodiments, the RB1 activity is determined when the cell line is cultured in a cell growth medium lacking or having reduced level of serum and/or one or more exogenous growth factors (e.g., as described infra). In some embodiments, the RB1 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the RB1 activity is determined at basal level (i.e., without growth factor stimulation). In some embodiments, the RB1 activity is determined following growth factor stimulation. In some embodiments, the RB1 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### SRC

SRC protein is one of the most extensively studied non-receptor protein tyrosine kinases and a key regulator of signal transduction implicated in fundamental cellular processes. SRC protein comprises the SH4 region, which contains the lipidation site (e.g., myristylation site) for membrane localization; the SH3 domain, which binds proline-rich sequences; the SH2 domain, which binds phosphotyrosine-containing sequences; the SH1 domain, which is the catalytic kinase domain and contains the substrate- and the ATP-binding site, as well as the autophosphorylation site (Tyr419 in human SRC); and a short C-terminal tail, which contains the negative-regulatory tyrosine residue (Tyr530 in human SRC). See Pelaz et al., Oncogene. 2022 November;41 (45): 4917-4928, the content of each of which is incorporated by reference in its entirety.

In some embodiments, the cell line of the disclosure comprises a genetic modification selected from the group consisting of: a) introduction of a recombinant nucleic acid encoding a knock-in SRC protein (e.g., a knock-in SRC protein with an activating mutation); b) introduction of one or more activating mutations to the endogenous SRC protein encoded by the SRC gene locus; c) overexpression of endogenous SRC protein; and d) any combination of the above.

In some embodiments, the expression level of the SRC protein in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold (including all ranges and subranges therebetween), compared to that of a control cell line. In some embodiments, the genetically modified cell line expresses the SRC protein at a level that is at least 2-fold of the expression level of the endogenous SRC protein in a control cell line. In some embodiments, the overexpressed SRC protein comprises one or more of the activating mutations of the disclosure.

In some embodiments, the disclosure provides a genetically modified cell line comprising a recombinant nucleic acid encoding a SRC protein (i.e., knock-in SRC protein). In some embodiments, the knock-in SRC protein comprises one or more activating mutations. This protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in SRC protein is derived from the same animal species as the genetically modified cell line. In some embodiments, the knock-in SRC protein is derived from a different animal species from the genetically modified cell line. In some embodiments, the genetically modified cell line comprises knock-in SRC protein ectopically expressed from constructs that are introduced into the cells, for example expressed from a plasmid, or other expression vector. In some embodiments, the recombinant nucleic acid is integrated into the cell's genome, and the expression is driven in that manner (e.g., homologous recombination, introduction mediated by CRISPR-based technology).

In some embodiments, the genetic modification comprises introducing one or more activating mutations to the endogenous SRC protein coding gene. In some embodiments, only one copy of the endogenous SRC gene/allele is activated (i.e., heterozygous activation). In some embodiments, both copies of the endogenous SRC gene/alleles are activated (i.e., homozygous activation).

In some embodiments, the genetic modification comprises introducing one or more mutations to the promoter region of the endogenous SRC gene which leads to overexpress of the endogenous SRC protein. In some embodiments, the mutation at the SRC promoter region creates a new binding site for a transcription factor. In some embodiments, the mutation at the SRC promoter region comprise introducing another promoter (e.g., a promoter of the disclosure) to the cell genome so that the SRC coding region is operably linked to the new promoter.

An exemplary SRC protein sequence is provided herein as SEQ ID NO: 64 (Bos taurus). In some embodiments, the SRC protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 64, including all ranges and subranges therebetween. In some embodiments, the SRC protein of the disclosure comprises or consists of an amino acid sequence having up to 1, 2, 3, 4, 5, 6, 7, 8, 9, or 10 amino acids different from the amino acid sequence according to SEQ ID NO: 64. In some embodiments, the SRC protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 64, including all ranges or subranges therebetween. In some embodiments, the SRC protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 64, including all ranges and subranges therebetween, wherein the sequence difference with SEQ ID NO: 64 consists of conservative amino acid substitutions (other than the one or more activating mutations of the disclosure).

In some embodiments, the SRC protein (knock-in and/or endogenous) of the disclosure comprises one or more activating mutations. In some embodiments, the genetic modification comprises a knock-in gene encoding for a SRC protein with an activating mutation.

In some embodiments, the activating mutation of SRC protein comprises a single amino acid substitution. In some embodiments, the activating mutation of SRC protein comprises a single amino acid insertion or deletion. In some embodiments, the activating mutation of SRC protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues).

In some embodiments, the activation mutation comprises a mutation at the amino acid position corresponding to Y553 of SEQ ID NO: 64 (which in turn corresponds to Y530 of human SRC protein). In some embodiments, the activating mutation comprises an substitution corresponding to Y553F of SEQ ID NO: 64.

In some embodiments, the genetically modified cell line encodes a SRC protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure.

In some embodiments, the SRC activity in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to that of a control cell line. In some embodiments, the SRC activity in the genetically modified cell line is increased by at least 100% compared to that of a control cell line.

In some embodiments, the increase of SRC activity renders anoikis resistance to the genetically modified cell line and therefore facilitates its adaptation to suspension culture. In some embodiments, the increase of SRC activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the SRC activity is determined by the expression level of SRC protein in the cell. In some embodiments, the expression level of SRC protein is determined by Western blotting or ELISA using a specific antibody. In some embodiments, the SRC protein expression/accumulation level in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to that of a control cell line. In some embodiments, the SRC protein expression/accumulation level in the genetically modified cell line is increased by at least 100% compared to that of a control cell line.

In some embodiments, the SRC activity is determined by the corresponding kinase activity. In some embodiments, the kinase activity is determined using the method described in Irby et al., Nat Genet. 1999 February;21 (2): 187-90, the content of which is incorporated by reference in its entirety. In some embodiments, the SRC kinase activity in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to that of a control cell line. In some embodiments, the SRC kinase activity in the genetically modified cell line is increased by at least 100% compared to that of a control cell line.

In some embodiments, the SRC activity is determined when the cell line is cultured in a minimum regular growth media. In some embodiments, the SRC activity is determined when the cell line is cultured in a cell growth medium lacking or having reduced level of serum and/or one or more exogenous growth factors (e.g., as described infra). In some embodiments, the SRC activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the SRC activity is determined at basal level (i.e., without growth factor stimulation). In some embodiments, the SRC activity is determined following growth factor stimulation. In some embodiments, the SRC activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### TERT

Telomerase Reverse Transcriptase (TERT) is the catalytic component of the telomerase enzyme. This protein is responsible for the synthesis of telomeric DNA repeats from the RNA template located within TR. The TERT protein comprises four protein domains: the telomerase essential N-terminal (TEN) domain, the telomerase RNA binding domain (TRBD), the reverse transcriptase (RT) domain, and the C-terminal extension (CTE) domain. The central RT domain and the CTE domain contain motifs highly conserved in other RTs and DNA polymerases, while the TEN and TRBD domains are unique to the TERT protein. The overall structure of the RT and CTE domains, like all known DNA polymerases, resembles a right hand with finger, palm, and thumb subdomains. Like that of all other DNA polymerases, within the palm of the TERT protein lie three invariant aspartic acids. This aspartic acid triad coordinates the positioning of two magnesium atoms. This acid-metal chemistry for nucleotide addition is common to all DNA polymerases. The TERT CTE domain has a similar overall structure to viral RTs “thumb” domains and binds the 3′-end of the telomeric DNA to enhance DNA polymerization. The TEN and TRBD domain contains TR binding sites critical for ribonucleoprotein assembly. In addition, the TEN domain contains “anchor” sites for binding single-stranded telomeric DNA. This DNA anchor site is important for delaying the complete dissociation of the telomerase enzyme from the telomeric DNA, enhancing the number of telomeric DNA repeats synthesized onto the end of the chromosome.

Telomerase complex plays a key role in cancer formation by telomere dependent or independent mechanisms. Telomere maintenance mechanisms include complex TERT changes such as gene amplifications, TERT structural variants, TERT promoter germline and somatic mutations, TERT epigenetic changes, and alternative lengthening of telomere. TERT expression is regulated via multiple genetic and epigenetic alterations which affect telomerase activity, and telomerase activity via TERT expression has an impact on telomere length. See Dratwa et. al., Front Immunol. 2020 Nov. 19; 11:589929; Yuan et. al., Oncogene. 2019 August;38 (34): 6172-6183, the content of each of which is incorporated by reference in its entirety.

In some embodiments, the cell line of the disclosure comprises a genetic modification selected from the group consisting of: a) introduction of a recombinant nucleic acid encoding a knock-in TERT protein; b) introduction of one or more activating mutations to the endogenous TERT protein encoded by the TERT gene locus; c) overexpression of endogenous TERT protein; and d) any combination of the above.

In some embodiments, the expression level of the TERT protein in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold (including all ranges and subranges therebetween), compared to that of a control cell line. In some embodiments, the genetically modified cell line expresses the TERT protein at a level that is at least 2-fold of the expression level of the endogenous TERT protein in a control cell line. In some embodiments, the overexpressed TERT protein comprises one or more of the activating mutations of the disclosure.

In some embodiments, the disclosure provides a genetically modified cell line comprising a recombinant nucleic acid encoding a TERT protein (i.e., knock-in TERT protein). In some embodiments, the knock-in TERT protein comprises one or more activating mutations. This protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in TERT protein is derived from the same animal species as the genetically modified cell line. In some embodiments, the knock-in TERT protein is derived from a different animal species from the genetically modified cell line. In some embodiments, the genetically modified cell line comprises knock-in TERT protein ectopically expressed from constructs that are introduced into the cells, for example expressed from a plasmid, or other expression vector. In some embodiments, the recombinant nucleic acid is integrated into the cell's genome, and the expression is driven in that manner (e.g., homologous recombination, introduction mediated by CRISPR-based technology).

In some embodiments, the genetic modification comprises introducing one or more activating mutations to the endogenous TERT protein coding gene. In some embodiments, only one copy of the endogenous TERT gene/allele is activated (i.e., heterozygous activation). In some embodiments, both copies of the endogenous TERT gene/alleles are activated (i.e., homozygous activation).

In some embodiments, the genetic modification comprises introducing one or more mutations to the promoter region of the endogenous TERT gene which leads to overexpress of the endogenous TERT protein. In some embodiments, the mutation at the TERT promoter region creates a new binding site for a transcription factor. In some embodiments, the mutation at the TERT promoter region comprise introducing another promoter (e.g., a promoter of the disclosure) to the cell genome so that the TERT coding region is operably linked to the new promoter.

An exemplary TERT protein sequence is provided herein as SEQ ID NO: 48 (Bos taurus). In some embodiments, the TERT protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 48, including all ranges and subranges therebetween. In some embodiments, the TERT protein of the disclosure comprises or consists of an amino acid sequence having up to 1, 2, 3, 4, 5, 6, 7, 8, 9, or 10 amino acids different from the amino acid sequence according to SEQ ID NO: 48. In some embodiments, the TERT protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 48, including all ranges or subranges therebetween. In some embodiments, the TERT protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 48, including all ranges and subranges therebetween, wherein the sequence difference with SEQ ID NO: 48 consists of conservative amino acid substitutions (other than the one or more activating mutations of the disclosure).

In some embodiments, the TERT protein (knock-in and/or endogenous) of the disclosure comprises one or more activating mutations. In some embodiments, the genetic modification comprises a knock-in gene encoding for a TERT protein with an activating mutation.

In some embodiments, the activating mutation of TERT protein comprises a single amino acid substitution. In some embodiments, the activating mutation of TERT protein comprises a single amino acid insertion or deletion. In some embodiments, the activating mutation of TERT protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues).

In some embodiments, the genetically modified cell line encodes a TERT protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure.

In some embodiments, the TERT activity in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to that of a control cell line. In some embodiments, the TERT activity in the genetically modified cell line is increased by at least 100% compared to that of a control cell line.

In some embodiments, the increase of TERT activity renders anoikis resistance to the genetically modified cell line and therefore facilitates its adaptation to suspension culture. In some embodiments, the increase of TERT activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the TERT activity is determined by the expression level of TERT protein in the cell. In some embodiments, the expression level of TERT protein is determined by Western blotting or ELISA using a specific antibody. In some embodiments, the TERT protein expression/accumulation level in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to that of a control cell line. In some embodiments, the TERT protein expression/accumulation level in the genetically modified cell line is increased by at least 100% compared to that of a control cell line.

In some embodiments, the TERT activity is determined by the corresponding telomerase activity. In some embodiments, the telomerase activity is determined using the method described in Skvortsov et. al., Assays for detection of telomerase activity. Acta Naturae 2011, 3, 48-68, the content of which is incorporated by reference in its entirety. In some embodiments, the TERT telomerase activity in the genetically modified cell line is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, including all ranges and subranges therebetween, compared to that of a control cell line. In some embodiments, the TERT telomerase activity in the genetically modified cell line is increased by at least 100% compared to that of a control cell line.

In some embodiments, the TERT activity is determined when the cell line is cultured in a minimum regular growth media. In some embodiments, the TERT activity is determined when the cell line is cultured in a cell growth medium lacking or having reduced level of serum and/or one or more exogenous growth factors (e.g., as described infra). In some embodiments, the TERT activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the TERT activity is determined at basal level (i.e., without growth factor stimulation). In some embodiments, the TERT activity is determined following growth factor stimulation. In some embodiments, the TERT activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

p53

Tumor suppressor p53 protein is a transcription factor that regulates cell division (and prevents tumor formation) by stopping cells with mutated or damaged DNA from dividing and signaling for them to undergo apoptosis through transcriptional regulation. It activates a multitude of transcriptional targets in response to cellular stress or DNA damage.

A broad range of responses are coordinated by p53 including cell cycle arrest, DNA repair, altered metabolism, anti-oxidant effects, anti-angiogenic effects, autophagy, senescence, and apoptosis. p53 can inhibit cell cycle progression through the upregulation of p21 expression. p21 protein will then bind cyclin E/Cdk2 and cyclin D/Cdk4 resulting in G1 arrest of the cell cycle. p53 can also bring about cell cycle arrest at the G2/M phase through binding to other p53 target genes such as 14-3-30 and cdc25C. p53 plays a role in DNA repair through both halting the cell cycle to allow repair machinery to operate and directly through the activation of repair mechanisms and constantly surveys the genome for signs of DNA damage such as double-strand breaks. p53 also plays an active role in many different types of DNA repair including nucleotide excision repair, base excision repair, mismatch repair, and nonhomologous end-joining. The p53 gene (also known as TP53) is the single most frequently mutated gene in human cancer, with partial or complete loss of function occurring in over 50% of tumors. Mutations in p53 confer a selective advantage on the tumor cells, allowing them to evade cell cycle checkpoints, avoid apoptosis and senescence, and proliferate under conditions where normal cells cannot.

The p53 protein has several domains. At the N-terminus, there are two distinct transactivation domains (TADI and TADII) followed by the proline-rich domain (PD) and the DNA binding domain (DBD). The TADI and TADII are critical for p53 regulation as they provide binding sites for the transcriptional machinery and the negative regulator MDM2. The DBD is pivotal for the transcriptional activity of p53. At the C-terminus, there are an oligomerization domain (OD) and a lysine-rich regulatory domain (RD). The OD allows p53 to form a tetramer which is organized as a dimer of dimers.

The p53 protein may undergo several post-translational modifications. When the cell is confronted with stress, p53 ubiquitylation is suppressed and p53 accumulates in the nucleus, where it is activated and stabilized by post-translational modification including phosphorylation and acetylation. Phosphorylation of p53 occurs rapidly in response to cellular stress. p53 contains multiple serine and threonine residues that serve as phosphorylation sites for protein kinases. These kinases may include ATM/ATR, Chk1/Chk2, CK1, CK2, PKC, CDK1/2, DNA-PK, HIPK2, ERK2, p38, and JNK. p53 may be acetylated by p300/CBP and P300/CBP-associated factor (PCAF) (i, in response to gamma-irradiation and UV light) and TIP60 and hMOF (e.g., in response to DNA damage). Acetylation of p53 augments p53 DNA binding, aids in recruiting co-activators, and stabilizes p53 by inhibiting its ubiquitination by MDM2. See Bieging, et. al. (2014) Nature Reviews Cancer, 14 (5), 359-370, the content of which is incorporated by reference in its entirety.

In some embodiments, the cell line of the disclosure comprises a genetic modification selected from the group consisting of: a) knock-out of the endogenous p53 gene; b) knock-down of the endogenous p53 gene (e.g., via CRISPRi or RNAi); c) knock-in of a gene encoding a dominant negative p53 protein; d) inactivation of the endogenous p53 gene; and e) any combination thereof.

In some embodiments, the genetically modified cell line with p53 knock-down expresses the endogenous p53 protein at a level that is no more than 1%, no more than 2%, no more than 3%, no more than 5%, no more than 10%, no more than 15%, no more than 20%, no more than 25%, no more than 30%, no more than 35%, no more than 40%, no more than 45%, no more than 50%, no more than 55%, no more than 60%, no more than 65%, no more than 70%, no more than 75%, no more than 80%, no more than 85%, or no more than 90% of the expression level of the endogenous p53 protein in a control cell line. In some embodiments, the genetically modified cell line with p53 knock-down expresses the endogenous p53 protein at a level that is no more than 10% of the expression level of the endogenous p53 protein in a control cell line.

In some embodiments, the disclosure provides a genetically modified cell line comprising a recombinant nucleic acid encoding a p53 protein (i.e., knock-in p53 protein). In some embodiments, the knock-in p53 protein comprises one or more inactivating mutations. In some embodiments the knock-in p53 protein is a dominant negative form. Knock-in of the dominant negative protein can result in overall decreased function of a wild-type gene. Additionally, expressing a dominant negative protein can result in a phenotype that is similar to that of a knockout and/or knockdown. This dominant negative protein can be expressed in a nucleic acid within the control of any promoter (e.g., a promoter of the present disclosure). For example, a promoter can be a ubiquitous promoter. A promoter can also be an inducible promoter, tissue specific promoter, cell specific promoter, and/or developmental specific promoter. In some embodiments, the knock-in p53 protein is derived from the same animal species as the genetically modified cell line. In some embodiments, the knock-in p53 protein is derived from a different animal species from the genetically modified cell line. In some embodiments, the genetically modified cell line comprises knock-in p53 protein ectopically expressed from constructs that are introduced into the cells, for example expressed from a plasmid, or other expression vector. In some embodiments, the recombinant nucleic acid is integrated into the cell's genome, and the expression is driven in that manner (e.g. homologous recombination, introduction mediated by CRISPR-based technology).

In some embodiments, the genetic modification comprises inactivation of the endogenous p53 gene. In some embodiments, such inactivation comprises introducing one or more inactivating mutations to the endogenous p53 protein coding gene. In some embodiments, such inactivation comprises modifying the endogenous p53 gene so that it expresses a dominant negative p53 protein. In some embodiments, only one copy of the endogenous p53 gene/allele is inactivated (i.e., heterozygous inactivation). In some embodiments, both copies of the endogenous p53 gene/alleles are inactivated (i.e., homozygous inactivation).

An exemplary p53 protein sequence is provided herein as SEQ ID NO: 50 (Bos taurus). In some embodiments, the p53 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, at least 99.5%, or 100% sequence identity with SEQ ID NO: 50, including all ranges and subranges therebetween. In some embodiments, the p53 protein of the disclosure comprises or consists of an amino acid sequence having up to 1, 2, 3, 4, 5, 6, 7, 8, 9, or 10 amino acids different from the amino acid sequence according to SEQ ID NO: 50. In some embodiments, the p53 protein of the disclosure comprises or consists of an amino acid sequence sharing at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, sequence identity with SEQ ID NO: 50, including all ranges or subranges therebetween. In some embodiments, the p53 protein of the disclosure comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 50, including all ranges and subranges therebetween, wherein the sequence difference with SEQ ID NO: 50 consists of conservative amino acid substitutions (other than the one or more inactivating mutations of the disclosure).

In some embodiments, the p53 protein (knock-in and/or endogenous) of the disclosure comprises one or more inactivating mutations. In some embodiments, the p53 protein comprising the one or more inactivating mutations is a dominant negative protein. In some embodiments, the genetic modification comprises a knock in gene encoding for a p53 protein with a dominant negative inactivating mutation.

In some embodiments, the inactivating mutation of p53 protein comprises a single amino acid substitution. In some embodiments, the inactivating mutation of p53 protein comprises a single amino acid insertion or deletion. In some embodiments, the inactivating mutation of p53 protein comprises insertion, deletion, or substitution of multiple consecutive amino acid residues (e.g., 2 or more, 3 or more, 5 or more, 10 or more, 20 or more, 30 or more, 40 or more, or 50 or more amino acid residues). Non-limiting examples of p53 protein mutations are described in Stein et al., Carcinogenesis. 2020 Dec. 31;41 (12): 1635-1647; Rivlin et. al., Genes Cancer. 2011 April; 2 (4): 466-474, the content of each of which is incorporated in its entirety.

In some embodiments, the inactivating mutation of p53 protein comprises or consists of a mutation at the amino acid position corresponding to H171 of SEQ ID NO: 50. In some embodiments, the inactivating mutation corresponds to an amino acid substitution H171 of SEQ ID NO: 50. In some embodiments, the inactivating mutation corresponds to H171Q of SEQ ID NO: 50. In some embodiments, the p53 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 50, including all ranges and subranges therebetween.

In some embodiments, the inactivating mutation of p53 protein comprises or consists of a mutation at the amino acid position corresponding to R241 of SEQ ID NO: 50. In some embodiments, the inactivating mutation corresponds to an amino acid substitution R241 of SEQ ID NO: 50. In some embodiments, the inactivating mutation corresponds to R241W of SEQ ID NO: 50. In some embodiments, the p53 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 50, including all ranges and subranges therebetween.

In some embodiments, the inactivating mutation of p53 protein comprises or consists of a mutation at the amino acid position corresponding to R266 of SEQ ID NO: 50. In some embodiments, the inactivating mutation corresponds to an amino acid substitution R266 of SEQ ID NO: 50. In some embodiments, the inactivating mutation corresponds to R266H of SEQ ID NO: 50. In some embodiments, the p53 protein having such an inactivation mutation comprises or consists of an amino acid sequence sharing at least 60%, at least 65%, at least 70%, at least 75%, at least 80%, at least 85%, at least 90%, at least 95%, at least 96%, at least 97%, at least 98%, at least 99%, or at least 99.5% sequence identity with SEQ ID NO: 50, including all ranges and subranges therebetween.

In some embodiments, the genetically modified cell line encodes a p53 protein (endogenous and/or knock-in) that is inducibly expressed. Inducible approaches include, but are not limited to, regulation of transcription factor/promoter activity by ligand inducible transcription factor technology (e.g., tet-on, tet-off, RheoSwitch), site-directed recombination technology (e.g., Cre-LoxP, flp-FRT), transposon technology (e.g. Sleeping Beauty, PiggyBac), ligand binding receptor fusion technology (e.g., estrogen, progesterone, androgen, thyroid hormone, glucocorticoid hormone, tamoxifen ligand agonists), and transient transfection of extrachromosomal expression vectors comprising the recombinant nucleic acid of the disclosure. In some embodiments, the p53 activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p53 activity in the genetically modified cell line is reduced by at least 50% compared to that of a control cell line. In some embodiments, the p53 activity in the genetically modified cell line is reduced by 100% (i.e., the p53 activity is eliminated).

In some embodiments, the reduction of p53 activity renders anoikis resistance to the genetically modified cell line and therefore facilitates its adaptation to suspension culture. In some embodiments, the reduction of p53 activity renders resistance to programmed cell death that is induced in a corresponding control cell line due to reduction or absence of serum and/or one or more exogenous growth factors (e.g., serum deprivation) in the culture medium.

In some embodiments, such genetic modification is permanent. In some embodiments, such genetic modification is transient. In some embodiments, such genetic modification is reversible. In some embodiments, such genetic modification is conditional and/or inducible.

In some embodiments, the p53 activity is determined by the expression level of p53 protein in the cell. In some embodiments, the expression level of p53 protein is determined by Western blotting or ELISA using a specific antibody. In some embodiments, the p53 protein expression/accumulation level in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the p53 protein expression/accumulation level in the genetically modified cell line is reduced by at least 50% compared to that of a control cell line.

In some embodiments, the p53 activity is determined by the corresponding transactivation activity. In some embodiments, the transactivation activity is determined using the method described in Andreotti et. al., PLOS One. 2011; 6 (6): e20643, the content of which is incorporated by reference in its entirety. In some embodiments, the transactivation activity in the genetically modified cell line is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, or 100%, including all ranges and subranges therebetween, compared to that of a control cell line. In some embodiments, the transactivation activity in the genetically modified cell line is reduced by at least 50% compared to that of a control cell line.

In some embodiments, the p53 activity is determined when the cell line is cultured in a minimum regular growth media. In some embodiments, the p53 activity is determined when the cell line is cultured in a cell growth medium lacking or having reduced level of serum and/or one or more exogenous growth factors (e.g., as described infra). In some embodiments, the p53 activity is determined when the cell line is at about 30%, 35%, 40%, 45%, 50%, 55%, 60%, 65%, 70%, 75%, 80%, 85%, 90%, 95%, or 100% of its maximum viable cell density. In some embodiments, the p53 activity is determined at basal level (i.e., without growth factor stimulation). In some embodiments, the p53 activity is determined following growth factor stimulation. In some embodiments, the p53 activity is determined at about 5 minutes, about 10 minutes, about 15 minutes, about 20 minutes, about 30 minutes, about 60 minutes, about 2 hours, about 4 hours, about 6 hours, about 12 hours, about 24 hours, or longer than 24 hours, following growth factor stimulation. In some embodiments, the growth factor is an FGF (e.g., FGF-2). In some embodiments, the growth factor is EGF. In some embodiments, the concentration of the growth factor applied is about 1 ng/ml, about 2 ng/ml, about 5 ng/ml, about 10 ng/ml, about 20 ng/ml, about 50 ng/ml, or about 100 ng/ml, including all ranges and subranges therebetween.

### Combination of Genetic Modifications

In some embodiments, the cell line of the disclosure comprises a combination of two or more genetic modifications according to one of Embodiments 1-286 in Table 2 below.

TABLE 2
Combination of Genetic Modifications

| Embodiment | Genetic Modifications* |
|---|---|
| 1 | PTEN (RA) + CASP3 (RA) |
| 2 | PTEN (RA) + CASP8 (RA) |
| 3 | PTEN (RA) + CDH1 (RA) |
| 4 | PTEN (RA) + ITGB1 (RA) |
| 5 | PTEN (RA) + RB1 (RA) |
| 6 | PTEN (RA) + IGFBP4 (RA) |
| 7 | PTEN (RA) + p16 (RA) |
| 8 | PTEN (RA) + p18 (RA) |
| 9 | PTEN (RA) + p21 (RA) |
| 10 | PTEN (RA) + p27 (RA) |
| 11 | PTEN (RA) + SRC (IA) |
| 12 | CASP3 (RA) + CASP8 (RA) |
| 13 | CASP3 (RA) + CDH1 (RA) |
| 14 | CASP3 (RA) + ITGB1 (RA) |
| 15 | CASP3 (RA) + RB1 (RA) |
| 16 | CASP3 (RA) + IGFBP4 (RA) |
| 17 | CASP3 (RA) + p16 (RA) |
| 18 | CASP3 (RA) + p18 (RA) |
| 19 | CASP3 (RA) + p21 (RA) |
| 20 | CASP3 (RA) + p27 (RA) |
| 21 | CASP3 (RA) + SRC (IA) |
| 22 | CASP8 (RA) + CDH1 (RA) |
| 23 | CASP8 (RA) + ITGB1 (RA) |
| 24 | CASP8 (RA) + RB1 (RA) |
| 25 | CASP8 (RA) + IGFBP4 (RA) |
| 26 | CASP8 (RA) + p16 (RA) |
| 27 | CASP8 (RA) + p18 (RA) |
| 28 | CASP8 (RA) + p21 (RA) |
| 29 | CASP8 (RA) + p27 (RA) |
| 30 | CASP8 (RA) + SRC (IA) |
| 31 | CDH1 (RA) + ITGB1 (RA) |
| 32 | CDH1 (RA) + RB1 (RA) |
| 33 | CDH1 (RA) + IGFBP4 (RA) |
| 34 | CDH1 (RA) + p16 (RA) |
| 35 | CDH1 (RA) + p18 (RA) |
| 36 | CDH1 (RA) + p21 (RA) |
| 37 | CDH1 (RA) + p27 (RA) |
| 38 | CDH1 (RA) + SRC (IA) |
| 39 | ITGB1 (RA) + RB1 (RA) |
| 40 | ITGB1 (RA) + IGFBP4 (RA) |
| 41 | ITGB1 (RA) + p16 (RA) |
| 42 | ITGB1 (RA) + p18 (RA) |
| 43 | ITGB1 (RA) + p21 (RA) |
| 44 | ITGB1 (RA) + p27 (RA) |
| 45 | ITGB1 (RA) + SRC (IA) |
| 46 | RB1 (RA) + IGFBP4 (RA) |
| 47 | RB1 (RA) + p16 (RA) |
| 48 | RB1 (RA) + p18 (RA) |
| 49 | RB1 (RA) + p21 (RA) |
| 50 | RB1 (RA) + p27 (RA) |
| 51 | RB1 (RA) + SRC (IA) |
| 52 | IGFBP4 (RA) + p16 (RA) |
| 53 | IGFBP4 (RA) + p18 (RA) |
| 54 | IGFBP4 (RA) + p21 (RA) |
| 55 | IGFBP4 (RA) + p27 (RA) |
| 56 | IGFBP4 (RA) + SRC (IA) |
| 57 | p16 (RA) + p18 (RA) |
| 58 | p16 (RA) + p21 (RA) |
| 59 | p16 (RA) + p27 (RA) |
| 60 | p16 (RA) + SRC (IA) |
| 61 | p18 (RA) + p21 (RA) |
| 62 | p18 (RA) + p27 (RA) |
| 63 | p18 (RA) + SRC (IA) |
| 64 | p21 (RA) + p27 (RA) |
| 65 | p21 (RA) + SRC (IA) |
| 66 | p27 (RA) + SRC (IA) |
| 67 | PTEN (RA) + CASP3 (RA) + CASP8 |
| | (RA) |
| 68 | PTEN (RA) + CASP3 (RA) + CDH1 |
| | (RA) |
| 69 | PTEN (RA) + CASP3 (RA) + ITGB1 |
| | (RA) |
| 70 | PTEN (RA) + CASP3 (RA) + RB1 |
| | (RA) |
| 71 | PTEN (RA) + CASP3 (RA) + IGFBP4 |
| | (RA) |
| 72 | PTEN (RA) + CASP3 (RA) + p16 |
| | (RA) |
| 73 | PTEN (RA) + CASP3 (RA) + p18 |
| | (RA) |
| 74 | PTEN (RA) + CASP3 (RA) + p21 |
| | (RA) |
| 75 | PTEN (RA) + CASP3 (RA) + p27 |
| | (RA) |
| 76 | PTEN (RA) + CASP3 (RA) + SRC |
| | (IA) |
| 77 | PTEN (RA) + CASP8 (RA) + CDH1 |
| | (RA) |
| 78 | PTEN (RA) + CASP8 (RA) + ITGB1 |
| | (RA) |
| 79 | PTEN (RA) + CASP8 (RA) + RB1 |
| | (RA) |
| 80 | PTEN (RA) + CASP8 (RA) + IGFBP4 |
| | (RA) |
| 81 | PTEN (RA) + CASP8 (RA) + p16 |
| | (RA) |
| 82 | PTEN (RA) + CASP8 (RA) + p18 |
| | (RA) |
| 83 | PTEN (RA) + CASP8 (RA) + p21 |
| | (RA) |
| 84 | PTEN (RA) + CASP8 (RA) + p27 |
| | (RA) |
| 85 | PTEN (RA) + CASP8 (RA) + SRC |
| | (IA) |
| 86 | PTEN (RA) + CDH1 (RA) + ITGB1 |
| | (RA) |
| 87 | PTEN (RA) + CDH1 (RA) + RB1 |
| | (RA) |
| 88 | PTEN (RA) + CDH1 (RA) + IGFBP4 |
| | (RA) |
| 89 | PTEN (RA) + CDH1 (RA) + p16 (RA) |
| 90 | PTEN (RA) + CDH1 (RA) + p18 (RA) |
| 91 | PTEN (RA) + CDH1 (RA) + p21 (RA) |
| 92 | PTEN (RA) + CDH1 (RA) + p27 (RA) |
| 93 | PTEN (RA) + CDH1 (RA) + SRC (IA) |
| 94 | PTEN (RA) + ITGB1 (RA) + RB1 |
| | (RA) |
| 95 | PTEN (RA) + ITGB1 (RA) + IGFBP4 |
| | (RA) |
| 96 | PTEN (RA) + ITGB1 (RA) + p16 |
| | (RA) |
| 97 | PTEN (RA) + ITGB1 (RA) + p18 |
| | (RA) |
| 98 | PTEN (RA) + ITGB1 (RA) + p21 |
| | (RA) |
| 99 | PTEN (RA) + ITGB1 (RA) + p27 |
| | (RA) |
| 100 | PTEN (RA) + ITGB1 (RA) + SRC |
| | (IA) |
| 101 | PTEN (RA) + RB1 (RA) + IGFBP4 |
| | (RA) |
| 102 | PTEN (RA) + RB1 (RA) + p16 (RA) |
| 103 | PTEN (RA) + RB1 (RA) + p18 (RA) |
| 104 | PTEN (RA) + RB1 (RA) + p21 (RA) |
| 105 | PTEN (RA) + RB1 (RA) + p27 (RA) |
| 106 | PTEN (RA) + RB1 (RA) + SRC (IA) |
| 107 | PTEN (RA) + IGFBP4 (RA) + p16 |
| | (RA) |
| 108 | PTEN (RA) + IGFBP4 (RA) + p18 |
| | (RA) |
| 109 | PTEN (RA) + IGFBP4 (RA) + p21 |
| | (RA) |
| 110 | PTEN (RA) + IGFBP4 (RA) + p27 |
| | (RA) |
| 111 | PTEN (RA) + IGFBP4 (RA) + SRC |
| | (IA) |
| 112 | PTEN (RA) + p16 (RA) + p18 (RA) |
| 113 | PTEN (RA) + p16 (RA) + p21 (RA) |
| 114 | PTEN (RA) + p16 (RA) + p27 (RA) |
| 115 | PTEN (RA) + p16 (RA) + SRC (IA) |
| 116 | PTEN (RA) + p18 (RA) + p21 (RA) |
| 117 | PTEN (RA) + p18 (RA) + p27 (RA) |
| 118 | PTEN (RA) + p18 (RA) + SRC (IA) |
| 119 | PTEN (RA) + p21 (RA) + p27 (RA) |
| 120 | PTEN (RA) + p21 (RA) + SRC (IA) |
| 121 | PTEN (RA) + p27 (RA) + SRC (IA) |
| 122 | CASP3 (RA) + CASP8 (RA) + CDH1 |
| | (RA) |
| 123 | CASP3 (RA) + CASP8 (RA) + ITGB1 |
| | (RA) |
| 124 | CASP3 (RA) + CASP8 (RA) + RB1 |
| | (RA) |
| 125 | CASP3 (RA) + CASP8 (RA) + |
| | IGFBP4 (RA) |
| 126 | CASP3 (RA) + CASP8 (RA) + p16 |
| | (RA) |
| 127 | CASP3 (RA) + CASP8 (RA) + p18 |
| | (RA) |
| 128 | CASP3 (RA) + CASP8 (RA) + p21 |
| | (RA) |
| 129 | CASP3 (RA) + CASP8 (RA) + p27 |
| | (RA) |
| 130 | CASP3 (RA) + CASP8 (RA) + SRC |
| | (IA) |
| 131 | CASP3 (RA) + CDH1 (RA) + ITGB1 |
| | (RA) |
| 132 | CASP3 (RA) + CDH1 (RA) + RB1 |
| | (RA) |
| 133 | CASP3 (RA) + CDH1 (RA) + IGFBP4 |
| | (RA) |
| 134 | CASP3 (RA) + CDH1 (RA) + p16 |
| | (RA) |
| 135 | CASP3 (RA) + CDH1 (RA) + p18 |
| | (RA) |
| 136 | CASP3 (RA) + CDH1 (RA) + p21 |
| | (RA) |
| 137 | CASP3 (RA) + CDH1 (RA) + p27 |
| | (RA) |
| 138 | CASP3 (RA) + CDH1 (RA) + SRC |
| | (IA) |
| 139 | CASP3 (RA) + ITGB1 (RA) + RB1 |
| | (RA) |
| 140 | CASP3 (RA) + ITGB1 (RA) + |
| | IGFBP4 (RA) |
| 141 | CASP3 (RA) + ITGB1 (RA) + p16 |
| | (RA) |
| 142 | CASP3 (RA) + ITGB1 (RA) + p18 |
| | (RA) |
| 143 | CASP3 (RA) + ITGB1 (RA) + p21 |
| | (RA) |
| 144 | CASP3 (RA) + ITGB1 (RA) + p27 |
| | (RA) |
| 145 | CASP3 (RA) + ITGB1 (RA) + SRC |
| | (IA) |
| 146 | CASP3 (RA) + RB1 (RA) + IGFBP4 |
| | (RA) |
| 147 | CASP3 (RA) + RB1 (RA) + p16 (RA) |
| 148 | CASP3 (RA) + RB1 (RA) + p18 (RA) |
| 149 | CASP3 (RA) + RB1 (RA) + p21 (RA) |
| 150 | CASP3 (RA) + RB1 (RA) + p27 (RA) |
| 151 | CASP3 (RA) + RB1 (RA) + SRC (IA) |
| 152 | CASP3 (RA) + IGFBP4 (RA) + p16 |
| | (RA) |
| 153 | CASP3 (RA) + IGFBP4 (RA) + p18 |
| | (RA) |
| 154 | CASP3 (RA) + IGFBP4 (RA) + p21 |
| | (RA) |
| 155 | CASP3 (RA) + IGFBP4 (RA) + p27 |
| | (RA) |
| 156 | CASP3 (RA) + IGFBP4 (RA) + SRC |
| | (IA) |
| 157 | CASP3 (RA) + p16 (RA) + p18 (RA) |
| 158 | CASP3 (RA) + p16 (RA) + p21 (RA) |
| 159 | CASP3 (RA) + p16 (RA) + p27 (RA) |
| 160 | CASP3 (RA) + p16 (RA) + SRC (IA) |
| 161 | CASP3 (RA) + p18 (RA) + p21 (RA) |
| 162 | CASP3 (RA) + p18 (RA) + p27 (RA) |
| 163 | CASP3 (RA) + p18 (RA) + SRC (IA) |
| 164 | CASP3 (RA) + p21 (RA) + p27 (RA) |
| 165 | CASP3 (RA) + p21 (RA) + SRC (IA) |
| 166 | CASP3 (RA) + p27 (RA) + SRC (IA) |
| 167 | CASP8 (RA) + CDH1 (RA) + ITGB1 |
| | (RA) |
| 168 | CASP8 (RA) + CDH1 (RA) + RB1 |
| | (RA) |
| 169 | CASP8 (RA) + CDH1 (RA) + IGFBP4 |
| | (RA) |
| 170 | CASP8 (RA) + CDH1 (RA) + p16 (RA) |
| 171 | CASP8 (RA) + CDH1 (RA) + p18 (RA) |
| 172 | CASP8 (RA) + CDH1 (RA) + p21 (RA) |
| 173 | CASP8 (RA) + CDH1 (RA) + p27 (RA) |
| 174 | CASP8 (RA) + CDH1 (RA) + SRC |
| | (IA) |
| 175 | CASP8 (RA) + ITGB1 (RA) + RB1 |
| | (RA) |
| 176 | CASP8 (RA) + ITGB1 (RA) + IGFBP4 |
| | (RA) |
| 177 | CASP8 (RA) + ITGB1 (RA) + p16 |
| | (RA) |
| 178 | CASP8 (RA) + ITGB1 (RA) + p18 |
| | (RA) |
| 179 | CASP8 (RA) + ITGB1 (RA) + p21 |
| | (RA) |
| 180 | CASP8 (RA) + ITGB1 (RA) + p27 |
| | (RA) |
| 181 | CASP8 (RA) + ITGB1 (RA) + SRC |
| | (IA) |
| 182 | CASP8 (RA) + RB1 (RA) + IGFBP4 |
| | (RA) |
| 183 | CASP8 (RA) + RB1 (RA) + p16 (RA) |
| 184 | CASP8 (RA) + RB1 (RA) + p18 (RA) |
| 185 | CASP8 (RA) + RB1 (RA) + p21 (RA) |
| 186 | CASP8 (RA) + RB1 (RA) + p27 (RA) |
| 187 | CASP8 (RA) + RB1 (RA) + SRC (IA) |
| 188 | CASP8 (RA) + IGFBP4 (RA) + p16 |
| | (RA) |
| 189 | CASP8 (RA) + IGFBP4 (RA) + p18 |
| | (RA) |
| 190 | CASP8 (RA) + IGFBP4 (RA) + p21 |
| | (RA) |
| 191 | CASP8 (RA) + IGFBP4 (RA) + p27 |
| | (RA) |
| 192 | CASP8 (RA) + IGFBP4 (RA) + SRC |
| | (IA) |
| 193 | CASP8 (RA) + p16 (RA) + p18 (RA) |
| 194 | CASP8 (RA) + p16 (RA) + p21 (RA) |
| 195 | CASP8 (RA) + p16 (RA) + p27 (RA) |
| 196 | CASP8 (RA) + p16 (RA) + SRC (IA) |
| 197 | CASP8 (RA) + p18 (RA) + p21 (RA) |
| 198 | CASP8 (RA) + p18 (RA) + p27 (RA) |
| 199 | CASP8 (RA) + p18 (RA) + SRC (IA) |
| 200 | CASP8 (RA) + p21 (RA) + p27 (RA) |
| 201 | CASP8 (RA) + p21 (RA) + SRC (IA) |
| 202 | CASP8 (RA) + p27 (RA) + SRC (IA) |
| 203 | CDH1 (RA) + ITGB1 (RA) + RB1 |
| | (RA) |
| 204 | CDH1 (RA) + ITGB1 (RA) + IGFBP4 |
| | (RA) |
| 205 | CDH1 (RA) + ITGB1 (RA) + p16 (RA) |
| 206 | CDH1 (RA) + ITGB1 (RA) + p18 (RA) |
| 207 | CDH1 (RA) + ITGB1 (RA) + p21 (RA) |
| 208 | CDH1 (RA) + ITGB1 (RA) + p27 (RA) |
| 209 | CDH1 (RA) + ITGB1 (RA) + SRC (IA) |
| 210 | CDH1 (RA) + RB1 (RA) + IGFBP4 |
| | (RA) |
| 211 | CDH1 (RA) + RB1 (RA) + p16 (RA) |
| 212 | CDH1 (RA) + RB1 (RA) + p18 (RA) |
| 213 | CDH1 (RA) + RB1 (RA) + p21 (RA) |
| 214 | CDH1 (RA) + RB1 (RA) + p27 (RA) |
| 215 | CDH1 (RA) + RB1 (RA) + SRC (IA) |
| 216 | CDH1 (RA) + IGFBP4 (RA) + p16 |
| | (RA) |
| 217 | CDH1 (RA) + IGFBP4 (RA) + p18 |
| | (RA) |
| 218 | CDH1 (RA) + IGFBP4 (RA) + p21 |
| | (RA) |
| 219 | CDH1 (RA) + IGFBP4 (RA) + p27 |
| | (RA) |
| 220 | CDH1 (RA) + IGFBP4 (RA) + SRC |
| | (IA) |
| 221 | CDH1 (RA) + p16 (RA) + p18 (RA) |
| 222 | CDH1 (RA) + p16 (RA) + p21 (RA) |
| 223 | CDH1 (RA) + p16 (RA) + p27 (RA) |
| 224 | CDH1 (RA) + p16 (RA) + SRC (IA) |
| 225 | CDH1 (RA) + p18 (RA) + p21 (RA) |
| 226 | CDH1 (RA) + p18 (RA) + p27 (RA) |
| 227 | CDH1 (RA) + p18 (RA) + SRC (IA) |
| 228 | CDH1 (RA) + p21 (RA) + p27 (RA) |
| 229 | CDH1 (RA) + p21 (RA) + SRC (IA) |
| 230 | CDH1 (RA) + p27 (RA) + SRC (IA) |
| 231 | ITGB1 (RA) + RB1 (RA) + IGFBP4 |
| | (RA) |
| 232 | ITGB1 (RA) + RB1 (RA) + p16 (RA) |
| 233 | ITGB1 (RA) + RB1 (RA) + p18 (RA) |
| 234 | ITGB1 (RA) + RB1 (RA) + p21 (RA) |
| 235 | ITGB1 (RA) + RB1 (RA) + p27 (RA) |
| 236 | ITGB1 (RA) + RB1 (RA) + SRC (IA) |
| 237 | ITGB1 (RA) + IGFBP4 (RA) + p16 |
| | (RA) |
| 238 | ITGB1 (RA) + IGFBP4 (RA) + p18 |
| | (RA) |
| 239 | ITGB1 (RA) + IGFBP4 (RA) + p21 |
| | (RA) |
| 240 | ITGB1 (RA) + IGFBP4 (RA) + p27 |
| | (RA) |
| 241 | ITGB1 (RA) + IGFBP4 (RA) + SRC |
| | (IA) |
| 242 | ITGB1 (RA) + p16 (RA) + p18 (RA) |
| 243 | ITGB1 (RA) + p16 (RA) + p21 (RA) |
| 244 | ITGB1 (RA) + p16 (RA) + p27 (RA) |
| 245 | ITGB1 (RA) + p16 (RA) + SRC (IA) |
| 246 | ITGB1 (RA) + p18 (RA) + p21 (RA) |
| 247 | ITGB1 (RA) + p18 (RA) + p27 (RA) |
| 248 | ITGB1 (RA) + p18 (RA) + SRC (IA) |
| 249 | ITGB1 (RA) + p21 (RA) + p27 (RA) |
| 250 | ITGB1 (RA) + p21 (RA) + SRC (IA) |
| 251 | ITGB1 (RA) + p27 (RA) + SRC (IA) |
| 252 | RB1 (RA) + IGFBP4 (RA) + p16 (RA) |
| 253 | RB1 (RA) + IGFBP4 (RA) + p18 (RA) |
| 254 | RB1 (RA) + IGFBP4 (RA) + p21 (RA) |
| 255 | RB1 (RA) + IGFBP4 (RA) + p27 (RA) |
| 256 | RB1 (RA) + IGFBP4 (RA) + SRC (IA) |
| 257 | RB1 (RA) + p16 (RA) + p18 (RA) |
| 258 | RB1 (RA) + p16 (RA) + p21 (RA) |
| 259 | RB1 (RA) + p16 (RA) + p27 (RA) |
| 260 | RB1 (RA) + p16 (RA) + SRC (IA) |
| 261 | RB1 (RA) + p18 (RA) + p21 (RA) |
| 262 | RB1 (RA) + p18 (RA) + p27 (RA) |
| 263 | RB1 (RA) + p18 (RA) + SRC (IA) |
| 264 | RB1 (RA) + p21 (RA) + p27 (RA) |
| 265 | RB1 (RA) + p21 (RA) + SRC (IA) |
| 266 | RB1 (RA) + p27 (RA) + SRC (IA) |
| 267 | IGFBP4 (RA) + p16 (RA) + p18 (RA) |
| 268 | IGFBP4 (RA) + p16 (RA) + p21 (RA) |
| 269 | IGFBP4 (RA) + p16 (RA) + p27 (RA) |
| 270 | IGFBP4 (RA) + p16 (RA) + SRC (IA) |
| 271 | IGFBP4 (RA) + p18 (RA) + p21 (RA) |
| 272 | IGFBP4 (RA) + p18 (RA) + p27 (RA) |
| 273 | IGFBP4 (RA) + p18 (RA) + SRC (IA) |
| 274 | IGFBP4 (RA) + p21 (RA) + p27 (RA) |
| 275 | IGFBP4 (RA) + p21 (RA) + SRC (IA) |
| 276 | IGFBP4 (RA) + p27 (RA) + SRC (IA) |
| 277 | p16(RA) + p18 (RA) + p21 (RA) |
| 278 | p16(RA) + p18 (RA) + p27 (RA) |
| 279 | p16(RA) + p18 (RA) + SRC (IA) |
| 280 | p16(RA) + p21 (RA) + p27 (RA) |
| 281 | p16 (RA) + p21 (RA) + SRC (IA) |
| 282 | p16 (RA) + p27 (RA) + SRC (IA) |
| 283 | p18 (RA) + p21 (RA) + p27 (RA) |
| 284 | p18 (RA) + p21 (RA) + SRC (IA) |
| 285 | p18 (RA) + p27 (RA) + SRC (IA) |
| 286 | p21 (RA) + p27 (RA) + SRC (IA) |

*“RA” means a genetic modification that results in reduced protein activity; “IA” means a genetic modification that results in increased protein activity.

In some embodiments, the cell line comprises (i) the combination of genetic modifications according to one of Embodiments 1-286 in Table 2; and (ii) a genetic modification resulting in increased protein activity of TERT.

In some embodiments, the cell line comprises (i) the combination of genetic modifications according to one of Embodiments 1-286 in Table 2; and (ii) a genetic modification resulting in reduced protein activity of p53.

In some embodiments, the cell line comprises (i) the combination of genetic modifications according to one of Embodiments 1-286 in Table 2; (ii) a genetic modification resulting in increased protein activity of TERT; and (iii) a genetic modification resulting in reduced protein activity of p53.

### Promoter Sequence

The disclosure also provides various promoters that can be used for modulating the expression of the PTEN protein (knock-in and/or endogenous).

In some embodiments, the genetically modified cell line of the disclosure comprises a recombinant nucleic acid comprising a promoter sequence operably linked to the open reading frame (ORF) of a knock-in gene. In some embodiments, the genetically modified cell line of the disclosure comprises a promoter sequence of the disclosure operably linked to the endogenous gene. In some embodiments, the promoter is a constitutive promoter, a tissue or cell specific promoter, an inducible promoter, or a combination thereof.

In some embodiments, the promoter is a constitutive promoter. In some embodiments, the constitutive promoter is selected from the group consisting of Proliferation Cell Nuclear Antigen core promoter (e.g., SEQ ID NO: 17), Actin promoter (e.g., SEQ ID NO: 18), Elongation Factor 1 alpha promoter (e.g., SEQ ID NO: 19), Ubiquitin promoter (e.g., SEQ ID NO: 20), and Phosphoglycerate Kinase promoter (e.g., SEQ ID NO: 21). In some embodiments, the constitutive promoter comprises or consists of a nucleotide sequence having at least 60%, at least 65%, at least 70%, at least 75%, at least 80%, at least 85%, at least 90%, at least 95%, at least 96%, at least 97%, at least 98%, at least 99%, at least 99.5%, or 100% identity to any one of SEQ ID NO: 17-21, including all ranges and subranges therebetween.

In some embodiments, the promoter is a myoblast specific promoter. In some embodiments, the myoblast specific promoter is selected from the group consisting of Myosin light chain 2 promoter (e.g., SEQ ID NO: 22), Creatine Kinase promoter (e.g., SEQ ID NO: 23), Insulin-like Growth Factor 1 promoter (e.g., SEQ ID NO: 24), Myogenin promoter (e.g., SEQ ID NO: 25), Pax3 promoter (e.g., SEQ ID NO: 26), SIX1 promoter (e.g., SEQ ID NO: 27) and Desmin promoter (e.g., SEQ ID NO: 28). In some embodiments, the myoblast specific promoter comprises or consists of a nucleotide sequence having at least 60%, at least 65%, at least 70%, at least 75%, at least 80%, at least 85%, at least 90%, at least 95%, at least 96%, at least 97%, at least 98%, at least 99%, at least 99.5%, or 100% identity to any one of SEQ ID NO: 22-28, including all ranges and subranges therebetween.

In some embodiments, the promoter is a fibroblast specific promoter.

In some embodiments, the promoter is an inducible promoter. Inducible promoters suitable for controlling protein expression include promoters responsive to exogenous agents (e.g., small molecule agents) or to physiological cues. Exemplary inducible promoters include, but are not limited to, the Tet-On system (Clontech; Chen H., et al., (2015) BMC Biotechnol. 15 (1): 4)), the RheoSwitch system (Intrexon; Sowa G., et al., (2011) Spine, 36 (10): E623-8), a hypoxia response element (HRE) that binds HIF-Ia and tetracycline response element (such as described by Gossen & Bujard (1992, Proc. Natl. Acad. Sci. USA 89:5547-551 and Dingermann et al. (1992), Mol Cell Biol. 12 (9): 4038-45); an ecdysone-inducible response element (No D et al., 1996, Proc. Natl. Acad. Sci. USA. 93:3346-3351), a metal-ion response element such as described by Mayo et al. (1982, Cell 29:99-108); Brinster et al. (1982, Nature 296:39-42) and Searle et al. (1985, Mol. Cell. Biol. 5:1480-1489); a heat shock response element such as described by Nouer et al. (in: Heat Shock Response, ed. Nouer, L., CRC, Boca Raton, Fla., ppI67-220, 1991); or a hormone response element such as described by Lee et al. (1981, Nature 294:228-232); Hynes et al. (Proc. Natl. Acad. Sci. USA 78:2038-2042, 1981); Klock et al. (Nature 329:734-736, 1987); and Israel and Kaufman (1989, Nucl. Acids Res. 17:2589-2604).

In some embodiments, the inducible promoter drives expression (or overexpression) of the target protein in the presence of, but not in the absence of, an exogenous agent. In some embodiments, the inducible promoter drives expression (or overexpression) of the target protein in the absence of, but not in the presence of, an exogenous agent. In some embodiments, the inducible promoter drives expression (or overexpression) of the target protein in the presence of, but not in the absence of, a physiological cue (e.g., hypoxia). In some embodiments, the inducible promoter drives expression (or overexpression) of the target protein in the absence of, but not in the presence of, a physiological cue (e.g., hypoxia). Therefore, in some embodiments, the target protein comprising one or more mutations is inducibly-expressed.

In some embodiments, the promoter may be a synthetic promoter. In some embodiments, the synthetic promoter combines the sequences of two or more promoters of the disclosure.

### Genomic Insertion Sites

The disclosure also provides various genomic insertion site that can be used for accommodating a knock-in gene. Non-limiting examples of a knock-in gene includes the gene encoding an active TERT or SRC protein or the gene encoding a dominant negative form of PTEN, CASP3, CASP8, CDH1, ITGB1, RB1, IGFBP4, p16, p18, p21, p27, or p53 protein.

In some embodiments, the genetically modified cell line of the disclosure comprises a recombinant nucleic acid comprising a knock-in gene, said recombinant nucleic acid having been inserted into a genomic insertion site of the cell. In some embodiments, the present disclosure teaches insertion of recombinant nucleic acids into genomic insertion sites.

In some embodiments, the genomic insertion site is a “safe harbor site”. In some embodiments, the safe harbor site is Rosa26 located in the bovine chromosome 22 between 17.25Kbp and 17.35Kbp. In some embodiments, the safe harbor site is Bos Taurus Neutral Site 1 (BTNSI) located between 50,898,000 and 50,899,000 bps on the Bos Taurus Chromosome. In some embodiments, the insertion is mediated by CRISPR technology using the sgRNA according to SEQ ID NO: 37, and the 5′ and/or 3′ homology arms according to SEQ ID NO: 38 and/or 39.

In some embodiments, the genomic insertion site encodes a functional protein. In some embodiments, the functional protein encoded within the genomic insertion site promotes anoikis of the cell line.

### Genetic Modification Methods

In one aspect, the present disclosure provides various genetic modification methods for producing genetically modified cell lines.

In some embodiments, the genetic modification is mediated via an engineered nuclease that recognizes and cleaves specific recognition sequences in the cell genomic DNA. In some embodiments, the engineered nuclease is based on CRISPR (Clustered Regularly Interspaced Short Palindromic Repeats) technology—e.g., a CRISPR-cas nuclease. In some embodiments, the engineered nuclease is a zinc finger nuclease, a meganuclease, or TALEN nuclease. Cleavage at such recognition sequences can allow for NHEJ at the cleavage site and homologous recombination of exogenous recombinant nucleic acid sequences directly into or near the cleavage site. In some embodiments, the genetic modification method comprises inducing a single stranded break or double stranded break in the target gene using the engineered nuclease. In some embodiments, the double-stranded or single-stranded break undergoes repair via a cellular repair process, such as by non-homologous end-joining (NHEJ) or homology-directed repair (HDR). In some embodiments, the repair process is error-prone and results in a frameshift mutation, a premature stop codon, or other types of mutations that disrupts the gene expression and/or protein function. In some embodiments, the genetic modification method comprises introducing an exogenous nucleic acid comprising the desirable genetic modification sequence, wherein the exogenous nucleic acid comprises a 5′ homology arm and a 3′ homology arm to promote recombination of the nucleic acid sequence into the cell genome at the nuclease cleavage site.

In some embodiments, engineered nucleases can be delivered into a cell in the form of protein or as a nucleic acid encoding the engineered nuclease. Such nucleic acid can be DNA (e.g., circular or linearized plasmid DNA or PCR products) or RNA. In some embodiments, mRNA encoding the engineered nuclease is delivered to the cell because this reduces the likelihood that the gene encoding the engineered nuclease will integrate into the genome of the cell. Such mRNA encoding an engineered nuclease can be produced using methods known in the art such as in vitro transcription. In some embodiments, the mRNA is capped using 7-methyl-guanosine. In some embodiments, the mRNA may be polyadenylated. Purified nuclease proteins can be delivered into cells to cleave genomic DNA, which allows for homologous recombination or non-homologous end-joining at the cleavage site with a sequence of interest, by a variety of different mechanisms known in the art. In some embodiments, engineered nuclease proteins, or DNA/mRNA encoding engineered nucleases, are coupled to a cell penetrating peptide or targeting ligand to facilitate cellular uptake.

In some embodiments, the exogenous nucleic acid (e.g, the recombinant nucleic acid comprising a knock-in gene) is introduced into a cell using a viral vector. Such vectors are known in the art and include lentiviral vectors, adenoviral vectors, and adeno-associated virus (AAV) vectors (reviewed in Vannucci, et al. (2013 New Microbiol. 36:1-22). Recombinant AAV vectors useful in the invention can have any serotype that allows for transduction of the virus into the cell and insertion of the nuclease gene into the cell genome. In some embodiments, the exogenous nucleic acid (e.g., the recombinant nucleic acid comprising a knock-in gene) is introduced into a cell using a non-viral method. In some embodiments, the non-viral method is electroporation. In some embodiments, the non-viral method is a liposome-based method.

In some embodiments, the genetic modification method of the disclosure allows introduction of a recombinant nucleic acid comprising a knock-in gene. In some embodiments, the genetic modification method of the disclosure allows introduction of one or more mutations to the endogenous protein encoded by the corresponding endogenous gene. In some embodiments, the genetic modification method of the disclosure results in reduced or no expression of the endogenous protein (e.g., via disrupting the ORF region of the gene or the endogenous promoter sequence of the gene locus).

In some embodiments, a recombinant nucleic acid comprising a knock-in gene may be inserted into the target cell genome by homologous recombination (e.g., homology-directed repair (HDR)). In some embodiments, HDR is initiated by cutting a target site of the cell chromosome using CRISPR technology. In some embodiments, single guide RNA (sgRNA) are designed to include target sequences in the cell chromosome which are adjacent to protospacer adjacent motifs (PAMs) which are recognized by Cas9 and enable the enzyme to cut the DNA sequence. In some embodiments, the recombinant nucleic acid comprises 5′ and 3′ homology arms matching the nucleotide sequences adjacent to the cut site as determined by the sgRNA and will allow for efficient and highly specific insertion of the recombinant nucleic acid into the locus.

In some embodiments, the genetic modification method of the disclosure comprises introducing a CRISPRi module to repress endogenous protein expression. In some embodiments, the CRISPRi module allows expression of 1) a dCas9 protein fused to one or more transcriptional repressors and 2) a gRNA that targets the promoter region of the endogenous gene locus. In some embodiments, the assembly of gRNA-dCas9 protein complex allows recruitment of the transcriptional repressors to the promoter region of the gene locus which in turn repress the expression of corresponding mRNA as well as protein activity.

In some embodiments, the genetic modification method comprises introducing an expression construct which down-regulates protein expression via RNAi to the cell line. In some embodiments, the construct expresses an RNAi molecule (e.g., shRNA) that specifically binds to and degrades the mRNA, therefore reducing the expression and activity of corresponding protein in the cell line.

### Genetically Modified Cell Lines

The disclosure provides cell lines comprising one or more genetic modifications compared to a control cell line without said genetic modification.

In some embodiments, the genetic modified cell line is derived from a metazoan cell. The metazoan cell may be a cell from a wide variety of animal species, including without limitation livestock cells, poultry cells, wild animal cells, aquatic species cells, arthropod species cells, or cells of other animals consumed by humans. Livestock includes without limitation cows, pigs, sheep, or goats. Poultry includes without limitation turkeys, chickens, or ducks. Other animals include without limitation deer. Aquatic species include fish but may also include other aquatic species. The cell lines and methods herein are not limited to any particular species disclosed herein and contemplate all metazoan cell lines that can be used to manufacture cell-based meat.

In some embodiments, the metazoan cell is a myoblast cell. In some embodiments, the metazoan cell is a stem cell, a fibroblast cell, a myogenic cell, a preadipocyte cell or an adipocyte cell. In some embodiments, the metazoan cell is a mesenchymal stem cell, a bone marrow derived cell, a cardiomyocytes (cells of the myocardium, heart), or a hepatocyte (liver cells, liver), or other cell types found in organ meat such as heart, kidney, or liver. In some embodiments, the genetically modified cell line derived from the metazoan cell has the same cell type as the metazoan cell.

In some embodiments, the genetically modified cell line is a poultry cell line. In some embodiments, the genetically modified cell line has a species identity of Gallus gallus, Meleagris gallopavo, Anas platyrhynchos, or Coturnix coturnix. In some embodiments, the genetically modified cell line is a livestock cell line. In some embodiments, the genetically modified cell line has a species identity of Bos taurus, Sus scrofa, capra aegagrus hircus or Ovis aries. In some embodiments, the genetically modified cell line has a species identity of Bos taurus, Bos indicus, or a hybrid thereof. In some embodiments, the genetically modified cell line is a seafood cell line. In some embodiments, the genetically modified cell line has a species identity of Salmo salar, Thunnus thynnus, Gadus morhua, Homarus americanus or Litopenaeus setiferus.

Cell types of the genetically modified cell line of the disclosure include but are not limited to skeletal muscle cells, myoblasts, myogenic cells, fibroblasts, mesenchymal stem cells, endothelial cells, adipose progenitor cells, adipoblasts, adipocytes, cardiomyocytes (cells of the myocardium, heart), hepatocytes (liver cells, liver), cell types found in organ meat such heart, kidney, or liver, or bone marrow derived immune cells such as macrophages.

In some embodiments, cells of the genetically modified cell line are primary stem cells or self-renewing stem cell lines. In some embodiments, cells of the genetically modified cell line are myoblasts, myocytes, fibroblasts, preadipocytes, induced pluripotent stem cells, hepatocytes, mesenchymal stem cells, adipocytes, embryonic stem cells or chondrocytes.

In some embodiments, cells of the genetically modified cell line are myogenic cells. In some embodiments, the cells are natively myogenic (e.g., are myogenic cells such as myoblasts, myocytes, satellite cells, side population cells, muscle derived stem cells, mesenchymal stem cells, myogenic pericytes, and mesoangioblasts that are cultured in the cultivation infrastructure). In some embodiments, the cells are not natively myogenic (e.g., are non-myogenic cells such as fibroblasts, preadipocytes, or non-myogenic stem cells that are cultured to become myogenic cells in the cultivation process).

In some embodiments, cells of the genetically modified cell line are somatic cells. In some embodiments, cells of the genetically modified cell line are not somatic cells.

In some embodiments, the genetically modified cell line is skeletal muscle lineage. Cells of the skeletal muscle lineage include myoblasts, myocytes, and skeletal muscle progenitor cells, also called myogenic progenitors, that include satellite cells, side population cells, muscle derived stem cells, mesenchymal stem cells, myogenic pericytes, and mesoangioblasts. In some embodiments, the genetically modified cell line is subcutaneous adipose tissue lineage. In some embodiments, the genetically modified cell line is connective tissue lineage.

In some embodiments, the genetically modified cell line is a myoblast cell line. In some embodiments, the genetically modified cell line is a bovine myoblast cell line. In some embodiments, the genetically modified cell line is a chicken myoblast cell line. In some embodiments, the genetically modified cell line is a primary bovine myoblast cell line. In some embodiments, the genetically modified cell line is a primary chicken myoblast cell line.

In some embodiments, the genetically modified cell line is a fibroblast cell line. In some embodiments, the genetically modified cell line is a bovine fibroblast cell line. In some embodiments, the genetically modified cell line is a chicken fibroblast cell line. In some embodiments, the genetically modified cell line is a primary bovine fibroblast cell line. In some embodiments, the genetically modified cell line is a primary chicken fibroblast cell line.

In some embodiments, the genetically modified cell line is a preadipocyte cell line. In some embodiments, the genetically modified cell line is a bovine preadipocyte cell line. In some embodiments, the genetically modified cell line is a chicken preadipocyte cell line. In some embodiments, the genetically modified cell line is a primary bovine preadipocyte cell line. In some embodiments, the genetically modified cell line is a primary chicken preadipocyte cell line.

In some embodiments, the genetically modified cells of the present disclosure will exhibit different properties compared to wildtype or control cells lacking said genetic modification. In some embodiments, these different properties can be used to screen the genetically modified cell line of the present disclosure.

In some embodiments, the genetically modified cell line of the present disclosure can be screened based on their ability to stay viable and/or proliferate in suspension culture. In some embodiments, the genetically modified cell line of the disclosure exhibits increased rate of proliferation and/or decreased differentiation compared to a control cell line in suspension culture. In some embodiments, the suspension culture comprises a cell growth medium lacking or having a reduced level of serum and/or one or more exogenous growth factors. In some embodiments, the one or more exogenous growth factors comprises an FGF. In some embodiments, the FGF is FGF-2. In some embodiments, the one or more growth factors are selected from the group consisting of FGF (e.g., FGF-2), EGF, IGF, insulin, and HGF. In some embodiments, the one or more growth factors are selected from the group consisting of FGF (e.g., FGF-2), EGF, IGF, insulin, HGF, TGF-β and TNF-α.

In some embodiments, the genetically modified cell lines are screened based on the increased rate of proliferation in suspension medium. In some embodiments, the genetically modified cell line displays increased rate of proliferation compared to a control cell line in suspension culture. In some embodiments, rate of proliferation is quantified by cell counts at regular intervals (e.g., once every 3 hours, once every 6 hours, once every 12 hours, once every 24 hours, once every 48 hours, once every 72 hours, once a week, once 2 weeks, once a month, or any ranges therebetween). In some embodiments, the rate of proliferation is higher by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, for the genetically modified cell line compared to a control cell line in suspension culture. In some embodiment, the suspension culture comprise a cell growth medium lacking or having reduced level of serum and/or one or more exogenous growth factors.

In some embodiments, the genetically modified cell lines are screened based on the increased metabolic activity in suspension culture. In some embodiments, the genetically modified cell line displays increased metabolic activity compared to a control cell line in suspension culture. In some embodiments, metabolic activity is quantified by ATP level (e.g., using CellTiter Glo assay (G9243, Promega)). In some embodiments, the metabolic activity is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold, for the genetically modified cell line compared to a control cell line in suspension culture. In some embodiment, the suspension culture comprise a cell growth medium lacking or having reduced level of serum and/or one or more exogenous growth factors. In some embodiments, higher metabolic activity indicates higher viability and/or higher proliferation rate. In some embodiments, ATP level is an indicator of number of cells and therefore can be measured over time to estimate cell proliferation.

In some embodiments, the genetically modified cell lines are screened based on the extended replicative capacity in suspension culture. In some embodiments, the genetically modified cell line displays extended replicative capacity compared to a control cell line in suspension culture. In some embodiments, the replicative capacity is extended by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold (including all ranges and subranges therebetween), for the genetically modified cell line compared to a control cell line in suspension culture. In some embodiment, the suspension culture comprise a cell growth medium lacking or having reduced level of serum and/or one or more exogenous growth factors.

In some embodiments, the genetically modified cell line of the disclosure comprises a change of expression level of one or more genes/biomarkers compared to a control cell line as a result of genetic modification. In some embodiments, the genetically modified cell lines are screened based on the expression level of the one or more genes/biomarkers, in adherent culture or in suspension culture. In some embodiments, the expression level is mRNA and/or protein expression level. In some embodiments, the one or more genes/biomarkers are related to proliferations and/or differentiation of the cell line. In some embodiments, the one or more genes/biomarkers are related to the epithelial-mesenchymal transition (EMT) process, such as E-cadherin, N-cadherin and vimentin. In some embodiments, such change is measured in a minimum regular cell growth medium. In some embodiments, such change is measured in a cell growth medium lacking or having reduced level of serum and/or one or more exogenous growth factors. In some embodiments, such change is measured in a suspension culture of the genetically modified cell line.

In some embodiments, the genetically modified cell line expresses a lower level of E-cadherin as compared to a control cell line. In some embodiments, the E-cadherin mRNA expression level of the genetically modified cell line (e.g., as determined by q-RT-PCR) is decreased by at least 10%, at least 20%, at least 30%, at least 40%, at least 50%, at least 60%, at least 70%, at least 80%, at least 90%, at least 95%, at least 97%, at least 98%, or at least 99% (including all ranges and subranges therebetween), as compared to that in a control cell line. In some embodiments, the E-cadherin protein expression level of the genetically modified cell line (e.g., as determined by Western blotting) is decreased by at least 10%, at least 20%, at least 30%, at least 40%, at least 50%, at least 60%, at least 70%, at least 80%, at least 90%, at least 95%, at least 97%, at least 98%, or at least 99% (including all ranges and subranges therebetween), as compared to that in a control cell line. In some embodiments, the cell lines are in a suspension culture comprising a minimum regular cell growth medium or a cell growth medium lacking or having reduced level of serum and/or one or more exogenous growth factors (e.g., as described infra).

In some embodiments, the genetically modified cell line expresses a higher level of N-cadherin as compared to a control cell line. In some embodiments, the N-cadherin mRNA expression level of the genetically modified cell line (e.g., as determined by q-RT-PCR) is increased by at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% (including all ranges and subranges therebetween), as compared to that in a control cell line. In some embodiments, the N-cadherin protein expression level of the genetically modified cell line (e.g., as determined by Western blotting) is increased by at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% (including all ranges and subranges therebetween), as compared to that in a control cell line. In some embodiments, the cell lines are in a suspension culture comprising a minimum regular cell growth medium or a cell growth medium lacking or having reduced level of serum and/or one or more exogenous growth factors (e.g., as described infra).

In some embodiments, the genetically modified cell line expresses a higher level of vimentin as compared to a control cell line. In some embodiments, the vimentin mRNA expression level of the genetically modified cell line (e.g., as determined by q-RT-PCR) is increased by at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% (including all ranges and subranges therebetween), as compared to that in a control cell line. In some embodiments, the vimentin protein expression level of the genetically modified cell line (e.g., as determined by Western blotting) is increased by at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000% (including all ranges and subranges therebetween), as compared to that in a control cell line. In some embodiments, the cell lines are in a suspension culture comprising a minimum regular cell growth medium or a cell growth medium lacking or having reduced level of serum and/or one or more exogenous growth factors (e.g., as described infra).

In some embodiments, the genetically modified cell line is a non-immortalized cell line.

In some embodiments, the genetically modified cell line is an immortalized cell line. In some embodiments, the disclosure provides methods for immortalizing primary cells isolated from an animal to increase the biomass of cultured cells generated or created from the isolated primary cells.

It has been discovered that cells may be directed to proliferate beyond a finite lifespan by manipulating the cell cycle and maintaining telomere length. Inserting certain genes that regulate the cell cycle into the genome of cells provides a method of expanding the proliferative potential of cells and immortalizing cells. Inserted genes may code for proteins that promote progression of the cell cycle to proliferate the cell line, extend the lifespan of the cell or prevent senescence. Genetic amendments for increased or indefinite progression of the cell cycle include those that initiate telomerase reverse transcriptase activation, suppress p53 and retinoblastoma protein function, and activate Ras or c-Myc proto-oncogenes. In some embodiments, the disclosure provides methods for immortalizing or extending the proliferative capacity of cells to achieve cell proliferation by inserting immortalization genes, cell cycle regulator genes, genes that enhance cell cycle progression or genes that prevent senescence into a genome of a cell. Thereafter, the proliferative capacity may be decreased, after sufficient production has occurred, by excising the inserted genes, for example, as disclosed in WO2020/237021. Such immortalized cell lines offer significant advantage for industrial application (e.g., production of a large quantity of cells for preparing meat-like food products) as they can be used repeatedly for production during long campaigns and optimized for large scale culturing conditions with minimal batch-to-batch variation.

In some embodiments, the disclosure utilizes proteins that can deregulate the skeletal muscle cell cycle to increase the total number of cell divisions possible, a strategy that immortalizes a cell type that has an otherwise limited number of mitotic cell divisions in vitro.

In a non-limiting example, a CRISPR/Cas9 genetic modification strategy may be used to insert expression cassettes comprising constitutively expressed genes that code for proteins that promote cell cycle progression, such as CDKs and cyclins, BMI-1, telomerase, SV40T, E6 and E7, and oncoproteins such as Ras or c-Myc, and/or that maintain telomere length, such as telomerase enzyme, at a specific gene locus in animal cells. Using CRISPR/Cas9 to insert an expression cassette into a specific gene locus allows the expression cassette to be targeted to a neutral locus or safe haven locus to reduce the risk of unpredicted endogenous regulation.

In some embodiments, genes used for immortalization may be genes that have been shown to regulate the cell cycle. Suitable genes include but are not limited to SV40T antigen, BMI-1, c-Myc, Ras, cyclin D, CDK4, and telomerase reverse transcriptase.

By way of further description, SV40T is an antigen expressed by the SV40 virus. SV40 is a double stranded DNA virus of rhesus monkey origin. This virus has a number of antigens, but its large tumor antigen (tag) plays a special role in regulating cell signaling pathways that induce cells to enter into S phase and undergo a DNA damage response that facilitates viral DNA replication. Tag also binds to and inactivates the p53 and pRB family of proteins, powerful tumor suppressors involved in cell cycle progression and apoptosis, to create an ideal environment permissive for viral replication. Tag can immortalize cell lines, giving them extended or infinite proliferation potential.

BMI-1 is a protein that works with c-Myc. It is a transcriptional repressor that prevents RNA polymerase activity. Down regulation of BMI-1 leads to up regulation of p16 and p19 tumor suppressors encoded by the ink4a gene locus. Overexpression of BMI-1 leads to immortalization in myogenic cells and down regulation of p16 and p19.

E6 and E7 are proteins from human papilloma virus type 16 (HPV16) E6 and E7 cooperate in mediating-cellular immortalization. They inactivate tumor suppressors p53 and pRB (retinoblastoma protein).

c-Myc is part of the Myc family of regulator genes that encode transcription factors that are expressed in the nucleus. c-Myc has capability to drive cell proliferation (upregulates cyclins, downregulates p21), but it also plays a very important role in regulating cell growth (upregulates ribosomal RNA and proteins), apoptosis, differentiation, and stem cell self renewal. c-Myc also recruits elongation factors (E2Fs).

In some embodiments, the present disclosure provides methods for extending the replicative capacity of metazoan somatic cells using targeted genetic amendments to abrogate inhibition of cell-cycle progression during replicative senescence. In some embodiments, clonal cell lines produced using such a method is suitable for scalable applications and industrial production of metazoan cell biomass. One application is to manufacture skeletal muscle for dietary consumption using cells from the poultry species Gallus gallus and the livestock species Bos taurus. As a non-limiting example, CRISPR/Cas9 may be used to knock out cell cycle inhibitors and expressing telomerase to promote cell cycle progression to develop skeletal muscle cell lines, as disclosure in WO2017/124100, the content of which is incorporated by reference in its entirety.

In some embodiments, the genetically modified cell line does not comprise a heterologous antibiotic resistance gene.

In some embodiments, the genetically modified cell line of the disclosure may be further engineered to have improved meat-like properties; for example, the cell line may be modified to overexpress a protein to improve the color or taste of the cell-based meat product. In some embodiments, the cell line may be modified in a way that generates or enhances the taste and smell of beef, bacon, pork, lamb, goat, turkey, duck, deer, yak, bison, chicken, or other desirable meat flavor in the food ingredient derived from such cells.

### Cell Suspension Culture

In one aspect, the present disclosure provides genetically modified cell lines capable of being adapted to grow in a suspension culture. In some embodiments, the genetically modified cell line has been adapted to grow in a suspension culture. In some embodiments, the suspension culture has a minimum viable cell density of about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 22 million, about 24 million, about 26 million, about 28 million, about 30 million, about 32 million, about 34 million, about 36 million, about 38 million, about 40 million, about 45 million, or about 50 million per milliliter (mL), including all ranges and subranges therebetween.

In some embodiments, the genetically modified cell line is stable in the suspension culture over multiple generations. A cell line is considered “stable” when it maintains its growth characteristics and/or viability over time.

In some embodiments, the growth characteristics are evaluated by the doubling time of cell number in a cell culture. In some embodiments, a cell line in suspension culture is considered “stable” if its doubling time does not change by more than 50% for a given number of generations in a minimum regular growth medium or a cell growth medium lacking or having reduced level of serum and/or one or more exogenous growth factors. For example, in some embodiments, a cell line is said to be stable for 20 generations if its doubling time does not change by more than 50% for 20 generations. In some embodiments, the measurement of doubling time is started when the cell line is at about 30% of its maximum viable cell density in the culture. In some embodiments, the measurement of doubling time is started when the cell line is at a specific viable cell density—for example, a viable cell density of about 0.1 million, about 0.2 million, about 0.3 million, about 0.4 million, about 0.5 million, about 0.6 million, about 0.7 million, about 0.8 million, about 0.9 million, about 1 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 22 million, about 24 million, about 26 million, about 28 million, about 30 million, about 32 million, about 34 million, about 36 million, about 38 million, about 40 million, about 45 million, or about 50 million, cells per milliliter (mL), including all ranges and subranges therebetween.

In some embodiments, the stability of the cell line is evaluated by the cell viability. In some embodiments, a cell line in suspension culture is considered “stable” if the cell viability does not change by more than 20% for a given number of generations in a minimum regular growth medium or a cell growth medium lacking or having reduced level of serum and/or one or more exogenous growth factors. For example, in some embodiments, a cell line is considered stable for 20 generations if the starting cell viability is 90% and the cell viability does not decrease to lower than 70% for 20 generations. In some embodiments, a cell line in suspension culture is considered “stable” if the cell viability is at or above a specific level—for example, 30%, 35%, 40%, 45%, 50%, 55%, 60%, 65%, 70%, 75%, 80%, 85%, 90%, 95%, 97%, 98%, or 99% (including all ranges and subranges therebetween)—for a given number of generations in a minimum regular growth medium or a cell growth medium lacking or having reduced level of serum and/or one or more exogenous growth factors. For example, in some embodiments, a cell line is considered stable for 20 generations if the cell viability is at or above 80% for 20 generations. In some embodiments, the cell viability is measured at a specific total cell density—for example, a total cell density of about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 22 million, about 24 million, about 26 million, about 28 million, about 30 million, about 32 million, about 34 million, about 36 million, about 38 million, about 40 million, about 45 million, or about 50 million, cells per milliliter (mL), including all ranges and subranges therebetween.

In some embodiments, the genetically modified cell line is stable in the suspension culture for at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 (including all ranges and subranges therebetween) generations. In some embodiments, the genetically modified cell line is stable in the suspension culture for at least 30 generations. In some embodiments, the corresponding control cell line (without said genetic modification) is unstable in the suspension culture. In some embodiments, the corresponding control cell line becomes unstable (e.g., significantly changes its growth characteristics) after less than 5, less than 10, less than 15, less than 20, less than 25, less than 30, less than 35, less than 40, less than 45, or less than 50 generations in the suspension culture. In some embodiments, the corresponding control cell line (without said genetic modification) is not viable in the suspension culture. In some embodiments, the cell growth medium of the suspension culture is a minimum regular growth medium. In some embodiments, the cell growth medium of the suspension culture lacks or has reduced level of serum and/or one or more exogenous growth factors (e.g., as described infra).

In some embodiments, the genetically modified cell population can reach a higher maximum viable cell density (VCD) compared to a control cell line in the cell culture. On the other hand, total cell density refers to the density of all cells in the cell population, including viable cells and non-viable cells (e.g., apoptotic cells). Viable cell density and total cell density can be determined using Trypan Blue staining and cell counting. In some embodiments, cells can be counted by one or more of the following methods: manual counting with a hemocytometer, automated cell counter (such as the ThermoFisher Countess), digital holographic microscopy (DHM), or DNA content measurement such as the ThermoFisher Cyquant assay.

In some embodiments, the genetically modified cell line can reach a maximum viable cell density of about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 22 million, about 24 million, about 26 million, about 28 million, about 30 million, about 32 million, about 34 million, about 36 million, about 38 million, about 40 million, about 45 million, about 50 million, about 55 million, about 60 million, about 65 million, about 70 million, about 75 million, about 80 million, about 85 million, about 90 million, about 95 million, or about 100 million, cells per milliliter (mL) in a suspension cell culture (including all ranges and subranges therebetween). In some embodiments, the genetically modified cell line can reach a maximum viable cell density of at least 0.1 million, at least 0.2 million, at least 0.3 million, at least 0.4 million, at least 0.5 million, at least 0.6 million, at least 0.7 million, at least 0.8 million, at least 0.9 million, at least 1 million, at least 1.5 million, at least 2 million, at least 3 million, at least 4 million, at least 5 million, at least 6 million, at least 7 million, at least 8 million, at least 9 million, at least 10 million, at least 11 million, at least 12 million, at least 13 million, at least 14 million, at least 15 million, at least 16 million, at least 17 million, at least 18 million, at least 19 million, at least 20 million, at least 22 million, at least 24 million, at least 26 million, at least 28 million, at least 30 million, at least 32 million, at least 34 million, at least 36 million, at least 38 million, at least 40 million, at least 45 million, at least 50 million, at least 55 million, at least 60 million, at least 65 million, at least 70 million, at least 75 million, at least 80 million, at least 85 million, at least 90 million, at least 95 million, or at least 100 million, cells per milliliter (mL) in a suspension cell culture (including all ranges and subranges therebetween). In some embodiments, the cell culture comprises a minimum regular cell growth medium or a cell growth medium lacking or having reduced level of serum and/or one or more exogenous growth factors (e.g., as described infra). In some embodiments, the genetically modified cell line can reach a maximum viable cell density of at least 20 million cells per milliliter (mL) in a cell growth medium lacking or having reduced level of serum and/or one or more exogenous growth factors.

In some embodiments, the genetically modified cell line can reach the maximum viable cell density faster than a control cell line in cell growth medium. In some embodiments, the genetically modified cell line is capable of reaching/reached maximum VCD at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold faster than the control cell line (including all ranges and subranges therebetween). In some embodiments, the cell culture comprises a minimum regular cell growth medium or a cell growth medium lacking or having reduced level of serum and/or one or more exogenous growth factors (e.g., as described infra). In some embodiments, the genetically modified cell line can reach the maximum viable cell density at least 20% faster in a cell growth medium lacking or having reduced level of serum and/or one or more exogenous growth factors.

In some embodiments, the genetically modified cell line is capable of reaching a specific viable cell density level faster than a control cell line in a suspension culture. In some embodiments, the specific viable cell density level is about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 25 million, about 30 million, about 35 million, about 40 million, about 45 million, or about 50 million, cells per milliliter (mL) (including all ranges and subranges therebetween). In some embodiments, the genetically modified cell line is capable of reaching the specific viable cell density level at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold faster than the control cell line (including all ranges and subranges therebetween). In some embodiments, the cell culture comprises a minimum regular cell growth medium or a cell growth medium lacking or having reduced level of serum and/or one or more exogenous growth factors (e.g., as described infra).

In some embodiments, the genetically modified cell line exhibits a cell viability of at least 50%, at least 55%, at least 60%, at least 65%, at least 70%, at least 75%, at least 80%, at least 85%, at least 90%, at least 95%, at least 97%, at least 98%, or at least 99%, at a specific total cell density level (including all ranges and subranges therebetween). In some embodiments, the genetically modified cell line exhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, higher cell viability than the control cell line at a specific total cell density level (including all ranges and subranges therebetween). In some embodiments, the specific total cell density level is about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, or about 20 million, about 22 million, about 24 million, about 26 million, about 28 million, about 30 million, about 32 million, about 34 million, about 36 million, about 38 million, about 40 million, about 45 million, about 50 million, about 55 million, about 60 million, about 65 million, about 70 million, about 75 million, about 80 million, about 85 million, about 90 million, about 95 million, or about 100 million, cells per milliliter (mL) (including all ranges and subranges therebetween). In some embodiments, the genetically modified cell line is capable of reaching a total cell density of at least 0.1 million, at least 0.2 million, at least 0.3 million, at least 0.4 million, at least 0.5 million, at least 0.6 million, at least 0.7 million, at least 0.8 million, at least 0.9 million, at least 1 million, at least 1.5 million, at least 2 million, at least 3 million, at least 4 million, at least 5 million, at least 6 million, at least 7 million, at least 8 million, at least 9 million, at least 10 million, at least 11 million, at least 12 million, at least 13 million, at least 14 million, at least 15 million, at least 16 million, at least 17 million, at least 18 million, at least 19 million, at least 20 million, at least 22 million, at least 24 million, at least 26 million, at least 28 million, at least 30 million, at least 32 million, at least 34 million, at least 36 million, at least 38 million, at least 40 million, at least 45 million, at least 50 million, at least 55 million, at least 60 million, at least 65 million, at least 70 million, at least 75 million, at least 80 million, at least 85 million, at least 90 million, at least 95 million, or at least 100 million, cells per milliliter (mL) (including all ranges and subranges therebetween). In some embodiments, the cell culture comprises a minimum regular cell growth medium or a cell growth medium lacking or having reduced level of serum and/or one or more exogenous growth factors (e.g., as described infra).

In some embodiments, the genetically modified cell line exhibits a doubling time of about 12-96 hours when the viable cell density of the cell line is at 30% of its maximum VCD in the suspension culture. In some embodiments, the doubling time is about 12-36, 24-48, 36-60, 48-72, 60-84, or 72-96 hours (including all ranges and subranges therebetween) when the viable cell density of the cell line is at 30% of its maximum VCD in the suspension culture. In some embodiments, the doubling time is about 12 hours, about 18 hours, about 24 hours, about 30 hours, about 36 hours, about 42 hours, about 48 hours, about 54 hours, about 60 hours, about 66 hours, about 72 hours, about 78 hours, about 84 hours, about 90 hours, or about 96 hours (including all ranges and subranges therebetween) when the viable cell density of the cell line is at 30% of its maximum VCD in the suspension culture. In some embodiments, the doubling time is less than 12 hours, less than 18 hours, less than 24 hours, less than 30 hours, less than 36 hours, less than 42 hours, less than 48 hours, less than 54 hours, less than 60 hours, less than 66 hours, less than 72 hours, less than 78 hours, less than 84 hours, less than 90 hours, or less than 96 hours (including all ranges and subranges therebetween) when the viable cell density of the cell line is at 30% of its maximum VCD in the suspension culture. In some embodiments, the suspension culture comprises a minimum regular cell growth medium or a cell growth medium lacking or having reduced level of serum and/or one or more exogenous growth factors (e.g., as described infra).

In some embodiments, the suspension culture of the genetically modified cell line comprises low or minimal levels of cell aggregates. In some embodiments, while smaller cell clusters (e.g., those that contain only a few cells) may be present in the suspension culture without significantly affecting cell viability, the presence of larger cell aggregates would negatively affect cell viability. In some embodiments, the suspension culture of the genetically modified cell line comprises less than 50%, less than 40%, less than 30%, less than 25%, less than 20%, less than 15%, less than 10%, less than 9%, less than 8%, less than 7%, less than 6%, less than 5%, less than 4%, less than 3%, less than 2%, or less than 1% (including all ranges and subranges therebetween) of cells in the form of cell aggregates when the cells are at a specific total cell density. In some embodiments, the cell aggregates contain at least 3, at least 4, at least 5, at least 6, at least 7, at least 8, at least 9, at least 10, at least 15, at least 20, at least 25, at least 30, at least 35, at least 40, at least 50, at least 60, at least 70, at least 80, at least 90, at least 100, at least 150, or at least 200 cells (including all ranges and subranges therebetween). In some embodiments, the cell aggregates contain at least 5 cells. In some embodiments, the cell aggregates have a diameter of greater than 100, greater than 150, greater than 200, greater than 250, greater than 300, greater than 350, greater than 400, greater than 450, or greater than 500 (including all ranges and subranges therebetween) microns. In some embodiments, the cell aggregates have a diameter of greater than 200 microns. In some embodiments, the specific total cell density level is about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 25 million, about 30 million, about 35 million, about 40 million, about 45 million, or about 50 million, cells per milliliter (mL) (including all ranges and subranges therebetween). In some embodiments, the suspension culture comprises a minimum regular cell growth medium or a cell growth medium lacking or having reduced level of serum and/or one or more exogenous growth factors (e.g., as described infra).

In some embodiments, the genetically engineered cells in the suspension culture exist mainly as single cells. In some embodiments, the suspension culture of the genetically modified cell line comprises at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% (including all ranges and subranges therebetween) of cells in the form of single cells when the cells are at a specific total cell density.

In some embodiments, the suspension culture of the genetically modified cell line comprises at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% (including all ranges and subranges therebetween) of cells in the form of single cells or cell clusters of less than 5 cells when the cells are at a specific total cell density. In some embodiments, the suspension culture of the genetically modified cell line comprises at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% (including all ranges and subranges therebetween) of cells in the form of single cells or cell aggregates of less than 200 microns in diameter when the cells are at a specific total cell density. In some embodiments, single cells have a diameter of less than 50, less than 45, less than 40, less than 35, less than 30, less than 25, less than 20, less than 15, less than 12, less than 10, less than 9, less than 8, less than 7, less than 6, or less than 5 microns (including all ranges and subranges therebetween). In some embodiments, single cells have a diameter of about 50, about 45, about 40, about 35, about 30, about 25, about 20, about 15, about 12, about 10, about 9, about 8, about 7, about 6, or about 5 microns (including all ranges and subranges therebetween). In some embodiments, the cell diameter is the average diameter of viable single cells. In some embodiments, single cells have a circularity of greater than 0.5, greater than 0.55, greater than 0.6, greater than 0.65, greater than 0.7, greater than 0.75, greater than 0.8, greater than 0.85, greater than 0.9, greater than 0.95, greater than 0.97, greater than 0.98, or greater than 0.99 (including all ranges and subranges therebetween). In some embodiments, the circularity is the average circularity of viable single cells. In some embodiments, the specific total cell density level is about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, about 20 million, about 25 million, about 30 million, about 35 million, about 40 million, about 45 million, or about 50 million, cells per milliliter (mL) (including all ranges and subranges therebetween). In some embodiments, the suspension culture comprises a minimum regular cell growth medium or a cell growth medium lacking or having reduced level of serum and/or one or more exogenous growth factors (e.g., as described infra).

In some embodiments, the suspension culture of the genetically modified cell line comprises low or minimal levels of adherent cells in the suspension culture. In some embodiments, the suspension culture of the genetically modified cell line comprises less than 50%, less than 40%, less than 30%, less than 25%, less than 20%, less than 15%, less than 10%, less than 9%, less than 8%, less than 7%, less than 6%, less than 5%, less than 4%, less than 3%, less than 2%, or less than 1% (including all ranges and subranges therebetween) of cells in the form of adherent cells when the cells are at a specific viable cell density. In some embodiments, the adherent cells are attached to the surface of the cell culture container (e.g., the container wall of a shaker flask or a bioreactor chamber). In some embodiments, the specific viable cell density level is about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, or about 20 million, cells per milliliter (mL) (including all ranges and subranges therebetween). In some embodiments, the suspension culture comprises a minimum regular cell growth medium or a cell growth medium lacking or having reduced level of serum and/or one or more exogenous growth factors (e.g., as described infra).

In some embodiments, the genetically modified cell line displays resistance to programed cell death (apoptosis and/or a non-apoptotic cell death mechanism). In some embodiments, the genetically modified cell line displays resistance to apoptosis (e.g., anoikis). In some embodiments, the genetically modified cell line displays resistance to programmed cell death induced by growth factor deprivation (such programmed cell death may be apoptotic or non-apoptotic). In some embodiments, the majority of the genetically modified cells are viable in the suspension culture. In some embodiments, at least 50%, at least 60%, at least 70%, at least 80%, at least 90%, at least 95%, at least 97%, at least 98%, or at least 99% (including all ranges and subranges therebetween) of cells are viable in the suspension culture of the genetically modified cell line. In some embodiments, the suspension culture of the genetically modified cell line comprises less than 50%, less than 40%, less than 30%, less than 25%, less than 20%, less than 15%, less than 10%, less than 9%, less than 8%, less than 7%, less than 6%, less than 5%, less than 4%, less than 3%, less than 2%, or less than 1% (including all ranges and subranges therebetween) apoptotic cells in the cell population when the cells are at a specific total cell density. In some embodiments, the percentage of apoptotic cells of the genetically modified cell line is at least 10%, at least 20%, at least 30%, at least 40%, at least 50%, at least 60%, at least 70%, at least 80%, at least 90%, at least 95%, at least 97%, at least 98%, or at least 99% lower than the control cell line without said genetic modification when the cell lines are at a specific total cell density in a suspension culture. In some embodiments, the specific total cell density level about 0.1 million, about 0.2 million, about 0.3 million, about 0.4 million, about 0.5 million, about 0.6 million, about 0.7 million, about 0.8 million, about 0.9 million, about 1 million, about 1.5 million, about 2 million, about 3 million, about 4 million, about 5 million, about 6 million, about 7 million, about 8 million, about 9 million, about 10 million, about 11 million, about 12 million, about 13 million, about 14 million, about 15 million, about 16 million, about 17 million, about 18 million, about 19 million, or about 20 million, cells per milliliter (mL) (including all ranges and subranges therebetween). In some embodiments, the suspension culture of the genetically modified cell line comprises less than 10% apoptotic cells in the cell population at a total cell density of about 10 million cells per milliliter (mL). In some embodiments, the suspension culture comprises a minimum regular cell growth medium or a cell growth medium lacking or having reduced level of serum and/or one or more exogenous growth factors (e.g., as described infra).

Methods of measures cell apoptosis are known in the art. Biomarkers for apoptotic cells include cytochrome C, caspase-3/7, DNA fragmentation and phosphatidylserine. Cytochrome C level may be measured by Western blotting or antibody staining. Caspase-3/7 level may be measured by Caspase-Glo® 3/7 Assay System offered by Promega. DNA fragmentation may be measured by TUNEL assays (e.g., using the DeadEnd™ Fluorometric TUNEL System by Promega). Phosphatidylserine level may be measured by Annexin V binding (e.g., using the Ab14085 Annexin V kit from Abcam). More descriptions of apoptosis biomarkers and their detection can be found in Ward et al., Br J Cancer. 2008 Sep. 16; 99 (6): 841-846, the content of which is incorporated by reference in its entirety.

In one aspect, the present disclosure provides genetically modified cell populations derived from the genetically modified cell line. In some embodiments, the genetically modified cell line is a somatic cell line. In some embodiments, the cell population is obtained via culturing the genetically modified cell line using a cell culture method. In some embodiments, the cell population is obtained via adherent cell culture. In some embodiments, the cell population is obtained via cell suspension culture. In some embodiments, the cell culture comprises a minimum regular cell growth medium. In some embodiments, the cell culture comprises a cell growth medium lacking or having reduced level of serum and/or one or more exogenous growth factors.

In some embodiments, the genetically modified cell population is substantially undifferentiated—for example, more than 70%, more than 75%, more than 80%, more than 85%, more than 90%, more than 95%, or 100% of the cell population is undifferentiated, including all ranges and subranges therebetween. In some embodiments, the genetically modified cell population is at least partially undifferentiated—for example, more than 30%, more than 35%, more than 40%, more than 45%, more than 50%, more than 55%, more than 60%, or more than 65%, of the cell population is undifferentiated, including all ranges and subranges therebetween. In some embodiments, the genetically modified cell population is substantially differentiated—for example, more than 70%, more than 75%, more than 80%, more than 85%, more than 90%, more than 95%, or 100% (including all ranges and subranges therebetween) of the cell population is differentiated.

In some embodiments, the genetically modified cell population remains undifferentiated (or substantially undifferentiated) in a cell growth medium lacking or having reduced level of serum and/or one or more exogenous growth factors, wherein the control cell line is substantially differentiated in the same cell growth medium. In some embodiments, the cell growth medium comprises 1% or less, 2% or less, 3% or less, 4% or less, 5% or less, 10% or less, 20% or less, 30% or less, 40% or less, 50% or less, 60% or less, 70% or less, 80% or less, 90% or less, or 95% or less (including all ranges and subranges therebetween) of serum (e.g., FBS) as compared to a minimum regular cell growth medium. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of one or more exogenous growth factors (e.g., FGF) as compared to a minimum regular cell growth medium.

In one aspect, the present disclosure provides clonal cell culture comprising: a) the genetically modified cell population of the disclosure, wherein the cell population is in contact with b) a cell growth medium lacking or having reduced level of serum and/or one or more exogenous growth factors. In some embodiments, the cell population is a somatic cell population. In some embodiments, the cell population exhibits increased proliferation and/or reduced differentiation compared to a control cell population grown in the cell growth medium.

In some embodiments, the genetically modified cell population in the clonal cell culture exhibits increased rate of proliferation compared to a control cell population in the cell growth medium. In some embodiments, rate of proliferation is quantified by cell counts at regular intervals. In some embodiments, the rate of proliferation is higher by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold (including all ranges and subranges therebetween), for the genetically modified cell population compared to a control cell population in the cell growth medium lacking or having reduced level of serum and/or one or more exogenous growth factors.

In some embodiments, the genetically modified cell population in the clonal cell culture exhibits decreased differentiation compared to a control cell population in the cell growth medium. In some embodiments, differentiation is quantified via cell morphology analysis using phase contrast microscopy to analyze for changes in the phenotype of the cell population. In some embodiments, the rate of differentiation is lower by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold (including all ranges and subranges therebetween), for the genetically modified cell population compared to a control cell population in the cell growth medium lacking or having reduced level of serum and/or one or more exogenous growth factors.

In some embodiments, the percentage of differentiated cells in the cell culture is lower by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, or at least 99% (including all ranges and subranges therebetween), for the genetically modified cell population compared to a control cell population in the cell growth medium lacking or having reduced level of serum and/or one or more exogenous growth factors.

In some embodiments, the genetically modified cell population in the clonal cell culture exhibits extended replicative capacity compared to a control cell population in the cell growth medium. In some embodiments, the replicative capacity is extended by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 1-fold, at least 2-fold, at least 3-fold, at least 4-fold, at least 5-fold, at least 7-fold, at least 10-fold, at least 15-fold, at least 20-fold, at least 30-fold, at least 40-fold, at least 50-fold, at least 70-fold, or at least 100-fold (including all ranges and subranges therebetween), for the genetically modified cell population compared to a control cell population in the cell growth medium lacking or having reduced level of serum and/or one or more exogenous growth factors.

In some embodiments, the cell population is cultivated in a cultivation infrastructure. A “cultivation infrastructure” refers to the environment in which the cell population (i.e., cellular biomass) are cultured. A cultivation infrastructure may be a tube, a cylinder, a flask, a petri-dish, a multi-well plate, a dish, a vat, an incubator, a bioreactor, an industrial fermenter and the like. A cultivation infrastructure may be a culture medium in which metazoan cells are cultured.

A cultivation infrastructure can be of any scale and support any volume of cell population and culturing reagents. In some embodiments, the scale of the cultivation infrastructure ranges from about 10 μL to about 100,000 L. In some embodiments, the cultivation infrastructure is about 10 μL, about 100 μL, about 1 mL, about 10 mL, about 100 mL, about 1 L, about 10 L, about 100 L, about 1000 L, about 10,000 L, or even about 100,000L, including all ranges and subranges therebetween.

In some embodiments, the cultivation infrastructure comprises a substrate. A cultivation infrastructure may comprise a permeable substrate (e.g. permeable to physiological solutions) or an impermeable substrate (e.g. impermeable to physiological solutions). In some embodiments, the cultivation infrastructure comprises a primary substrate, which can be a flat, concave, or convex substrate. In some embodiments, the cultivation infrastructure further comprises a secondary substrate, either introduced, or autologous, to direct cellular growth between the substrates, e.g., to direct attachment, proliferation and hypertrophy of cells on a plane perpendicular to the primary substrate. In some embodiments, the cultivation infrastructure comprises a hydrogel, a liquid cell culture medium, or soft agar.

In some embodiments, the cultivation infrastructure does not comprise a substrate to which cells can adhere.

The genetically modified cell population of the disclosure may grow in the cultivation infrastructure as adherent, non-adherent, or suspension cell culture. In some embodiments, the genetically modified cell population comprises primarily adherent cells (e.g., those cells that adhere to a substrate) in the cultivation infrastructure. In some embodiments, the genetically modified cell population comprises primarily non-adherent cells (e.g., those cells that do not adhere to a substrate) in the cultivation infrastructure. In some embodiments, the genetically modified cell population comprises both adherent and non-adherent cells in the cultivation infrastructure. In some embodiments, the genetically modified cell population comprises cells in suspension culture, e.g., as a self-adhering biomass, or single-cell suspension in a liquid medium in the cultivation infrastructure.

In some embodiments, the cultivation infrastructure is a bioreactor system. In some embodiments, the genetically modified cells are grown in bioreactor systems in a single cell suspension, in cell aggregates, on microcarriers, or undergo a biofabrication step where they are synthesized together into tissue. In some embodiments, the cell population is cultivated in a suspension culture. In some embodiment, the bioreactor system is a fed batch bioreactor. In some embodiments, the bioreactor system has a scale of at least 500-liter, at least 1,000-liter, at least 2,000-liter, at least 5,000-liter, at least 10,000-liter, at least 20,000-liter, or at least 50,000-liter. In some embodiments, the bioreactor system has a scale of at least 20,000-liter.

A bioreactor system is typically scalable for large-scale cell culture and is optimized for biomass production. The bioreactor system comprises a stirring element for agitation of the contents of the reactor chamber which helps to keep the cells in suspension. A temperature jacket provides temperature control to these cells. Oftentimes, the bioreactor system comprises at least one sensor for monitoring the reactor chamber. The at least one sensor is usually in communication with a control unit (e.g., a computer). Compressed air, Oxygen may be sterilely introduced (sparged) into the bioreactor to control dissolved oxygen content. Carbon dioxide and a suitable base may be introduced in the reactor to control pH. Fresh medium may be added into the bioreactor via at least one input port. Fresh medium is sometimes maintenance medium, differentiation medium, steatotic medium, proliferation medium, or any other medium formulation disclosed herein.

The cells may be grown until they reach a desired biomass. The desired biomass may be a biomass reached once the cells are no longer able to proliferate or may be the maximum biomass the cells can reach in a given culture size and culture conditions. In some embodiments, the maximum biomass is reached when the cell population reaches the maximum viable cell density. Alternatively, the desired biomass may be the biomass at which sufficient cells have been produced to form a cell-based meat product.

In some embodiments, the cellular biomass is cultivated as a single-cell suspension culture. In some embodiments, the cellular biomass is cultivated in a suspension culture and forms self-adherent aggregates. A self-adherent aggregate refers to masses of viable cells suspended in a physiological liquid medium (e.g., suspension culture) aggregated due to, for example, their (1) adherence to each other (e.g., cadherin cell adhesion) (2) adherence to a basement membrane or other extracellular matrix secreted by the cells (e.g., integrin cell adhesion) or (3) a combination of both. In some embodiments, the cell aggregates have poor nutrient uptake and a reduced responsiveness to growth factors, and therefore cause reduction of proliferation rate and/or maximum viable cell density. In some embodiments, the genetically modified cell line of the disclosure is able to grow in suspension conditions while minimizing the requirement for serum/growth factors, and therefore overcoming the adverse effect of aggregate formation. As a result, in some embodiments, the genetically modified cell line can achieve a higher proliferation rate and/or maximum viable cell density in suspension culture (e.g., in a bioreactor).

### Cell Growth Medium

In one aspect, the disclosure provides cell growth mediums for culturing the genetically modified cell line. In some embodiments, the cell growth medium lacks or has reduced level of serum and/or one or more exogenous growth factors. In some embodiments, the cell growth medium comprises one or more ingredients that facilitates cell growth in suspension culture—for example, lipid/lipid precursor, surface active agent/surfactant.

As discussed above, cells may form cell aggregates in suspension culture and the presence of lipids in the culture medium may decrease or eliminate the amount of cell aggregates in the suspension culture. In some embodiments, the cell growth medium comprises a lipid and/or lipid precursor such as choline, oleic acid, linoleic acid, ethanolamine, or phosphoethanolamine. In some embodiments, the lipid and/or lipid precursor is derived from a natural source (e.g., a plant-derived lipid mixture). In some embodiments, the lipid and/or lipid precursor is chemically synthesized. In some embodiments, the amount of the lipid and/or lipid precursor in the cell growth medium is between 0.01% and 2% by weight. In some embodiments, the amount of the lipid and/or lipid precursor in the cell growth medium is between 0.01% and 0.05%, between 0.02% and 0.1%, between 0.05% and 0.15%, between 0.1% and 0.3%, between 0.2% and 0.4%, between 0.3% and 0.5%, between 0.4% and 0.6%, between 0.5% and 0.7%, between 0.6% and 0.8%, between 0.7% and 0.9%, between 0.8% and 1.0%, between 0.8% and 1.2%, between 1.0% and 1.4%, between 1.2% and 1.6%, between 1.4% and 1.8%, between 1.6% and 2.0%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0% by weight (including all ranges and subranges therebetween).

In some embodiments, the cell growth medium comprises a surface active agent and/or a surfactant. In some embodiments, the surface active agent and/or surfactant protects the cells from the negative effects of agitation and aeration. In some embodiments, the surface-active agent and/or the surfactant is nonionic. In some embodiments, the surfactant is a PLURONICS surfactant. The PLURONICS are a series of nonionic surfactants with the general structure HO(CH2CH2O)ₐ(CH(CH3)CH2OH)_{b}(CH2CH2O)_{c}H where b is at least 15 and (CH2CH2O)a+c is varied from 20% to 90% by weight. The PLURONICS are also known, for example, as poloxamers; methyl oxirane polymers, polymer with oxirane; and polyethylenepolypropylene glycols, polymers. In some embodiments, the surfactant is PLURONIC F68. In some embodiments, the amount of the surface active agent and/or surfactant in the cell growth medium is between 0.01% and 2% by weight. In some embodiments, the amount of the surface active agent and/or surfactant in the cell growth medium is between 0.01% and 0.05%, between 0.02% and 0.1%, between 0.05% and 0.15%, between 0.1% and 0.3%, between 0.2% and 0.4%, between 0.3% and 0.5%, between 0.4% and 0.6%, between 0.5% and 0.7%, between 0.6% and 0.8%, between 0.7% and 0.9%, between 0.8% and 1.0%, between 0.8% and 1.2%, between 1.0% and 1.4%, between 1.2% and 1.6%, between 1.4% and 1.8%, between 1.6% and 2.0%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0% by weight (including all ranges and subranges therebetween). As discussed above, traditional in vitro myoblast cultures are supplemented with serum and/or one or more exogenous growth factors in the culture medium (i.e., cell growth medium) to maintain proliferation and inhibit differentiation. As used herein, “minimum regular cell growth medium” refers to a culture medium that comprises the minimum levels of serum and/or exogenous growth factors that allow a control cell line to proliferate and maintain a substantially undifferentiated status. In some embodiments, the minimum regular cell growth medium allows the control cell line to proliferate and maintain a substantially undifferentiated status up until the control cell line is at a viable cell density level of about 20%, 30%, 40%, 50%, 60%, 70%, 80%, or 90% (including all ranges and subranges therebetween) of its maximum VCD in that cell growth medium. In some embodiments, the minimum regular cell growth medium allows the control cell line to proliferate and maintain a substantially undifferentiated status up until the control cell line is at a viable cell density level of about 70% of its maximum VCD in that cell growth medium. A non-limiting example of a minimum regular cell growth medium is composed of DMEM (Dulbecco's Modified Eagle Medium) with 20% (v/v) FBS and 2 ng/ml FGF-2.

Exemplary serum includes fetal bovine serum (FBS) and horse serum. Exemplary growth factors include fibroblast growth factor (FGF), epidermal growth factor (EGF), insulin-like growth factor (IGF), insulin, hepatocyte growth factor (HGF), transforming growth factor beta (TGF-β), tumor necrosis factor alpha (TNF-α), leukemia inhibitory factor (LIF), and/or interleukin-6 (IL6).

Fibroblast growth factor (FGF) family contains 22 members, designated FGF-1 through FGF-23 except FGF-15 (i.e., FGF-1, FGF-2, FGF-3, FGF-4, FGF-5, FGF-6, FGF-7, FGF-8, FGF-9, FGF-10, FGF-11, FGF-12, FGF-13, FGF-14, FGF-16, FGF-17, FGF-18, FGF-19, FGF-20, FGF-21, FGF-22, and FGF-23). All FGFs except four members (FGF-11, FGF-12, FGF-13 and FGF-14) bind to four transmembrane tyrosine kinase receptors-FGFR1, FGFR2, FGFR3, and FGFR4. Among the family members, FGF-1 is also known as acidic fibroblast growth factor, and FGF-2 is also known as basic fibroblast growth factor. In some embodiments, the FGF supplemented in the myoblast culture medium is FGF-2.

In some embodiments, the cell growth medium lacks or has reduced levels of serum and/or one or more exogenous growth factors. In some embodiments, the one or more exogenous growth factors comprises an FGF. In some embodiments, the FGF is FGF-2. In some embodiments, the one or more growth factors are selected from the group consisting of FGF (e.g., FGF-2), EGF, IGF, insulin, and HGF. In some embodiments, the one or more growth factors are selected from the group consisting of FGF (e.g., FGF-2), EGF, IGF, insulin, HGF, TGF-β and TNF-α.

In some embodiments, the cell growth medium comprises no or reduced level of serum. In some embodiments, the cell growth medium comprises no serum. In some embodiments, the cell growth medium comprises 0.1% or less, 0.2% or less, 0.3% or less, 0.5% or less, 0.7% or less, 1% or less, 2% or less, 3% or less, 4% or less, 5% or less, 6% or less, 7% or less, 8% or less, 9% or less, 10% or less, 11% or less, 12% or less, 13% or less, 14% or less, 15% or less, 16% or less, 17% or less, 18% or less, 19% or less, or 20% or less of serum (by volume) in the total medium, including all ranges and subranges therebetween. In some embodiments, the cell growth medium comprises 1% or less, 2% or less, 3% or less, 4% or less, 5% or less, 10% or less, 20% or less, 30% or less, 40% or less, 50% or less, 60% or less, 70% or less, 80% or less, 90% or less, or 95% or less (including all ranges and subranges therebetween) of serum as compared to a minimum regular cell growth medium. In some embodiments, the serum is FBS.

In some embodiments, the cell growth medium comprises no or reduced level of FGF. In some embodiments, the cell growth medium comprises no exogenous FGF. In some embodiments, the exogenous FGF is FGF-2. In some embodiments, the cell growth medium has a reduced level of FGF and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous FGF, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous FGF as compared to a minimum regular cell growth medium.

In some embodiments, the cell growth medium comprises no or reduced level of EGF. In some embodiments, the cell growth medium comprises no exogenous EGF. In some embodiments, the cell growth medium has a reduced level of EGF and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous EGF, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous EGF as compared to a minimum regular cell growth medium.

In some embodiments, the cell growth medium comprises no or reduced level of IGF. In some embodiments, the cell growth medium comprises no exogenous IGF. In some embodiments, the cell growth medium has a reduced level of IGF and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous IGF, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous IGF as compared to a minimum regular cell growth medium.

In some embodiments, the cell growth medium comprises no or reduced level of insulin. In some embodiments, the cell growth medium comprises no exogenous insulin. In some embodiments, the cell growth medium has a reduced level of insulin and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, no more than 100 ng/ml, no more than 200 ng/ml, no more than 300 ng/ml, no more than 400 ng/ml, no more than 500 ng/ml, no more than 600 ng/ml, no more than 700 ng/ml, no more than 800 ng/ml, no more than 900 ng/ml, no more than 1 μg/ml, no more than 2 μg/ml, no more than 3 μg/ml, no more than 5 μg/ml, no more than 7 μg/ml, or no more than 10 μg/ml, of exogenous insulin, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous insulin as compared to a minimum regular cell growth medium.

In some embodiments, the cell growth medium comprises no or reduced level of HGF. In some embodiments, the cell growth medium comprises no exogenous HGF. In some embodiments, the cell growth medium has a reduced level of HGF and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous HGF, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous HGF as compared to a minimum regular cell growth medium.

In some embodiments, the cell growth medium comprises no or reduced level of TGF-β. In some embodiments, the cell growth medium comprises no exogenous TGF-β. In some embodiments, the cell growth medium has a reduced level of TGF-β and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous TGF-β, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous TGF-β as compared to a minimum regular cell growth medium.

In some embodiments, the cell growth medium comprises no or reduced level of TNF-α. In some embodiments, the cell growth medium comprises no exogenous TNF-α. In some embodiments, the cell growth medium has a reduced level of TNF-α and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous TNF-α, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous TNF-α as compared to a minimum regular cell growth medium.

In some embodiments, the cell growth medium comprises no or reduced level of LIF. In some embodiments, the cell growth medium comprises no exogenous LIF. In some embodiments, the cell growth medium has a reduced level of LIF and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous LIF, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous LIF as compared to a minimum regular cell growth medium.

In some embodiments, the cell growth medium comprises no or reduced level of IL6. In some embodiments, the cell growth medium comprises no exogenous IL6. In some embodiments, the cell growth medium has a reduced level of IL6 and comprises no more than 0.01 ng/ml, no more than 0.02 ng/ml, no more than 0.03 ng/ml, no more than 0.05 ng/ml, no more than 0.07 ng/ml, no more than 0.1 ng/ml, no more than 0.2 ng/ml, no more than 0.3 ng/ml, no more than 0.5 ng/ml, no more than 0.7 ng/ml, no more than 1 ng/ml, no more than 2 ng/ml, no more than 3 ng/ml, no more than 5 ng/ml, no more than 7 ng/ml, no more than 10 ng/ml, no more than 15 ng/ml, no more than 20 ng/ml, no more than 30 ng/ml, no more than 40 ng/ml, no more than 50 ng/ml, no more than 60 ng/ml, no more than 70 ng/ml, no more than 80 ng/ml, no more than 90 ng/ml, or no more than 100 ng/ml of exogenous IL6, including all ranges and subranges therebetween. In some embodiments, the cell growth medium comprises 1% or less, 2% or less, 3% or less, 5% or less, 10% or less, 20% or less, 30% or less, 40% or less, 50% or less, 60% or less, 70% or less, 80% or less, 90% or less, or 95% or less (including all ranges and subranges therebetween) of exogenous IL6 as compared to a minimum regular cell growth medium.

In some embodiments, the cell growth medium lacks or has a reduced level of serum and one or more exogenous growth factors. In some embodiments, the cell growth medium lacks or has a reduced level of serum and FGF. In some embodiments, the cell growth medium comprises neither serum nor FGF. In some embodiments, the FGF is FGF-2.

In some embodiments, the cell growth medium lacks or has a reduced level of serum and at least one exogenous growth factor selected from the group consisting of FGF, EGF, IGF, insulin, and HGF. In some embodiments, the cell growth medium lacks or has a reduced level of serum and at least one exogenous growth factor selected from the group consisting of FGF, EGF, IGF, insulin, HGF, TGF-β and TNF-α. In some embodiments, the cell growth medium lacks or has a reduced level of at least two exogenous growth factors selected from the group consisting of FGF, EGF, IGF, insulin, and HGF. In some embodiments, the cell growth medium lacks or have reduced level of at least two exogenous growth factors selected from the group consisting of FGF, EGF, IGF, insulin, HGF, TGF-β and TNF-α.

### Differentiation

In some embodiments, the present disclosure provides differentiated cells derived from the genetically modified cell line of the disclosure. In some embodiments, the disclosure provides methods for causing the genetically modified cell population to transition from a less differentiated state to a more differentiated state. In some embodiments, the genetically modified cell population are myogenic cells, and the method further comprises causing the cells to differentiate into myoblasts or multinucleated myotubes. In some embodiments, the genetically modified cells are fibroblasts or adipogenic cells, mesenchymal stem cells, bone marrow derived cells, cardiomyocytes, hepatocytes, or other cell types found in organ meat, which achieve a more differentiated state through use of the methods described herein.

In some embodiments, once a sufficient amount of the genetically modified cell population has been obtained, the cells can be induced to differentiate.

In some embodiments, differentiation comprises withdrawal of the culture medium that supports the viability, survival, growth or expansion of the cell population. Withdrawal may comprise physical removal of the culture medium or altering the composition of the culture medium, for example, by addition of components that would facilitate differentiation of the cell population or by depletion of components that support proliferation of the cell population.

In some embodiments, differentiation is induced by changes in cell density. In some embodiments, differentiation is induced by changes in availability of one or more nutrient factors and/or growth factor. In some embodiments, differentiation is induced by upregulating the expression of myocyte specific genes, for example myogenin and Troponin-T.

### Genetically Modified Cells as a Food Ingredient

In one aspect, the present disclosure teaches using the genetically modified cells of the disclosure as an ingredient of a food product. In some embodiments, substantially undifferentiated cells are used as a food ingredient. The common practice in the alternative meat field is to use adherent cell culture to produce a cell-based meat product. In some embodiments, the reduced PTEN activity renders resistance to programmed cell death (apoptotic and/or non-apoptotic) and phenotypical advantages for suspension cell culture. In some embodiments, the resistance to programmed cell death comprises anoikis resistance. In some embodiments, the genetically modified cells are resistant to programmed cell death induced by growth factor deprivation. In some embodiments, such cells derived from suspension culture are used as an ingredient to produce food products with meat-like taste. In some embodiments, the cells are combined with other ingredients (e.g., a plant-based food ingredient), to produce food products with meat-like taste.

In some embodiments, the genetically modified cell population is substantially undifferentiated—for example, more than 70%, more than 75%, more than 80%, more than 85%, more than 90%, more than 95%, or 100% of the cell population is undifferentiated, including all ranges and subranges therebetween. In some embodiments, the genetically modified cell population is at least partially undifferentiated—for example, more than 30%, more than 35%, more than 40%, more than 45%, more than 50%, more than 55%, more than 60%, or more than 65%, of the cell population is undifferentiated, including all ranges and subranges therebetween. In some embodiments, the genetically modified cell population is substantially differentiated—for example, more than 70%, more than 75%, more than 80%, more than 85%, more than 90%, more than 95%, or 100% (including all ranges and subranges therebetween) of the cell population is differentiated.

In some embodiments, the genetically modified cell population has undergone one or more food processing steps selected from heating, refrigerating, freezing, and flavoring. In some embodiments, the genetically modified cell population is processed as a raw, uncooked food ingredient, or as a cooked food ingredient. In some embodiments, the ingredient comprises a genetically modified cell population that has been heated (e.g., cooked). In some embodiments, the food ingredient comprising the genetically modified cell population has undergone one or more food processing steps selected from baking, roasting, broiling, sautéing, braising, steaming, poaching, grilling, frying (e.g., deep-frying, pan-frying), impingement cooking, boiling, stewing, simmering, microwaving, and sous vide cooking. In some embodiments, the food ingredient comprising the genetically modified cell population has undergone one or more flavoring steps. Non-limiting examples of flavoring steps include smoking, marinating and glazing.

In some embodiments, the food processing step comprises exposing the genetically modified cell population to superphysological temperatures (e.g., heating) that would not support the viability, survival, expansion and/or differentiation of the cell population. In some embodiments, exposing the cell population to superphysiological temperatures comprises fully or partially cooking the cell population, for example, by heating the cell population to a temperature of about 100° F. to about 600° F., about 100° F. to about 550° F., about 100° F. to about 500° F., about 100° F. to about 450° F., about 100° F. to about 400° F., about 100° F. to about 350° F., about 100° F. to about 300° F., about 100° F. to about 250° F., about 100° F. to about 200° F. or about 100° F. to about 150° F., or by heating the cell population to a temperature of at least 100° F., at least 125° F., at least 150° F., at least 175° F., at least 200° F., at least 225° F., at least 250° F., at least 275° F., at least 300° F., at least 325° F., at least 350° F., at least 375° F., at least 400° F., at least 425° F., at least 450° F., at least 475° F., at least 500° F., at least 525° F., at least 550° F., at least 575° F., or at least 600° F., including all ranges and subranges therebetween. In some embodiments, the cell population is exposed to superphysological temperatures for at least 15 seconds, at least 30 seconds, at least 1 minute, at least 2 minutes, at least 3 minutes, at least 5 minutes, at least 10 minutes, at least 20 minutes, at least 30 minutes, or at least 60 minutes, including all ranges and subranges therebetween.

In some embodiments, the food processing step comprises exposing the genetically modified cell population to sub-physiological temperatures that would not support the expansion and/or differentiation of the cell population. Sub-physiological temperatures include a temperature of about 15° C. (about 59° F.) or lower, about 10° C. (about 50° F.) or lower, about 0° C. to about 15° C. (about 32° F. to about 59° F.), about 0° C. to −15° C. (about 32° F. to about 5° F.), about −15° C. to about 15° C. (about 5° F. to about 59° F.), about 0° C. to −213° C. (about 32° F. to about −350° F.), about −30° C. to about −100° C. (about −22° F. to about −148° F.), about −50° C. to about −90° C. (about −58° F. to about −130° F.), or about −170° C. to about −190° C. (about −274° F. to about −310° F.), including all ranges and subranges therebetween.

In some embodiments, the genetically modified cell population is cooled to a temperature of about 2° C. to about 8° C. (about 35° F. to about 46.5° F.). In some embodiments, the genetically modified cell population is frozen, for example, by cooling to a temperature of about 32° F. or lower, e.g. about 32° F. to about 0° F., about 32° F. to about −10° F., about 32° F. to about −20° F., about 32° F. to about −30° F., about 32° F. to about −40° F., about 32° F. to about −50° F., about 32° F. to about −60° F., about 32° F. to about −70° F., about 32° F. to about −80° F., and the like. In some embodiments, the genetically modified cell population is exposed to sub-physiological temperatures as low as about −300° F. to about −350° F., such as the liquid nitrogen temperature of about −321° F.

### Cell-based Meat Product

In one aspect, the present disclosure provides cell-based meat products comprising the cell population of the disclosure. In some embodiments, the cell-based meat product comprises a plant-based protein or product. In some embodiments, the cell population has undergone one or more food processing steps selected from heating, refrigerating, freezing, and smoking.

Cell-based meat products refer to meat products that contain animal cells grown outside the animal in bioreactor systems or other similar production systems. Cell-based meat products can take numerous forms and be used in different ways. Manufactured animal cells can be used as ingredients to foods containing a high percentage of vegetable material, or they can be produced in enough biomass to be the primary (or sole) ingredient in the food. Cell-based meat products may also contain other ingredients or additives, including but not limited to preservatives.

The cell-based meat products of the disclosure may comprise tissue engineered products, genetically modified cell population of the disclosure blended with plant-based protein/product, or substantially pure genetically modified cell population of the disclosure. In some embodiments, cell-based meat products include cultured animal cells (e.g., genetically modified cells of the disclosure) that may or may not be combined with plant-based protein/product or other food additives or ingredients, may result in unstructured ground meat products, such as ground beef, or may be tissue engineered/synthesized into structured tissue such as bacon or steak. Sources of plant-based protein/product which may be used include, without limitation, peas, chickpeas, mung beans, kidney beans, fava beans, cowpeas, pine nuts, rice, corn, potato, and sesame. Cell-based meat can be structured into living tissue that can be matured in a bioreactor, or nonliving tissue as the end product.

The genetically modified cells of the disclosure may be combined with one or more other ingredients for the production of cell-based meat products. In some embodiments, such cell-based meat products result in processed foods suitable for animal or human consumption. In some embodiments, the cell-based meat products are foods/consumable products that are either completely or partially composed of the genetically modified cells of the disclosure. In some embodiments, the cell-based meat product is substantially composed of plant-based protein(s)/food (e.g., soy, pea, beans) or plant cells and has a smaller amount of the genetically modified cells in the mixture. In some embodiments, a cell-based meat product comprises recombinantly expressed animal proteins (e.g., hemoglobin, myoglobin, egg white proteins, milk proteins) and also comprises the genetically modified cell of the disclosure. In some embodiments, a cell-based meat product comprises an engineered tissue and the genetically modified cells described herein.

In some embodiments, the present disclosure provides cell-based meat products comprising genetically modified cell population of the disclosure. In some embodiments, the cell-based meat product further comprises plant-based protein/product. In some embodiments, the genetically modified cell population and plant-based protein/product of the cell-based meat product are bound together by one or more binding agents, which can produce cell-based meat products that have one or more similar or superior attributes compared to animal meat. In some embodiments, the cell-based meat product incorporates one or more edible fibrous components, which can help achieve a textural heterogeneity and fibrousness in the cell-based meat product that resembles the heterogeneity and texture of animal meat. In some embodiments, the cell-based meat product incorporates one or more flavoring agents, which can help mimic the sensory properties of ground meat.

In some embodiments, the cell-based meat products comprise at least 1%, at least 2%, at least 3%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% (including all ranges and subranges therebetween) by weight of the genetically modified cell population of the disclosure. In some embodiments, the genetically modified cell population is substantially undifferentiated—for example, more than 70%, more than 75%, more than 80%, more than 85%, more than 90%, more than 95%, or 100% of the cell population is undifferentiated, including all ranges and subranges therebetween. In some embodiments, the genetically modified cell population is at least partially undifferentiated—for example, more than 30%, more than 35%, more than 40%, more than 45%, more than 50%, more than 55%, more than 60%, or more than 65%, of the cell population is undifferentiated, including all ranges and subranges therebetween. In some embodiments, the genetically modified cell population is substantially differentiated—for example, more than 70%, more than 75%, more than 80%, more than 85%, more than 90%, more than 95%, or 100% (including all ranges and subranges therebetween) of the cell population is differentiated.

In some embodiments, the cell-based meat product resembles ground animal meat (e.g., ground beef, ground chicken, ground turkey, ground lamb, or ground pork), as well as cuts of meat and fish. In some embodiments, the cell-based meat product comprises a burger patty. In some embodiments, the cell-based meat product is principally or entirely composed of ingredients derived from non-animal sources. In some embodiments, the cell-based meat product is composed of ingredients partially derived from animal sources but supplemented with ingredients derived from non-animal sources.

In some embodiments, the cell-based meat product comprises about 1% to about 90% (e.g., about 1% to about 30%, about 3% to about 40%, about 5% to about 60%, or about 10% to about 80%) by weight of the genetically modified cell population of the disclosure; optionally, about 0% to about 95% of one or more plant-based protein/product (e.g., about 10% to about 90%, about 40% to about 80%); about 0% to about 40% (e.g., about 15% to about 25%) by weight of an optional carbohydrate-based gel; about 0% to about 35% by weight of an optional non-animal fat (e.g., about 10% to about 15%); about 0% to about 10% by weight of an optional flavoring agent (e.g., about 0.00001% to about 5%); about 0% to about 15% (e.g., about 2% to about 15% or about 2% to about 10%) by weight of an optional binding agent; and about 0% to about 4% (e.g., about 0.05% to about 1%, or about 0.2% to about 2%) by weight of an optional iron complex such as a heme-containing protein and/or an iron salt.

The amount of flavoring agents can vary depending on the type of flavoring agent. In some embodiments, a flavoring agent can be about 0.5% to about 7% of the cell-based meat product. For example, a flavoring agent such as a mixture of flavor precursors can be about 0.5% to about 7% of the cell-based meat product (e.g., about 1% to about 3%; about 3% to about 6%; about 4% to about 7%). In some embodiments, a flavoring agent such as a flavoring compound can be about 0.00001% to about 2% of the cell-based meat product.

In some embodiments, the cell-based meat product is in the form of a meat dough. In some embodiments, the meat dough is prepared by mixing the genetically modified cell population of the disclosure with one or more plant protein/product and an optional edible fibrous component, an optional flavoring agent, and optional non-animal fat, and adding an aqueous component such as water or a broth to the mixture and kneading or otherwise mixing, manually or mechanically, to form a dough. The aqueous component can be heated before adding to the mixture of plant protein and fibrous component. Once the meat dough is formed, the meat dough can be heated (e.g., steamed or boiled) to a temperature ranging from 150° F. to 250° F. (e.g., 160° F. to 240° F., 170° F. to 230° F., 180° F. to 220° F., or 190° F. to 212° F.). For example, a meat dough can be steamed by placing in a rice cooker, steam cabinet, or tunnel steamer. A meat dough can be heated by applying dry heat, for example, by placing in a bread maker or oven, or by immersing in hot water or broth. Boiling in broth can improve the meat dough flavor because beneficial flavors and off-flavor masking agents can be absorbed into the dough. Texture properties may also be modulated by choice of the cooking method.

In some embodiments, the plant protein of the cell-based meat product comprises wheat gluten, a dehydrin protein, an albumin, a globulin, or a zein, or mixtures thereof.

In some embodiments, the cell-based meat product comprises one or more edible fibrous components. In some embodiments, the edible fibrous component of the cell-based meat product comprises plant fibers from carrot, bamboo, pea, broccoli, potato, sweet potato, corn, whole grains, alfalfa, kale, celery, celery root, parsley, cabbage, zucchini, green beans, kidney beans, black beans, red beans, white beans, beets, cauliflower, nuts, apple skins, oats, wheat, or psyllium, or a mixture thereof. In some embodiments, the edible fibrous component comprises an extruded mixture of isolated plant proteins. In some embodiments, the extruded mixture comprises wheat gluten and soy protein isolate, and optionally further comprises a flavoring agent (e.g., a flavoring such as yeast extract, a protein hydrolysate, or an oil; a flavor compound; or a flavor precursor). In some embodiments, the edible fibrous component comprises a solution-spun protein fiber (e.g., a solution-spun protein fiber containing a prolamin such as corn zein, pea prolamin, kafirin, secalin, hordein, avenin, or a mixture thereof).

In some embodiments, the cell-based meat product comprises one or more fats. In some embodiments, the fat is a non-animal fat, an animal fat, or a mixture of non-animal and animal fat. In some embodiments, the fat comprise an algal oil, a fungal oil, corn oil, olive oil, soy oil, peanut oil, walnut oil, almond oil, sesame oil, cottonseed oil, rapeseed oil, canola oil, safflower oil, sunflower oil, flax seed oil, palm oil, palm kernel oil, coconut oil, babassu oil, shea butter, mango butter, cocoa butter, wheat germ oil, borage oil, black currant oil, sea-buckhorn oil, macadamia oil, saw palmetto oil, conjugated linoleic oil, arachidonic acid enriched oil, docosahexaenoic acid (DHA) enriched oil, eicosapentaenoic acid (EPA) enriched oil, palm stearic acid, sea-buckhorn berry oil, macadamia oil, saw palmetto oil, or rice bran oil; or margarine or other hydrogenated fats. In some embodiments, the fat comprises algal oil. In some embodiments, the fat comprises the flavoring agent and/or the isolated plant protein (e.g., a conglycinin protein).

In some embodiments, the cell-based meat product comprises one or more flavoring agents. In some embodiments, the flavoring agent is selected from the group consisting of a vegetable extract, a fruit extract, an acid, an antioxidant, a carotenoid, a lactone, and any combinations thereof. In some embodiments, the antioxidant is epigallocatechin gallate. In some embodiments, the carotenoid is lutein, 3-carotene, zeaxanthin, trans-(3-apo-8′-carotenal) lycopene, or canthaxanthin. In some embodiments, the vegetable extract is from a cucumber or tomato. In some embodiments, the fruit extract is from a melon or pineapple.

In some embodiments, the cell-based meat product comprises a carbohydrate based gel. In some embodiments, the carbohydrate based gel of the cell-based meat product have a melting temperature between about 45° C. and about 85° C. In some embodiments, the carbohydrate-based gel comprises agar, pectin, carrageenan, konjac, alginate, chemically-modified agarose, or mixtures thereof.

In some embodiments, the cell-based meat product comprises one or more binding agents. In some embodiments, the binding agent comprises an isolated plant protein (e.g., a RuBisCO, an albumin, a gluten, a conglycinin, or mixtures thereof). In some embodiments, the denaturation temperature of the binding agent is between about 40° C. and about 80° C. In some embodiments, the binding agent is a carbohydrate based gel that becomes firm upon cooking to 140° F. to 190° F. In some embodiments, the cell-based meat product comprise at least about 0.01%, between about 0.01% and about 15%, between about 0.1% and about 10%, between about 0.25% and about 7%, between about 0.25% and about 5%, between about 0.5% and about 4.5%, between about 1% and about 4%, between about 1.5% and about 3.5%, between about 2% and about 3%, between about 1% and about 2.5%, between about 2% and about 2.5%, between about 0.5% and about 2%, or between about 5% and about 10% (including all ranges and subranges therebetween) by weight of binding agents. Examples of suitable binding agents include but are not limited to purees (e.g., bean puree, sweet potato puree, pumpkin puree, applesauce, yam puree, banana puree, plan-tain puree, date puree, prune puree, fig puree, zucchini puree, carrot puree, coconut puree), native or modified starches (e.g., starches from grains, starches from tuber, potato starch, sweet potato starch, corn starch, waxy corn starch, tapioca starch, tapioca, arrowroot starch, taro starch, pea starch, chickpea starch, rice starch, waxy rice starch, lentil starch, barley starch, sorghum starch, wheat starch, and physical or chemical modifications thereof [including, e.g., pre-gelatinized starch, acetylated starch, phosphate bonded starch, carboxymethylat-ed starch, hydroxypropylated starch]), flours derived from grains or legumes or roots (e.g., from taro, banana, jackfruit, konjac, lentil, fava, lupin bean, pea, bean, rice, wheat, barley, rye, corn, sweet rice, soy, teff, buckwheat, amaranth, chickpea, sorghum, almond, chia seed, flaxseed, potato, tapioca, potato), protein isolates (e.g., from potato, soy, pea, lentil, chickpea, lupin, oat, canola, wheat), hydrolyzed protein isolates (e.g., hydrolyzed pea protein isolate, hydrolyzed soy protein isolate), protein concentrates (e.g. from algae, lentil, pea, soy, chickpea, rice, hemp, fava bean, pigeon pea, cowpea, vital wheat gluten), beta-glucans (e.g., from bacteria (e.g., curdlan), oat, rye, wheat, yeast, barley, algae, mushroom), gums (e.g., xanthan gum, guar gum, locust bean gum, gellan gum, gum arabic, vegetable gum, tara gum, tragacanth gum, konjac gum, fenugreek gum, gum karaya, gellan gum, high-acetyl gellan gum, low-acetyl gellan gum), native or relatively folded (i.e., not fully in the native functional state but not fully denatured) proteins (e.g., fava protein, lentil protein, pea protein, ribulose-1,5-bisphosphate carboxylase/oxygenase [Rubisco], chickpea protein, mung bean protein, pigeon pea protein, lupin bean protein, soybean protein, white bean protein, black bean protein, navy bean protein, adzuki bean protein, sunflower seed protein), polysaccharides and modified polysaccharides (e.g., methylcellulose, hydroxypropyl methylcellulose, carboxymethyl cellulose, maltodextrin, carrageenan and its salts, kelp and kelp extracts, alginic acid and its salts, agar, agarose, agaropectin, pectin, alginate), nut and seed butters (e.g., almond butter, cashew butter, hazelnut butter, macadamia nut butter, peanut butter, pecan butter, pistachio butter, walnut butter, pumpkin seed butter, sesame seed butter, soybean butter, sunflower seed butter), enzymes (e.g., trans-glutaminase, thio-oxidoreductase), prolamin proteins (e.g., Zein protein), gelatin, egg protein, potato flakes, okra, tubers, fibers (e.g., psyllium husk), and derivatives and combinations thereof. In some embodiments, the carbohydrate based gel comprises methylcellulose, hydroxypropylmethyl cellulose, guar gum, locust bean gum, xanthan gum, or a mixture thereof. In some embodiments, the binding agent comprises egg albumin or collage. In some embodiments, the cell-based meat products comprise between about 0.1% and about 4%, between about 0.25% and about 1.5%, between about 0.5% and about 1.25%, between about 0.75% and about 1%, between about 1% and about 1.5%, between about 1.5% and about 2%, between about 2% and about 2.5%, between about 2.5% and about 3%, or between about 3% and about 4% by weight of starch. In some embodiments, the cell-based meat products comprise between about 0.5% and about 5%, between about 1% and about 4%, between about 2% and about 3%, between about 1% and about 2%, between about 3% and about 4%, between about 4% and about 5%, between about 0.5% and about 1.5%, or between about 1% and about 1.5% (including all ranges and subranges therebetween) by weight of methyl cellulose.

Suitable binding agents and suitable amounts of such binding agents can be identified by titrating different binding agents against the cohesiveness, binding, and malleability of uncooked meat-like food products, or against the cohesiveness and binding of cooked meat-like food products. The presence and distribution of carbohydrates used as binders in a meat-like food product provided herein can be determined by methods known in the art, such as, for example, methods that involve microscopic observation using brightfield, fluorescence, or phase contrast microscopy of thin strips of refrigerated meat-like food product stained with a natural or fluorescent dye that selectively stains carbohydrates.

In some embodiments, the cell-based meat product further comprises proteins, lipids, carbohydrates, cells, or other ingredients derived from one or more plant or modified plant sources. In some embodiments, the cell-based meat product further comprises plant-based protein or product. Examples of suitable plants include but are not limited to spermatophytes (spermatophyta), acrogymnospermae, angiosperms (magnoliophyta), ginkgoidae, pinidae, mesangiospermae, cycads, Ginkgo, conifers, gnetophytes, Ginkgo biloba, cypress, junipers, thuja, cedarwood, pines, angelica, caraway, coriander, cumin, fennel, parsley, dill, dandelion, helichrysum, marigold, mugwort, safflower, camomile, lettuce, wormwood, calendula, citronella, sages, thyme, chia seed, mustard, olive, coffee, capsicum, eggplant, paprika, cranberry, kiwi, vegetable plants (e.g., carrot, celery), tagetes, tansy, tarragon, sunflower, wintergreen, basil, hyssop, lavender, lemon verbena, marjoram, melissa, patchouli, pennyroyoal, peppermint, rosemary, sesame, spearmint, primroses, samara, pepper, pimento, potato, sweet potato, tomato, blueberry, nightshades, petunia, morning glory, lilac, jasmin, honeysuckle, snapdragon, psyllium, wormseed, buckwheat, amaranth, chard, quinoa, spinach, rhubarb, jojoba, cypselea, chlorella, manila, hazelnut, canola, kale, bok choy, rutabaga, frankincense, myrrh, elemi, hemp, pumpkin, squash, curcurbit, manioc, dalbergia, legume plants (e.g., alfalfa, lentils, beans, clovers, peas, fava coceira, frijole bola roja, frijole negro, lespedeza, licorice, lupin, mesquite, carob, soybean, peanut, tamarind, wisteria, cassia, chickpea, garbanzo, fenugreek, green pea, yellow pea, snow pea, yellow pea, lima bean, fava bean), geranium, flax, pomegranate, cotton, okra, neem, fig, mulberry, clove, eucalyptus, tea tree, niaouli, fruiting plants (e.g, apple, apricot, peach, plum, pear, nectarine), strawberry, blackberry, raspberry, cherry, prune, rose, tangerine, citrus (e.g., grapefruit, lemon, lime, orange, bitter orange, mandarin), mango, citrus bergamot, buchu, grape, broccoli, brussels, sprout, camelina, cauliflower, rape, rapeseed (canola), turnip, cabbage, cucumber, watermelon, honeydew melon, zucchini, birch, walnut, cassava, baobab, allspice, almond, breadfruit, sandalwood, macadamia, taro, tuberose, aloe vera, garlic, onion, shallot, vanilla, yucca, vetiver, galangal, barley, corn, curcuma aromatica, galangal, ginger, lemon grass, oat, palm, pineapple, rice, rye, sorghum, triticale, turmeric, yam, bamboo, barley, cajuput, canna, cardamom, maize, oat, wheat, cinnamon, sassafras, lindera benzoin, bay laurel, avocado, ylang-ylang, mace, nutmeg, moringa, horsetail, oregano, cilantro, chervil, chive, aggregate fruits, grain plants, herbal plants, leafy vegetables, non-grain legume plants, nut plants, succulent plants, land plants, water plants, delbergia, millets, drupes, schizocarps, flowering plants, non-flowering plants, cultured plants, wild plants, trees, shrubs, flowers, grasses, herbaceous plants, brushes, lianas, cacti, green algae, tropical plants, subtropical plants, temperate plants, and derivatives and crosses thereof.

In some embodiments, the plant is selected from alfalfa, bamboo, barley, beets, black beans, broccoli, cabbage, canola, carrot, cauliflower, celery, celery root, chickpeas, corn, cotton, cow peas, fava beans, flax, garbanzo beans, green beans, kale, kidney beans, lupin, mung beans, navy beans, northern beans, nuts, oats, parsley, pearl millet, peas, pine nuts, pinto beans, potato, quinoa, red beans, rice, sesame, soy, spelt, sugarbeet, sunflowers, sweet potato, tobacco, wheat, white beans, whole grains, wild rice, zucchini, and a mixture thereof.

Modified plant sources may be obtained from a variety of sources including but not limited to commercial cell banks (e.g., ATCC, collaborative sources), or can be generated from natural plants by methods known in the art, including selection, mutation, or gene manipulation. Selection generally involves continuous multiplication and steady increase in dilution rates under selective pressure. Mutation generally involves selection after exposure to mutagenic agents. Gene manipulation generally involves genetic engineering (e.g., gene splicing, insertion of deletions or modifications by homologous recombination) of target genes. A modified plant source may produce a non-native protein, carbohydrate, lipid, or other compound, or produce a non-native amount of a native protein, carbohydrate, lipid, or other compound. In some embodiments, the modified plant source expresses higher or lower levels of a native protein or metabolic pathway compound. In other such embodiments, the modified plant source expresses one or more novel recombinant proteins, RNAs, or metabolic pathway components derived from another plant, algae, microbe, or fungus. In other embodiments, the modified plant source has an increased nutraceutical content compared to its native state. In yet other embodiments, the modified plant source has more favorable growth and production characteristics compared to its native state. In some such embodiments, the modified plant source has an increased specific growth rate compared to its native state. In other such embodiments, the modified plant source can utilize a different carbon source than its native state.

### Kit and Articles of Manufacture

The present disclosure provides kits for generating the genetically modified cell line. The kits may comprise a recombinant nucleic acid that can generate the genetic modification upon introduction into a corresponding parental cell, and/or an agent for introducing the recombinant nucleic acid into the corresponding parental cell. In some embodiments, the kit further comprises a cultivation infrastructure.

The present disclosure provides kits for maintaining or growing the genetically modified cell line. The kits may comprise a culture medium for maintaining and/or growing the genetically modified cell line. In some embodiments, the kit further comprises a cultivation infrastructure.

The present disclosure also provides articles of manufacture comprising any one of the compositions, cell lines, products or kits of the disclosure.

### Methods

In one aspect, the present disclosure provides methods of adapting a metazoan cell line to suspension culture, comprising the steps of: (a) introducing a genetic modification to the cell line, wherein said genetic modification results in 1) reduced protein activity of at least one of PTEN, CASP3, CASP8, CDH1, ITGB1, RB1, IGFBP4, p21, p27, p16, and p18; 2) increased protein activity of SRC; 3) increased protein activity of TERT; and/or 4) reduced protein activity of p53, compared to a control cell line without said genetic modifications; and (b) introducing the cell line to suspension culture.

In some embodiments, the present disclosure provides methods of adapting the genetically modified cell line for suspension culture.

The introduction of cells to suspension culture may be done by methods which are known in the art. In some embodiments, the cells of an adherent culture are removed from their growth surface using a cell scraper and then placed in a vessel, such as a shake flask or a spinner flask, in which the culture is constantly agitated. In some embodiments, the cells of a culture are removed from the growth surface using an enzyme (e.g., trypsin), followed by the inactivation of the trypsin, or by removal of the trypsin by washing the cells, and then placing the cells in suspension culture in a vessel. In some embodiments, cells cultured in adherent culture are dislodged from substratum by non-enzymatic procedures, such as by gentle tapping of the culture vessel or by treatment with solutions containing divalent ion chelators. In some embodiments, the divalent ion chelator is ethylenediaminetetraacetic acid (EDTA) or ethylene glycol-bis (β-aminoethyl ether)-N,N,N′,N′-tetraacetic acid (EGTA).

In some embodiments, the introduction of cells to suspension culture comprises an acute treatment. In some embodiments, the acute treatment comprises treating the cells with a small molecule. In some embodiments, the small molecule is selected from the group consisting of a Rho-associated kinase (ROCK) inhibitor, an inhibitor of microtubule polymerization (e.g., nocodazole) and a myosin inhibitor (e.g., blebbistatin). In some embodiments, the acute treatment is performed before the cells are moved into suspension culture. In some embodiments, the acute treatment is performed right after the cells are moved into suspension culture.

In some embodiments, the cells are maintained in suspension by shaking, rocking, agitating, rolling, stirring, or any combination thereof.

In some embodiments, the disclosure provides methods of adapting the cells from growth in medium comprising regular serum to a cell growth medium lacking or having a reduced level of serum and/or one or more exogenous growth factors. In some embodiments, the method comprises directly transferring the cells from a regular cell growth medium to a cell growth medium lacking (or having reduced levels of) serum and/or one or more growth factors. In some embodiments, the method comprises gradually weaning the cells from a regular cell growth medium to a cell growth medium lacking (or having reduced levels of) serum and/or one or more growth factors. In some embodiments, weaning the cells comprise reducing the level of the serum and/or one or more growth factors to about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5% (including all ranges and subranges therebetween) of the concentration of the serum and/or one or more growth factors in the previous passage. In some embodiments, weaning the cells comprise reducing the level of the serum and/or one or more growth factors to about 75% of the concentration of the serum and/or one or more growth factors in the previous passage.

In some embodiments, the disclosure provides methods of monitoring and removing cell aggregates. In some embodiments, the disclosure provides a method for selecting and maintaining a suspension culture adapted cell line that has low or minimal tendency of forming cell aggregates (i.e., cell clumps). In some embodiments, cells originally derived from an adherent cell line have a tendency to grow as cell aggregates in suspension culture. Cultures with varying levels of cell aggregation may display different growth kinetics. The control of aggregate size is an important issue. Cell death and necrosis may occur within aggregates. Severe aggregation may result in poor cell growth as a result of limitations in space and metabolic diffusion. Both biomass measurement and aggregation quantification are important in determining cell growth and behavior in an aggregated suspension culture. Therefore, in one aspect, the disclosure provides methods of assessing the degree of cell aggregation in a suspension culture.

The presence of cell aggregates or clumps in the culture may be determined by methods known in the art. For example, the level of cell aggregates may be quantified using Vi-Cell (Beckman Coulter) or flow cytometry. Alternatively, the presence of aggregates may be visualized microscopically or by use of a cell sizing apparatus such as a COULTER COUNTER (Beckman Coulter) or an AccuSizer 780/SPOS Single Particle Optical Sizer (Entegris). Other automated methods for quantitating cell aggregation are known in the art (Neelamegham et al., Ann. Biomed. Eng. 25 (1): 180-9 (1997); Tsao et al., Biotechnol. Prog. 16:809-814 (2000)).

In some embodiments, the amount of cell aggregates in the suspension culture may be reduced by adding a lipid mixture to the culture. Addition of a chemically defined lipid mixture may avoid the introduction of animal products to the culture.

In some embodiments, during suspension adaptation of the genetically modified cell line, cells not associated with cell aggregates are selectively retained. The selective retention of cells not associated with cell aggregates may be done by methods which are well known in the art. In some embodiments, the agitation of the suspension culture can be stopped for a time period (e.g., about 1, 2, 3, 4, 5, 7, 8, 9, 10, 15, 20, 25, 30, 40, 50, or 60 minutes, including all ranges and subranges therebetween) to allow large cell aggregates to settle to the bottom of the culture vessel. In some embodiments, the upper portion of the culture (e.g., the upper about 50%, 60%, 70%, 80%, 90%, or 95% of the volume of the culture, including all ranges and subranges therebetween), which contains individual cells and cells in smaller aggregates, is then drawn off and subcultured in a new vessel. In some embodiments, after the agitation is stopped for a given time period, the bottom of the culture (e.g., the bottom about 5%, 10%, 20%, 30%, 40%, or 50% of the volume of the culture, including all ranges and subranges therebetween) is drawn off from the bottom of the vessel. The remaining culture volume that contains individual cells and smaller cell aggregates is subcultured. The desired cell population may be enriched by multiple rounds of selection e.g., by repeating the procedure. The resulting cells will exhibit less clumping or less of a degree of cell aggregation than the non-adapted cells in the same suspension culture medium.

The degree of culture clumping or aggregation during culturing may be monitored by particle, i.e., cell aggregate, size measurement. In some embodiments, the size measurement is performed using an AccuSizer 780/SPOS Single Particle Optical Sizer. In this instrument, individual particles are passed by a laser beam and the amount of light blocked by each particle is measured. The amount of light blocked corresponds to the cross-sectional area of the particle and thus the cell clump or cell aggregate size. The distribution profile of single cells and cell clumps is reported in a tabular form or as a histogram. The optical sizer is able to detect particle sizes ranging from individual cells e.g., 10 to 15 microns in diameter, to cell aggregates up to 400 microns in diameter. In some embodiments, the monitoring of cell aggregation or the degree of cell aggregation is performed by the method disclosed in Tsao et al. (Biotechnol. Prog. 16:809-814 (2000)).

In one aspect, the present disclosure provides methods of increasing maximum viable cell density (VCD) of a metazoan cell line, comprising introducing one or more genetic modifications to the cell line. In some embodiments, the method increases VCD of the cell line in a cell growth medium lacking (or having reduced levels of) serum and/or one or more exogenous growth factors. In some embodiments, the growth factor comprises a FGF. In some embodiments, the cell line is in a suspension culture. In some embodiments, the cell line is in an adherent culture.

In one aspect, the present disclosure provides methods of producing a clonal cell culture, comprising the step of placing the genetically modified cell line of the disclosure in a cell growth medium lacking (or having reduced levels of) serum and/or one or more exogenous growth factors. In some embodiments, the growth factor comprises a FGF. In some embodiments, the method further comprises firstly generating the genetically modified cell line by a genetic modification method.

In one aspect, the present disclosure provides methods of producing a differentiated cell culture, comprising the steps of: a) placing the genetically modified cell line of the disclosure in a cell growth medium lacking (or having reduced levels of) exogenous serum and/or FGF to generate a cell population. In some embodiments, the method further comprises the step of b) inducing myogenic differentiation of the cell population. In some embodiments, the method further comprises firstly generating the genetically modified cell line by a genetic modification method.

In one aspect, the present disclosure provides methods of generating the genetically modified cell line of the disclosure, comprising introducing a recombinant nucleic acid into a corresponding parental cell to cause the genetic modification.

In one aspect, the present disclosure provides methods of producing a cell-based meat product, the method comprising combining one or more plant-based products with the genetically modified cell population of the disclosure. In some embodiments, the cell-based meat product is substantially based on plant-based products. In some embodiments, the cell-based meat product produced by combining genetically modified cell population with a plant-based product has an increased meat like flavor, meat-like aroma, and/or meat-like color as compared to a plant-based product without the genetically modified cell population. In some embodiments, the cell-based meat product has increased protein level compared to a plant-based product without the genetically modified cell population of the disclosure. In some embodiments, one or more additional food additives may be included in the cell-based meat product.

While the disclosure is described primarily in terms of food production, the cell lines and methods of the disclosure are suitable for use in other applications where cell proliferation may be required or helpful, such as growing tissue and organisms that may be useful for treatment of disease or other conditions.

### Deposit Information

The deposit of all the cell lines mentioned in Example 2 below are maintained by Eat SCiFi Inc., 1933 Davis Street, Suite 244, San Leandro, CA 94577, USA. These cell lines include the ‘005’ cell line, ‘048’ cell line, ‘050’ cell line, ‘115’ cell line, ‘115-CASP3’ cell line, ‘115-PTEN’ cell line, ‘115-CASP8’ cell line, ‘115-CDH1’ cell line, ‘050’ cell line, ‘091’ cell line, ‘093’ cell line, ‘094’ cell line, ‘098’ cell line, ‘099’ cell line, ‘113’ cell line, ‘100’ cell line, ‘101’ cell line, ‘102’ cell line, ‘103’ cell line, ‘104’ cell line, ‘105’ cell line, ‘106’ cell line, ‘107’ cell line, ‘3+PTEN KO’ cell line, ‘1+CASP3 KO’ cell line, ‘2+PTEN KO’ cell line, ‘2+CASP3 KO’ cell line, ‘2+CDH1 KO’ cell line, ‘3+CDH1 KO’ cell line, ‘3+CASP8 KO’ cell line, “117” cell line, ‘205’ cell line, ‘207’ cell line, ‘208’ cell line, ‘209’ cell line, ‘210’ cell line, ‘217’ cell line, ‘218’ cell line, ‘219’ cell line, ‘220’ cell line, ‘221’ cell line, ‘222’ cell line, ‘356’ cell line, the ‘416’ cell line, the ‘541’ cell line, the ‘542’ cell line, the ‘584’ cell line, the ‘586’ cell line, the ‘591’ cell line, the ‘594’ cell line, the ‘597’ cell line, the ‘599’ cell line, the ‘616’ cell line, the ‘624’ cell line, the ‘681’ cell line, the ‘697’ cell line, the ‘978’ cell line, the ‘980’ cell line, the ‘982’ cell line, and the ‘983’ cell line. In addition, a sample of these cell lines (e.g., the ‘591’ cell line) of this disclosure will be deposited with an International Depositary Authority as established under the Budapest Treaty according to 37 CFR 1.803 (a) (1). Applicant will deposit the cells at the American Type Culture Collection (ATCC), located at Historic District, 10801 University Blvd, Manassas, VA 20110, United States.

To satisfy the enablement requirements of 35 U.S.C. 112, and to certify that the deposit of the isolated cell line of the present disclosure meets the criteria set forth in 37 C.F.R. 1.801-1.809 and Manual of Patent Examining Procedure (MPEP) 2402-2411.05, Applicants hereby make the following statements regarding the deposited cell line(s):
1. During the pendency of this application, access to the disclosure will be afforded to the Commissioner upon request;
2. All restrictions on availability to the public will be irrevocably removed upon granting of the patent under conditions specified in 37 CFR 1.808;
3. The deposit will be maintained in a public repository for a period of 30 years or 5 years after the last request or for the effective life of the patent, whichever is longer;
4. A test of the viability of the biological material at the time of deposit will be conducted by the public depository under 37 C.F.R. 1.807; and
5. The deposit will be replaced if it should ever become unavailable.

Access to this deposit will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the variety will be irrevocably removed by affording access to a deposit of the cell line(s) with the ATCC.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1: Adaption of Fibroblasts and Myoblasts for Suspension Culture

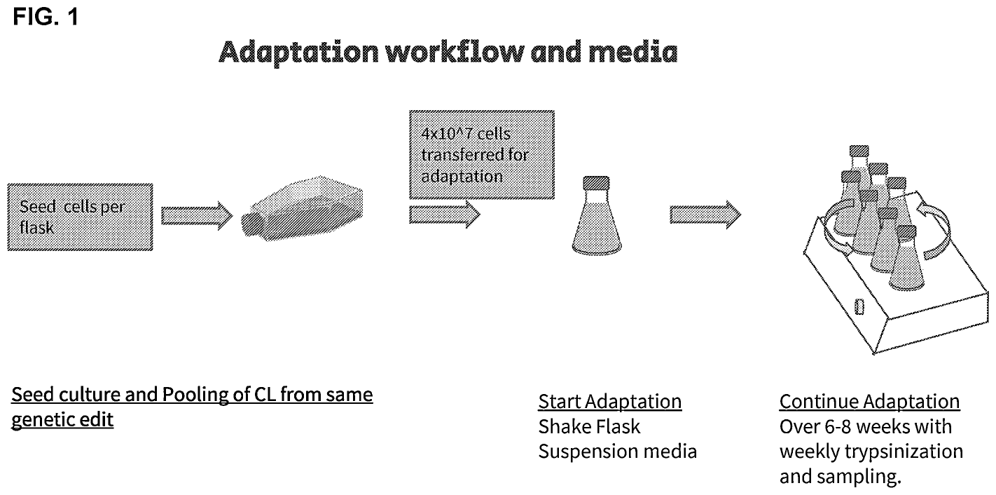

As outlined in FIG. 1, About 4e7 cells of a given cell line were resuspended in 40 mL growth media for final seed density of 1e6 cells/mL and added to a shake flask (125 mL). It was then incubated at 37C, 5% CO2, 100 rpm.

Once every week, the shaker flask was removed from the incubator, the cell culture material was collected in 50 mL sterile falcon tube and spun down (500 g for 5 min). Almost no cells were attached to the shaker flask. To break down the clumps forming during adaptation, the supernatant was discarded, the pellet was resuspended in 1 mL recombinant trypsin (trypLE) and incubated for 30 min. After incubation, the trypsinized mixture was neutralized with 10 mL of growth media. This cell culture material was then sampled for cell counting using the Cellaca-Trypan Blue method, as well as for proliferation using BrdU assay.

The rest of the cell culture material (˜10 mL) was diluted with fresh growth media and added back to the shake flask and incubator. The amount of fresh media to be added was determined by the cell counts, for a targeted cell density at 1e6 cells/mL in shake flask. The adaptation process was continued every week for 8 weeks.

Growth media was: DMEM/F12 95%, FBS 5%, Glutamine-200 mM 1%, 0.01% Pluronic and growth factor cocktail.

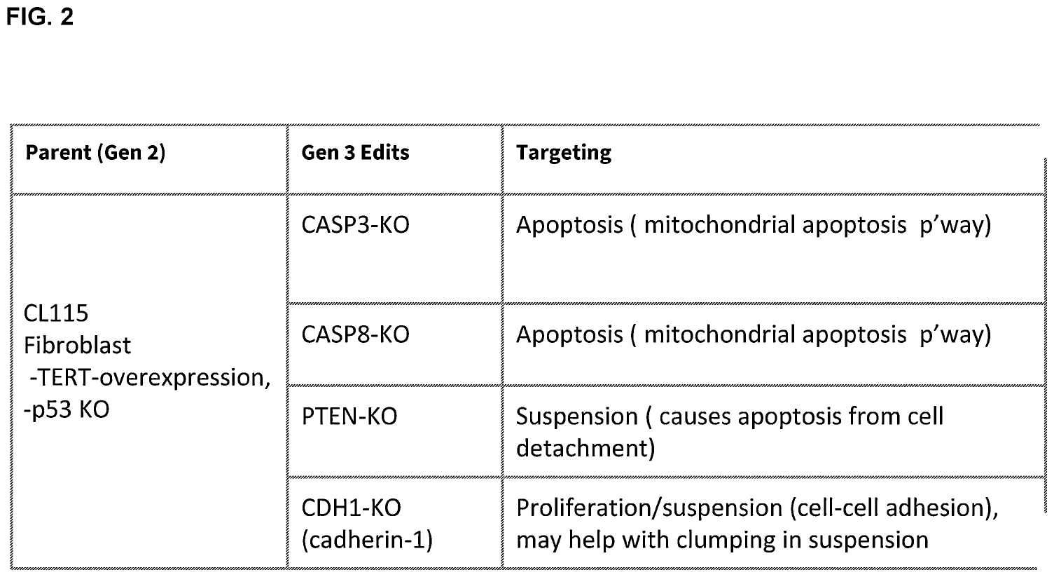

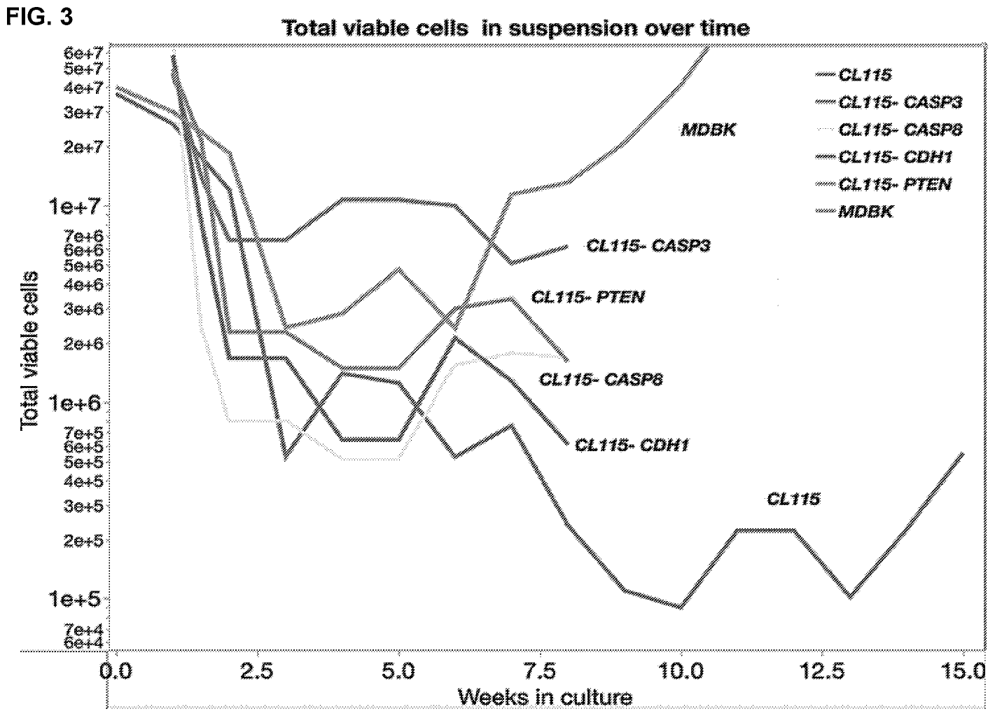

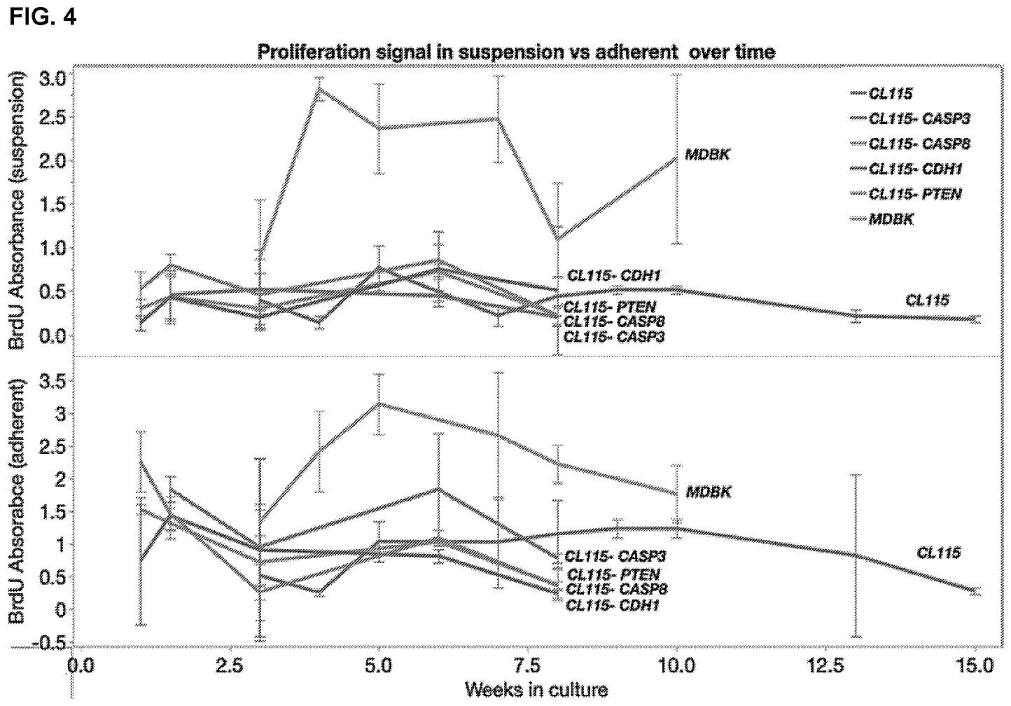

As shown in FIG. 2, bovine fibroblasts were genetically modified to 1) overexpress TERT protein via introducing a knock-in TERT; 2) knock-out endogenous p53 gene (p53-KO); and 3) individually knock-out each one of the endogenous CASP3, CASP8, PTEN and CDH1 genes. Compared to the parental fibroblast cell line with TERT-overexpression and p53-KO, the fibroblast cell lines with an additional knock-out of CASP3, CASP8, PTEN or CDH1 gene all showed higher cell viability in suspension culture (FIG. 3), and the fibroblast cell lines with CASP3 or PTEN knock-out also exhibited particularly dispersed phenotype. MDBK cells were used as a positive control here. All these genetically modified cell lines displayed similar proliferation signals in suspension culture (FIG. 4). Adherent proliferation assays were also performed to measure reversible quiescence.

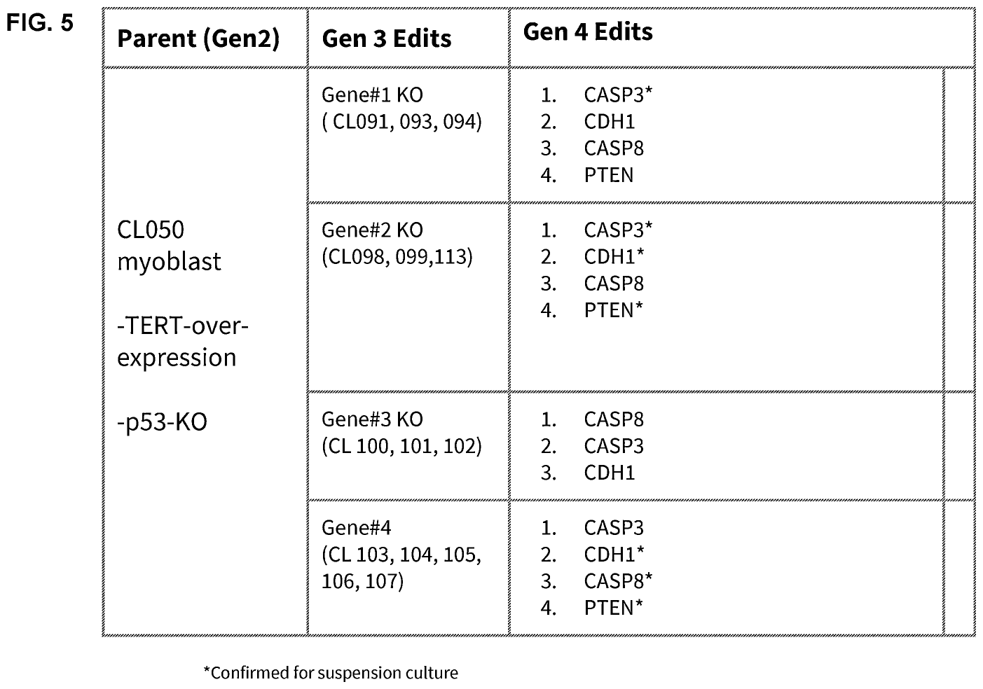

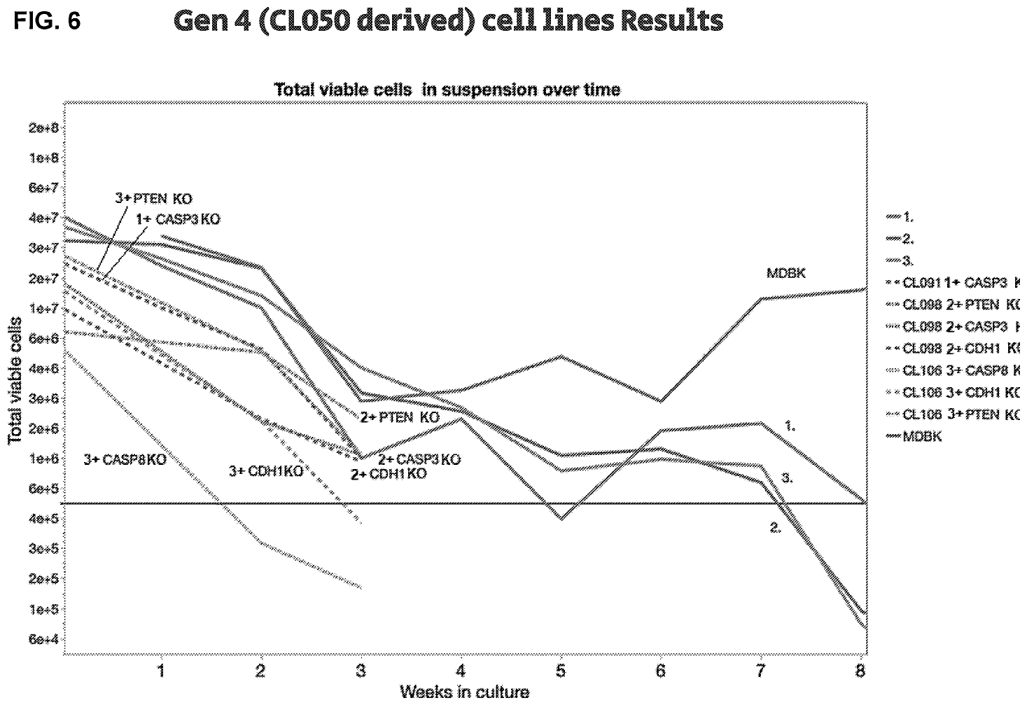

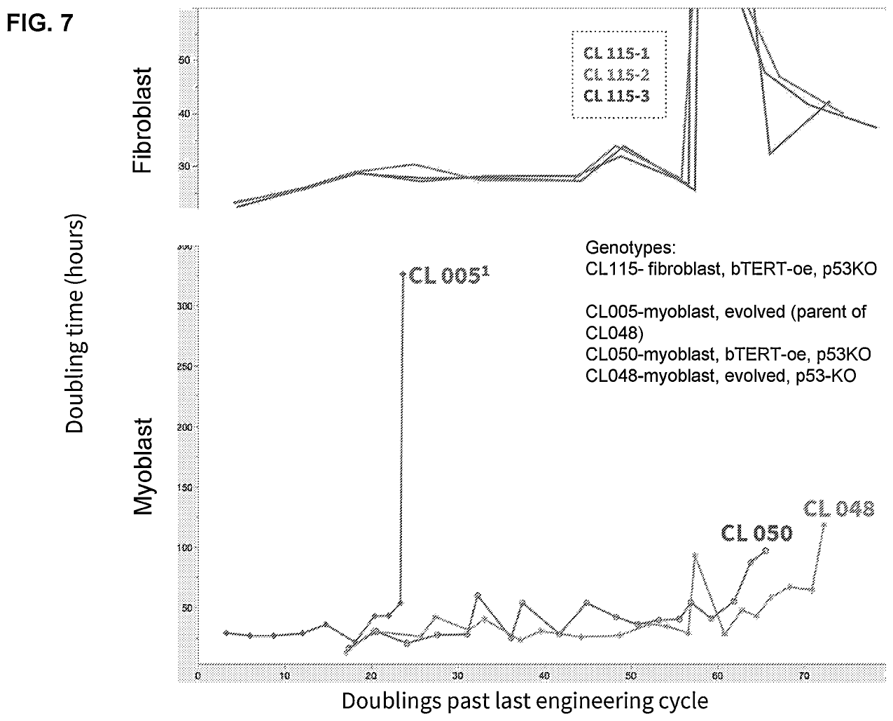

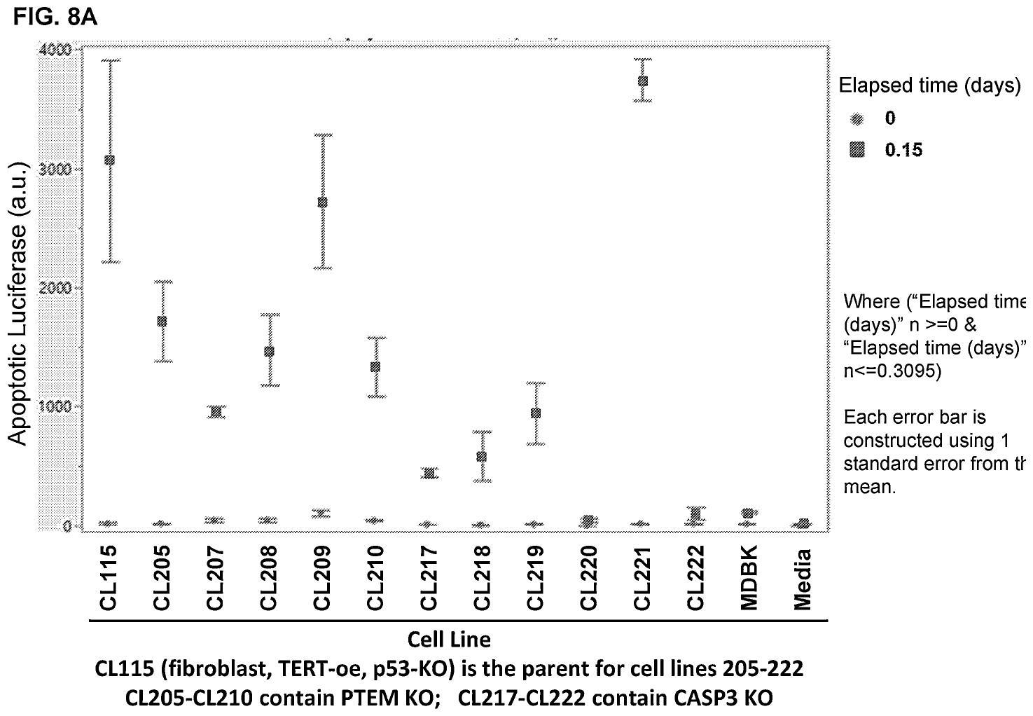

As shown in FIG. 5, bovine myoblasts with different genetical background (including CL-091, -093, -094, -098 to -107, and -113) were genetically modified to knock-out one of the endogenous CASP3, CASP8, PTEN and CDH1 genes. These bovine myoblasts share the genetic modifications of i) TERT protein overexpression via introducing a knock-in TERT; and ii) knock-out endogenous p53 gene (p53-KO), inherited from the parental cell line CL050. Suspension culture ability had been confirmed in many of these myoblast cell lines at the time of filing (as indicated by “*” in FIG. 5), and the validation of other genetically modified cell lines are ongoing. Viability of these myoblast cell lines were shown in FIG. 6. Indicated cell lines were continuously passaged in appropriate media. At each passage cell generation and doubling time were determined by counting cell in culture. When cells stop growing, their doubling time increases dramatically. Compared to the parental cell lines, myoblasts comprising TERT overexpression and/or p53-KO displayed improved proliferative capacity in suspension culture based on the cell doubling time (FIG. 7).

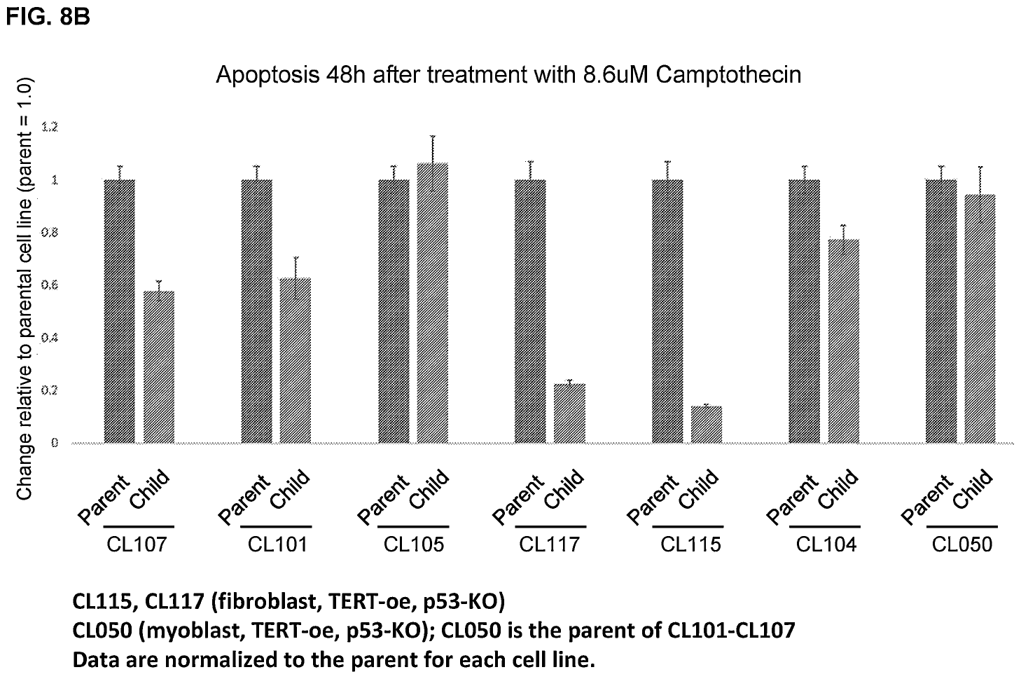

In FIG. 8A, indicated fibroblast cell lines growing in adherent state were forced into suspension through trypsinization at the beginning (0 days) and after 4 hrs (0.15 days) in suspension state, and apoptosis was measured by standard methods. Compared to the parental fibroblast cell line CL115 (with TERT over-expression and p53-KO), the cell lines with additional PTEN or CASP3 knock-out displayed lower level of apoptosis. Similarly, compared to the parental myoblast cell line CL050 (with TERT over-expression and p53-KO), the cell lines with additional gene knock-out displayed lower level of apoptosis when the cell lines growing in adherent state were treated with DNA damaging agent camptothecin and measured for apoptosis by standard methods 48 hours afterwards (FIG. 8B).

### Example 2: Further Screening of Fibroblasts for Suspension Culture

Over 150 bovine fibroblast cell lines were evaluated for suspension growth in a serum screening pipeline in plates, and the performance of the cell lines varied depending on the genetic makeup. Briefly, all suspension cell lines were subject to plate screen at the same starting concentration (typically 1e∧5-2e∧5 cells/ml) and the numbers of cells were measured after 4 days, in which the top 20% performers were promoted to batch study (in shaker flasks) for 7-8 days. The top 25% performers in the batch study were promoted to tank screening at a quarterly schedule. Cell performance was evaluated based on direct CyQUANT analysis.

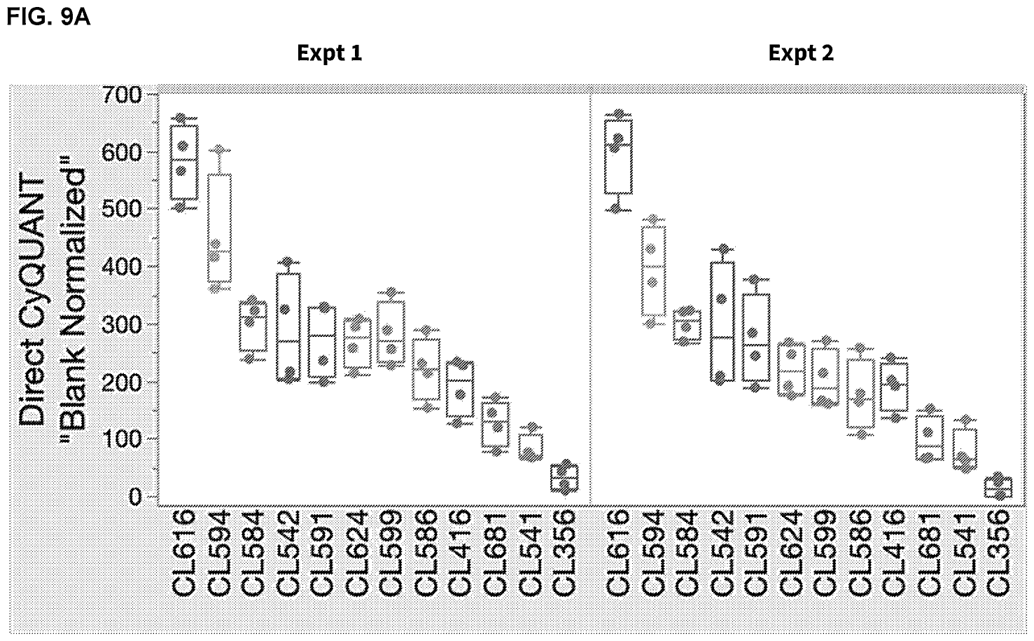

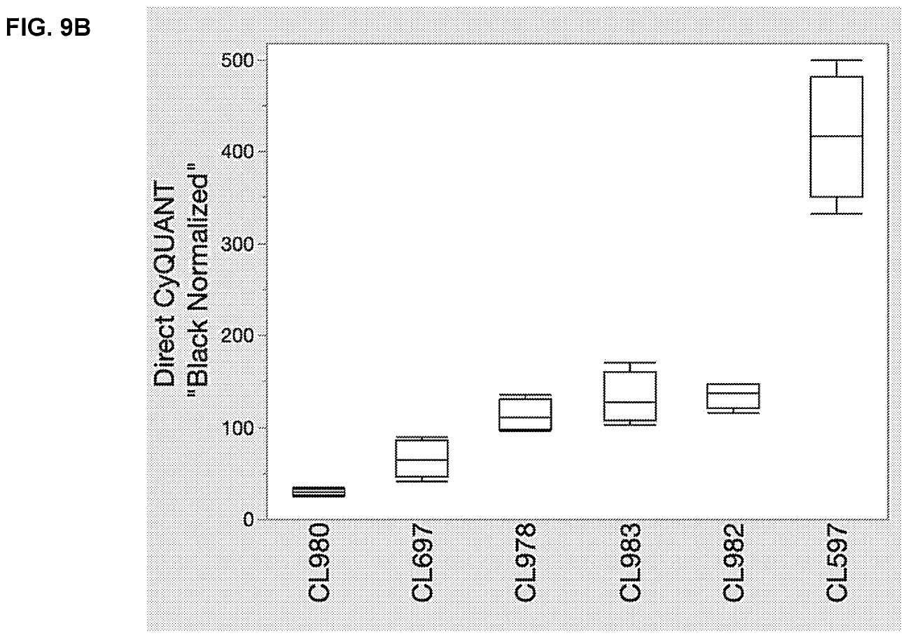

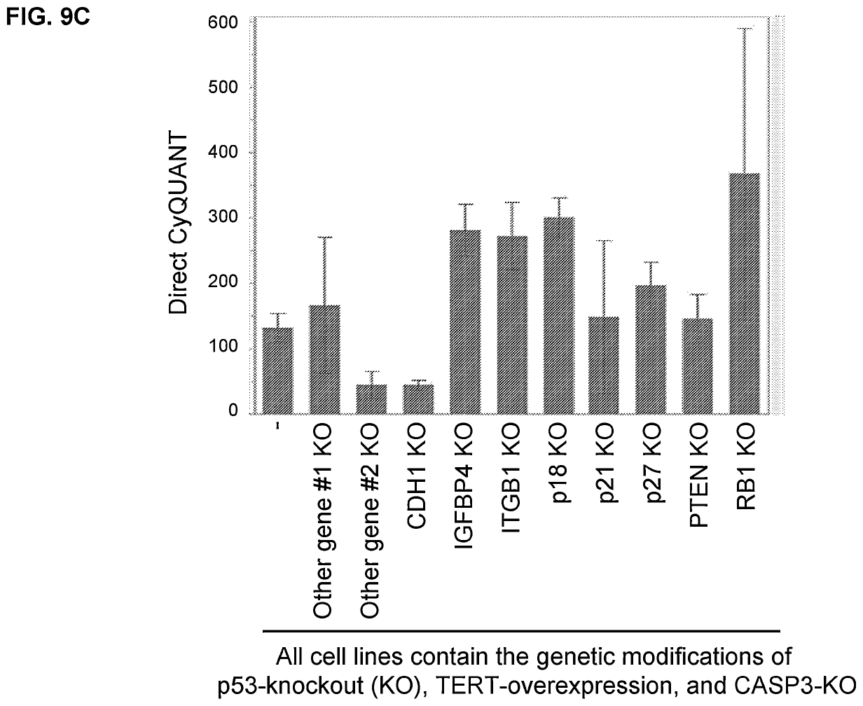

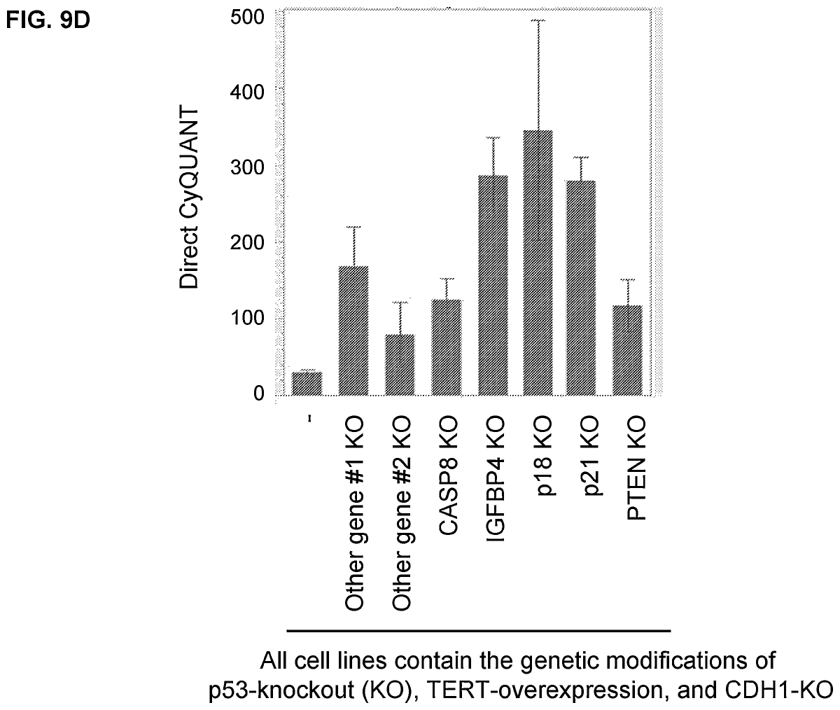

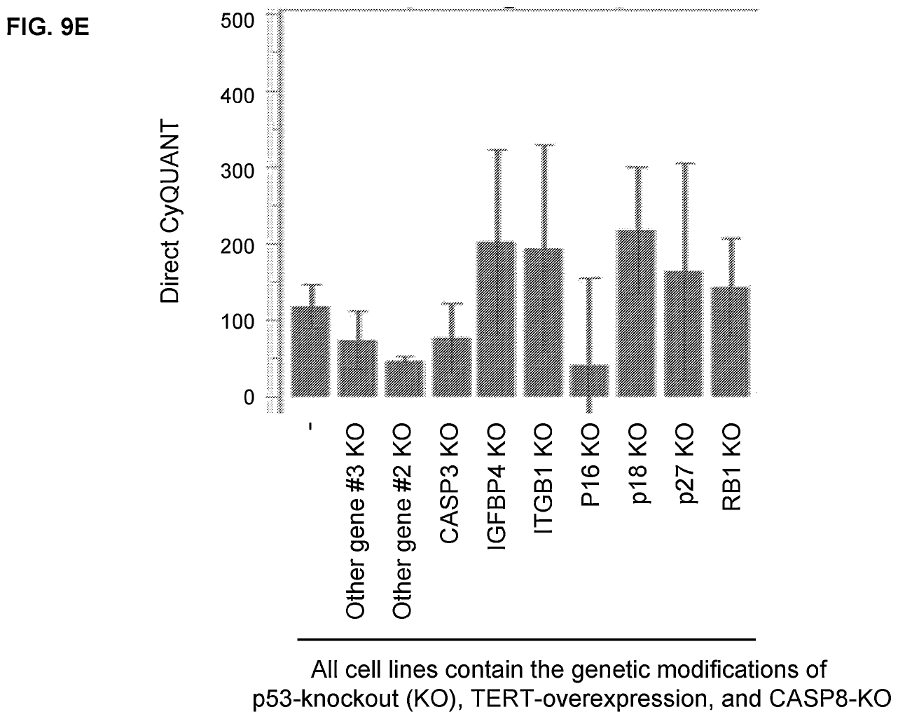

FIGS. 9A and 9B show representative results of direct CYQUANT analysis of cells screened in PSv1. Two replicate cryovials were tested for each cell line, and the mean coefficient of variation by cell line averaged to ˜15%. The data in plate screening demonstrated reproducible differential performance between cell lines. The genetic makeup of the cell lines are summarized in Table 3 below.

TABLE 3
Genetic Makeup of Cell Lines.

| Cell line | Genotype (all contain p53-knockout and |
|---|---|
| (CL) | TERT-overexpression Modifications)* |
| 616 | PTEN-KO, p27-KO |
| 594 | Constitutively active SRC Y553F |
| | overexpression |
| 584 | CDH1-KO, p21-KO |
| 542 | CDH1-KO, IGFBP4-KO |
| 591 | CASP3-KO, ITGB1-KO |
| 624 | CASP3-KO, IGFBP4-KO |
| 599 | CASP3-KO, p18-KO |
| 586 | CASP3-KO, p27-KO |
| 416 | PTEN-KO |
| 681 | CASP3-KO, p27-KO (different isolate) |
| 541 | CDH1-KO, PTEN-KO |
| 356 | CDH1-KO |
| 980 | CDH1-KO |
| 697 | PTEN-KO, p16-KO |
| 978 | — |
| 983 | CASP3-KO |
| 982 | PTEN-KO |
| 597 | CDH1-KO, p16-KO |

KO: knock-out.

FIG. 9C shows representative results of direct CYQUANT analysis of cell lines containing (i) the genetic modifications of p53-knockout (KO), TERT-overexpression, and CASP3-KO; and (ii) the indicated modification. FIG. 9D shows results of direct CYQUANT analysis of cell lines containing (i) the genetic modifications of p53-KO, TERT-overexpression, and CDH1-KO; and (ii) the indicated modification. FIG. 9E shows results of direct CYQUANT analysis of cell lines containing (i) the genetic modifications of p53-KO, TERT-overexpression, and CASP8-KO; and (ii) the indicated modification. CYQUANT analysis was performed after cells were grown in plates for 4 days. The experiments in FIG. 9C-9E were performed in the same experimental setup so their data could be compared across figures. These results show that various specific combinations of genetic modifications significantly increased cell counts.

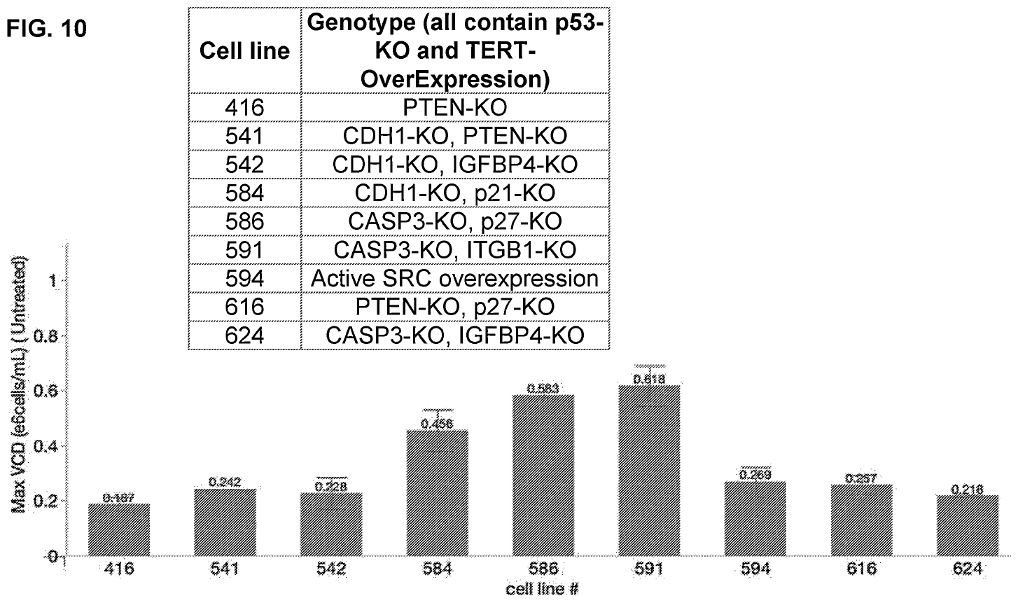

FIG. 10 shows the maximum viable cell density of selected cell lines during the suspension growth in bioreactors. Nearly all cell lines exhibited improved suspension culture phenotypes over controls. Among the cell lines tested, the ‘591’ cell line (CASP3-knockout and ITGB1-knockout in a genetic background of p53-knockout and TERT-overexpression) displayed the highest maximum viable cell density. The ‘586’ and ‘584’ cell lines also performed well.

According to additional internal studies (data not shown), we also have reasons to believe that the p53-knockout background may not be necessary for the improved suspension culture performance in the indicated cell lines. In addition, the TERT-overexpression may be optional in selected cell lines. Further tests are conducted to analyze the impact of p53 and/or TERT modifications on the suspension culture performance.

### Example 3: Culture of Engineered Myoblast Cell Line in Bioreactor

An industrial bioprocess involving mammalian cell culture typically requires a serum free suspension growth format. The industrial standard for this is the adaptation of Chinese hamster ovarian (CHO) cells to suspension growth in a bioreactor, and serum reduction is an established method to adapt CHO cells to this growth format. Likewise, the modified cells described in this disclosure is adapted to suspension format and serum free medium, either sequentially or concurrently. For serum free adaptation, the cells are incubated in a 37 Celsius incubator with 5% CO2 and passaged every 2-4 days based on cell density. Viable cell density is determined using Trypan Blue staining and cell counting by one or more of the following methods: manual counting with a hemocytometer, automated cell counter (such as the ThermoFisher Countess), digital holographic microscopy (DHM), or DNA content measurement such as the ThermoFisher Cyquant assay. Proliferation rate is calculated based on viable cell density. Once a stable proliferation rate and viability is achieved, the FBS concentration of the medium is reduced to 75% of the FBS concentration of the previous passage. Each time stable growth rate is achieved, the serum level in growth medium is reduced until serum level is eliminated.

For adaptation to suspension conditions, cells are cultured in shake flasks and incubated on an orbital shaker at a rate that allows for gas mixing but prevents shear stress (e.g., about 130 rpm). Cells are passaged every 2-4 days depending on viable cell density. Cell aggregates or clumps if formed during this process will be broken down by a mechanical process of pipetting up and down of full volume. If serum free medium adaptation is taking place concurrently, the concentration of FBS in the growth medium is reduced to 75% of previous concentration, as described above. Cells are considered completely adapted once cells grow at a stable rate at high viability (>80%) in serum free and suspension conditions.

The adapted cells are then cryopreserved in serum free media with cryoprotectants. The cryopreserved cells are thawed in a shake flask with serum free media. The thawed cell culture is incubated at 37 Celsius with 5% CO2 at 130 rpm on an orbital shaker. The cell culture is then passaged every 3-4 days and scaled up in volume. Once the necessary volume is achieved, the cells are used as inoculate in a bioreactor run.

The bioreactors are sampled and tested using the following methods. Viable cell density and viability of the culture are measured, for example, using a cell impermeable stain such as Trypan blue combined with cell counting methods: manual counting with a hemocytometer, automated cell counter (such as the ThermoFisher Countess), digital holographic microscopy (DHM), or by DNA content measurement such as the ThermoFisher Cyquant assay. Offline pH, gas and nutrient analysis is done, for example, by BioProfile Flex by Nova biomedical, YSI 2900. Glucose and glutamine consumption as well as lactate and ammonia accumulation is determined, for example, by assays such as Megazyme D-Glucose-HT, L-Glutamine/Ammonia and L-Lactate kits and the Promega Uptake-Glo and Lactate-Glo. The proliferation rate and viable cell density are recorded regularly at each total cell density level for both genetically modified cells and control cells without the genetic modification. The growth curve is plotted, and maximum viable cell density and the time to reach maximum VCD are calculated accordingly.

At the end of the bioprocess run, the biomass is harvested and further processed.

### Example 4: Preparation of Cell-Based Meat Product

In one set of preparation, the cultivated beef muscle cells harvested in Example 3 are added before grinding to prepare a slider. Briefly, cultivated bovine myoblast cells are mixed with plant proteins (e.g., soy and pea), flavoring agents (e.g., salt and citric acid), starch, emulsion and water until homogeneously dispersed. A cooking oil is then added to the mixture and mixed until homogeneously dispersed. The mixture is then grinded for cooking and/or freezing for long-term storage.

In another set of preparation, the cultivated beef muscle cells harvested in Example 3 are added after grinding to prepare a slider. Briefly, plant proteins (e.g., soy and pea), flavoring agents (e.g., salt and citric acid), starch, emulsion water, and a cooking oil are mixed until homogeneously dispersed and grinded. Cultivated bovine myoblast cells are then added to the mixture and mixed until homogeneously dispersed. The mixture is processed to form a patty, which is cooked and/or frozen for long-term storage.

### Example 5: Additional Cell Lines

Additional cell lines envisioned by the present disclosure include those comprising two or more genetic modifications according to one of Embodiments 1-286 in Table 2. The cell lines may further comprise genetic modification(s) resulting in increased protein activity of TERT and/or reduced protein activity of p53. These cell lines may be subject to the screening process described in Examples 1-2, cultured as described in Example 3, and/or used for preparation of cell-based meat product as described in Example 4.

### Further Numbered Embodiments

Further numbered embodiments of the present disclosure are provided as follows:

Embodiment 1. A clonal, metazoan cell line comprising a genetic modification resulting in:
(A) reduced protein activity of at least one of i) Phosphatase and Tensin Homolog (PTEN), ii) Caspase 3 (CASP3), iii) Caspase 8 (CASP8), iv) Cadherin 1 (CDH1), v) Integrin beta-1 (ITGB1), vi) Retinoblastoma-associated protein (RB1), vii) Insulin-like growth factor-binding protein 4 (IGFBP4), and viii) a Cyclin-dependent kinase inhibitor; and/or
(B) increased protein activity of SRC tyrosine-protein kinase (SRC), compared to a control cell line without said genetic modification.

Embodiment 2. The cell line of Embodiment 1, comprising a genetic modification resulting in increased protein activity of Telomerase Reverse Transcriptase (TERT).

Embodiment 3. The cell line of Embodiment 1 or 2, comprising a genetic modification resulting in reduced protein activity of Tumor Suppressor p53 (p53).

Embodiment 4. The cell line of any one of Embodiments 1-3, comprising the genetic modifications resulting in:
(i) reduced protein activity of CASP3; and
(ii) reduced protein activity of ITGB1;
compared to the control cell line without said genetic modifications.

Embodiment 5. The cell line of any one of Embodiments 1-3, comprising the genetic modifications resulting in:
(i) reduced protein activity of CASP3; and
(ii) reduced protein activity of Cyclin-dependent kinase inhibitor 1B (p27);
   compared to the control cell line without said genetic modifications.

Embodiment 6. The cell line of any one of Embodiments 1-3, comprising the genetic modifications resulting in:
(i) reduced protein activity of CDH1; and
(ii) reduced protein activity of Cyclin-dependent kinase inhibitor 1A (p21);
   compared to the control cell line without said genetic modifications.

Embodiment 7. The cell line of any one of Embodiments 1-3, comprising the genetic modifications resulting in:
(i) reduced protein activity of CDH1; and
(ii) reduced protein activity of Cyclin-dependent kinase inhibitor 2A (p16);
   compared to the control cell line without said genetic modification(s).

Embodiment 8. The cell line of any one of Embodiments 4-7, wherein the cell line comprises the genetic modification(s) resulting in:
(iii) increased protein activity of TERT; and/or
(iv) reduced protein activity of p53,
   compared to the control cell line without said genetic modifications.

Embodiment 9. The cell line of any one of Embodiments 1-8, wherein the Cyclin-dependent kinase inhibitor comprises one or more of Cyclin-dependent kinase inhibitor 1A (p21), Cyclin-dependent kinase inhibitor 1B (p27), Cyclin-dependent kinase inhibitor 2A (p16), and Cyclin-dependent kinase 4 inhibitor C (p18).

Embodiment 10. A clonal, metazoan cell line comprising:
i) a genetic modification resulting in increased protein activity of TERT; and/or
ii) a genetic modification resulting in reduced protein activity of p53,
compared to a control cell line without said genetic modification.

Embodiment 11. The cell line of Embodiment 10, comprising both i) and ii).

Embodiment 12. The cell line of any one of Embodiments 1-11, comprising a genetic modification resulting in reduced protein activity of PTEN.

Embodiment 13. The cell line of any one of Embodiments 1-12, comprising a genetic modification resulting in reduced protein activity of CASP3.

Embodiment 14. The cell line of any one of Embodiments 1-13, comprising a genetic modification resulting in reduced protein activity of CASP8.

Embodiment 15. The cell line of any one of Embodiments 1-14, comprising a genetic modification resulting in reduced protein activity of CDH1.

Embodiment 16. The cell line of any one of Embodiments 1-15, comprising a genetic modification resulting in reduced protein activity of ITGB1.

Embodiment 17. The cell line of any one of Embodiments 1-16, comprising a genetic modification resulting in reduced protein activity of RB1.

Embodiment 18. The cell line of any one of Embodiments 1-17, comprising a genetic modification resulting in reduced protein activity of IGFBP4.

Embodiment 19. The cell line of any one of Embodiments 1-18, comprising a genetic modification resulting in reduced protein activity of the Cyclin-dependent kinase inhibitor.

Embodiment 20. The cell line of any one of Embodiments 1-19, comprising a genetic modification resulting in reduced protein activity of Cyclin-dependent kinase inhibitor 1A (p21).

Embodiment 21. The cell line of any one of Embodiments 1-20, comprising a genetic modification resulting in reduced protein activity of, Cyclin-dependent kinase inhibitor 1B (p27).

Embodiment 22. The cell line of any one of Embodiments 1-21, comprising a genetic modification resulting in reduced protein activity of Cyclin-dependent kinase inhibitor 2A (p16).

Embodiment 23. The cell line of any one of Embodiments 1-22, comprising a genetic modification resulting in reduced protein activity of Cyclin-dependent kinase 4 inhibitor C (p18).

Embodiment 24. The cell line of any one of Embodiments 1-23, comprising a genetic modification resulting in increased protein activity of SRC.

Embodiment 25. The cell line of any one of Embodiments 1-24, wherein the cell line is capable of sustained suspension culture.

Embodiment 26. The cell line of Embodiment 25, wherein the cell line is capable of reaching a viable cell density (VCD) of at least 0.4 million, at least 0.5 million, at least 0.6 million, at least 0.7 million, at least 0.8 million, at least 0.9 million, at least 1 million, at least 2 million, at least 5 million, at least 10 million, 15 million or 20 million cells per mL in the suspension culture.

Embodiment 27. The cell line of any one of Embodiments 1-26, wherein the cell line is in suspension culture.

Embodiment 28. The cell line of Embodiment 27, wherein the cell line is stable in the suspension culture for at least 20, 30, 40, 50, or 60 generations.

Embodiment 29. The cell line of any one of Embodiments 1-28, wherein the control cell line without at least one of said genetic modifications is unstable or less stable in a suspension culture.

Embodiment 30. The cell line of any one of Embodiments 1-29, wherein the control cell line without at least one of said genetic modifications is not viable in a suspension culture.

Embodiment 31. The cell line of any one of Embodiments 25-30, wherein the cell line is capable of reaching at least 10%, 20%, 30%, 40%, 50%, 70%, 100%, 150%, 200%, 300%, or 400% higher maximum viable cell density (VCD) compared to the control cell line without at least one of said genetic modifications in the suspension culture.

Embodiment 32. The cell line of any one of Embodiments 25-31, wherein the cell line exhibits at least 5%, 10%, 20%, 30%, or 40% higher cell viability than the control cell line without at least one of said genetic modifications at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture.

Embodiment 33. The cell line of any one of Embodiments 25-32, wherein the cell line exhibits a cell viability of at least 70%, at least 75%, at least 80%, at least 85%, or at least 90% at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture.

Embodiment 34. The cell line of any one of Embodiments 25-33, wherein the cell line is capable of reaching maximum VCD at least 10%, 20%, 30%, 40%, 50%, 70%, 100%, 150%, 200%, 300%, or 400% faster than the control cell line without at least one of said genetic modifications in the suspension culture.

Embodiment 35. The cell line of any one of Embodiments 25-34, wherein the cell line is capable of reaching a viable cell density of 0.4 million, 0.5 million, 0.6 million, 0.7 million, 0.8 million, 0.9 million, 1 million, 2 million, 5 million, 10 million, 15 million, 20 million, 25 million, 30 million, 35 million, 40 million, 45 million, or 50 million, cells per mL in the suspension culture at least 10%, 20%, 30%, 40%, 50%, 70%, 100%, 150%, 200%, 300%, or 400% faster than the control cell line without at least one of said genetic modifications.

Embodiment 36. The cell line of any one of Embodiments 25-35, wherein the cell line exhibits a doubling time of less than 12 hours, 18 hours, 24 hours, 36 hours, or 48 hours when the viable cell density of the cell line is at 50% of its maximum VCD in the suspension culture.

Embodiment 37. The cell line of any one of Embodiments 25-36, wherein the cell line exhibits increased proliferation rate and/or decreased differentiation rate compared to the control cell line without at least one of said genetic modifications.

Embodiment 38. The cell line of any one of Embodiments 25-37, wherein the proliferation rate of the cell line is at least 20% higher than the control cell line without at least one of said genetic modifications when both cell lines are at a viable cell density of about 50%, 60%, 70%, 80%, 90%, or 95% of their maximum VCD.

Embodiment 39. The cell line of any one of Embodiments 25-38, wherein the differentiation rate of the cell line is at least 20% lower than the control cell line without at least one of said genetic modifications when both cell lines are at a viable cell density of about 50%, 60%, 70%, 80%, 90%, or 95% of their maximum VCD.

Embodiment 40. The cell line of any one of Embodiments 25-39, wherein cells of the suspension culture of the cell line comprises less than 50%, 40%, 30%, 20%, 10%, 5%, 3%, 2%, or 1% of cells in the form of cell aggregates having a diameter of greater than 100, 200, 300 or 400 microns.

Embodiment 41. The cell line of Embodiment 40, wherein the cell aggregates have a diameter of greater than 200 microns.

Embodiment 42. The cell line of any one of Embodiments 25-41, wherein cells of the suspension culture of the cell line comprises at least 50%, 60%, 70%, 80%, or 90% of cells in the form of single cells.

Embodiment 43. The cell line of Embodiment 42, wherein the single cells have an average diameter of less than 30, 25, 20, 15, 10, or 5 microns.

Embodiment 44. The cell line of Embodiment 42 or 43, wherein the single cells have an average circularity of greater than 0.5, 0.55, 0.6, 0.65, 0.7, 0.75, 0.8, 0.85, 0.9 or 0.95.

Embodiment 45. The cell line of any one of Embodiments 25-44, wherein the cell line exhibits increased anoikis resistance compared to the control cell line without at least one of said genetic modifications.

Embodiment 46. The cell line of Embodiment 45, wherein the percentage of apoptotic cells of the cell line is at least 10%, 20%, 30%, 40%, 50%, 60%, 70%, 80%, 90%, 95%, 97%, or 99% lower than the control cell line without at least one of said genetic modifications when both cell lines are at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture.

Embodiment 47. The cell line of Embodiment 45 or 46, wherein cells in the suspension culture of the cell line comprise less than 50%, 40%, 30%, 20%, 10%, 5%, 3%, 2%, or 1% of apoptotic cells.

Embodiment 48. The cell line of Embodiment 47, wherein the apoptotic cells are characterized using a biomarker selected from the group consisting of cytochrome C, caspase-3/7, DNA fragmentation and phosphatidylserine.

Embodiment 49. The cell line of any one of Embodiments 25-48, wherein less than 30%, 20%, 10%, 5%, 3%, 2% or 1% of cells in the suspension culture of the cell line are adherent cells.

Embodiment 50. The cell line of any one of Embodiments 1-49, wherein expression level of E-cadherin in the cell line is decreased by at least 30%, at least 50%, at least 70%, or at least 90%, compared to the control cell line without at least one of said genetic modifications.

Embodiment 51. The cell line of any one of Embodiments 1-50, wherein expression level of N-cadherin in the cell line is increased by at least 20%, at least 50%, at least 100%, or at least 2-fold, compared to the control cell line without at least one of said genetic modifications.

Embodiment 52. The cell line of any one of Embodiments 1-51, wherein expression level of vimentin in the cell line is increased by at least 20%, at least 50%, at least 100%, or at least 2-fold, compared to the control cell line without at least one of said genetic modifications.

Embodiment 53. The cell line of any one of Embodiments 1-52, wherein the cell line is a myoblast cell line.

Embodiment 54. The cell line of any one of Embodiments 1-52, wherein the cell line is a fibroblast cell line.

Embodiment 55. The cell line of any one of Embodiments 1-52, wherein the cell line is a preadipocyte cell line.

Embodiment 56. The cell line of any one of Embodiments 1-55, wherein the cell line lineage is skeletal muscle, subcutaneous adipose tissue, or connective tissue.

Embodiment 57. The cell line of any one of Embodiments 1-55, wherein the cell line lineage is skeletal muscle.

Embodiment 58. The cell line of any one of Embodiments 1-57, wherein the cell line is an immortalized cell line.

Embodiment 59. The cell line of any one of Embodiments 1-58, wherein the cell line species identity is selected from the group consisting of Bos Taurus, Sus scrofa, Ovis aries, Capra hircus, Oryctolagus cuniculus, Gallus gallus, Anas platyrhynchos, and Meleagris gallopavo.

Embodiment 60. The cell line of any one of Embodiments 1-58, wherein the cell line species identity is Bos taurus, Bos indicus, or a hybrid thereof.

Embodiment 61. The cell line of any one of Embodiments 1-58, wherein the cell line species identity is Gallus gallus.

Embodiment 62. The cell line of any one of Embodiments 25-61, wherein the suspension cell culture comprises a minimum regular cell growth medium.

Embodiment 63. The cell line of any one of Embodiments 25-62, wherein the suspension cell culture comprises a cell growth medium lacking (or having reduced levels of) serum and/or one or more growth factors.

Embodiment 64. The cell line of Embodiment 63, wherein the one or more growth factors are selected from the group consisting of fibroblast growth factor (FGF), epidermal growth factor (EGF), insulin-like growth factor (IGF), insulin, hepatocyte growth factor (HGF), transforming growth factor beta (TGF-β), tumor necrosis factor alpha (TNF-α), leukemia inhibitory factor (LIF) and interleukin-6 (IL6).

Embodiment 65. The cell line of Embodiment 63 or 64, wherein the cell growth medium comprises no exogenous FGF, EGF, IGF, HGF, TGF-β, TNF-α, LIF and/or IL6.

Embodiment 66. The cell line of any one of Embodiments 63-65, wherein the cell growth medium comprises no serum.

Embodiment 67. The cell line of any one of Embodiments 1-66, wherein the genetic modification resulting in reduced protein activity comprises knocking-out of a corresponding endogenous gene.

Embodiment 68. The cell line of Embodiment 67, wherein the genetic modification is a homozygous knock-out of the corresponding endogenous gene.

Embodiment 69. The cell line of Embodiment 67, wherein the genetic modification is a heterozygous knock-out of the corresponding endogenous gene.

Embodiment 70. The cell line of any one of Embodiments 67-69, wherein the knock-out comprises a deletion of all or a part of coding region of the corresponding endogenous gene.

Embodiment 71. The cell line of any one of Embodiments 67-70, wherein the knock-out comprises a substitution of all or a part of coding region of the corresponding endogenous gene with one or more nucleotides and/or an insertion of one or more nucleotides to the coding region of the corresponding endogenous gene.

Embodiment 72. The cell line of any one of Embodiments 67-71, wherein the knock-out comprises a premature stop codon in the coding region of the corresponding endogenous gene.

Embodiment 73. The cell line of any one of Embodiments 67-72, wherein the knock-out comprises a frameshift mutation in the coding region of the corresponding endogenous gene.

Embodiment 74. The cell line of any one of Embodiments 1-73, wherein the genetic modification comprises knock-down of the corresponding endogenous gene.

Embodiment 75. The cell line of Embodiment 74, wherein expression level of the endogenous PTEN, CASP3, CASP8, CDH1, p53, ITGB1, RB1, IGFBP4, and/or the Cyclin-dependent kinase inhibitor protein in the cell line is no more than 50%, 40%, 30%, 20%, 10%, 5%, 3%, or 1% compared to the control cell line without at least one of said genetic modifications.

Embodiment 76. The cell line of Embodiment 74 or 75, wherein the knock-down comprises an antisense molecule in the cell line, wherein the antisense molecule downregulates the expression of the endogenous PTEN, CASP3, CASP8, CDH1, p53, ITGB1, RB1, IGFBP4, and/or the Cyclin-dependent kinase inhibitor gene via RNA interference (RNAi).

Embodiment 77. The cell line of Embodiment 76, wherein the antisense molecule is selected from the group consisting of a siRNA, a shRNA, and a miRNA.

Embodiment 78. The cell line of any one of Embodiments 74-77, wherein the knock-down comprises deleting all or a part of the promoter sequence of the corresponding endogenous gene.

Embodiment 79. The cell line of any one of Embodiments 74-78, wherein the knock-down comprises disrupting the promoter sequence of the corresponding endogenous gene by inserting, deleting and/or mutating one or more nucleotides.

Embodiment 80. The cell line of any one of Embodiments 1-79, wherein the genetic modification results in the expression of an endogenous PTEN, CASP3, CASP8, CDH1, p53, ITGB1, RB1, IGFBP4, and/or the Cyclin-dependent kinase inhibitor protein comprising an inactivating mutation.

Embodiment 81. The cell line of any one of Embodiments 1-80, wherein the cell line comprises a knock-in PTEN, CASP3, CASP8, CDH1, p53, ITGB1, RB1, IGFBP4, and/or the Cyclin-dependent kinase inhibitor allele encoding a knock-in PTEN, CASP3, CASP8, CDH1, p53, ITGB1, RB1, IGFBP4, and/or the Cyclin-dependent kinase inhibitor protein comprising an inactivating mutation.

Embodiment 82. The cell line of Embodiment 80 or 81, wherein the PTEN, CASP3, CASP8, CDH1, p53, ITGB1, RB1, IGFBP4, and/or the Cyclin-dependent kinase inhibitor protein comprising the inactivating mutation is a dominant negative mutant protein.

Embodiment 83. The cell line of any one of Embodiments 2-82, wherein the cell line expresses a knock-in TERT and/or SRC protein.

Embodiment 84. The cell line of any one of Embodiments 2-83, wherein the cell line expresses an endogenous TERT and/or SRC protein.

Embodiment 85. The cell line of Embodiment 83 or 84, wherein expression level of the endogenous TERT and/or SRC protein in the cell line is increased by at least 50%, at least 100%, at least 2-fold, or at least 5-fold, compared to the control cell line without said genetic modification.

Embodiment 86. The cell line of any one of Embodiments 83-85, wherein expression level of total TERT and/or SRC protein in the cell line is increased by at least 50%, at least 100%, at least 2-fold, or at least 5-fold, compared to the control cell line without said genetic modification.

Embodiment 87. The cell line of any one of Embodiments 83-86, wherein the genetic modification comprises disrupting a targeting sequence of a microRNA within endogenous TERT and/or SRC gene locus.

Embodiment 88. The cell line of any one of Embodiments 83-87, wherein the cell line expresses a TERT and/or SRC protein with an activating mutation.

Embodiment 89. The cell line of any one of Embodiments 1-88, wherein the PTEN protein comprises a polypeptide sequence having at least 80%, at least 85%, at least 90%, at least 95%, or 100% identity to SEQ ID NO: 1, or wherein the corresponding gene encoding PTEN encodes a polynucleotide sequence having at least 80%, at least 85%, at least 90%, at least 95%, or 100% identity to SEQ ID NO: 9.

Embodiment 90. The cell line of any one of Embodiments 1-89, wherein the CASP3 protein comprises a polypeptide sequence having at least 80%, at least 85%, at least 90%, at least 95%, or 100% identity to SEQ ID NO: 42, or wherein the corresponding gene encoding CASP3 encodes a polynucleotide sequence having at least 80%, at least 85%, at least 90%, at least 95%, or 100% identity to SEQ ID NO: 43.

Embodiment 91. The cell line of any one of Embodiments 1-90, wherein the CASP8 protein comprises a polypeptide sequence having at least 80%, at least 85%, at least 90%, at least 95%, or 100% identity to SEQ ID NO: 44, or wherein the corresponding gene encoding CASP8 encodes a polynucleotide sequence having at least 80%, at least 85%, at least 90%, at least 95%, or 100% identity to SEQ ID NO: 45.

Embodiment 92. The cell line of any one of Embodiments 1-91, wherein the CDH1 protein comprises a polypeptide sequence having at least 80%, at least 85%, at least 90%, at least 95%, or 100% identity to SEQ ID NO: 46, or wherein the corresponding gene encoding CDH1 encodes a polynucleotide sequence having at least 80%, at least 85%, at least 90%, at least 95%, or 100% identity to SEQ ID NO: 47.

Embodiment 93. The cell line of any one of Embodiments 2-92, wherein the TERT protein comprises a polypeptide sequence having at least 80%, at least 85%, at least 90%, at least 95%, or 100% identity to SEQ ID NO: 48, or wherein the corresponding gene encoding TERT encodes a polynucleotide sequence having at least 80%, at least 85%, at least 90%, at least 95%, or 100% identity to SEQ ID NO: 49.

Embodiment 94. The cell line of any one of Embodiments 3-93, wherein the p53 protein comprises a polypeptide sequence having at least 80%, at least 85%, at least 90%, at least 95%, or 100% identity to SEQ ID NO: 50, or wherein the corresponding gene encoding p53 encodes a polynucleotide sequence having at least 80%, at least 85%, at least 90%, at least 95%, or 100% identity to SEQ ID NO: 51.

Embodiment 95. The cell line of any one of Embodiments 1-94, wherein the ITGB1 protein comprises a polypeptide sequence having at least 80%, at least 85%, at least 90%, at least 95%, or 100% identity to SEQ ID NO: 52, or wherein the corresponding gene encoding ITGB1 encodes a polynucleotide sequence having at least 80%, at least 85%, at least 90%, at least 95%, or 100% identity to SEQ ID NO: 53.

Embodiment 96. The cell line of any one of Embodiments 1-95, wherein the IGFBP4 protein comprises a polypeptide sequence having at least 80%, at least 85%, at least 90%, at least 95%, or 100% identity to SEQ ID NO: 54, or wherein the corresponding gene encoding IGFBP4 encodes a polynucleotide sequence having at least 80%, at least 85%, at least 90%, at least 95%, or 100% identity to SEQ ID NO: 55.

Embodiment 97. The cell line of any one of Embodiments 1-96, wherein the RB1 protein comprises a polypeptide sequence having at least 80%, at least 85%, at least 90%, at least 95%, or 100% identity to SEQ ID NO: 66, or wherein the corresponding gene encoding RB1 encodes a polynucleotide sequence having at least 80%, at least 85%, at least 90%, at least 95%, or 100% identity to SEQ ID NO: 67.

Embodiment 98. The cell line of any one of Embodiments 1-97, wherein the SRC protein comprises a polypeptide sequence having at least 80%, at least 85%, at least 90%, at least 95%, or 100% identity to SEQ ID NO: 64, or wherein the corresponding gene encoding SRC encodes a polynucleotide sequence having at least 80%, at least 85%, at least 90%, at least 95%, or 100% identity to SEQ ID NO: 65.

Embodiment 99. The cell line of any one of Embodiments 6 and 8-98, wherein the p21 protein comprises a polypeptide sequence having at least 80%, at least 85%, at least 90%, at least 95%, or 100% identity to SEQ ID NO: 56, or wherein the corresponding gene encoding p21 encodes a polynucleotide sequence having at least 80%, at least 85%, at least 90%, at least 95%, or 100% identity to SEQ ID NO: 57.

Embodiment 100. The cell line of any one of Embodiments 5 and 9-99, wherein the p27 protein comprises a polypeptide sequence having at least 80%, at least 85%, at least 90%, at least 95%, or 100% identity to SEQ ID NO: 58, or wherein the corresponding gene encoding p27 encodes a polynucleotide sequence having at least 80%, at least 85%, at least 90%, at least 95%, or 100% identity to SEQ ID NO: 59.

Embodiment 101. The cell line of any one of Embodiments 7-100, wherein the p16 protein comprises a polypeptide sequence having at least 80%, at least 85%, at least 90%, at least 95%, or 100% identity to SEQ ID NO: 60, or wherein the corresponding gene encoding p16 encodes a polynucleotide sequence having at least 80%, at least 85%, at least 90%, at least 95%, or 100% identity to SEQ ID NO: 61.

Embodiment 102. The cell line of any one of Embodiments 9-101, wherein the p18 protein comprises a polypeptide sequence having at least 80%, at least 85%, at least 90%, at least 95%, or 100% identity to SEQ ID NO: 62, or wherein the corresponding gene encoding p18 encodes a polynucleotide sequence having at least 80%, at least 85%, at least 90%, at least 95%, or 100% identity to SEQ ID NO: 63.

Embodiment 103. A method of cell culturing, comprising culturing cells derived from the cell line of any one of Embodiments 1-102.

Embodiment 104. The method of Embodiment 103, wherein the cell culturing is suspension culturing.

Embodiment 105. A cell population derived from the cell line of any one of Embodiments 1-102.

Embodiment 106. The cell population of Embodiment 105, wherein the cell population is substantially undifferentiated.

Embodiment 107. The cell population of Embodiment 106, wherein more than 70% of the cell population is undifferentiated.

Embodiment 108. The cell population of Embodiment 105, wherein the cell population is substantially differentiated.

Embodiment 109. The cell population of Embodiment 108, wherein more than 70% of the cell population is differentiated.

Embodiment 110. A clonal, suspension cell culture comprising the cell population according to any one of Embodiments 105-109.

Embodiment 111. The clonal, suspension cell culture of Embodiment 110, wherein the cell population is in contact with a cell growth medium lacking or having reduced level of serum and/or one or more exogenous growth factors.

Embodiment 112. The clonal, suspension cell culture of Embodiment 110 or 111, wherein the cell population has a viable cell density (VCD) that is at least 20% higher than the maximum VCD of a control cell population without at least one of said genetic modifications.

Embodiment 113. The clonal, suspension cell culture of any one of Embodiments 110-112, wherein the cell population exhibits a maximum VCD that is at least 10%, 20%, 30%, 40%, 50%, 70%, 100%, 150%, 200%, 300%, or 400% higher than the maximum VCD of a control cell population without at least one of said genetic modifications.

Embodiment 114. The clonal, suspension cell culture of any one of Embodiments 110-113, wherein the cell population is capable of reaching maximum VCD at least 10%, 20%, 30%, 40%, 50%, 70%, 100%, 150%, 200%, 300%, or 400% faster than a control cell population without at least one of said genetic modifications.

Embodiment 115. The clonal, suspension cell culture of any one of Embodiments 110-114, wherein the cell population is at or capable of reaching a viable cell density of at least 0.4 million, at least 0.5 million, at least 0.6 million, at least 0.7 million, at least 0.8 million, at least 0.9 million, at least 1 million, 2 million, 5 million, 10 million, 15 million or 20 million cells per mL.

Embodiment 116. The clonal, suspension cell culture of any one of Embodiments 110-115, wherein the cell population exhibits at least 5%, 10%, 15%, 20%, 30%, or 40% higher cell viability than a control cell population without at least one of said genetic modifications at a viable cell density of at least 0.4 million, at least 0.5 million, at least 0.6 million, at least 0.7 million, at least 0.8 million, at least 0.9 million, at least 1 million, 2 million, 5 million, 10 million, 15 million or 20 million cells per mL.

Embodiment 117. The clonal, suspension cell culture of any one of Embodiments 110-116, wherein the cell population is capable of reaching a viable cell density of at least 0.4 million, at least 0.5 million, at least 0.6 million, at least 0.7 million, at least 0.8 million, at least 0.9 million, 1 million, 2 million, 5 million, 10 million, 15 million or 20 million cells per mL at least 10%, 20%, 30%, 40%, 50%, 70%, 100%, 150%, 200%, 300%, or 400% faster than a control cell population without at least one of said genetic modifications.

Embodiment 118. A method of generating the cell line of any one of Embodiments 1-102, comprising introducing a recombinant nucleic acid into a corresponding parental cell to cause the genetic modification.

Embodiment 119. A method of increasing maximum viable cell density of a metazoan cell line in a suspension culture, comprising introducing a genetic modification to the cell line, wherein said genetic modification results in:
(i) reduced protein activity of at least one of PTEN, CASP3, CASP8, CDH1, ITGB1, RB1, IGFBP4, p21, p27, p16, and p18;
(ii) increased protein activity of SRC;
(iii) increased protein activity of TERT; and/or
(iv) reduced protein activity of p53,
   compared to a control cell line without at least one of said genetic modifications in the suspension culture.

Embodiment 120. A method of adapting a metazoan cell line to suspension culture, comprising the steps of:
(a) introducing a genetic modification to the cell line, wherein said genetic modification results in
   (i) reduced protein activity of at least one of PTEN, CASP3, CASP8, CDH1, ITGB1, RB1, IGFBP4, p21, p27, p16, and p18;
   (ii) increased protein activity of SRC;
   (iii) increased protein activity of TERT; and/or
   (iv) reduced protein activity of p53,
   compared to a control cell line without at least one of said genetic modifications; and
(b) introducing the cell line to suspension culture.

Embodiment 121. The method of Embodiment 120, further comprising weaning the cell line from a regular cell growth medium to a cell growth medium lacking (or having reduced levels of) serum and/or one or more growth factors.

Embodiment 122. The method of Embodiment 120 or 121, further comprising the step of: (c) monitoring and removing cell aggregates.

Embodiment 123. The method of any one of Embodiments 120-122, wherein the step (b) comprises an acute treatment of the cell line.

Embodiment 124. The method of Embodiment 123, wherein the acute treatment comprises treating the cell line with a small molecule selected from the group consisting of a Rho-associated kinase (ROCK) inhibitor, an inhibitor of microtubule polymerization (e.g., nocodazole) and a myosin inhibitor (e.g., blebbistatin).

Embodiment 125. A cell-based meat product comprising:
a) the cell population of any one of Embodiments 105-109; and
b) a plant-based protein or product.

Embodiment 126. The cell-based meat product of Embodiment 125, wherein the cell population has undergone one or more food processing steps selected from heating, refrigerating, freezing, and smoking.

Embodiment 127. The cell-based meat product of Embodiments 125 or 126, wherein the plant product is selected from alfalfa, bamboo, barley, beets, black beans, broccoli, cabbage, canola, carrot, cauliflower, celery, celery root, chickpeas, corn, cotton, cow peas, fava beans, flax, garbanzo beans, green beans, kale, kidney beans, lupin, mung beans, navy beans, northern beans, nuts, oats, parsley, pearl millet, peas, pine nuts, pinto beans, potato, quinoa, red beans, rice, sesame, soy, spelt, sugarbeet, sunflowers, sweet potato, tobacco, wheat, white beans, whole grains, wild rice, zucchini, and a mixture thereof.

Embodiment 128. The cell-based meat product of any one of Embodiments 125-127, further comprising a binding agent, a carbohydrate-based gel, a non-animal fat, and/or a flavoring agent.

Embodiment 129. An ingredient of a food product, comprising the cell population of any one of Embodiments 105-109, wherein the cell population has undergone one or more food processing steps selected from heating, refrigerating, freezing, and smoking.

Embodiment 130. The ingredient of Embodiment 129, wherein said cell population has been heated at least 70 Celsius temperature for at least 10 minutes.

Embodiment 131. The ingredient of Embodiment 129 or 130, wherein the cell population is grilled.

Embodiment 132. The ingredient of Embodiment 129 or 130, wherein the cell population is boiled.

Embodiment 133. The ingredient of Embodiment 129 or 130, wherein the cell population is fried.

Embodiment 134. The ingredient of Embodiment 129 or 130, wherein the cell population is baked.

Embodiment 135. A kit for generating the cell line of any one of Embodiments 1-102, comprising:
(a) a recombinant nucleic acid that can generate the genetic modification upon introduction into a corresponding parental cell;
(b) an agent for introducing the recombinant nucleic acid into the corresponding parental cell.

Embodiment 136. The cell line, cell population, clonal, suspension cell culture, or method of any one of Embodiments 1-124, wherein VCD, apoptosis, adherent cells, and other measures of suspension culture are assessed in cultures of at least 5 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL, 500 mL, 525 mL, 550 mL, 575 mL, 600 mL, 625 mL, 650 mL, 675 mL, 700 mL, 725 mL, 750 mL, 775 mL, 800 mL, 825 mL, 850 mL, 875 mL, 900 mL, 925 mL, 950 mL, 975 mL, 1000 mL, 1.5 L, 2 L, 2.5 L, 3 L, 3.5 L, 4 L, 4.5 L, 5 L, 5.5 L, 6 L, 6.5 L, 7L, 7.5 L, 8 L, 8.5 L, 9 L, 9.5 L, 10 L, 50 L, 100 L, 200 L, 300 L, 400 L, 500 L, 600 L, 700 L, 800 L, 900 L, 1000 L, 1500 L, 2000 L, 2500 L, 3000 L, 3500 L, 4000 L, 4500 L, 5000 L, 5500 L, 6000 L, 6500 L, 7000 L, 7500 L, 8000 L, 8500 L, 9000 L, 9500 L, or 1000 L.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

TABLE OF SEQUENCE LISTING

| SEQ ID | | |
|---|---|---|
| NO | Name | Species |
| 1 | PTEN (Protein) | Bos taurus |
| 2 | PTEN (Protein) | Sus scrofa |
| 3 | PTEN (Protein) | Ovis aries |
| 4 | PTEN (Protein) | Capra hircus |
| 5 | PTEN (Protein) | Oryctolagus |
| | | cuniculus |
| 6 | PTEN (Protein) | Gallus gallus |
| 7 | PTEN (Protein) | Anas platyrhynchos |
| 8 | PTEN (Protein) | Meleagris |
| | | gallopavo |
| 9 | PTEN (Polynucleotide) | Bos taurus |
| 10 | PTEN (Polynucleotide) | Sus scrofa |
| 11 | PTEN (Polynucleotide) | Ovis aries |
| 12 | PTEN (Polynucleotide) | Capra hircus |
| 13 | PTEN (Polynucleotide) | Oryctolagus |
| | | cuniculus |
| 14 | PTEN (Polynucleotide) | Gallus gallus |
| 15 | PTEN (Polynucleotide) | Anas platyrhynchos |
| 16 | PTEN (Polynucleotide) | Meleagris |
| | | gallopavo |
| 17 | Bovine PCNA promoter | Bos taurus |
| 18 | Actin promoter | Bos taurus |
| 19 | Elongation Factor 1 alpha promoter | Bos taurus |
| 20 | Ubiquitin promoter | Bos taurus |
| 21 | Phosphoglycerate Kinase promoter | Bos taurus |
| 22 | Myosin light chain 2 promoter | Bos taurus |
| 23 | Creatine Kinase promoter | Bos taurus |
| 24 | Insulin-like Growth Factor 1 | Bos taurus |
| | promoter | |
| 25 | Myogenin promoter | Bos taurus |
| 26 | Pax3 promoter | Bos taurus |
| 27 | SIX1 promoter | Bos taurus |
| 28 | Desmin promoter | Bos taurus |
| 29 | PTEN (D77A) (Protein) | Bos taurus |
| 30 | PTEN (C109A), (Protein) | Bos taurus |
| 31 | PTEN (D77A, C109A), (Protein) | Bos taurus |
| 32 | PTEN (D77A) DNA | Bos taurus |
| 33 | PTEN (C109A) DNA | Bos taurus |
| 34 | PTEN (D77A, C109A) DNA | Bos taurus |
| 35 | PTEN forward primer | |
| 36 | PTEN reverse primer | |
| 37 | Rosa26 sgRNA | |
| 38 | Rosa26 Homology Arm, 5′ | |
| 39 | Rosa26 Homology Arm, 3′ | |
| 40 | Neomycin resistance cassette | |
| 41 | Insertion (Rosa26) , PTEN | |
| | D77A | |
| 42 | CASP3 (Protein) | Bos taurus |
| 43 | CASP3 (Polynucleotide) | Bos taurus |
| 44 | CASP8 (Protein) | Bos taurus |
| 45 | CASP8 (Polynucleotide) | Bos taurus |
| 46 | CDH1 (Protein) | Bos taurus |
| 47 | CDH1 (Polynucleotide) | Bos taurus |
| 48 | TERT (Protein) | Bos taurus |
| 49 | TERT (Polynucleotide) | Bos taurus |
| 50 | p53 (Protein) | Bos taurus |
| 51 | p53 (Polynucleotide) | Bos taurus |
| 52 | ITGB1 (Protein) | Bos taurus |
| 53 | ITGB1 (Polynucleotide) | Bos taurus |
| 54 | IGFBP4 (Protein) | Bos taurus |
| 55 | IGFBP4 (Polynucleotide) | Bos taurus |
| 56 | P21 (Protein) | Bos taurus |
| 57 | P21 (Polynucleotide) | Bos taurus |
| 58 | P27 (Protein) | Bos taurus |
| 59 | P27 (Polynucleotide) | Bos taurus |
| 60 | P16 (Protein) | Bos taurus |
| 61 | P16 (Polynucleotide) | Bos taurus |
| 62 | P18 (Protein) | Bos taurus |
| 63 | P18 (Polynucleotide) | Bos taurus |
| 64 | SRC (Protein) | Bos taurus |
| 65 | SRC (Polynucleotide) | Bos taurus |
| 66 | RB1 (Protein) | Bos taurus |
| 67 | RB1 (Polynucleotide) | Bos taurus |

What is claimed is:

1. A clonal, metazoan cell line comprising a genetic modification resulting in:
(A) reduced protein activity of at least one of i) Phosphatase and Tensin Homolog (PTEN), ii) Caspase 3 (CASP3), iii) Caspase 8 (CASP8), iv) Cadherin 1 (CDH1), v) Integrin beta-1 (ITGB1), vi) Retinoblastoma-associated protein (RB1), vii) Insulin-like growth factor-binding protein 4 (IGFBP4), and viii) a Cyclin-dependent kinase inhibitor; and/or
(B) increased protein activity of SRC tyrosine-protein kinase (SRC),
compared to a control cell line without said genetic modification.

2. The cell line of claim 1, comprising a genetic modification resulting in increased protein activity of Telomerase Reverse Transcriptase (TERT).

3. The cell line of claim 1 or 2, comprising a genetic modification resulting in reduced protein activity of Tumor Suppressor p53 (p53).

4. The cell line of any one of claims 1-3, comprising the genetic modifications resulting in:
(i) reduced protein activity of CASP3; and
(ii) reduced protein activity of ITGB1;
compared to the control cell line without said genetic modifications.

5. The cell line of any one of claims 1-3, comprising the genetic modifications resulting in:
(i) reduced protein activity of CASP3; and
(ii) reduced protein activity of Cyclin-dependent kinase inhibitor 1B (p27);
compared to the control cell line without said genetic modifications.

6. The cell line of any one of claims 1-3, comprising the genetic modifications resulting in:
(i) reduced protein activity of CDH1; and
(ii) reduced protein activity of Cyclin-dependent kinase inhibitor 1A (p21);
compared to the control cell line without said genetic modifications.

7. The cell line of any one of claims 1-3, comprising the genetic modifications resulting in:
(i) reduced protein activity of CDH1; and
(ii) reduced protein activity of Cyclin-dependent kinase inhibitor 2A (p16);
compared to the control cell line without said genetic modifications.

8. The cell line of any one of claims 4-7, wherein the cell line comprises the genetic modification(s) resulting in:
(iii) increased protein activity of TERT; and/or
(iv) reduced protein activity of p53,
compared to the control cell line without said genetic modification(s).

9. The cell line of any one of claims 1-8, wherein the Cyclin-dependent kinase inhibitor comprises one or more of Cyclin-dependent kinase inhibitor 1A (p21), Cyclin-dependent kinase inhibitor 1B (p27), Cyclin-dependent kinase inhibitor 2A (p16), and Cyclin-dependent kinase 4 inhibitor C (p18).

10. A clonal, metazoan cell line comprising:
i) a genetic modification resulting in increased protein activity of TERT; and/or
ii) a genetic modification resulting in reduced protein activity of p53,
compared to a control cell line without said genetic modification.

11. The cell line of claim 10, comprising both i) and ii).

12. The cell line of any one of claims 1-11, comprising a genetic modification resulting in reduced protein activity of PTEN.

13. The cell line of any one of claims 1-12, comprising a genetic modification resulting in reduced protein activity of CASP3.

14. The cell line of any one of claims 1-13, comprising a genetic modification resulting in reduced protein activity of CASP8.

15. The cell line of any one of claims 1-14, comprising a genetic modification resulting in reduced protein activity of CDH1.

16. The cell line of any one of claims 1-15, comprising a genetic modification resulting in reduced protein activity of ITGB1.

17. The cell line of any one of claims 1-16, comprising a genetic modification resulting in reduced protein activity of RB1.

18. The cell line of any one of claims 1-17, comprising a genetic modification resulting in reduced protein activity of IGFBP4.

19. The cell line of any one of claims 1-18, comprising a genetic modification resulting in reduced protein activity of the Cyclin-dependent kinase inhibitor.

20. The cell line of any one of claims 1-19, comprising a genetic modification resulting in reduced protein activity of Cyclin-dependent kinase inhibitor 1A (p21).

21. The cell line of any one of claims 1-20, comprising a genetic modification resulting in reduced protein activity of, Cyclin-dependent kinase inhibitor 1B (p27).

22. The cell line of any one of claims 1-21, comprising a genetic modification resulting in reduced protein activity of Cyclin-dependent kinase inhibitor 2A (p16).

23. The cell line of any one of claims 1-22, comprising a genetic modification resulting in reduced protein activity of Cyclin-dependent kinase 4 inhibitor C (p18).

24. The cell line of any one of claims 1-23, comprising a genetic modification resulting in increased protein activity of SRC.

25. The cell line of any one of claims 1-24, wherein the cell line is capable of sustained suspension culture.

26. The cell line of claim 25, wherein the cell line is capable of reaching a viable cell density (VCD) of at least 0.4 million, at least 0.5 million, at least 0.6 million, at least 0.7 million, at least 0.8 million, at least 0.9 million, at least 1 million, at least 2 million, at least 5 million, at least 10 million, 15 million or 20 million cells per mL in the suspension culture.

27. The cell line of any one of claims 1-26, wherein the cell line is in suspension culture.

28. The cell line of claim 27, wherein the cell line is stable in the suspension culture for at least 20, 30, 40, 50, or 60 generations.

29. The cell line of any one of claims 1-28, wherein the control cell line without at least one of said genetic modifications is unstable or less stable in a suspension culture.

30. The cell line of any one of claims 1-29, wherein the control cell line without at least one of said genetic modifications is not viable in a suspension culture.

31. The cell line of any one of claims 25-30, wherein the cell line is capable of reaching at least 10%, 20%, 30%, 40%, 50%, 70%, 100%, 150%, 200%, 300%, or 400% higher maximum viable cell density (VCD) compared to the control cell line without at least one of said genetic modifications in the suspension culture.

32. The cell line of any one of claims 25-31, wherein the cell line exhibits at least 5%, 10%, 20%, 30%, or 40% higher cell viability than the control cell line without at least one of said genetic modifications at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture.

33. The cell line of any one of claims 25-32, wherein the cell line exhibits a cell viability of at least 70%, at least 75%, at least 80%, at least 85%, or at least 90% at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture.

34. The cell line of any one of claims 25-33, wherein the cell line is capable of reaching maximum VCD at least 10%, 20%, 30%, 40%, 50%, 70%, 100%, 150%, 200%, 300%, or 400% faster than the control cell line without at least one of said genetic modifications in the suspension culture.

35. The cell line of any one of claims 25-34, wherein the cell line is capable of reaching a viable cell density of 0.4 million, 0.5 million, 0.6 million, 0.7 million, 0.8 million, 0.9 million, 1 million, 2 million, 5 million, 10 million, 15 million, 20 million, 25 million, 30 million, 35 million, 40 million, 45 million, or 50 million, cells per mL in the suspension culture at least 10%, 20%, 30%, 40%, 50%, 70%, 100%, 150%, 200%, 300%, or 400% faster than the control cell line without at least one of said genetic modifications.

36. The cell line of any one of claims 25-35, wherein the cell line exhibits a doubling time of less than 12 hours, 18 hours, 24 hours, 36 hours, or 48 hours when the viable cell density of the cell line is at 50% of its maximum VCD in the suspension culture.

37. The cell line of any one of claims 25-36, wherein the cell line exhibits increased proliferation rate and/or decreased differentiation rate compared to the control cell line without at least one of said genetic modifications.

38. The cell line of any one of claims 25-37, wherein the proliferation rate of the cell line is at least 20% higher than the control cell line without at least one of said genetic modifications when both cell lines are at a viable cell density of about 50%, 60%, 70%, 80%, 90%, or 95% of their maximum VCD.

39. The cell line of any one of claims 25-38, wherein the differentiation rate of the cell line is at least 20% lower than the control cell line without at least one of said genetic modifications when both cell lines are at a viable cell density of about 50%, 60%, 70%, 80%, 90%, or 95% of their maximum VCD.

40. The cell line of any one of claims 25-39, wherein cells of the suspension culture of the cell line comprises less than 50%, 40%, 30%, 20%, 10%, 5%, 3%, 2%, or 1% of cells in the form of cell aggregates having a diameter of greater than 100, 200, 300 or 400 microns.

41. The cell line of claim 40, wherein the cell aggregates have a diameter of greater than 200 microns.

42. The cell line of any one of claims 25-41, wherein cells of the suspension culture of the cell line comprises at least 50%, 60%, 70%, 80%, or 90% of cells in the form of single cells.

43. The cell line of claim 42, wherein the single cells have an average diameter of less than 30, 25, 20, 15, 10, or 5 microns.

44. The cell line of claim 42 or 43, wherein the single cells have an average circularity of greater than 0.5, 0.55, 0.6, 0.65, 0.7, 0.75, 0.8, 0.85, 0.9 or 0.95.

45. The cell line of any one of claims 25-44, wherein the cell line exhibits increased anoikis resistance compared to the control cell line without at least one of said genetic modifications.

46. The cell line of claim 45, wherein the percentage of apoptotic cells of the cell line is at least 10%, 20%, 30%, 40%, 50%, 60%, 70%, 80%, 90%, 95%, 97%, or 99% lower than the control cell line without at least one of said genetic modifications when both cell lines are at a total cell density of about 0.4 million, about 0.5 million, about 0.6 million, about 0.7 million, about 0.8 million, about 0.9 million, about 1 million, 2 million, 5 million, 10 million, 15 million or 20 million cells per mL in the suspension culture.

47. The cell line of claim 45 or 46, wherein cells in the suspension culture of the cell line comprise less than 50%, 40%, 30%, 20%, 10%, 5%, 3%, 2%, or 1% of apoptotic cells.

48. The cell line of claim 47, wherein the apoptotic cells are characterized using a biomarker selected from the group consisting of cytochrome C, caspase-3/7, DNA fragmentation and phosphatidylserine.

49. The cell line of any one of claims 25-48, wherein less than 30%, 20%, 10%, 5%, 3%, 2% or 1% of cells in the suspension culture of the cell line are adherent cells.

50. The cell line of any one of claims 1-49, wherein expression level of E-cadherin in the cell line is decreased by at least 30%, at least 50%, at least 70%, or at least 90%, compared to the control cell line without at least one of said genetic modifications.

51. The cell line of any one of claims 1-50, wherein expression level of N-cadherin in the cell line is increased by at least 20%, at least 50%, at least 100%, or at least 2-fold, compared to the control cell line without at least one of said genetic modifications.

52. The cell line of any one of claims 1-51, wherein expression level of vimentin in the cell line is increased by at least 20%, at least 50%, at least 100%, or at least 2-fold, compared to the control cell line without at least one of said genetic modifications.

53. The cell line of any one of claims 1-52, wherein the cell line is a myoblast cell line.

54. The cell line of any one of claims 1-52, wherein the cell line is a fibroblast cell line.

55. The cell line of any one of claims 1-52, wherein the cell line is a preadipocyte cell line.

56. The cell line of any one of claims 1-55, wherein the cell line lineage is skeletal muscle, subcutaneous adipose tissue, or connective tissue.

57. The cell line of any one of claims 1-55, wherein the cell line lineage is skeletal muscle.

58. The cell line of any one of claims 1-57, wherein the cell line is an immortalized cell line.

59. The cell line of any one of claims 1-58, wherein the cell line species identity is selected from the group consisting of Bos Taurus, Sus scrofa, Ovis aries, Capra hircus, Oryctolagus cuniculus, Gallus gallus, Anas platyrhynchos, and Meleagris gallopavo.

60. The cell line of any one of claims 1-58, wherein the cell line species identity is Bos taurus, Bos indicus, or a hybrid thereof.

61. The cell line of any one of claims 1-58, wherein the cell line species identity is Gallus gallus.

62. The cell line of any one of claims 25-61, wherein the suspension cell culture comprises a minimum regular cell growth medium.

63. The cell line of any one of claims 25-62, wherein the suspension cell culture comprises a cell growth medium lacking (or having reduced levels of) serum and/or one or more growth factors.

64. The cell line of claim 63, wherein the one or more growth factors are selected from the group consisting of fibroblast growth factor (FGF), epidermal growth factor (EGF), insulin-like growth factor (IGF), insulin, hepatocyte growth factor (HGF), transforming growth factor beta (TGF-β), tumor necrosis factor alpha (TNF-α), leukemia inhibitory factor (LIF) and interleukin-6 (IL6).

65. The cell line of claim 63 or 64, wherein the cell growth medium comprises no exogenous FGF, EGF, IGF, HGF, TGF-β, TNF-α, LIF and/or IL6.

66. The cell line of any one of claims 63-65, wherein the cell growth medium comprises no serum.

67. The cell line of any one of claims 1-66, wherein the genetic modification resulting in reduced protein activity comprises knocking-out of a corresponding endogenous gene.

68. The cell line of claim 67, wherein the genetic modification is a homozygous knock-out of the corresponding endogenous gene.

69. The cell line of claim 67, wherein the genetic modification is a heterozygous knock-out of the corresponding endogenous gene.

70. The cell line of any one of claims 67-69, wherein the knock-out comprises a deletion of all or a part of coding region of the corresponding endogenous gene.

71. The cell line of any one of claims 67-70, wherein the knock-out comprises a substitution of all or a part of coding region of the corresponding endogenous gene with one or more nucleotides and/or an insertion of one or more nucleotides to the coding region of the corresponding endogenous gene.

72. The cell line of any one of claims 67-71, wherein the knock-out comprises a premature stop codon in the coding region of the corresponding endogenous gene.

73. The cell line of any one of claims 67-72, wherein the knock-out comprises a frameshift mutation in the coding region of the corresponding endogenous gene.

74. The cell line of any one of claims 1-73, wherein the genetic modification comprises knock-down of the corresponding endogenous gene.

75. The cell line of claim 74, wherein expression level of the endogenous PTEN, CASP3, CASP8, CDH1, p53, ITGB1, RB1, IGFBP4, and/or the Cyclin-dependent kinase inhibitor protein in the cell line is no more than 50%, 40%, 30%, 20%, 10%, 5%, 3%, or 1% compared to the control cell line without at least one of said genetic modifications.

76. The cell line of claim 74 or 75, wherein the knock-down comprises an antisense molecule in the cell line, wherein the antisense molecule downregulates the expression of the endogenous PTEN, CASP3, CASP8, CDH1, p53, ITGB1, RB1, IGFBP4, and/or the Cyclin-dependent kinase inhibitor gene via RNA interference (RNAi).

77. The cell line of claim 76, wherein the antisense molecule is selected from the group consisting of a siRNA, a shRNA, and a miRNA.

78. The cell line of any one of claims 74-77, wherein the knock-down comprises deleting all or a part of the promoter sequence of the corresponding endogenous gene.

79. The cell line of any one of claims 74-78, wherein the knock-down comprises disrupting the promoter sequence of the corresponding endogenous gene by inserting, deleting and/or mutating one or more nucleotides.

80. The cell line of any one of claims 1-79, wherein the genetic modification results in the expression of an endogenous PTEN, CASP3, CASP8, CDH1, p53, ITGB1, RB1, IGFBP4, and/or the Cyclin-dependent kinase inhibitor protein comprising an inactivating mutation.

81. The cell line of any one of claims 1-80, wherein the cell line comprises a knock-in PTEN, CASP3, CASP8, CDH1, p53, ITGB1, RB1, IGFBP4, and/or the Cyclin-dependent kinase inhibitor allele encoding a knock-in PTEN, CASP3, CASP8, CDH1, p53, ITGB1, RB1, IGFBP4, and/or the Cyclin-dependent kinase inhibitor protein comprising an inactivating mutation.

82. The cell line of claim 80 or 81, wherein the PTEN, CASP3, CASP8, CDH1, p53, ITGB1, RB1, IGFBP4, and/or the Cyclin-dependent kinase inhibitor protein comprising the inactivating mutation is a dominant negative mutant protein.

83. The cell line of any one of claims 2-82, wherein the cell line expresses a knock-in TERT and/or SRC protein.

84. The cell line of any one of claims 2-83, wherein the cell line expresses an endogenous TERT and/or SRC protein.

85. The cell line of claim 83 or 84, wherein expression level of the endogenous TERT and/or SRC protein in the cell line is increased by at least 50%, at least 100%, at least 2-fold, or at least 5-fold, compared to the control cell line without said genetic modification.

86. The cell line of any one of claims 83-85, wherein expression level of total TERT and/or SRC protein in the cell line is increased by at least 50%, at least 100%, at least 2-fold, or at least 5-fold, compared to the control cell line without said genetic modification.

87. The cell line of any one of claims 83-86, wherein the genetic modification comprises disrupting a targeting sequence of a microRNA within endogenous TERT and/or SRC gene locus.

88. The cell line of any one of claims 83-87, wherein the cell line expresses a TERT and/or SRC protein with an activating mutation.

89. The cell line of any one of claims 1-88, wherein the PTEN protein comprises a polypeptide sequence having at least 80%, at least 85%, at least 90%, at least 95%, or 100% identity to SEQ ID NO: 1, or wherein the corresponding gene encoding PTEN encodes a polynucleotide sequence having at least 80%, at least 85%, at least 90%, at least 95%, or 100% identity to SEQ ID NO: 9.

90. The cell line of any one of claims 1-89, wherein the CASP3 protein comprises a polypeptide sequence having at least 80%, at least 85%, at least 90%, at least 95%, or 100% identity to SEQ ID NO: 42, or wherein the corresponding gene encoding CASP3 encodes a polynucleotide sequence having at least 80%, at least 85%, at least 90%, at least 95%, or 100% identity to SEQ ID NO: 43.

91. The cell line of any one of claims 1-90, wherein the CASP8 protein comprises a polypeptide sequence having at least 80%, at least 85%, at least 90%, at least 95%, or 100% identity to SEQ ID NO: 44, or wherein the corresponding gene encoding CASP8 encodes a polynucleotide sequence having at least 80%, at least 85%, at least 90%, at least 95%, or 100% identity to SEQ ID NO: 45.

92. The cell line of any one of claims 1-91, wherein the CDH1 protein comprises a polypeptide sequence having at least 80%, at least 85%, at least 90%, at least 95%, or 100% identity to SEQ ID NO: 46, or wherein the corresponding gene encoding CDH1 encodes a polynucleotide sequence having at least 80%, at least 85%, at least 90%, at least 95%, or 100% identity to SEQ ID NO: 47.

93. The cell line of any one of claims 2-92, wherein the TERT protein comprises a polypeptide sequence having at least 80%, at least 85%, at least 90%, at least 95%, or 100% identity to SEQ ID NO: 48, or wherein the corresponding gene encoding TERT encodes a polynucleotide sequence having at least 80%, at least 85%, at least 90%, at least 95%, or 100% identity to SEQ ID NO: 49.

94. The cell line of any one of claims 3-93, wherein the p53 protein comprises a polypeptide sequence having at least 80%, at least 85%, at least 90%, at least 95%, or 100% identity to SEQ ID NO: 50, or wherein the corresponding gene encoding p53 encodes a polynucleotide sequence having at least 80%, at least 85%, at least 90%, at least 95%, or 100% identity to SEQ ID NO: 51.

95. The cell line of any one of claims 1-94, wherein the ITGB1 protein comprises a polypeptide sequence having at least 80%, at least 85%, at least 90%, at least 95%, or 100% identity to SEQ ID NO: 52, or wherein the corresponding gene encoding ITGB1 encodes a polynucleotide sequence having at least 80%, at least 85%, at least 90%, at least 95%, or 100% identity to SEQ ID NO: 53.

96. The cell line of any one of claims 1-95, wherein the IGFBP4 protein comprises a polypeptide sequence having at least 80%, at least 85%, at least 90%, at least 95%, or 100% identity to SEQ ID NO: 54, or wherein the corresponding gene encoding IGFBP4 encodes a polynucleotide sequence having at least 80%, at least 85%, at least 90%, at least 95%, or 100% identity to SEQ ID NO: 55.

97. The cell line of any one of claims 1-96, wherein the RB1 protein comprises a polypeptide sequence having at least 80%, at least 85%, at least 90%, at least 95%, or 100% identity to SEQ ID NO: 66, or wherein the corresponding gene encoding RB1 encodes a polynucleotide sequence having at least 80%, at least 85%, at least 90%, at least 95%, or 100% identity to SEQ ID NO: 67.

98. The cell line of any one of claims 1-97, wherein the SRC protein comprises a polypeptide sequence having at least 80%, at least 85%, at least 90%, at least 95%, or 100% identity to SEQ ID NO: 64, or wherein the corresponding gene encoding SRC encodes a polynucleotide sequence having at least 80%, at least 85%, at least 90%, at least 95%, or 100% identity to SEQ ID NO: 65.

99. The cell line of any one of claims 6 and 8-98, wherein the p21 protein comprises a polypeptide sequence having at least 80%, at least 85%, at least 90%, at least 95%, or 100% identity to SEQ ID NO: 56, or wherein the corresponding gene encoding p21 encodes a polynucleotide sequence having at least 80%, at least 85%, at least 90%, at least 95%, or 100% identity to SEQ ID NO: 57.

100. The cell line of any one of claims 5 and 9-99, wherein the p27 protein comprises a polypeptide sequence having at least 80%, at least 85%, at least 90%, at least 95%, or 100% identity to SEQ ID NO: 58, or wherein the corresponding gene encoding p27 encodes a polynucleotide sequence having at least 80%, at least 85%, at least 90%, at least 95%, or 100% identity to SEQ ID NO: 59.

101. The cell line of any one of claims 7-100, wherein the p16 protein comprises a polypeptide sequence having at least 80%, at least 85%, at least 90%, at least 95%, or 100% identity to SEQ ID NO: 60, or wherein the corresponding gene encoding p16 encodes a polynucleotide sequence having at least 80%, at least 85%, at least 90%, at least 95%, or 100% identity to SEQ ID NO: 61.

102. The cell line of any one of claims 9-101, wherein the p18 protein comprises a polypeptide sequence having at least 80%, at least 85%, at least 90%, at least 95%, or 100% identity to SEQ ID NO: 62, or wherein the corresponding gene encoding p18 encodes a polynucleotide sequence having at least 80%, at least 85%, at least 90%, at least 95%, or 100% identity to SEQ ID NO: 63.

103. A method of cell culturing, comprising culturing cells derived from the cell line of any one of claims 1-102.

104. The method of claim 103, wherein the cell culturing is suspension culturing.

105. A cell population derived from the cell line of any one of claims 1-102.

106. The cell population of claim 105, wherein the cell population is substantially undifferentiated.

107. The cell population of claim 106, wherein more than 70% of the cell population is undifferentiated.

108. The cell population of claim 105, wherein the cell population is substantially differentiated.

109. The cell population of claim 108, wherein more than 70% of the cell population is differentiated.

110. A clonal, suspension cell culture comprising the cell population according to any one of claims 105-109.

111. The clonal, suspension cell culture of claim 110, wherein the cell population is in contact with a cell growth medium lacking or having reduced level of serum and/or one or more exogenous growth factors.

112. The clonal, suspension cell culture of claim 110 or 111, wherein the cell population has a viable cell density (VCD) that is at least 20% higher than the maximum VCD of a control cell population without at least one of said genetic modifications.

113. The clonal, suspension cell culture of any one of claims 110-112, wherein the cell population exhibits a maximum VCD that is at least 10%, 20%, 30%, 40%, 50%, 70%, 100%, 150%, 200%, 300%, or 400% higher than the maximum VCD of a control cell population without at least one of said genetic modifications.

114. The clonal, suspension cell culture of any one of claims 110-113, wherein the cell population is capable of reaching maximum VCD at least 10%, 20%, 30%, 40%, 50%, 70%, 100%, 150%, 200%, 300%, or 400% faster than a control cell population without at least one of said genetic modifications.

115. The clonal, suspension cell culture of any one of claims 110-114, wherein the cell population is at or capable of reaching a viable cell density of at least 0.4 million, at least 0.5 million, at least 0.6 million, at least 0.7 million, at least 0.8 million, at least 0.9 million, at least 1 million, 2 million, 5 million, 10 million, 15 million or 20 million cells per mL.

116. The clonal, suspension cell culture of any one of claims 110-115, wherein the cell population exhibits at least 5%, 10%, 15%, 20%, 30%, or 40% higher cell viability than a control cell population without at least one of said genetic modifications at a viable cell density of at least 0.4 million, at least 0.5 million, at least 0.6 million, at least 0.7 million, at least 0.8 million, at least 0.9 million, at least 1 million, 2 million, 5 million, 10 million, 15 million or 20 million cells per mL.

117. The clonal, suspension cell culture of any one of claims 110-116, wherein the cell population is capable of reaching a viable cell density of at least 0.4 million, at least 0.5 million, at least 0.6 million, at least 0.7 million, at least 0.8 million, at least 0.9 million, 1 million, 2 million, 5 million, 10 million, 15 million or 20 million cells per mL at least 10%, 20%, 30%, 40%, 50%, 70%, 100%, 150%, 200%, 300%, or 400% faster than a control cell population without at least one of said genetic modifications.

118. A method of generating the cell line of any one of claims 1-102, comprising introducing a recombinant nucleic acid into a corresponding parental cell to cause the genetic modification.

119. A method of increasing maximum viable cell density of a metazoan cell line in a suspension culture, comprising introducing a genetic modification to the cell line, wherein said genetic modification results in:
(i) reduced protein activity of at least one of PTEN, CASP3, CASP8, CDH1, ITGB1, RB1, IGFBP4, p21, p27, p16, and p18;
(ii) increased protein activity of SRC;
(iii) increased protein activity of TERT; and/or
(iv) reduced protein activity of p53,
compared to a control cell line without at least one of said genetic modifications in the suspension culture.

120. A method of adapting a metazoan cell line to suspension culture, comprising the steps of:
(a) introducing a genetic modification to the cell line, wherein said genetic modification results in
(i) reduced protein activity of at least one of PTEN, CASP3, CASP8, CDH1, ITGB1, RB1, IGFBP4, p21, p27, p16, and p18;
(ii) increased protein activity of SRC;
(iii) increased protein activity of TERT; and/or
(iv) reduced protein activity of p53,
compared to a control cell line without at least one of said genetic modifications; and
(b) introducing the cell line to suspension culture.

121. The method of claim 120, further comprising weaning the cell line from a regular cell growth medium to a cell growth medium lacking (or having reduced levels of) serum and/or one or more growth factors.

122. The method of claim 120 or 121, further comprising the step of:
(c) monitoring and removing cell aggregates.

123. The method of any one of claims 120-122, wherein the step (b) comprises an acute treatment of the cell line.

124. The method of claim 123, wherein the acute treatment comprises treating the cell line with a small molecule selected from the group consisting of a Rho-associated kinase (ROCK) inhibitor, an inhibitor of microtubule polymerization (e.g., nocodazole) and a myosin inhibitor (e.g., blebbistatin).

125. A cell-based meat product comprising:
a) the cell population of any one of claims 105-109; and
b) a plant-based protein or product.

126. The cell-based meat product of claim 125, wherein the cell population has undergone one or more food processing steps selected from heating, refrigerating, freezing, and smoking.

127. The cell-based meat product of claim 125 or 126, wherein the plant product is selected from alfalfa, bamboo, barley, beets, black beans, broccoli, cabbage, canola, carrot, cauliflower, celery, celery root, chickpeas, corn, cotton, cow peas, fava beans, flax, garbanzo beans, green beans, kale, kidney beans, lupin, mung beans, navy beans, northern beans, nuts, oats, parsley, pearl millet, peas, pine nuts, pinto beans, potato, quinoa, red beans, rice, sesame, soy, spelt, sugarbeet, sunflowers, sweet potato, tobacco, wheat, white beans, whole grains, wild rice, zucchini, and a mixture thereof.

128. The cell-based meat product of any one of claims 125-127, further comprising a binding agent, a carbohydrate-based gel, a non-animal fat, and/or a flavoring agent.

129. An ingredient of a food product, comprising the cell population of any one of claims 105-109, wherein the cell population has undergone one or more food processing steps selected from heating, refrigerating, freezing, and smoking.

130. The ingredient of claim 129, wherein said cell population has been heated at least 70 Celsius temperature for at least 10 minutes.

131. The ingredient of claim 129 or 130, wherein the cell population is grilled.

132. The ingredient of claim 129 or 130, wherein the cell population is boiled.

133. The ingredient of claim 129 or 130, wherein the cell population is fried.

134. The ingredient of claim 129 or 130, wherein the cell population is baked.

135. A kit for generating the cell line of any one of claims 1-102, comprising:
(a) a recombinant nucleic acid that can generate the genetic modification upon introduction into a corresponding parental cell;
(b) an agent for introducing the recombinant nucleic acid into the corresponding parental cell.

136. The cell line, cell population, clonal, suspension cell culture, or method of any one of claims 1-124, wherein VCD, apoptosis, adherent cells, and other measures of suspension culture are assessed in cultures of at least 5 mL, 10 mL, 15 mL, 20 mL, 25 mL, 30 mL, 35 mL, 40 mL, 45 mL, 50 mL, 55 mL, 60 mL, 65 mL, 70 mL, 75 mL, 80 mL, 85 mL, 90 mL, 95 mL, 100 mL, 125 mL, 150 mL, 175 mL, 200 mL, 225 mL, 250 mL, 275 mL, 300 mL, 325 mL, 350 mL, 375 mL, 400 mL, 425 mL, 450 mL, 475 mL, 500 mL, 525 mL, 550 mL, 575 mL, 600 mL, 625 mL, 650 mL, 675 mL, 700 mL, 725 mL, 750 mL, 775 mL, 800 mL, 825 mL, 850 mL, 875 mL, 900 mL, 925 mL, 950 mL, 975 mL, 1000 mL, 1.5 L, 2 L, 2.5 L, 3 L, 3.5 L, 4 L, 4.5 L, 5 L, 5.5 L, 6 L, 6.5 L, 7L, 7.5 L, 8 L, 8.5 L, 9L, 9.5 L, 10 L, 50 L, 100 L, 200 L, 300 L, 400 L, 500 L, 600 L, 700 L, 800 L, 900 L, 1000 L, 1500 L, 2000 L, 2500 L, 3000 L, 3500 L, 4000 L, 4500 L, 5000 L, 5500 L, 6000 L, 6500 L, 7000 L, 7500 L, 8000 L, 8500 L, 9000 L, 9500 L, or 1000 L.


SEQUENCE LISTING (67 sequences)

SEQ ID NO: 1 - protein, 388 aa, Bos taurus
MTAIIKEIVSRNKRRYQEDGFDLDLTYIYPNIIAMGFPAERLEGVYRNNIDDVVRCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEESSNPEASSSTSVTPDVSDNEPDHYRYSDTTDSDPENEPFDEDQHTQITKV

SEQ ID NO: 2 - protein, 403 aa, Sus scrofa
MTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGGLCDQEIDSICSIERADNDKEYLVLTLTKNDLDKANKDKANRYFSPNFKVKLYFTKTVEEPSNPEASSSTSVTPDVSDNEPDHYRYSDTTDSDPENEPFDEDQHTQITKV

SEQ ID NO: 3 - protein, 403 aa, Ovis aries
MTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEESSNPEASSSTSVTPDVSDNEPDHYRYSDTTDSDPENEPFDEDQHTQITKV

SEQ ID NO: 4 - protein, 388 aa, Capra hircus; identical to SEQ ID NO: 1

SEQ ID NO: 5 - protein, 403 aa, Oryctolagus cuniculus [sequence omitted]

SEQ ID NO: 6 - protein, 403 aa, Gallus gallus
MTAIIKEIVSRNKRRYQEDGFDLDLTYIYPNIIAMGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVYQLKVKIFTSPSGPSRREDKYMYFEFPQPLPVCGDIKVEFFHKQNKMLKKDKMFHFWVNTFFIGLDENSDKVENGSLVADQELDGIFSTERSDNDKEYLILTLTKNDLDKANKDKANRYFSPNFKVKLFFTKTVEEPSNPEASSSTSVTPDVSDNEPDHYRYSDTTDSDPENEPFDEDQHTQITKV

SEQ ID NO: 7 - protein, 403 aa, Anas platyrhynchos; identical to SEQ ID NO: 6

SEQ ID NO: 8 - protein, 369 aa, Meleagris gallopavo
MGFPAERLEGVYRNNIDDVVRFLDSKHKNHYKIYNLCAERHYDTAKFNCRVAQYPFEDHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVYQLKVKIFTSPSGPSRREDKYMYFEFPQPLPVCGDIKVEFFHKQNKMLKKDKMFHFWVNTFFIGLDENSDKVENGSLVADQELDGIFSTERSDNDKEYLILTLTKNDLDKANKDKANRYFSPNFKVKLFFTKTVEEPSNPEASSSTSVTPDVSDNEPDHYRYSDTTDSDPENEPFDEDQHTQITKV

SEQ ID NO: 9 - DNA, 1167 bp, Bos taurus
  source 1..1167: mol_type = other DNA
atgacagccatcatcaaagagatcgttagcagaaacaaaaggagatatcaagaggatggattcgacttagacttgacctatatttatccaaacattattgctatgggctttcctgcagaaagacttgaaggtgtatacaggaacaatatagatgatgtagtaagatgtgctgaaagacattatgataccgccaaatttaattgcagagttgcacagtatccctttgaagaccataatccaccacagttagaacttatcaaacccttttgtgaagatcttgaccaatggctaagtgaagatgacaatcatgttgcagcaattcactgtaaagctggaaagggacgaactggtgtaatgatttgtgcatatttgttacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggtggaacttgcaatcctcagtttgtggtctgccagctaaaggtgaagatatattcctccaattcaggacccacacgacgggaagacaagttcatgtactttgagttccctcagccattgcctgtgtgtggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagagcgtgcagataatgacaaggaatatctagtactcactttaacaaaaaatgatctcgacaaagcaaataaagacaaggccaaccgatacttttctccaaattttaaggtgaagctgtacttcacaaaaacagtagaggagtcatcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 10 - DNA, 1212 bp, Sus scrofa
  source 1..1212: mol_type = other DNA
atgacagccatcatcaaagagatcgttagcagaaacaaaaggagatatcaagaggatggattcgacttagacttgacctatatttatccaaacattattgctatggggtttcctgcagaaagacttgaaggcgtatacaggaacaatattgatgatgtagtaaggtttttggattcaaagcataaaaaccattacaagatatacaatctatgtgctgaaagacattatgataccgccaaatttaactgcagagttgcacagtatcctttcgaagaccataatccaccacagctagaacttatcaaacctttttgtgaagatcttgaccaatggctaagtgaagatgacaatcatgttgcagcaattcactgtaaagctggaaagggacgaactggtgtaatgatttgtgcatatttgttacatcggggtaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccattgcctgtgtgtggtgacatcaaagtagagttcttccacaaacagaacaagatgctaaaaaaggacaaaatgtttcacttttgggtaaatacattcttcataccaggaccagaggaaacctcagaaaaagtagaaaatggaggtctgtgtgatcaagaaattgatagtatttgcagtatagaacgtgcagataatgacaaggaatatctagtactcactttaacaaaaaatgatctcgacaaagcaaataaagacaaggccaaccgatacttttctccaaattttaaggtgaagctgtacttcacaaaaacagtagaggagccatcaaatccagaggctagcagttcaacttctgtaacaccagatgttagtgacaatgaacctgatcattatagatattctgacaccactgactctgatccagaaaatgaaccttttgatgaagatcagcatacacaaattacaaaagtttga

SEQ ID NO: 11 - DNA, 1212 bp, Ovis aries
  source 1..1212: mol_type = other DNA
atgacagccatcatcaaagagatcgttagcagaaacaaaaggagatatcaagaggatggattcgacttagacttgacctatatttatccaaacattattgctatggggtttcctgcagaaagacttgaaggcgtgtacaggaacaatatagacgatgtagtcaggtttttggattcaaagcataaaaaccattacaagatatacaatctatgtgctgaaagacattatgataccgccaaatttaattgcagagttgcacagtatccctttgaagaccataatccaccacagttagaacttatcaaacccttttgtgaagatcttgaccaatggctaagtgaagatgacaatcatgttgcagcaattcactgtaaagctggaaagggacgaactggtgtaatgatttgtgcatatttgttacatcggggcaaatttttaaaggcacaagaggccctagatttctatggggaagtaaggaccagagacaaaaagggagtaactattcccagtcagaggcgctatgtgtattattatagctacctgttaaagaatcatctggattacagaccagtggcactgttgtttcacaagatgatgtttgaaactattcccatgttcagtggtggaacttgcaatcctcagtttgtggtctgccagctaaaggtgaagatatattcctccaattcaggacccacacgacgggaagacaagttcatgtactttgagttccctcagccgctgcctgtgtgtggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagagcgtgcagataatgacaaggaatatctagtgctcactttaacaaaaaatgatctcgacaaagcaaataaagacaaggccaaccgatacttttctccaaattttaaggtgaagctgtacttcacaaaaacagtagaggagtcatcaaatccagaggctagcagttcaacgtctgtaacaccagatgtcagtgacaatgaacctgatcattacagatattctgacaccactgactctgacccagagaatgaaccttttgatgaagatcagcatacacaaattacaaaagtctga

SEQ ID NO: 12 - DNA, 1167 bp, Capra hircus
  source 1..1167: mol_type = other DNA
atgacagccatcatcaaagagatcgttagcagaaacaaaaggagatatcaagaggatggattcgacttagacttgacctatatttatccaaacattattgctatggggtttcctgcagaaagacttgaaggcgtgtacaggaacaatatagacgatgtagtcagatgtgctgaaagacattatgataccgccaaatttaattgcagagttgcacagtatccctttgaagaccataatccaccacagttagaacttatcaaacccttttgtgaagatcttgaccaatggctaagtgaagatgacaatcatgttgcagcaattcactgtaaagctggaaagggacgaactggtgtaatgatttgtgcatatttgttacatcggggcaaatttttaaaggcacaagaggccctagatttctatggggaagtaaggaccagagacaaaaagggagtaactattcccagtcagaggcgctatgtgtattattatagctacctgttaaagaatcatctggattacagaccagtggcactgttgtttcacaagatgatgtttgaaactattccaatgttcagtggtggaacttgcaatcctcagtttgtggtctgccagctaaaggtgaagatatattcctccaattcaggacccacacgacgggaagacaagttcatgtactttgagttccctcagccgttgcctgtgtgtggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagagcgtgcagataatgacaaggaatatctagtactcactttaacaaaaaatgatcttgacaaagcaaataaagacaaggccaaccgatacttttctccaaattttaaggtgaagctctacttcacaaaaacagtagaggagtcatcaaatccagaggctagcagttcaacgtctgtaacaccagatgtcagtgacaatgaacctgatcattacagatattctgacaccactgactctgacccagagaatgaaccttttgatgaagatcagcatacacaaattacaaaagtctga

SEQ ID NO: 13 - DNA, 1212 bp, Oryctolagus cuniculus
  source 1..1212: mol_type = other DNA
atgacagccatcatcaaagagatcgttagcagaaacaaaaggagatatcaagaggatggattcgacttagacttgacctatatttatccaaatattattgctatgggatttcctgcagaaagacttgaaggcgtatacaggaacaatattgatgatgtagtaaggtttttggattcaaagcataaaaaccattacaagatatacaatctatgtgctgaaaggcattatgacaccgccaaatttaactgcagagttgcacagtatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatttgtgcatatttattacatcggggcaaatttttaaaggcacaagaagccctagatttttatggagaagtaaggaccagagacaaaaagggagtaactattcccagtcagagacgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtacttcgagttccctcagccgttgcctgtgtgtggtgacatcaaagtagagttcttccacaaacagaacaagatgcttaaaaaggacaaaatgtttcacttttgggtaaatacgttctttataccaggaccagaggaaacctcagaaaaagtagaaaatggaagtctttgtgatcaagaaattgatagtatttgcagtatagaacgtgcagataacgacaaagaatatctagtacttactttaacaaaaaatgatcttgataaagcaaataaagacaaggcaaaccgatacttttctccaaattttaaggtgaagctgtatttcacaaaaacagtagaggagccatcaaatccagaggctagcagttcaacttctgtaacgccagatgttagtgacaatgaacctgatcattatagatattctgacaccactgactctgatccagagaatgaaccttttgatgaagatcagcatacacaaattacaaaagtctga

SEQ ID NO: 14 - DNA, 1212 bp, Gallus gallus
  source 1..1212: mol_type = other DNA
atgaccgccatcatcaaagagatcgtcagcaggaacaaaaggcgctaccaggaggatggcttcgacctggacctgacctatatttatccaaacataattgctatgggttttcctgcggagaggcttgaaggagtataccggaacaatattgatgatgtagtaaggtttttggattcaaagcataaaaaccattacaagatatacaatctatgtgctgaaagacattatgacaccgccaaatttaactgcagagttgcacagtacccctttgaagaccataacccaccacagttagagcttatcaaacctttttgtgaagatcttgaccaatggctaagtgaagatgacaatcatgttgcagcaatccactgtaaagctggaaagggacgaactggtgtaatgatatgtgcatatttgttgcatcgaggcaagtttttaaaggctcaagaagccctagatttctatggggaagtaaggaccagagacaagaagggagtcacaattcccagtcagagacgctacgtgtactactatagctacctgttaaagaatcaccttgattacagaccagtggcactgctgtttcacaagatgatgtttgaaacaattcccatgttcagtggcggaacttgcaatcctcagtttgtggtgtaccagctaaaggtaaagatattcacctccccttcaggaccctcaagacgtgaagacaagtatatgtactttgaattccctcaacctttgccggtatgcggtgatatcaaagtggagttcttccacaaacagaacaagatgttgaaaaaggacaaaatgtttcacttttgggtaaatacattcttcataggactggatgaaaattcagacaaagtagaaaatggaagtctagttgcagatcaggaacttgatggtattttcagtacagagcgctcagataatgacaaggaatatttaatccttacattaacaaaaaatgatctagacaaagcaaataaagacaaagccaacagatatttttctccaaacttcaaggtgaagctatttttcacaaaaacagtagaagagccatcaaatcccgaggctagcagttcaacttctgtaacaccagatgttagtgacaatgaacctgatcattacagatactctgacaccactgactctgatccagagaatgaaccttttgatgaagatcagcatacacagattacaaaagtctga

SEQ ID NO: 15 - DNA, 1212 bp, Anas platyrhynchos
  source 1..1212: mol_type = other DNA
atgaccgccatcatcaaagagatcgtcagcaggaacaaaaggcgctaccaggaggatggcttcgacctggacctgacctatatctatccaaacataattgctatgggttttcctgcagagaggcttgaaggagtgtaccggaacaatattgatgatgtagtaaggtttttggattcaaagcataaaaaccattacaagatatacaatctgtgtgctgaaagacattatgacaccgccaaatttaactgcagagttgcacagtacccttttgaagaccataacccaccacagcttgagctcattaaacctttttgtgaagatcttgaccaatggctaagtgaagatgacaatcatgttgcagcaatccactgtaaagctggaaagggacgaactggtgtaatgatatgcgcatatctgttgcatagaggcaagtttttaaaggctcaagaagctctagatttctatggggaagtaaggaccagagataagaagggagtaacaattcccagtcagagacgctacgtgtactattatagctacctgttaaagaatcacctggattacagaccagtggcactgctgtttcacaagatgatgtttgaaacaattcccatgttcagcggtggaacttgcaatcctcagtttgtggtgtaccagctaaaggtaaagatattcacctccccttcaggaccctcaagacgtgaagacaagtatatgtactttgaattccctcaacctttgccggtatgcggtgatatcaaagtggagtttttccacaaacagaacaagatgttgaaaaaggacaaaatgtttcacttttgggtaaatacattcttcataggactggatgaaaattcagacaaagtagaaaatggaagtctagttgcagatcaggaacttgatggtattttcagtacagagcgctcagataatgataaggaatatttaatccttacattaacaaaaaatgatctagacaaagcaaataaagacaaagccaacagatatttctctccaaacttcaaggtgaagctatttttcacaaaaacagtagaagagccatcaaatccagaggctagcagttcaacttctgtaacgccagatgttagtgacaatgaacctgatcattatagatactctgacaccactgactctgatccagagaatgaaccttttgatgaagatcagcatacgcagattacaaaagtctga

SEQ ID NO: 16 - DNA, 1110 bp, Meleagris gallopavo
  source 1..1110: mol_type = other DNA
atgggttttcctgcggagaggcttgaaggagtataccggaacaatattgatgatgtagtaaggtttttggattcaaagcataaaaaccattacaagatatacaatctatgtgctgaaagacattatgacaccgccaaatttaactgcagagttgcacagtacccttttgaagaccataacccaccacagttagagcttatcaaacctttttgtgaagatcttgaccaatggctaagtgaagatgacaatcatgttgcagcaatccactgtaaagctggaaagggacgaactggtgtaatgatatgtgcatatttgttgcatcgaggcaagtttttaaaggctcaagaagccctagatttctatggggaagtaaggaccagagacaagaagggagtcacaattcccagtcagagacgctacgtgtactactatagctacctgttaaagaatcaccttgattacagaccagtggcactgctgtttcacaagatgatgtttgaaacaattcccatgttcagtggcggaacttgcaatcctcagtttgtggtgtaccagctaaaagtaaagatattcacctccccttcaggaccctcaagacgtgaagacaaatatatgtactttgaattccctcaacctttgccggtatgcggtgatatcaaagtggagttcttccacaaacagaacaagatgttgaaaaaggacaaaatgtttcacttttgggttaatacattcttcataggactggatgaaaattcagacaaagtagaaaatggaagtctagttgcagatcaggaacttgatggtattttcagtacagagcgctcagataatgacaaggaatatttaatccttacattaacaaaaaatgatctagacaaagcaaataaagacaaagccaacagatatttttctccaaacttcaaggtgaagctatttttcacaaaaacagtagaagagccatcaaatccagaggctagcagttcaacttctgtaacaccagatgttagtgacaatgaacctgatcattacagatactctgacaccactgactctgatccagagaatgaaccttttgatgaagatcagcatacacagattacaaaagtctga

SEQ ID NO: 17 - DNA, 412 bp, Bos taurus
  source 1..412: mol_type = other DNA
cactcagaaggactcctctaacgggtgccggaacataacaaaatggccaggactccagcttacgcctatctgctgggccaacgaagcgctgcgctcctgccccgcccccatgcccgcatcctccaatggatgttccgggtggggcgggtttcgtgcagtgcacggacgggattggctttacagtctctccgcccaggcagcaggagggcagcgtgatgacgtatcaacaaggggcgcgggggcgtagcgcgcgctaccagacgcggcagtattaaacggctccaggcgcggctagctgttagaggcttccttcgtctcaggcgttcatagtcgtgttccgcctctcgctgtcatcctctgaagcctaagccggctacactttcctcagtccttctactagccgtgtcattgc

SEQ ID NO: 18 - DNA, 254 bp, Bos taurus
  source 1..254: mol_type = other DNA
cccagggcggccaacgccaaaactctctccctcgtcttcctcaatctcgctctcgctctctcccagtccatctctttcttttttttgcaaaaggaggggagaggggggtaaaaaaaaaaaatgctgcactgtgcggcgaggccggtgagtgagcggcgcggggccaatcagcgcccgccgtttcgaaagttgccttttatggctcgagcggccgcggcggcgccctatataacccagcggcgcgaggcgcagcc

SEQ ID NO: 19 - DNA, 1198 bp, Bos taurus
  source 1..1198: mol_type = other DNA
tcacgacacctaattggaaaggggtggaggtgtgggaaactttaaaccactgtccggaagctgaagaaaaagctacgatccaaactccaagggcaaattccaaggagcattacaactagcgccaagctggcccaactcaagtctccacccataagctccaccgtccagattcccctcccccaccccatgtgtcgctctggaaccacgaggcatcgaggggccaggacggcacccggtccgccgcggggggcgggggggtgtcccaaggaagcggcgccaggcagggacgggcccgggccctttgtgcgggtgactcaccggccgctcccctcccgggccgccgcgtcctccattttgaagccgtgcagaggggccggggagcggccatctttccgcgcacgcaactggtgccgggcggggcggcgctctgctcgcccggccgccacggctctcccgccccgcctcggcccagcgaggccgctatggcatgtgcgccgggaaagcaggcccgcgctctctgcgcctcgcggccgccccctaccccgaatcccgacgagcgccgagaagcccagcctgtcagcccccggccggccccggatgaaccgccccggccgaaaaacccgcggctgtgtggagttgttcaatagtgtgtgtgtggcggcggggtggggggaacagagcggcaaagcgcggcaggatccacacgcagcggccacggtgccacagcccaggcctcaacccaagcacgaggcgaagggactgtgagaaaagcgcaaggcctcgaactctccccacccccccaccctctcacaccccgaaattccggtgaagaacatggtgaaaatgcccacgggcgcggcgggagatacacgcacgcgaaggccgcggccgtcggcggaggctgggcccaccgaagccacacccggcactcacctgtgtcccggcggcaaacccgttgcgaaaaagaacgttcacggagactacggcacttatatacggttctccccccaccctcggggaaaagggcggagccagcacacgacaccactttcccagattaccccgcgccgcctccaccaggcaccagttcaatctccgtcccctccccccacgtctcggggaccgtgggcgatgtacgctcagcccactgacgagcgctgggcgccccgcgcatgctcctctagacccagaggaggac

SEQ ID NO: 20 - DNA, 1326 bp, Bos taurus
  source 1..1326: mol_type = other DNA
ccggacgcccagataaatccgtgccgggttttcgcgcctgccgctccccgcctccttccgggcaagtcgaggtccggctgcttccaatgtcttctgttcaacgaacgcgctgatccctccgctcggccgctcaggacggccgcccgctccctagcagaatcggtcccaaagccccagtcttcataagagagcgtcttaagaggagaatgagtcgattcttggggactgcttttctctttagagggtgggaggcctgcgaaaagtagtcccttcctggctttttgcggaagaatccatcggggtggggttacccatgactatataaggaagccccgggtgtggcgcaggcagttgcgtccggaccgggagttcagtcttcgttcttctgtgttcgctgctgacaccaccactaattggtgaggaggggtcccgggggtcttgcggccgcgcagccgctggggacgcgggtggttcaccggcctactgcgggccgaggggcattcggggcctacaattcccccgggcgcgcgagcaacagggtggccctgaggggtaggaacgcggcaaaatggcggccgtccctgatccctcatgggagaaggcaggctctgaaggtggttctgaggtgggacgcgaggatggggggtagggaggggactgcgagtgtcaagtgaacgcgagaggcccgtaatctctccatggcggcgggattcagctttctgggggacgagggttgagtgacgtgtgagggagtcgattattaggtcacggtttgacaggtgcgactactcgatttcccgtcgcagtggtggtggtggtgttcagtcgctcagtcgtgtcttactttgcaagcctatgggctacagcacgacaagctttcttgtctttcgggggctacttactgcagacgccactgagccgtcctacgcccgcgatgggggaggggacgcgctggccaccgcagttaggagacatcagcccttctgtgggctcataatctagggtggggccatctcccggcgtgtatgtggggaggtgtttactcgtcttgggacacagggtttgggcctggggtggacgctgcagtgttgacaaaggacagacatccagtgagggggggaggataagtgaggcccattaattgatttgggtcaggcttccctgcttctctctctcaagtacctgaaactgtttttgagctttttaagcttgtgagttgttgggcaagttagttgtttggctggtacgtagtgaagtttttaacattaggcgttggttgagccgtgaattaactgctaaatggtggggctctttttatgagtccac

SEQ ID NO: 21 - DNA, 473 bp, Bos taurus
  source 1..473: mol_type = other DNA
aattcagctgcagcgagaggccaggggatgagacagcggcagagactgagaccgactcagggtccgaacgcgtggaaaatgcctagcacctcacgggctgggtcgccactcctcccgccccgcccctttccggccgccgcgcccggctctcggcgcccagtggccgctctagaacgcggcccgaaggcgcccgctggctgccattgcaccgaggcgctgtcagtcagcgagcgcgattgtgggacgtgcagctgccgccagtcacgtccagcacgcagcgagcggctgggaactttcgtgactagaggcaggaggcggtgccgccggactaagcggcggcgggatctggagccagttggcgccgaggggcggacacggctcgggttcctgcggccgcaccggaggcagctgcagagccccgcaaacccatggcagcctcggggaaaacgtcaccccacatcccatggaattcg

SEQ ID NO: 22 - DNA, 293 bp, Bos taurus
  source 1..293: mol_type = other DNA
cccccagaagaatcccacagtctgcccagctctcttcagcctgggaacaataaatacttcctccccacgtttaaaaataaccccatgaccacttttggcagtggtaggtgaggcagacaccacctaaggcctccccaccccatgccgttcttctgaagtaagggcagctcagtcgccacccggtgacacgtgggacccagaaaggcattcaaggttaaagagctgggcggctggtgcacagggagagatgctgctcgcttgctgcagcgcttgggcagtcggcacttgtgctt

SEQ ID NO: 23 - DNA, 1087 bp, Bos taurus
  source 1..1087: mol_type = other DNA
tgtatcctggaggtgacactgacccaaaggaagggccagcagccgtggccttatatagggggacctaggctgtgctattggctgggaggggttgtccccagggaggagttctggagggcagctgagcctctcacctccctgctcccgcaccatacccaccctgaacccaggcatgctttgcgcccagccccatggccttgtatgggctgagccgccccaggggctgggctcacgcccccccaccaagctgataccctgaccaagtgctgagttgggctggcggtggggacagctggccccccgccgggaacatgggacggtggctgggaacgtcccaagcacgtagaagccaccgtcccctggcccctgagtctccttcagccaccagcccttcctgtggccctcggctccccctgtgccccctgggccctcacccctacccgagtcccagcggcacctcggggaaccaaggtgtggctccaggaggagcagataccctgtgagctggaggaagcagaggccgggagaaggaccaattgcaatggagggcagtctaaccccagaaacccgcgcttgggcgccccttcctcctagggactgagtggaggggaaactgaggctggctgagttggagagattcaaactcagggcaagggtgggaggcggtggtcaccctcccccccacttaaccctcgccccctcggtgccactgggagatttccgctgtgcgggggacagggagggtctcggtcgccgggaaaggagttatttttagagggcgctttgctgtctcctctgccgcaaaggccgagacccgaggcgctgggtatgggggggttcgggggcaggtgttggaggggggttcgccacgtgtcctggttaattataaccgggcgtctcgggtgtcctcgggcctcgcctccttacaagggcagccacggggccctgggtggccgctcccacctcccggggaagggggctgggcagcgacgccgttcagagcgcgggcgccagactcgcgcagatctgggcagagcaagcgctctcggcgcctcggctgtgagcaaggacttccagggctgagagg

SEQ ID NO: 24 - DNA, 1051 bp, Bos taurus
  source 1..1051: mol_type = other DNA
gcttctgaagtgcaaagtctggaaatgaattggttagcaggaataatgaggcaaaaagaaatccagggagatgggagatgttgagagcaatgtcacatttcaattttgaggactttattccattgcgcaggctctatctgctctgaatttagcagtgacagtgagatttagcaaacagaagagggatttagagaaaatcctcacatttatctacaaaacacagacactgtagacaggaaacagctgggggaacgcttgccttctctctctctccctcttctggcaaagttatcgagtaaggacttttttgggcatggtgacaaataacatcatacctttgcattttaaaactagagcacagaagcatttttttcccttaaaagaatgtgtgttagtgacaagaggagcagacattaaaatactgatgctgtcctctgattaactttctaccgggcgtgaagacacacacatctgctaatacaccttacccgtatgaaaggaatattcgtggaggatgggcgagcaattttatattccagagccaacacaaaatttaaaaaacaagaaatgaaaaaaagaccatctcaagcctactctatgattttaaccctcaaacgactggggtaaactagaatggaagacagcgctgggatgacccctcgtcctggttgccaagtgaggggaatgatctcatttcctagaacctggaaaggggcaggcagctttgtaattggctcccctcaggacactgtctcatgctttttgcccacgacagggcaaagattttgctgagctgtaaagccaactgaaaaaataaaatcaaatctttttcttccttccttctactctgctgcttggcccccaccccgacccccaccccaccccaggggaagggcatctgaggggcaaatgatttttccattgttcatatttttctgcataacttgaacctgtgtgtgtgtgtgtgtgtgtgtatttgtgtgtgtgtgcacatgagtgcattttctttcccccatttttgtgtattctaaataacaccagctggctagca

SEQ ID NO: 25 - DNA, 216 bp, Bos taurus
  source 1..216: mol_type = other DNA
tcatacagctccatggggtcagaagagacttgttcctgccaccagcccccaagctcctgcggcccctcacgccaactgctgggtgccatttaaaccctccccgctggcatggaaccagagataaatatagccaacgccacagaaacctgagcccccctctaagctgttgctgcacatcaagacgtttacagtggattagacgtgattccccttccc

SEQ ID NO: 26 - DNA, 276 bp, Bos taurus
  source 1..276: mol_type = other DNA
gtgagcacctttgccagtagccccagctgaggggccgatctgctagactcgcaccaaactcgtccgccctgggcttgggatcctgcactcaagggactcctctggagcctgtggacttggatctattagcgcccccctccccgcgccccgcctccctctctggccttttttgggggaggagctctccgagatccggagagttcccgagggtcacccgccgcactgtgctcgctttttcgtctcgccttcacctggatataatttgcgagcgaagct

SEQ ID NO: 27 - DNA, 720 bp, Bos taurus
  source 1..720: mol_type = other DNA
tggtttctgttctccctgcagctgccttaaagcgcgcagcgttctcggcgcgctgattggctgcgggcggcgcctatccggggctggccagcccgcgcgccgccgggccgggctgcgaggccgcgccgccccgcgcggggagggcgaggctgtacagcgagggatagggagtggtgggaggagagggaccgcgagtggtgagaagcgaagtggggaggagggagggaccggtgcttcccagggacggggggcggtgggagtgggtgagcgcgtcaggaccttgcaacgtgagccccctctgctgtgtcacctaagggcagccgcgcacatctttgcacaaatcaatggctccagaccttctaattcccctcctctccctccgtatctctgtccctgtcctttgcagacttgccatttcccggtgagctcatatttaattaccttaatgttggaatgaataagtaatggattgatgaatagcttatggaacgcgataaataatacaagcgtagaccagtcctctgggctgcagcccctgcaagaatggcttctctcctggctggcttcagctccaggagccaaacctaatttcctcccctcttctctcctgcccccaccgccctccaaagcgcacaaatactcccagctgtatagcgcttccctcattctcaaaaggccacgactatttgggaccaagagaagaattgtgtagagcatagt

SEQ ID NO: 28 - DNA, 402 bp, Bos taurus
  source 1..402: mol_type = other DNA
gaccagggaggcaaggactgaaagggggctggccgggggctccagacatgtttgcgcccgggtgaagcggctggcaggcctgcccaccttctgccctcctaggtccgccagcctctcccacaccccgcaggggcccccacccacccgcccgctgaggggctcagtcctcctgcgggggagctggcctccccgcccccacgccaggggccgccctttcctggcaggacagcgggatcctgcagctgtcaggggaggggcggcgggggctgatgtcaggagggatacaaatagtgcggacggccgggggccctgtctcccctcgccacatccactctccggccggccgcccgccgcctccttctccgcgccgcccagcctcgcccgcgccgccactatgagcca

SEQ ID NO: 29 - protein, 388 aa, Bos taurus
MTAIIKEIVSRNKRRYQEDGFDLDLTYIYPNIIAMGFPAERLEGVYRNNIDDVVRCAERHYDTAKFNCRVAQYPFEAHNPPQLELIKPFCEDLDQWLSEDDNHVAAIHCKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEESSNPEASSSTSVTPDVSDNEPDHYRYSDTTDSDPENEPFDEDQHTQITKV

SEQ ID NO: 30 - protein, 388 aa, Bos taurus
MTAIIKEIVSRNKRRYQEDGFDLDLTYIYPNIIAMGFPAERLEGVYRNNIDDVVRCAERHYDTAKFNCRVAQYPFEDHNPPQLELIKPFCEDLDQWLSEDDNHVAAIHAKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEESSNPEASSSTSVTPDVSDNEPDHYRYSDTTDSDPENEPFDEDQHTQITKV

SEQ ID NO: 31 - protein, 388 aa, Bos taurus
MTAIIKEIVSRNKRRYQEDGFDLDLTYIYPNIIAMGFPAERLEGVYRNNIDDVVRCAERHYDTAKFNCRVAQYPFEAHNPPQLELIKPFCEDLDQWLSEDDNHVAAIHAKAGKGRTGVMICAYLLHRGKFLKAQEALDFYGEVRTRDKKGVTIPSQRRYVYYYSYLLKNHLDYRPVALLFHKMMFETIPMFSGGTCNPQFVVCQLKVKIYSSNSGPTRREDKFMYFEFPQPLPVCGDIKVEFFHKQNKMLKKDKMFHFWVNTFFIPGPEETSEKVENGSLCDQEIDSICSIERADNDKEYLVLTLTKNDLDKANKDKANRYFSPNFKVKLYFTKTVEESSNPEASSSTSVTPDVSDNEPDHYRYSDTTDSDPENEPFDEDQHTQITKV

SEQ ID NO: 32 - DNA, 1167 bp, Bos taurus
  source 1..1167: mol_type = other DNA
atgacagccatcatcaaagagatcgttagcagaaacaaaaggagatatcaagaggatggattcgacttagacttgacctatatttatccaaacattattgctatgggctttcctgcagaaagacttgaaggtgtatacaggaacaatatagatgatgtagtaagatgtgctgaaagacattatgataccgccaaatttaattgcagagttgcacagtatccctttgaagctcataatccaccacagttagaacttatcaaacccttttgtgaagatcttgaccaatggctaagtgaagatgacaatcatgttgcagcaattcactgtaaagctggaaagggacgaactggtgtaatgatttgtgcatatttgttacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggtggaacttgcaatcctcagtttgtggtctgccagctaaaggtgaagatatattcctccaattcaggacccacacgacgggaagacaagttcatgtactttgagttccctcagccattgcctgtgtgtggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagagcgtgcagataatgacaaggaatatctagtactcactttaacaaaaaatgatctcgacaaagcaaataaagacaaggccaaccgatacttttctccaaattttaaggtgaagctgtacttcacaaaaacagtagaggagtcatcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 33 - DNA, 1167 bp, Bos taurus
  source 1..1167: mol_type = other DNA
atgacagccatcatcaaagagatcgttagcagaaacaaaaggagatatcaagaggatggattcgacttagacttgacctatatttatccaaacattattgctatgggctttcctgcagaaagacttgaaggtgtatacaggaacaatatagatgatgtagtaagatgtgctgaaagacattatgataccgccaaatttaattgcagagttgcacagtatccctttgaagaccataatccaccacagttagaacttatcaaacccttttgtgaagatcttgaccaatggctaagtgaagatgacaatcatgttgcagcaattcacgctaaagctggaaagggacgaactggtgtaatgatttgtgcatatttgttacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggtggaacttgcaatcctcagtttgtggtctgccagctaaaggtgaagatatattcctccaattcaggacccacacgacgggaagacaagttcatgtactttgagttccctcagccattgcctgtgtgtggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagagcgtgcagataatgacaaggaatatctagtactcactttaacaaaaaatgatctcgacaaagcaaataaagacaaggccaaccgatacttttctccaaattttaaggtgaagctgtacttcacaaaaacagtagaggagtcatcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 34 - DNA, 1167 bp, Bos taurus
  source 1..1167: mol_type = other DNA
atgacagccatcatcaaagagatcgttagcagaaacaaaaggagatatcaagaggatggattcgacttagacttgacctatatttatccaaacattattgctatgggctttcctgcagaaagacttgaaggtgtatacaggaacaatatagatgatgtagtaagatgtgctgaaagacattatgataccgccaaatttaattgcagagttgcacagtatccctttgaagctcataatccaccacagttagaacttatcaaacccttttgtgaagatcttgaccaatggctaagtgaagatgacaatcatgttgcagcaattcacgctaaagctggaaagggacgaactggtgtaatgatttgtgcatatttgttacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggtggaacttgcaatcctcagtttgtggtctgccagctaaaggtgaagatatattcctccaattcaggacccacacgacgggaagacaagttcatgtactttgagttccctcagccattgcctgtgtgtggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagagcgtgcagataatgacaaggaatatctagtactcactttaacaaaaaatgatctcgacaaagcaaataaagacaaggccaaccgatacttttctccaaattttaaggtgaagctgtacttcacaaaaacagtagaggagtcatcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 35 - DNA, 21 bp, synthetic construct
  source 1..21: mol_type = other DNA
atgacagccatcatcaaagag

SEQ ID NO: 36 - DNA, 25 bp, synthetic construct
  source 1..25: mol_type = other DNA
tcagacttttgtaatttgtgtatgc

SEQ ID NO: 37 - DNA, 20 bp, synthetic construct
  source 1..20: mol_type = other DNA
cgctgcataaaataagtcct

SEQ ID NO: 38 - DNA, 814 bp, synthetic construct
  source 1..814: mol_type = other DNA
gtcagtgaaagtggcttcgcgcgggcgtcctaccacccaccccttccttcgggggagtcggtttacccgccgcctgcttggcttcggcatctgattggctgctgaagctcagggaacggccccttgttattggctcgggtcccaaatgagcgaaaccactgcgcgggtcggcggggaggcggtcgttggtacgatcctccccgagacccagcgccgcagtgtctggccccgcgcccctgcgcaacgtggcaggaagcgcgcgctggaggtgggggcggactgccgggccgaggattctgggtggtggcgattgcggctccgccctgggcgcccgctgcctgaaggacaagactagcccgacctgctcctggacccgtggggctgaagggaggagtgggggtcggtgccgctggcttgtgggtgggaggtgcatgttctccaaaaatcggcgcgagctgcaatcctgaggtggctgcagtggaggaggcggagagaaggccgcacccttctcagcagggggaggggagtgccgcaatacctttatgggagttctctgctgcctcctgtctcctaaggaccgccctgggcctagaagaatccctccctcccccgcgatctcgtcatcgcctccatgtcgagtctcgattatgggcgggattcttttgcccaggcttaaccttattcctgggcgttgtcctgcaggggatcgagcaggtataagacttagaggacgaacccaatttcttttatcttcccacaggcttgagtttgtgtcacaaaataattataatggggtggtggagtgaaatgaagt

SEQ ID NO: 39 - DNA, 800 bp, synthetic construct
  source 1..800: mol_type = other DNA
ttttatgcagcgagactgcgagttactacttcttaacatccttttgttgcatattttccaggagattgagagagaggttaaaagcttgatctcctgaattttatactctccccatttgagacagttgagaaataggttaaaggcatgctctcttgagttccccatttgagaccgttgctacaccgccaaaacagagcattttagattagatcttaaaaatttaattccccacccttgcattctcagagtcaggccttttagcaactctcactcatactttcagcccattttctgtttgttacacttgctcatcttgtcccagtcataccattggctttctcctctcctgtttttggtatcccggtgagtcatgaaaccagacaggtttcaccaccaattaaggctacccagctcgagcataggcttcactcttgcccagaaatgcatttattcctcttttatggatattctggagtctttaccttgattttcatttaattttttaacctcagctgggattctactgaccctcttaatagtccagatgatcttgacgactgctttgctgagaaccggacgtgaggttcagcaacatctcttttatatccttagaatacctttcaacccattttcattgatatgcttatgagttagtaatcaagctcagttgccataaggctagtatccttcgaactaggatctcttgtctctggtatctgctgatacaactttcatatgtgtccaggacagtagttctcatacaaagataacagcatggaagtaaccgatccaactccttt

SEQ ID NO: 40 - DNA, 795 bp, synthetic construct
  source 1..795: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 41 - DNA, 4283 bp, synthetic construct
  source 1..4283: mol_type = other DNA
gtcagtgaaagtggcttcgcgcgggcgtcctaccacccaccccttccttcgggggagtcggtttacccgccgcctgcttggcttcggcatctgattggctgctgaagctcagggaacggccccttgttattggctcgggtcccaaatgagcgaaaccactgcgcgggtcggcggggaggcggtcgttggtacgatcctccccgagacccagcgccgcagtgtctggccccgcgcccctgcgcaacgtggcaggaagcgcgcgctggaggtgggggcggactgccgggccgaggattctgggtggtggcgattgcggctccgccctgggcgcccgctgcctgaaggacaagactagcccgacctgctcctggacccgtggggctgaagggaggagtgggggtcggtgccgctggcttgtgggtgggaggtgcatgttctccaaaaatcggcgcgagctgcaatcctgaggtggctgcagtggaggaggcggagagaaggccgcacccttctcagcagggggaggggagtgccgcaatacctttatgggagttctctgctgcctcctgtctcctaaggaccgccctgggcctagaagaatccctccctcccccgcgatctcgtcatcgcctccatgtcgagtctcgattatgggcgggattcttttgcccaggcttaaccttattcctgggcgttgtcctgcaggggatcgagcaggtataagacttagaggacgaacccaatttcttttatcttcccacaggcttgagtttgtgtcacaaaataattataatggggtggtggagtgaaatgaagttaagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatcactcagaaggactcctctaacgggtgccggaacataacaaaatggccaggactccagcttacgcctatctgctgggccaacgaagcgctgcgctcctgccccgcccccatgcccgcatcctccaatggatgttccgggtggggcgggtttcgtgcagtgcacggacgggattggctttacagtctctccgcccaggcagcaggagggcagcgtgatgacgtatcaacaaggggcgcgggggcgtagcgcgcgctaccagacgcggcagtattaaacggctccaggcgcggctagctgttagaggcttccttcgtctcaggcgttcatagtcgtgttccgcctctcgctgtcatcctctgaagcctaagccggctacactttcctcagtccttctactagccgtgtcattgcatgacagccatcatcaaagagatcgttagcagaaacaaaaggagatatcaagaggatggattcgacttagacttgacctatatttatccaaacattattgctatgggctttcctgcagaaagacttgaaggtgtatacaggaacaatatagatgatgtagtaagatgtgctgaaagacattatgataccgccaaatttaattgcagagttgcacagtatccctttgaagctcataatccaccacagttagaacttatcaaacccttttgtgaagttcttgaccaatggctaagtgaagatgacaatcatgttgcagcaattcactgtaaagctggaaagggacgaactggtgtaatgatttgtgcatatttgttacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggtggaacttgcaatcctcagtttgtggtctgccagctaaaggtgaagatatattcctccaattcaggacccacacgacgggaagacaagttcatgtactttgagttccctcagccattgcctgtgtgtggtgacatcaaagtagagttcttccacaaacagaacaagatgctaaaaaaggacaaaatgtttcacttttgggtaaacacattcttcataccaggaccagaggaaacctcagaaaaagtagaaaatggaagtctatgtgatcaagaaattgatagtatttgcagtatagagcgtgcagataatgacaaggaatatctagtactcactttaacaaaaaatgatctcgacaaagcaaataaagacaaggccaaccgatacttttctccaaattttaaggtgaagctgtacttcacaaaaacagtagaggagtcatcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgattttatgcagcgagactgcgagttactacttcttaacatccttttgttgcatattttccaggagattgagagagaggttaaaagcttgatctcctgaattttatactctccccatttgagacagttgagaaataggttaaaggcatgctctcttgagttccccatttgagaccgttgctacaccgccaaaacagagcattttagattagatcttaaaaatttaattccccacccttgcattctcagagtcaggccttttagcaactctcactcatactttcagcccattttctgtttgttacacttgctcatcttgtcccagtcataccattggctttctcctctcctgtttttggtatcccggtgagtcatgaaaccagacaggtttcaccaccaattaaggctacccagctcgagcataggcttcactcttgcccagaaatgcatttattcctcttttatggatattctggagtctttaccttgattttcatttaattttttaacctcagctgggattctactgaccctcttaatagtccagatgatcttgacgactgctttgctgagaaccggacgtgaggttcagcaacatctcttttatatccttagaatacctttcaacccattttcattgatatgcttatgagttagtaatcaagctcagttgccataaggctagtatccttcgaactaggatctcttgtctctggtatctgctgatacaactttcatatgtgtccaggacagtagttctcatacaaagataacagcatggaagtaaccgatccaactccttt

SEQ ID NO: 42 - protein, 275 aa, Bos taurus
MENTENSVDSKSIKTSETKILHGSKSMDSGISLEESYKMDYPEMGLCIIINNKNFHENTGMACRSGTDVDAANLRETFMNLKYEVRIKNDLTCKEMLELMSNVSKEDHSKRSSFICVLLSHGEEGIIFGTNGPVNLKKLASFFRGDYCRSLTGKPKLFIIQACRGTELDCGIETDSGAEDDMACQKIPVEADFLYAYSTAPGYFSWRNAKNGSWFIQALCEMLKKHAHRLELMHILTRVNRKVAIEYESFSTDSAFHAKKQIPCIMSMLTKELYF

SEQ ID NO: 43 - DNA, 828 bp, Bos taurus
  source 1..828: mol_type = other DNA
atggagaacactgaaaattcagtggattcaaaatccattaaaacttcagagacaaagatcttacatggaagcaaatcaatggactctggaatatccttggaagagagttacaaaatggattatcctgaaatgggtttatgtataataatcaataataagaacttccacgaaaatactggcatggcctgtcgatctggtacagacgtggatgcagcaaacctcagggaaacattcatgaacttgaaatacgaagtcaggattaaaaatgatcttacatgtaaagaaatgttggaattaatgagcaacgtttctaaagaagaccatagcaaaaggagcagttttatttgtgtgcttctaagccatggtgaagaaggaatcatttttggaaccaacggacccgtcaatttgaaaaaattagcaagtttcttcagaggggactattgcagaagtctgactggaaaacccaaacttttcattattcaggcctgccgaggcacagaactggactgtggtattgagacagacagtggtgctgaggatgacatggcctgtcagaaaataccagttgaggcagacttcttgtacgcatattctacagcacctggttacttttcctggcgaaatgcaaagaacggatcctggttcatccaggctctttgtgagatgctgaaaaagcacgctcacaggctcgagctcatgcacattcttactcgggttaaccgaaaggtagcaatagaatatgagtccttttctaccgattctgcttttcatgcaaagaaacagattccttgtattatgtctatgctcacaaaagaactgtatttctaa

SEQ ID NO: 44 - protein, 485 aa, Bos taurus
MALTQCLYNIGEQLGSDDLAALKFLSRDHIPYRKQEPIKDALMLFQRLQEKRMLEENNLSFLKELLFRVNRLDLLLNYLYTSEEEMKRELQIPGRAQISAYRILLFQISEDVNKVELKDFKFFLSQEIAKCKLDDDMTLLDIFVEMEKRTILGEENLDTLKRICEQVNKSLLKKIYDYEELRKERRMSLERDPYAFSNDMSQSLPEEGYSKMPAMSDSPEQDSELQTSDTVYRMTSKPRGYCLIFNNYDFSIARKQVPELHDLKDRTGTDFDADALDKTFRELHFEIVHYKDLTAKGICEVLESYQKKDHKNKDCFICCILTHGNKGIIYGSDGQEASIYELTSYFTGLKCPSLIGKPKIFFIQACQGDKYQKGVAVETDSEQMEAYLEVDSSPQKRYIPDEADFLLGMATVKNYVSYRNIWNGAWYIQSLCQNLRERCPRGEDILTILTKVNFEVSKLDDKQRMAKQMPQPTFTLRKKLFFPLN

SEQ ID NO: 45 - DNA, 1458 bp, Bos taurus
  source 1..1458: mol_type = other DNA
atggcgctcacccaatgtctctataatattggggagcagctgggcagtgacgacctggctgccctcaagttcctaagccgggaccacatcccatataggaagcaggaacccatcaaggatgccttgatgctattccagaggctccaagaaaagagaatgttggaggaaaacaatctgtccttcttgaaggagctgcttttccgagtgaatagactggatctgctgcttaactacttgtataccagcgaggaggagatgaagagggagcttcagatacccggcagggcccagatctctgcctacaggattctgcttttccagatttcagaagatgtgaacaaagtagaattgaaggactttaagttttttttgagccaggagattgccaaatgtaagctggatgatgacatgactttgcttgatattttcgtggagatggagaagaggaccatcctaggggaagagaacttggacaccctgaaaagaatctgtgagcaggtcaacaagagcttgctgaagaaaatctatgattatgaagaattaagaaaagagagaagaatgagccttgaaagagatccatatgcattttcaaatgacatgtcacaatcgcttccagaggagggctactctaagatgccggccatgtcagactctccagaacaggacagtgagttgcagacatctgacacagtttaccgaatgacaagcaaacctcggggatactgtttgatctttaacaattatgattttagcatagcacggaagcaggtgcctgaacttcacgaccttaaggataggactggaacagactttgatgcagacgctttggataagacctttcgtgaacttcattttgagatagtgcactacaaagacctaacagcaaagggaatctgtgaggttctagaatcctaccagaagaaggaccacaaaaacaaagactgcttcatctgctgcatcctcacccacggaaacaagggcatcatctatggctccgatgggcaagaagcctccatctatgagctgacctcttacttcactggcttaaagtgcccttcccttattggcaaacccaaaatcttttttattcaggcttgccaaggggacaagtaccagaaaggtgtagccgttgagaccgactcagaacagatggaagcctatttagaggtggactcatcacctcagaagagatatatcccagatgaggctgactttctgctggggatggccactgtgaaaaactatgtttcctaccgaaacatctggaatggggcctggtatatccagtcactttgccagaatctgagagaaagatgtcctaggggtgaagacattctgaccatcctaaccaaggtgaacttcgaagtaagcaaactggatgacaagcaacgtatggccaaacagatgccacaacctactttcacactgcgaaaaaaactcttctttcctcttaattga

SEQ ID NO: 46 - protein, 882 aa, Bos taurus
MGPWSRSLSALCCCCRCNPWLCREPEPCIPGFGAESYTFTVPRRNLERGRVLGRVSFEGCAGLPRTVYVSDDTRFKVHTDGVLTVRRPVHLHRPELSFLVHAWDSTHRKLSTKVTLEVSAHHHHHHSHHDSPSGTQTEVLTFPGPHHGLRRQKRDWVIPPISCPENEKGPFPKSLVQIKSNKEKETQVFYSITGQRADTPPVGVFIIERETGWLKVTQPLDREQIAKYILFSHAVSSNGQAIEEPMEIVITVTDQNDNKPQFTQEVFKASALEGALPGTSVMQVTATDIDDEVNTYTAAIGYTIPAQDPMLPHNKMFTINKETGVISVLTTGLDRESFPTYTLMVQAADLNGEGLSTTATAVITVLDTNDNAPRFNPTTYVGSVPENEANVAITTLTVTDADDPNTPAWEAVYTVLNDNEKQFIVVTDPVTNEGTLKTAKGLDFEAKQQYILYVAVTNVAPFEVTLPTSTATVTVDVIDVNEAPIFVPPQKRVEVPEDFGVGLEITSYTAREPDTFMEQKITYRIWRDTANWLEINPETGAISTRAELDREDVDHVKNSTYTALIIATDNGSPPATGTGTLLLFLDDVNDNGPVPEPRTMDFCQRNPEPHIININDPDLPPNTSPFTAELTHGASVNWTIEYNDQERESLILKPKKTLELGDHKINLKLIDNQNKDQVTTLDVHVCDCDGIVSNCRKARPAEAGLQVPAILGILGGILAFLILILLLLLLVRRRRVVKEPLLPPEDDTRDNVYYYDEEGGGEEDQDFDLSQLHRGLDARPEVTRNDVAPTLMSVPQYRPRPANPDEIGNFIDENLKAADSDPTAPPYDSLLVFDYEGSGSEAATLSSLNSSESDQDQDYDYLNEWGNRFKKLADMYGGGEDD

SEQ ID NO: 47 - DNA, 2649 bp, Bos taurus
  source 1..2649: mol_type = other DNA
atgggcccttggagccgcagcctctctgcgctctgctgctgctgcaggtgtaatccgtggctctgccgggagccggagccctgcattcctggctttggtgctgagagttacacgttcaccgtgccccggcggaacttggagagagggcgagtcctaggcagagtgagttttgaaggatgtgctggcctaccaaggacagtctatgtttctgatgacacccgattcaaagtgcacacagatggcgtgcttacagtcagacgacctgtacaccttcatcgtccagagctaagttttcttgtccatgcctgggactccacccacaggaagctctccaccaaagtgacactggaggtatcagcgcaccaccaccaccaccacagtcatcatgactctccctctggaacccagacagaagtgctcacatttcctggcccccaccatggtctcaggagacagaagagagactgggttattcctcctatcagctgcccagaaaatgagaaaggcccatttcctaagtcgctggtccagatcaaatctaacaaggagaaagaaacccaagttttctacagcatcactggccaacgagctgatacaccccctgtcggtgtttttattattgaaagagaaacaggatggttaaaagtgacacagcctctggatagagaacagattgccaagtacattctcttctctcatgccgtgtcttcaaatggacaagccattgaagagcctatggagattgtgatcaccgtgaccgaccagaatgacaacaagccccagttcacccaggaggtcttcaaggcgtctgccctggaaggcgctcttccaggaacctctgtgatgcaggtcacggccacagatatagatgacgaggtgaacacctacaccgctgccatcggttacacaatcccagcccaagatcccatgctgccgcacaacaaaatgttcaccatcaacaaggaaacaggcgtcatcagtgtgctcaccaccgggctggaccgtgagagttttcccacatacaccctgatggtccaagcagcagaccttaacggcgaaggcttgagcacaactgcaacggccgtgatcacagtcttggacaccaatgataatgctcccagattcaacccaaccacgtacgtggggtcggtgcctgagaacgaggctaatgtggccatcaccacactcacagtgactgatgccgacgaccccaacaccccggcatgggaggctgtttacacagtattaaatgataacgagaagcaatttatcgtcgtcacagacccagtcaccaatgaaggcactctgaaaacagctaagggcttggattttgaggccaagcagcagtacatcctgtacgtggcagtgacaaatgtggccccctttgaagtcactctccccacttccacagccaccgtcactgtggatgtgatagatgtgaatgaagcccccatctttgtgcctcctcaaaagagagtggaagtgcccgaggactttggcgtgggcctggagatcacatcctatactgcccgggagccagacacatttatggaacagaagatcacgtatcggatttggagggacactgccaactggctggagattaatccagaaacgggtgccatttccactcgggctgagttggacagagaggatgtcgatcatgtgaagaacagcacgtacacggccctcattatagccactgacaatggttctccacctgccactgggacaggcaccctgctcttgttcctcgatgatgtgaatgacaatggccccgtaccagaaccccggaccatggacttctgccagaggaatcctgagcctcatatcatcaacatcaatgatcctgatctccctccgaacacctccccctttacagcagaactgacacatggggcgagtgtcaattggaccattgagtacaatgaccaagaacgtgagtctctgattttgaagccaaagaaaaccttagagctgggtgaccacaaaatcaatctcaagctcatagacaaccagaacaaagaccaggtgaccacacttgatgtgcacgtgtgtgactgtgatgggatcgtcagcaactgcaggaaggcacggcctgctgaagcaggattgcaagttcccgccatcctggggatccttggaggcatccttgcttttctgatccttattttgctgcttctgctacttgttcggaggagaagggtggtcaaagagcccttactgcccccagaagatgacacccgggacaatgtgtattactatgatgaagaaggaggtggagaagaagatcaggactttgacttgagccagttacataggggcctggatgctcggcctgaagtgactcgcaatgacgtggcaccaaccctcatgagtgtgccccagtaccgaccccgccctgccaatcctgatgaaattggaaactttattgatgaaaacctgaaggcagctgatagtgaccccactgccccaccctatgactctctgctggtgtttgattatgaaggaagtggttccgaagctgctactctgagctccctgaactcctcagagtcagaccaagaccaggactatgactacctgaatgaatggggcaatcgcttcaagaagctggcggacatgtatggaggcggcgaggacgactag

SEQ ID NO: 48 - protein, 1125 aa, Bos taurus
MPRAPRCRAVRALLRASYRQVLPLAAFVRRLRPQGHRLVRRGDPAAFRALVAQCLVCVPWDAQPPPAAPSFRQVSCLKELVARVVQRLCERGARNVLAFGFTLLAGARGGPPVAFTTSVRSYLPNTVTDTLRGSGAWGLLLHRVGDDVLTHLLSRCALYLLVPPTCAYQVCGPPLYDLRAAAAAARRPTRQVGGTRAGFGLPRPASSNGGHGEAEGLLEARAQGARRRRSSARGRLPPAKRPRRGLEPGRDLEGQVARSPPRVVTPTRDAAEAKSRKGDVPGPCRLFPGGERGVGSASWRLSPSEGEPGAGACAETKRFLYCSGGGEQLRRSFLLCSLPPSLAGARTLVETIFLDSKPGPPGAPRRPRRLPARYWQMRPLFRKLLGNHARSPYGALLRAHCPLPASAPRAGPDHQKCPGVGGCPSERPAAAPEGEANSGRLVQLLRQHSSPWQVYGLLRACLRRLVPAGLWGSRHNERRFLRNVKKLLSLGKHGRLSQQELTWKMKVQDCAWLRASPGARCVPAAEHRQREAVLGRFLHWLMGAYVVELLRSFFYVTETTFQKNRLFFFRKRIWSQLQRLGVRQHLDRVRLRELSEAEVRQHQEARPALLTSRLRFVPKPGGLRPIVNVGCVEGAPAPPRDKKVQHLSSRVKTLFAVLNYERARRPGLLGASVLGMDDIHRAWRAFVLPLRARGPAPPLYFVKVDVVGAYDALPQDKLAEVIANVLQPQENTYCVRHCAMVRTARGRMRKSFKRHVSTFSDFQPYLRQLVEHLQAMGSLRDAVVIEQSCSLNEPGSSLFNLFLHLVRSHVIRIGGRSYIQCQGIPQGSILSTLLCSFCYGDMENKLFPGVQQDGVLLRLVDDFLLVTPHLTRARDFLRTLVRGVPEYGCQVNLRKTVVNFPVEPGALGGAAPLQLPAHCLFPWCGLLLDTRTLEVHGDHSSYARTSIRASLTFTQGFKPGRNMRRKLLAVLQLKCHGLFLDLQVNSLQTVFTNVYKIFLLQAYRFHACVLQLPFSQPVRSSPAFFLQVIADTASRGYALLKARNAGASLGARGAAGLFPSEAAQWLCLHAFLLKLARHRVTYSRLLGALRTARARLHRQLPGPTRAALEAAADPALTADFKTILD

SEQ ID NO: 49 - DNA, 3378 bp, Bos taurus
  source 1..3378: mol_type = other DNA
atgccgcgcgcgcccaggtgccgggccgtgcgcgcccttctgcgggccagctaccggcaggtgctgcccctggccgccttcgtacggcgcctgcggccccagggccaccggcttgtgcggcgcggggacccggcggccttccgcgcgctggtggctcagtgcttggtgtgcgtgccctgggacgcgcagccgccccctgccgccccgtccttccgccaggtgtcctgcctgaaggagctggtggccagagtcgtgcagaggctctgcgagcgcggcgcgaggaacgtgctggccttcggcttcacgctgctggccggggcccgcggcgggccgcccgtggccttcacgaccagcgtacgcagctacctgcccaacacggtaaccgacacgctgcgcggcagcggcgcctgggggctgctgctgcaccgcgtgggcgacgacgtgctcacccacctgctgtcgcgctgcgcgctctacctgctggtgcccccgacctgcgcctaccaggtgtgtgggccgccgctctatgacctccgcgccgccgccgccgccgctcgtcggcccacgcggcaagtgggcgggacccgggcgggcttcggactcccgcgcccggcctcgtcgaacggcggccacggggaggccgaaggactcctggaggcgcgggcccagggcgcgaggcggcgtcgcagtagcgcgcggggacgactgcctccagccaagaggcccaggcgcggcctggagcccgggcgggatctcgaagggcaggtggcccgcagcccgccccgcgtggtgacacctacccgagacgctgcggaagccaagtctcggaagggcgacgtgcccgggccctgccgcctcttcccgggcggcgagcggggtgtcggctccgcgtcctggcggctgtcaccctcggagggcgagccgggtgccggagcttgcgctgagaccaagaggttcctttactgctccggcggtggcgaacagctgcgccgctccttcctgctctgctccctgcctcccagcctggccggggcgcggacactcgtggaaaccatctttctggactcgaagcccgggccgccaggggctccccgccggccgcgccgcctgcccgcgcgctactggcagatgcggcccctgttccggaaactgcttgggaaccacgcgcggagcccctatggcgcgctgctcagggcgcactgcccgctgccggcctctgcgccccgggcggggccagaccatcagaagtgccctggtgttgggggctgcccctctgagaggccggccgctgcccccgagggcgaggcgaactcagggcgcctggtccagctgctccgccagcacagcagcccctggcaggtgtacgggctcctgcgggcctgtcttcgccgcctggtgcccgccggcctctggggctcccggcacaacgagcggcgcttcctgcggaacgtgaagaagctcctctccctggggaagcacggcaggctctcgcagcaggagctcacgtggaagatgaaggtgcaggactgcgcctggctgcgcgcgagcccaggggctcgctgcgtgcccgccgcggagcaccgccagcgcgaggccgtcctgggtcgcttcctgcactggctgatgggcgcctacgtggtggagctgctcaggagcttcttctacgtcacagagaccacgttccagaagaaccggctcttcttcttccggaagcgcatctggagccagctgcagcgcctgggcgtcagacaacacttagaccgtgtgcggcttcgagaactgtcagaagcagaggtcaggcagcaccaggaggccaggccggctctgctgacatccaggctccgtttcgtccccaagcccggcgggctgcggcccatcgtgaacgtgggctgtgttgagggcgccccggcaccgcccagagacaagaaggtgcagcatctcagctcacgggtcaagacgctgttcgcggtgctgaactacgagcgagctcggcggcctggcctcctgggggcctcggtgctgggcatggacgacatccacagggcctggcgggccttcgtgctgcccctgagggcccggggcccagcccccccgctctacttcgtcaaggtggacgtggtgggggcctacgatgccctcccccaggataagctggcagaggtgatcgctaacgtgctgcagccgcaggagaatacgtactgcgtgcgccactgcgccatggtccggactgcgcgcgggcgcatgcgcaagtccttcaagagacacgtgtccaccttctcggacttccagccgtacctgaggcagctcgtggagcatctgcaggcgatgggctccctgagggacgccgtggtcatcgagcagagctgctccctgaacgagcctggcagcagcctcttcaacctcttcctgcacctggtccgcagccacgtcatcaggatcgggggcaggtcctacatccagtgtcaggggatcccccagggctccatcctgtccaccctgctctgcagcttctgctatggggacatggagaacaagctcttccctggagtccagcaggacggggtgcttctgcgcctggtggacgacttcctgctggtcaccccacacctgacgcgggccagagacttcctcaggacgctggtgcgcggtgtgcctgagtatggctgccaggtgaacctgcggaagacggtggtgaacttccccgtggagcccggggccctgggcggcgcggcgcccctgcagctgccggcccactgcctgttcccctggtgcggcctgctgctggatacccgcaccctggaggtgcatggcgaccactccagttatgcccggacgtccatcagagcgagtctcaccttcacccagggcttcaagcccgggaggaacatgcgtcgcaagctgttggcggtcttgcagctcaagtgccatgggctcttcctggacctgcaggtgaacagtctgcagacggtcttcacaaacgtttacaagatattcctgctgcaggcctacaggttccacgcctgcgtgctgcagctgcccttcagccagccggtcaggagcagccccgcgttctttctccaggtcatcgccgacaccgcatcccgcggctacgccctcctgaaagccaggaacgcaggggcgtcactgggggccaggggcgccgccggcctgttcccgtctgaagctgcgcagtggctgtgtctccacgccttcctgctcaagctggctcgccaccgtgtcacctacagccgcctgctgggggccctccggacagcccgagcacggctgcaccggcagctcccggggcccacacgggccgccctggaggcggcggccgaccccgccctgaccgcagacttcaagaccatcttggactga

SEQ ID NO: 50 - protein, 386 aa, Bos taurus
MEESQAELNVEPPLSQETFSDLWNLLPENNLLSSELSAPVDDLLPYTDVATWLDECPNEAPQMPEPSAPAAPPPATPAPATSWPLSSFVPSQKTYPGNYGFRLGFLQSGTAKSVTCTYSPSLNKLFCQLAKTCPVQLWVDSPPPPGTRVRAMAIYKKLEHMTEVVRRCPHHERSSDYSDGLAPPQHLIRVEGNLRAEYLDDRNTFRHSVVVPYESPEIDSECTTIHYNFMCNSSCMGGMNRRPILTIITLEDSCGNLLGRNSFEVRVCACPGRDRRTEEENLRKKGQSCPEPPPRSTKRALPTNTSSSPQPKKKPLDGEYFTLQIRGFKRYEMFRELNDALELKDALDGREPGESRAHSSHLKSKKRPSPSCHKKPMLKREGPDSD

SEQ ID NO: 51 - DNA, 1161 bp, Bos taurus
  source 1..1161: mol_type = other DNA
atggaagaatcacaggcagaactcaatgtggagccccctctgagtcaggagacattttccgacttgtggaacctacttcctgaaaataaccttctgtcctccgagctctccgcacccgtggatgacctgctcccgtacacagatgttgccacctggctggatgaatgtccgaatgaagcgccccaaatgccagagccttctgccccagctgccccgccaccagccaccccagcaccagccacctcctggcccctgtcgtcctttgtcccctcccagaagacctaccctggcaattacggtttccgtctagggttcctgcaatccggaacagccaagtccgtgacctgcacgtattccccttcccttaacaagctgttctgccagctggccaagacctgcccagtgcagctgtgggtcgactcgccacccccgcccggcacccgcgtccgcgccatggccatctacaagaagttggagcacatgacggaggttgtgaggcgttgtccccaccatgagcgctcctctgactatagcgatggtctggcccctcctcagcaccttatccgggtggaagggaatttacgcgcggagtatttggacgaccggaacacctttagacacagtgtggtggtgccctatgagtcccccgagatcgactctgagtgcaccaccatccactacaatttcatgtgtaacagctcctgcatggggggcatgaaccggcggcccatcctcaccatcatcacactggaagactcttgtggtaacctgctgggacggaacagctttgaggtgcgtgtttgtgcctgtcctgggagagaccgccgcactgaggaagaaaatcttcgcaagaaggggcagtcttgccccgagccaccccctaggagcactaagcgagcactgcctaccaacaccagctcctctccacagccaaagaagaaaccactggatggagagtacttcactcttcagatccgtgggtttaaacgctatgagatgttccgagagctgaatgatgccttggagctgaaggatgctctggatggaagggaaccaggggaaagcagggctcactctagccacctgaagtctaagaagaggccttctccctcctgccataaaaaaccaatgctcaagagagagggacctgactcagactga

SEQ ID NO: 52 - protein, 802 aa, Bos taurus
MMNLQLIFWIGLISSVCCVFGQADENRCLKANAKSCGECIQAGPNCGWCTNSTFLQEGMPTSARCDDLEALKKKGCHPNDIENPRGSKDIKKNKNVTNRSKGTAEKLQPEDITQIQPQQLVLQLRSGEPQTFTLKFKRAEDYPIDLYYLMDLSYSMKDDLENVKSLGTDLMNEMRRITSDFRIGFGSFVEKTVMPYISTTPAKLRNPCTNEQNCTSPFSYKNVLSLTDKGEVFNELVGKQRISGNLDSPEGGFDAIMQVAVCGSLIGWRNVTRLLVFSTDAGFHFAGDGKLGGIVLPNDGQCHLENDVYTMSHYYDYPSIAHLVQKLSENNIQTIFAVTEEFQPVYKELKNLIPKSAVGTLSANSSNVIQLIIDAYNSLSSEVILENSKLPEGVTINYKSYCKNGVNGTGENGRKCSNISIGDEVQFEISITANKCPNKNSETIKIKPLGFTEEVEIILQFICECECQGEGIPGSPKCHDGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTTSCMAVNGQICNGRGVCECGACKCTDPKFQGPTCEMCQTCLGVCAEHKECVQCRAFNKGEKKDTCAQECSHFNITKVENRDKLPQPGQVDPLSHCKEKDVDDCWFYFTYSVNGNNEATVHVVETPECPTGPDIIPIVAGVVAGIVLIGLALLLIWKLLMIIHDRREFAKFEKEKMNAKWDTQENPIYKSPINNFKNPNYGRKAGL

SEQ ID NO: 53 - DNA, 2409 bp, Bos taurus
  source 1..2409: mol_type = other DNA
atgatgaatttacaactgattttctggatcggattgatcagttcagtttgctgtgtgtttggccaagcagatgaaaacagatgtttgaaagcaaatgccaaatcatgtggggagtgtatacaagcagggccaaattgtggatggtgcacaaattcaacatttttacaagaaggtatgcctacatctgctcgatgtgatgatttagaagccttaaaaaagaagggctgtcatccaaatgacattgaaaatcctagaggctccaaagatataaagaaaaataaaaatgtaacgaatcgaagcaaaggaacagcagagaagctgcagccagaagatatcactcaaatccagccacagcagttggttctgcagttacgatcaggggagccacagacattcacactaaaattcaagcgggctgaagactatcccattgatctctactacctcatggacctctcctattccatgaaagacgacttggagaatgtgaaaagtctgggaacagatttgatgaatgaaatgaggaggatcacttcagacttccgaattgggtttggttctttcgtggagaaaactgtgatgccttacattagcacaacaccagctaaactcaggaacccttgcacaaatgaacagaactgcacgagcccatttagctacaaaaatgtgctcagccttactgataaaggggaagtatttaatgaacttgttggtaagcagcgcatatctggaaatttggattcaccagaaggtggctttgatgcaatcatgcaagtcgcagtttgtggatccctgattggttggaggaatgtgacacggctgctggtgttttccacggatgctgggtttcacttcgctggagatgggaaacttggtggcatcgttttaccgaatgatggacagtgtcacctggaaaatgatgtgtacacaatgagccattattacgattatccttctattgctcaccttgtccagaaactaagcgaaaataacattcagacgatttttgcagttactgaagaatttcagcctgtttacaaggaactgaaaaatttgatccctaagtcagcggtaggaacattatctgcaaattccagcaatgtgattcagttgatcatcgatgcgtacaattccctttcttcagaagtcattttggaaaacagcaaattgccggaaggagtaacaataaattacaagtcttactgcaagaatggggtgaatggaacaggggaaaatgggagaaaatgctctaatatttccattggggatgaggttcagtttgaaattagcataactgcaaataaatgtccaaataagaactctgaaaccattaaaattaagcctctgggcttcactgaggaagtggagattatccttcagttcatctgtgaatgtgagtgccagggtgaaggcatcccaggaagccccaagtgccacgacggcaacgggaccttcgagtgtggggcctgcaggtgcaacgagggacgtgtcgggagacactgtgaatgtagcacagatgaggtgaacagcgaagacatggatgcctactgcaggaaagaaaacagttcagaaatctgtagcaacaatggagagtgtgtctgtggacagtgtgtgtgtaggaagagggacaatacaaatgaaatttattctggcaaattctgcgagtgtgataatttcaactgtgatagatccaatggcttaatatgtggaggaaatggtgtttgcaagtgtcgagtgtgtgagtgcaaccctaactacaccggcagcgcctgtgactgttctctggataccacttcctgcatggccgtgaatggacagatctgcaatggccgcggcgtctgcgagtgtggtgcctgtaagtgtacagaccccaagttccaagggccaacctgtgagatgtgtcagacctgccttggagtctgtgctgagcataaagaatgtgttcagtgccgagccttcaataaaggagagaagaaagacacatgtgctcaggaatgttcacatttcaacattaccaaggttgaaaatcgggacaaattgccccagccaggccaggtggatcccttgtcccactgtaaggagaaggacgttgacgactgctggttctacttcacatactccgtgaatgggaacaatgaggccactgttcatgttgtagagactccagagtgccccactggtccagacattattccaattgtagcaggtgtggttgctggaattgttcttattggccttgcattgctgctgatttggaagcttttaatgatcattcatgacagaagggaatttgccaaatttgagaaggaaaaaatgaatgccaaatgggacacgcaagaaaatccgatttacaagagtcctattaataatttcaagaacccaaactatggacgtaaagctggtctctaa

SEQ ID NO: 54 - protein, 258 aa, Bos taurus
MLSLCLMAALLLAAGPGPSLGDEAIHCPPCSEEKLARCRPPVGCEELVREPGCGCCATCALGKGMPCGVYTPRCGSGLRCYPPRGVEKPLHTLVHGQGVCMELAEIEAIQESLQPSDKDEGDHPNNSFSPCSAHDRKCLQKHLAKIRDRSTSGGKMKVIGAPREEARPVPQGSCQSELHRALERLAASQSRTHEDLYIIPIPNCDRNGNFHPKQCHPALDGQRGKCWCVDRKTGVKLPGGLEPKGELDCHQLADSFRE

SEQ ID NO: 55 - DNA, 777 bp, Bos taurus
  source 1..777: mol_type = other DNA
atgctgtccctctgcctcatggccgcgctgctgctggccgccgggcccgggccgagcctgggcgacgaagccatccactgcccgccctgctccgaggagaagctggcgcgctgccgcccccccgtgggctgtgaggagctggtgcgagagccgggctgcggctgttgcgccacttgcgccctgggcaaagggatgccctgtggggtgtacaccccccgctgcggctccggcctgcgctgctacccgccccggggcgtggagaagcccctgcacacgctggtgcacggacaaggcgtgtgcatggagctggcagagatcgaggccatccaggaaagcctgcagccctctgacaaggacgagggcgaccaccccaacaacagcttcagcccctgcagcgcccacgaccgcaagtgcctgcagaagcacttggccaaaattcgagaccggagcaccagtgggggcaagatgaaggtcatcggggcgccccgagaggaagcccggcctgtgccccagggctcctgccagagtgagctgcaccgggcgctggagcggctggccgcctcacagagccgcacccacgaagacctttacatcattcccatccccaactgcgaccgcaacggcaacttccaccccaagcagtgccacccggccctggatgggcagcgcggcaagtgctggtgtgtggaccggaagacgggagtgaagcttccggggggcctggagccgaagggggagctggactgccaccagttggctgacagcttccgagagtga

SEQ ID NO: 56 - protein, 216 aa, Bos taurus
MEVLLRTSRAGSRGPDALGHSGAAGWKGPESLNHRHTWRSATCQPEQLYSLSTGAMSELSRDAHQIPRSSKACRCLFGPVDSEQLRQDCDALMASCVQEARERWNFDFVTETPLEGDFAWERVRGLGLPKLYLPVGPRDDLGGGKRPSPSSALLQGTSQEDHLDLSLSCTLVTRSPERPEGTPGGPGPSQGRKRRQTSMTDFYHSKRRLICSKRKP

SEQ ID NO: 57 - DNA, 651 bp, Bos taurus
  source 1..651: mol_type = other DNA
atggaagttctgctgagaacttctagagctggaagcagaggtcctgatgcccttgggcatagtggagctgcaggatggaaagggcctgagtccctgaatcatcgtcacacctggaggtcagctacttgccaaccagaacaactgtattcattatccacaggtgccatgtctgagctgtccagggacgcgcatcaaatcccccgcagcagcaaggcctgccgctgcctctttggtccagtggacagcgagcagctgcgccaggactgcgacgccctcatggccagctgcgtgcaggaggcccgagagcggtggaacttcgactttgtcaccgagacgccgctggagggggacttcgcctgggagcgcgtgaggggcctcggcctgcccaagctctacctgcccgtggggccccgagacgacctgggagggggcaagcggccgagcccctcatccgccctgctgcagggcacgtctcaggaggaccacttggacctgtcgctgtcctgcaccctcgtgactcgctcccccgagcggcccgaggggacccccggtgggcctggcccctcccagggccggaaacggcggcagaccagcatgacagatttctaccactccaaacgcagactgatctgctccaagaggaagccctaa

SEQ ID NO: 58 - protein, 198 aa, Bos taurus
MSNVRVSNGSPSLERMDARQAEYPKPSACRNLFGPVNHEELTRDLEKHCRDMEEASQRKWNFDFKNHKPLEGKYEWQEVEKGSLPEFYHRPPRPPKGACKVPAQEGQDASGARPAVPLLGSQANPEDTHLVDQKTDAPDSQTGLAEQCPGIRKRPAADDSSPQNKRANRTEENVSDGSPNAGSVEQTPKKPGLRRRQT

SEQ ID NO: 59 - DNA, 597 bp, Bos taurus
  source 1..597: mol_type = other DNA
atgtcaaacgtgcgagtgtctaacgggagtccgagcctggagcggatggacgccagacaggcggagtaccccaagccctcggcttgcagaaacctctttggcccggtcaatcacgaagagttaacccgggacttggagaagcactgcagagacatggaagaggccagccagcgcaagtggaattttgattttaagaatcacaagcccctggagggcaagtacgagtggcaggaggtagaaaagggcagcctgcccgagttctaccacagacccccgcggccacccaaaggcgcctgcaaggtgccggcgcaggaaggccaggatgccagcggggcccgcccggcggtccctttacttgggtctcaggcaaacccagaggacacgcatttggtcgatcagaagactgatgccccggacagccagaccgggttagcggagcagtgccctgggataaggaagcgacctgccgcagatgattcctctcctcaaaacaaaagagccaacagaacagaagaaaatgtttcagacggttcccccaacgccggttcagtggagcagacgcccaagaagcctggcctcagaaggcgtcagacgtaa

SEQ ID NO: 60 - protein, 152 aa, Bos taurus
METSADLLAAAAALGWAEEVRALLEAGASANAPNRYGRSAIQVMMMGSARVAELLLLHGADPNCADPATLTRPVHDAAREGFLDTLVALHRAGAQLDVRDAWGRLPVDLAEERGHRDVARYLRAAAEDTEGGSHASADSAEGPADSSDLKKD

SEQ ID NO: 61 - DNA, 459 bp, Bos taurus
  source 1..459: mol_type = other DNA
atggagacctcggccgacttgttggccgccgccgccgcgctgggctgggctgaggaggtgcgggcgctgctcgaggcgggggcgtcggccaacgcgccgaaccgttacggtcgaagcgcgattcaggtgatgatgatgggcagcgcccgcgtggccgagctgctgctgctccacggcgcggaccccaactgcgcggaccccgccaccctcacccgaccggtgcacgacgccgcccgggagggcttcctggacacgctggtggccctgcaccgagctggagcgcagctggatgtgcgcgatgcttggggccgcctgcccgtggacctggcggaggagcggggccaccgcgacgtcgcccggtacctgcgcgcggctgcagaggacacggaaggcggtagccatgccagcgcagattccgcggaaggtccggcagacagctcggacctaaagaaagattga

SEQ ID NO: 62 - protein, 168 aa, Bos taurus
MAEPWGNELASAAARGDLEQLTSLLQNNVNVNAQNGFGRTALQVMKLGNPEIARRLLLRGANPDLKDRTGFAVIHDAARAGFLDTLQTLLEFQADVNIEDNEGNLPLHLAAKEGHLPVVEFLVKHTACKVGHRNHQGDTACDLARLYRRNEVVSLMEGNRAEGAANLQ

SEQ ID NO: 63 - DNA, 507 bp, Bos taurus
  source 1..507: mol_type = other DNA
atggccgagccttgggggaacgagttggcgtccgcagctgccaggggggacctagagcaacttactagtttgttgcaaaataatgtaaacgtcaatgcacaaaatggatttggaaggactgcgctgcaggttatgaaacttggaaatccggagattgccaggagactactacttagaggtgctaatcccgatttgaaagaccgaactggtttcgctgtcattcatgatgcggccagagcaggtttcctggacactttacagactttgctggagtttcaagctgatgttaacatcgaggataatgaagggaacctgcctttgcacttggctgccaaagaaggccacctccccgtggtggagttccttgtgaagcacacggcctgcaaagtggggcatcggaaccatcagggggacaccgcctgcgacttggccaggctctaccggagaaacgaggtcgttagcctgatggagggaaaccgggcggagggagctgcgaatctgcaatga

SEQ ID NO: 64 - protein, 559 aa, Bos taurus
MGSNKSKPKDASQRRRSLESAENTHGGGGGGGGGGGAFPSSQTPSKPASSDGHRGPNTAFAPAAAEPKLFGGFNSSDTVTSPQRAGPLSGGVTTFVALYDYESRTETDLSFKKGERLQIVNNTRKVDVSQTWFTFRWLQREGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGESLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKDPEERPTFEYLQAFLEDYFTSTEPQYQPGENL

SEQ ID NO: 65 - DNA, 1680 bp, Bos taurus
  source 1..1680: mol_type = other DNA
atgggcagcaacaagagcaagcccaaggatgccagccagcggcgccgcagcctggagtctgctgagaacacccacggaggcggcggcggcggcggcggcggtgggggtgccttcccctcctcacagacccccagcaagccagcctcctctgatggccaccgcggccccaacacggccttcgcccctgcagccgctgagcccaagctgttcggaggcttcaactcctcggacacagtcacctccccgcagagggcggggcccctgtctggcggagtgaccaccttcgtggccctctatgactatgagtcgcggacagagactgacctgtctttcaagaaaggggagcggcttcagattgtcaacaacacgaggaaggtggatgtcagccagacctggttcacattcagatggctgcaaagagagggagactggtggctggcccactcgctcagcacaggacagacgggctacatccccagcaactacgtggcaccctctgactccatccaggctgaagagtggtactttggcaagatcaccagacgggagtcagagcggttactgctcaatgcagagaacccaagagggaccttcctagtgcgagaaagcgagaccacgaaaggcgcctactgcctctcggtgtctgacttcgacaacgccaagggcctcaacgtgaagcactacaaaatccgcaagctggacagcggaggcttctacatcacctcccgcacccagttcaacagcctgcagcagctggtggcctactactccaaacacgctgacggcctctgccaccgcctcaccactgtgtgccccacgtccaagccgcagactcaaggcttggccaaagatgcctgggagatcccccgggagtcgctgcggctggaggtcaagctgggccagggctgcttcggagaggtgtggatgggtacctggaacggcaccaccagggtggccatcaaaaccctgaagcctggtacgatgtctccggaggccttcctgcaggaggcccaggttatgaagaaactgaggcatgaaaaactcgtgcagctgtacgccgtggtgtccgaagagcccatctacattgtcacggagtacatgagcaaggggagtttgctggactttctcaagggggagacaggcaagtacctgcggctgcctcagctggtggacatggctgcacagatcgcctcaggcatggcttatgtggaacggatgaactatgtccaccgagacctccgggctgccaacatcctggttggagagagcctcgtatgcaaagtggctgacttcgggctggctcgactcattgaagacaacgaatacacagcccggcaaggtgccaaattccccatcaagtggacagctccggaagctgccctctatggccggttcaccatcaaatcagacgtgtggtcctttgggatcctgctgacggagctcacaacaaagggacgggtgccctaccctgggatggtgaaccgggaggtgctggaccaggtggagcggggctaccggatgccctgccctcctgagtgtcccgagtcattgcatgacctgatgtgccagtgctggcggaaggacccggaggaacggcccaccttcgagtacctgcaggccttcctggaggactatttcacgtccaccgagccccagtaccagcccggagagaacctatag

SEQ ID NO: 66 - protein, 928 aa, Bos taurus
MPPKTPRRAAAAAAAAAAEPPPPPLPPPPEEDPEQDSGSEDLPLARLEFEETEEPDFTALCQKLKLPDHVRERAWLTWEKVSSVDGVLEGYVQKKKELWGICIFIAAVDLDEMPFTFTELQKNIETSVYKFFDLLKEIDTSTKVDNAMSRLLKKYNVLCALYSKLERTCELIYLTQPSSSISTEINSMLVLKVSWITFLLAKGQVLQMEDDLVISFQLMLCVLDYFIKLSPPALLKDPYKTAVIPINGSPRTPRRGQNRSARIAKQLENDTRIIEVLCKEHECNIDEVKNVYFKNFIPFMNSLGIVASNGLPEVENLSKQYEEIYLKNKDLDARLFLDHDKTLQADPTDSFEMQRTPRKSNPDEEVNMILPQTPVRTVMNTIQQLMMILNSASDQPSENLISYFNNCTVNPKDSILKRVKDIGDVFKEKFAKAVGQGCMEIGSQRYKLGVRLYYRVMESMLKSEEERLSIQNFSKLLNDNIFHMSLLACALEVVMATYSRSMSQSLDTGTDLSFPWILNVFNLKAFDFYKVIESFIKAEANLTREMIKHLERCEHRIMESLAWLSDSPLFDLIKQAKDREGPVDHFEPACTLNLPLQNNHTAADMYLSPVRSPKKKGPTPRVNSTPNSEAQATSAFQTQKPLKSTSLSLFYKKVYRLAYLRLNTLCARFLSDHPELEHIIWTLFQHTLQNEYELMRDRHLDQIMMCSMYGICKVKNIDLKFKIIVTAYKDLPHAVQETFKRVLIREEEYDSIIVFYNSVFMQRLKTNILQYASTRPPTLSPIPHIPRSPYKFSSSPLRIPGGNIYISPLNNPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNESVDTSNREEK

SEQ ID NO: 67 - DNA, 2787 bp, Bos taurus
  source 1..2787: mol_type = other DNA
atgccgcccaaaaccccccggagagcagcagcagccgccgccgccgccgccgcggaacccccgccgccgcccctgccgccgccccctgaggaggaccccgagcaggacagcggctccgaggacctgcctctggccaggcttgagtttgaagaaactgaagaacctgattttactgcattatgtcagaaattaaagctaccagatcatgtcagagaaagagcttggttaacttgggaaaaagtttcatctgtggatggagtattggaaggttatgttcaaaagaaaaaggaactgtggggaatctgtatttttatcgcagcagttgacttagatgagatgccgttcactttcactgagctacagaaaaacattgaaaccagtgtctataaattctttgatttactaaaagaaatcgataccagtaccaaagttgataatgctatgtcaagactgttgaagaagtataatgtgttgtgtgcactctacagcaaattagaaaggacatgtgagcttatatatttgacacaacccagcagttcgatatctactgaaataaattctatgttggtgctaaaagtttcttggatcacatttttattagctaaaggacaagtattacaaatggaagatgatctggtgatctcatttcaattaatgctgtgtgtccttgactattttatcaaactctcacctcctgcattgctcaaagacccatacaaaacagctgtaatacctattaatggttcacctcgaacaccaaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggaattgtagcttctaatggacttccagaggtggaaaatctctctaaacaatatgaagaaatttaccttaaaaacaaagatctagatgcaagattatttctggatcatgataaaactcttcaggctgatcctacagacagttttgaaatgcaaagaacaccacgaaaaagtaaccctgatgaagaggtgaatatgattcttccacagactccagttagaactgttatgaatactatccaacaattaatgatgatcttaaattcagcaagtgatcaaccatcggaaaatctgatttcctattttaataattgtacagtgaatccaaaggacagtatcctgaaaagagtgaaggatattggggacgtctttaaagagaaatttgctaaagctgtgggacaaggatgtatggaaattggatcacagcgatacaaacttggagtccgattgtattaccgagtaatggaatccatgcttaaatcagaagaagaacgattatccattcaaaattttagcaaactcctgaatgacaacatctttcatatgtctttgttggcatgcgctcttgaggttgtaatggctacctatagcagaagtatgtctcagagcctggatactggaacagatctatccttcccatggattctgaatgtatttaacttaaaagcttttgatttttacaaagtgattgaaagtttcatcaaagcagaagccaacctgaccagagaaatgattaaacatttagaacgatgtgaacatcgaatcatggagtcccttgcctggctctcagattcacctttatttgatcttattaaacaagcaaaggaccgagaaggaccagttgatcactttgaacctgcctgtactctcaaccttcctctccagaataatcacactgcagcagatatgtatctttctcctgtaagatccccaaagaaaaaagggccaactccacgtgtaaattctactccaaattcagaggcacaagcaacctcagccttccagactcagaagccattgaaatctacctccctttcactcttctacaaaaaagtgtatcgactagcatatcttcgactgaacaccctgtgtgcacgctttctgtctgaccacccagaactagagcatatcatctggacccttttccagcacacactgcaaaatgagtatgaactcatgagagacaggcatttggaccagattatgatgtgttctatgtatggcatatgcaaagtgaagaatatagaccttaaattcaaaatcattgtcacagcatacaaagatcttcctcatgctgttcaggagacattcaaacgtgttttgattagagaagaggagtatgattccattatcgtattctataattcggtcttcatgcagagactgaaaacaaatattttgcagtacgcttccaccaggccccctaccttgtcaccgattcctcacattcctcgaagtccttacaagttttctagttcacctttacggattcctggggggaacatctatatatcacccctaaataatccatataaaatttcagaaggtctgccgacaccaacaaaaatgactccaagatcaagaatcttagtatcaatcggtgaatcatttgggacttcagagaagttccagaaaataaatcaaatggtatgtaacagcgaccgcgtgctcaaaagaagtgctgaaggaagcaaccctcctaaaccactgaaaaaactgcgctttgacatcgaaggatccgatgaagcagatggaagtaaacatctcccaggggaatccaaatttcaacagaaactggcagaaatgacatctactcgaacacgaatgcaaaagcagaaaatgaatgagagcgtggatacctcaaacagggaagaaaagtga